(19) United States
(12) Patent Application Publication
(10) Pub. No.: US 2012/0082623 A1
(43) Pub. Date: Apr. 5, 2012

(54) SHH Regulation and Methods Thereof

(76) Inventor: Andreas H. Kottmann, Bronx, NY (US)

(21) Appl. No.: 13/250,236

(22) Filed: Sep. 30, 2011

Related U.S. Application Data
(63) Continuation-in-part of application No. PCT/US2010/029229, filed on Mar. 30, 2010.
(60) Provisional application No. 61/164,736, filed on Mar. 30, 2009.

(51) Int. Cl. A61K 49/00; A61K 31/4418; A61K 31/496; A61K 31/4184; A61K 31/4545; A61K 31/395; A61K 31/4436; A61K 31/5377; A61K 31/396; A61P 25/00; A61P 25/28; A61P 25/30; A61P 25/18; A61P 25/16; A61P 25/32; A61K 31/4355

(52) U.S. Cl. 424/9.2; 514/278; 514/357; 514/254.05; 514/394; 514/344; 514/318; 514/254.01; 514/183; 514/337; 514/234.2

(57) ABSTRACT

The invention provides for methods of upregulating endogenous GDNF by inhibiting Shh signaling. The invention further provides a method for increasing the production of cholinergic neurons and dopamine neurons by subventricular zone (SVZ) neurogenesis in a subject. The invention further provides methods for treating a neurodegenerative disorder in a subject.

This application is a continuation-in-part of International Application Number PCT/US2010/029229, filed on Mar. 30, 2010, which claims priority to Provisional Application 61/164,736, filed on Mar. 30, 2009.

### GOVERNMENT SUPPORT

The work described herein was supported in whole, or in part, by National Institute of Health Grant No. R21NS056312-01a1. Thus, the United States Government has certain rights to the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Neurodegenerative diseases such as Amyotrophic lateral sclerosis (ALS), and Parkinson's disease (PD) cause the progressive loss of neuronal function, with severely debilitating consequences. GDNF is a target-secreted neuroprotective, neurotrophic, and neuromodulatory factor. Neuroprotective agents are highly sought after, with hundreds of potential drugs under clinical trials. Currently, there are no marketed neuroprotective drug products that target the Shh pathway.

In translational stem cell research, particular interest has been devoted to neural precursor/stem cells resident in regions that display neurogenesis in adult mammals. This is due to the promise that neuronal stem cells resident in the adult brain can be coaxed into replenishing brain tissue with functional neurons and glia that are lost in neurodegenerative disease. Many neurodegenerative diseases lead to changes in the cytoarchitecture and qualitative outcome of neurogenesis in the subventricular zone (SVZ), pointing to pathological as well as adaptive and corrective functional alterations in the SVZ dependent on the specific disease.

Knowledge of the regulatory mechanisms that impinge on neurogenesis in the adult brain appear to provide the most straight forward guidance to those biochemical processes whose pharmacological manipulation can change the qualitative outcome of neurogenesis towards neurons that are needed for replacement in disease.

### SUMMARY OF THE INVENTION

In various aspects, the invention is directed to upregulation of endogenous glial cell-derived neurotrophic factor (GDNF) by the inhibition of Sonic Hedgehog (Shh) signaling. One aspect of the invention provides for a method for neuroprotection of neurons in a subject afflicted with or at risk of developing a neurodegenerative disorder. The method comprises administering to a subject an effective amount of a Shh antagonist that increases glial cell-derived neurotrophic factor (GDNF), thereby protecting the neurons. In one embodiment, the GDNF is endogenous GDNF. In another embodiment, the neurodegenerative disorder comprises Parkinson's Disease (PD), Amyotrophic Lateral Sclerosis (ALS), Alzheimer's Disease (AD), or Supra Nuclear Palsy, spinocereballar ataxias, multiple system atrophy, or corticobasal degeneration. In some embodiments, the antagonist is cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination of the listed Shh antagonists. In other embodiments, the method comprises measuring the concentration of acetylcholine in the extracellular fluid of the brain after treatment with a Shh antagonist (Nirogi et al., Biomed Chromatogr. 2010, 24(1):39-48) and/or measuring the concentration of GDNF in the extracellular fluid of the brain, serum, cerebrospinal fluid, or a combination thereof (Okragly et al., Exp Neurol. 1997 June; 145(2 Pt 1):592-6; Lundborg et al., J Neuroimmunol. 2010 Mar. 30; 220(1-2):108-13; Blasko et al., Dement Geriatr Cogn Disord. 2006; 21(1):9-15). In some embodiments, the method comprises determining whether treatment with a Shh antagonist increased GDNF levels as compared to the subject's GDNF levels prior to treatment with a Shh antagonist. In other embodiments, the method comprises determining whether treatment with a Shh antagonist decreased the subject's acetylcholine levels as compared to the subject's acetylcholine levels prior to treatment with a Shh antagonist. In some embodiments, measuring the concentration of acetylcholine in the extracellular fluid of the brain comprises liquid chromatography mass spectrometry of brain microdialysis samples.

An aspect of the invention further provides a method of decreasing axonal degeneration in a subject afflicted with or at risk of developing a neurodegenerative disorder, where the method comprises administering to a subject an effective amount of a Shh antagonist that increases glial cell-derived neurotrophic factor (GDNF), thereby decreasing axonal degeneration. In one embodiment, the GDNF is endogenous GDNF. In another embodiment, the neurodegenerative disorder comprises Parkinson's Disease (PD), Amyotrophic Lateral Sclerosis (ALS), Alzheimer's Disease (AD), or Supra Nuclear Palsy, spinocereballar ataxias, multiple system atrophy, or corticobasal degeneration. In some embodiments, the antagonist is cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination of the listed Shh antagonists. In other embodiments, the method comprises measuring the concentration of acetylcholine in the extracellular fluid of the brain after treatment with a Shh antagonist (Nirogi et al., Biomed Chromatogr. 2010, 24(1):39-48) and/or measuring the concentration of GDNF in the extracellular fluid of the brain, serum, cerebrospinal fluid, or a combination thereof (Okragly et al., Exp Neurol. 1997 June; 145(2 Pt 1):592-6; Lundborg et al., J Neuroimmunol. 2010 Mar. 30; 220(1-2):108-13; Blasko et al., Dement Geriatr Cogn Disord. 2006; 21(1):9-15). In some embodiments, the method comprises determining whether treatment with a Shh antagonist increased GDNF levels as compared to the subject's GDNF levels prior to treatment with a Shh antagonist. In other embodiments, the method comprises determining whether treatment with a Shh antagonist decreased the subject's acetylcholine levels as compared to the subject's acetylcholine levels prior to treatment with a Shh antagonist. In some embodiments, measuring the concentration of acetylcholine in the extracellular fluid of the brain comprises liquid chromatography mass spectrometry of brain microdialysis samples.

One aspect of the invention provides for a method for treating a subject afflicted with or at risk of developing a neurodegenerative disorder, where the method comprises administering to a subject an effective amount of a Shh antagonist that increases glial cell-derived neurotrophic factor (GDNF), thereby treating the subject. In one embodiment, the GDNF is endogenous GDNF. In another embodiment, the neurodegenerative disorder comprises Parkinson's Disease (PD), Amyotrophic Lateral Sclerosis (ALS), Alzheimer's Disease (AD), or Supra Nuclear Palsy, spinocereballar ataxias, multiple system atrophy, or corticobasal degeneration. In some embodiments, the antagonist is cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination of the listed Shh antagonists. In other embodiments, the method comprises measuring the concentration of acetylcholine in the extracellular fluid of the brain after treatment with a Shh antagonist (Nirogi et al., Biomed Chromatogr. 2010, 24(1):39-48) and/or measuring the concentration of GDNF in the extracellular fluid of the brain, serum, cerebrospinal fluid, or a combination thereof (Okragly et al., Exp Neurol. 1997 June; 145(2 Pt 1):592-6; Lundborg et al., J Neuroimmunol. 2010 Mar. 30; 220(1-2):108-13; Blasko et al., Dement Geriatr Cogn Disord. 2006; 21(1):9-15). In some embodiments, the method comprises determining whether treatment with a Shh antagonist increased GDNF levels as compared to the subject's GDNF levels prior to treatment with a Shh antagonist. In other embodiments, the method comprises determining whether treatment with a Shh antagonist decreased the subject's acetylcholine levels as compared to the subject's acetylcholine levels prior to treatment with a Shh antagonist. In some embodiments, measuring the concentration of acetylcholine in the extracellular fluid of the brain comprises liquid chromatography mass spectrometry of brain microdialysis samples.

An aspect of the invention provides for a method for treating a subject afflicted with or at risk of developing an addiction, the method comprising administering to a subject an effective amount of a Shh antagonist that increases glial cell-derived neurotrophic factor (GDNF), thereby treating the subject. In one embodiment, the GDNF is endogenous GDNF. In another embodiment, the addiction is an addiction to cocaine, alcohol, heroine, methadone, amphetamine, ketamine, or a combination thereof. In other embodiments, the method comprises measuring the concentration of acetylcholine in the extracellular fluid of the brain after treatment with a Shh antagonist (Nirogi et al., Biomed Chromatogr. 2010, 24(1):39-48) and/or measuring the concentration of GDNF in the extracellular fluid of the brain, serum, cerebrospinal fluid, or a combination thereof (Okragly et al., Exp Neurol. 1997 June; 145(2 Pt 1):592-6; Lundborg et al., J Neuroimmunol. 2010 Mar. 30; 220(1-2):108-13; Blasko et al., Dement Geriatr Cogn Disord. 2006; 21(1):9-15). In some embodiments, the method comprises determining whether treatment with a Shh antagonist increased GDNF levels as compared to the subject's GDNF levels prior to treatment with a Shh antagonist. In other embodiments, the method comprises determining whether treatment with a Shh antagonist decreased the subject's acetylcholine levels as compared to the subject's acetylcholine levels prior to treatment with a Shh antagonist. In some embodiments, measuring the concentration of acetylcholine in the extracellular fluid of the brain comprises liquid chromatography mass spectrometry of brain microdialysis samples.

One aspect of the invention provides a method of decreasing cholinergic tone in a subject afflicted with or at risk of developing a neurodegenerative disorder. In one embodiment, the method comprises administering to the subject an effective amount of a Shh antagonist; and measuring the concentration of acetylcholine in the extracellular fluid of the brain. In one embodiment, the GDNF is endogenous GDNF. In another embodiment, the neurodegenerative disorder comprises Parkinson's Disease (PD), Amyotrophic Lateral Sclerosis (ALS), Alzheimer's Disease (AD), or Supra Nuclear Palsy, spinocereballar ataxias, multiple system atrophy, or corticobasal degeneration. In some embodiments, the antagonist is cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination of the listed Shh antagonists. In other embodiments, the method comprises measuring the concentration of acetylcholine in the extracellular fluid of the brain after treatment with a Shh antagonist (Nirogi et al., Biomed Chromatogr. 2010, 24(1):39-48) and/or measuring the concentration of GDNF in the extracellular fluid of the brain, serum, cerebrospinal fluid, or a combination thereof (Okragly et al., Exp Neurol. 1997 June; 145(2 Pt 1):592-6; Lundborg et al., J Neuroimmunol. 2010 Mar. 30; 220(1-2):108-13; Blasko et al., Dement Geriatr Cogn Disord. 2006; 21(1):9-15). In some embodiments, the method comprises determining whether treatment with a Shh antagonist increased GDNF levels as compared to the subject's GDNF levels prior to treatment with a Shh antagonist. In other embodiments, the method comprises determining whether treatment with a Shh antagonist decreased the subject's acetylcholine levels as compared to the subject's acetylcholine levels prior to treatment with a Shh antagonist. In some embodiments, measuring the concentration of acetylcholine in the extracellular fluid of the brain comprises liquid chromatography mass spectrometry of brain microdialysis samples.

One aspect of the invention provides a method of decreasing cholinergic tone in a subject afflicted with a hypercholinergic disease. In one embodiment, the method comprises administering to the subject an effective amount of a Shh antagonist; and measuring the concentration of acetylcholine in the extracellular fluid of the brain. In one embodiment, the GDNF is endogenous GDNF. In another embodiment, the hypercholinergic disease comprises Parkinson's Disease (PD), Amyotrophic Lateral Sclerosis (ALS), Alzheimer's Disease (AD), or Supra Nuclear Palsy, spinocereballar ataxias, multiple system atrophy, or corticobasal degeneration. In some embodiments, the antagonist is cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination of the listed Shh antagonists. In other embodiments, the method comprises measuring the concentration of acetylcholine in the extracellular fluid of the brain after treatment with a Shh antagonist (Nirogi et al., Biomed Chromatogr. 2010, 24(1):39-48) and/or measuring the concentration of GDNF in the extracellular fluid of the brain, serum, cerebrospinal fluid, or a combination thereof (Okragly et al., Exp Neurol. 1997 June; 145(2 Pt 1):592-6; Lundborg et al., J Neuroimmunol. 2010 Mar. 30; 220(1-2):108-13; Blasko et al., Dement Geriatr Cogn Disord. 2006; 21(1):9-15). In some embodiments, the method comprises determining whether treatment with a Shh antagonist increased GDNF levels as compared to the subject's GDNF levels prior to treatment with a Shh antagonist. In other embodiments, the method comprises determining whether treatment with a Shh antagonist decreased the subject's acetylcholine levels as compared to the subject's acetylcholine levels prior to treatment with a Shh antagonist. In some embodiments, measuring the concentration of acetylcholine in the extracellular fluid of the brain comprises liquid chromatography mass spectrometry of brain microdialysis samples.

One aspect of the invention further provides a method for treating a subject afflicted with or at risk of developing a dopaminergic-related psychiatric condition, where the method comprising administering to a subject an effective amount of a Shh agonist that decreases glial cell-derived neurotrophic factor (GDNF), thereby treating the subject. In one embodiment, the GDNF is endogenous GDNF. In another embodiment, the agonist is purmorphamine or SAG. In a further embodiment, the condition comprises schizophrenia, bipolar affective disorder, of attention deficit hyperactivity disorder (ADHD). In other embodiments, the method comprises measuring the concentration of acetylcholine in the extracellular fluid of the brain after treatment with a Shh agonist (Nirogi et al., Biomed Chromatogr. 2010, 24(1):39-48) and/or measuring the concentration of GDNF in the extracellular fluid of the brain, serum, cerebrospinal fluid, or a combination thereof (Okragly et al., Exp Neurol. 1997 June; 145(2 Pt 1):592-6; Lundborg et al., J Neuroimmunol. 2010 Mar. 30; 220(1-2):108-13; Blasko et al., Dement Geriatr Cogn Disord. 2006; 21(1):9-15). In some embodiments, the method comprises determining whether treatment with a Shh agonist decreased GDNF levels as compared to the subject's GDNF levels prior to treatment with a Shh agonist. In other embodiments, the method comprises determining whether treatment with a Shh agonist increased the subject's acetylcholine levels as compared to the subject's acetylcholine levels prior to treatment with a Shh agonist. In some embodiments, measuring the concentration of acetylcholine in the extracellular fluid of the brain comprises liquid chromatography mass spectrometry of brain microdialysis samples.

One aspect of the invention provides for a method of increasing cholinergic tone in a subject afflicted with or at risk of developing a dopaminergic-related psychiatric condition. In one embodiment, the method comprises administering to the subject an effective amount of a Shh agonist and measuring the concentration of acetylcholine in the extracellular fluid of the brain. In one embodiment, the GDNF is endogenous GDNF. In another embodiment, the agonist is purmorphamine or SAG. In a further embodiment, the condition comprises schizophrenia, bipolar affective disorder, of attention deficit hyperactivity disorder (ADHD). In other embodiments, the method comprises measuring the concentration of acetylcholine in the extracellular fluid of the brain after treatment with a Shh agonist (Nirogi et al., Biomed Chromatogr. 2010, 24(1):39-48) and/or measuring the concentration of GDNF in the extracellular fluid of the brain, serum, cerebrospinal fluid, or a combination thereof (Okragly et al., Exp Neurol. 1997 June; 145(2 Pt 1):592-6; Lundborg et al., J Neuroimmunol. 2010 Mar. 30; 220(1-2):108-13; Blasko et al., Dement Geriatr Cogn Disord. 2006; 21(1):9-15). In some embodiments, the method comprises determining whether treatment with a Shh agonist decreased GDNF levels as compared to the subject's GDNF levels prior to treatment with a Shh agonist. In other embodiments, the method comprises determining whether treatment with a Shh agonist increased the subject's acetylcholine levels as compared to the subject's acetylcholine levels prior to treatment with a Shh agonist. In some embodiments, measuring the concentration of acetylcholine in the extracellular fluid of the brain comprises liquid chromatography mass spectrometry of brain microdialysis samples.

In various aspects, the invention is directed to therapeutic replacement of neurons lost in neurodegenerative diseases, such as dopamine neurons in Parkinson's Disease, and cholinergic neurons in Alzheimer's Disease and Supra Nuclear Palsy.

One aspect of the invention provides a method for increasing the production of cholinergic neurons by subventricular zone (SVZ) neurogenesis in a subject in need thereof, the method comprising administering to the subject an effective amount of a cholinotoxin to increase Shh expression in adult dopamine neurons, thereby increasing the production of cholinergic neurons. The dopamine neurons can be mesencephalic dopamine neurons. The cholinotoxin can be, for example, AF64A.

Another aspect of the invention provides for a method for treating a neurodegenerative disorder in a subject in need thereof, the method comprising administering to the subject an effective amount of a cholinotoxin to increase Shh expression in adult dopamine neurons, wherein increased Shh expression increased the production of cholinergic neurons, thereby treating the neurodegenerative disorder. The dopamine neurons can be mesencephalic dopamine neurons. The cholinotoxin can be, for example, AF64A. The neurodegenerative disorder can be Alzheimer's Disease or Supra Nuclear Palsy.

One aspect of the invention provides for a method for increasing the production of dopamine neurons by subventricular zone (SVZ) neurogenesis in a subject in need thereof, the method comprising administering to the subject an effective amount of a Shh antagonist that decreases Shh expression in adult dopamine neurons, wherein increased Shh expression increased the production of dopamine neurons in the olfactory bulb, thereby treating the neurodegenerative disorder. The dopamine neurons can be mesencephalic dopamine neurons. The neurodegenerative disorder can be Parkinson's Disease or Amyotrophic Lateral Sclerosis. The Shh antagonist can be cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination thereof.

A further aspect provides for a method for treating a neurodegenerative disorder in a subject in need thereof, the method comprising administering to the subject an effective amount of a compound that decreases Shh expression in adult dopamine neurons, thereby increasing the production of dopamine neurons. The dopamine neurons can be mesencephalic dopamine neurons. The compound can be a Shh antagonist, e.g., cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination thereof.

An aspect of the invention provides for a method for increasing the production of dopamine neurons in the olfactory bulb in a subject in need thereof, where the method comprises administering to the subject an effective amount of a Shh antagonist that decreases Shh expression in adult dopamine neurons, thereby increasing the production of dopamine neurons in the olfactory bulb. The dopamine neurons can be mesencephalic dopamine neurons. The Shh antagonist can be cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination thereof.

One aspect of the invention provides for a method for regenerating neurons in the SVZ of a subject afflicted with a neurodegenerative disorder, the method comprising administering to the subject an effective amount of a compound that modulates Shh expression in adult dopamine neurons, thereby regenerating neurons. In one aspect, Shh expression is increased in dopaminergic neurons. A cholinotoxin compound, such as AF64A, can be used to increase Shh expression. An increase in Shh expression, thus, induces the production of cholinergic neurons. In some aspects, the neurodegenerative disorder is Alzheimer's Disease or Supra Nuclear Palsy. In one aspect, Shh expression is decreased in dopaminergic neurons. A compound that decreases Shh expression can be used to induce the production of dopamine neurons. In some aspects, the neurodegenerative disorder associated with decreased dopamine neurons in the adult brain is Parkinson's Disease. The compound can be an Shh antagonist, e.g., cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination thereof.

One aspect of the invention provides for a method for screening compounds for the treatment of a neurological disease of the basal ganglia. The method comprises (a) administering a compound into a non-human animal with genetic ablation of Shh from mesencephalic DA neurons; (b) observe locomotion of the animal; and (c) determine if there is a locomotion deficit as compared to a non-human animal without genetic ablation of Shh from mesencephalic DA neurons. One further aspect of the invention provides for a method for testing the efficacy of a compound used for the treatment of a neurological disease of the basal ganglia, the method comprising: (a) administering a compound into a non-human animal with genetic ablation of Shh from mesencephalic DA neurons; (b) observe locomotion of the animal; and (c) determine if there is a locomotion deficit as compared to a non-human animal without genetic ablation of Shh from mesencephalic DA neurons. In one embodiment, the neurological disease of the basal ganglia is Parkinson's Disease, Huntington's Disease, a movement disorder, or a combination of any of the referenced neurological diseases. In another embodiment, the non-human animal is a mouse or a rat. In some embodiments, the locomotion deficit comprises reduction in gait length, an increases in gait variability, a reduction in break time, movement fluidity, bradykinesia, or a combination of the listed deficits. Movement disorders encompass a wide variety of neurological conditions affecting motor control and muscle tone. These conditions are typified by the inability to control certain bodily actions. Accordingly, these conditions pose a significant quality of life issue for patients. Nonlimiting examples of movement disorders include dyskinesias, dystonias, myoclonus, chorea, tics, and tremor. Thus, according to the invention, a progressive genetic model of PD (such as the non-human animal with genetic ablation of Shh from mesencephalic DA neurons) can be used for the purposes of drug screening or validation of already existing drugs marketed for other indications.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A is a schematic showing LoxP flanked Shh-nlacZ conditional ablation allele encoding a bicistronic mRNA for Shh and nuclear lacZ.

FIGS. 1B-E are photographic images showing Shh expression in dopaminergic cells of the SNpc revealed by immunohistochemical colocalization of TH [FIG. 1D, at high (FIG. 1C) and low (FIG. 1B) magnification] and βGal [FIG. 1E and in FIG. 1B and FIG. 1C] in a Shh-nlacZ mouse.

FIG. 1F is a schematic of a sagittal view of the mouse brain depicting the lateral wall of the ventricle (area in grey, CC, corpus callosum; RMS, rostral migratory stream, olfac-tory bulb; adapted from Garcia-Verdugo et al. (1998)).

FIG. 1G is a schematic of the summary of the relation ship of precursor cells in the SVZ (self renewing stem cells (B-cells) give rise to rapid amplifying cells (C-cells) which differentiate into migrating neuroblasts (A-cells) and key references for the characterization of dopamine (DA) and Shh action in subventricular zone (SVZ) neurogenesis.

FIGS. 1H-L are photographic images of immunohistochemical costaining for β-gal and TH on coronal sections of the striatum of Shh-nlacZ, Ptc 1-lacZ and Gli-nlacZ mice, respectively. There is no expression of Shh in the SVZ (FIG. 1H). Ptc-1 is expressed in the CPu, SVZ and LS (FIG. 1I). Gli1 is expressed in the SVZ and in scattered cells in the CPu and NA (FIG. 1K). FIG. 1L depicts a scheme for the identification of structures in FIGS. 1D-F. Abbreviations: LS: lateral septum; CPu: caudate putamen; SVZ, subventricular zone; aca, anterior comissure).

FIGS. 2A-B are photographic images of immunohistochemical staining for βGal and TH on coronal sections of the SNpc and VTA of mice heterozygous for the conditional ShhIRESnlacZ allele (see FIG. 1A) and either Dat-Cre− (FIG. 2A) or Dat-Cre+ (FIG. 2B). Expression of Shh as revealed by βgal immunoreactivity is strongly reduced in DA neurons in Dat-Cre+ mice as compared to Dat-Cre− mice. The figure shows a conditional deletion of Shh from mesencephalic DA− neurons.

FIG. 2C is a graph depicting quantification of TH and β-Gal double positive cells (left axis) in vMB as a whole, SNpc, VTA, Retrorubral Field (RRF) of Shh L/+, Dat-Cre+ (black bars) vs. Shh L/+, Dat-Cre− (white bars) mice. The number of βGal+ cells in the MeA is plotted in the same graph (right axis). The efficiency of Cre mediated ablation of Shh is about 80% and specific for DA neurons (p<0.05, t-test, averages±SEM are shown, 2 mice of each genotype, 5 sections spaced evenly encompassing the whole anterior-posterior extent of the mesencephalic DA nuclei, left and right hemispheres analyzed separately).

FIGS. 2D-E are photographic images of whole mount (“glass-brain”) preparations, ventral view, to assess qualitatively the tissue specificity of Cre recombination: The overall pattern of x-Gal stained nuclei remains unaltered with the exception of the absence of staining in the DA neurons of the vMB (right-hand side arrows). Left-hand side arrows point to the MeA.

FIGS. 2F-I are photographic images of chromogenic immunohistochemical stainings of TH in the striatum and SNpc of control animals (F, H) and animals with homozygous ablation of Shh from DA neurons (FIG. 2G and FIG. 2I). No changes in the pattern of fiber- or soma-staining as a function of Shh ablation from DA neurons were observed at 6 weeks of age.

FIG. 2K is a scheme depicting the Dat::Cre and that Shh produced in the mesencephalon is transported through axonal collaterals to the SVZ.

FIG. 3A is a graph depicting a Rum-Almond Test. Mice were single caged and habituated to a neutral odor probe over night. The next day animals were exposed to a total of six consecutives rum-odor-probes for 20 seconds each over a 30 minute period followed by a final exposure to an almond odor probe. All exposure trials were video recorded. Motor activity was assessed from tapes by an observer blinded to genotype and test order. Control animals (squares) increased locomotor activity upon exposure to the new odor whereas animals with conditional ablation of Shh (diamonds) did not. This demonstrates olfactory deficit in the absence of Shh expression by DA neurons.

FIG. 3B is a schematic depicting SHH that is secreted from the Notochord (N) and floor plate (FP) forming a gradient from ventral to dorsal along the midline. The Pax6 expressing precursor domain is curtailed ventrally by Shh signaling, which in turn allows the differentiation of several ventral cell identities.

FIGS. 3C-D are fluorescent images showing significance of the inhibition of Pax6 expression by Shh: ventral cell types, like motor neurons recognized by the expression of Isl1,2, only emerge in ventral areas of the neural tube from which Pax6 expression is absent. In animals with ablation of Shh produced by crossing the conditional Shh allele into HSP90::Cre animals, the Pax6 domain expands to the ventral midline blocking the differentiation of ventral cell types. This demonstrates the altered cyto-architecture of the olfactory bulb in the absence of Shh expression by DA neurons.

FIGS. 3E-K are photographic images showing in situ hybridization and immunohistochemistry for Pax6 and Dat in the adult olfactory bulb revealing an increase in the numbers of Pax6 expressing, DA-neurons of the periglomerular layer in the absence of Shh expression from DA neurons of the mesencephalon.

FIGS. 3L-N show that BrdU labeling in the SVZ has decreased proliferation in animals with conditional Shh ablation in DA neurons. This finding in combination with the observation of a greater number of Pax6 positive cells in the olfactory bulb (FIG. 3M) is consistent with alterations in cell fate determination in the SVZ in the animals with Shh ablation in DA neurons. FIGS. 3M-N: The grey bars depict controls, and the black bars depict the mutant mice.

FIGS. 4A-D shows results from the unilateral injection of the cholinotoxin ethylcholine mustard aziridium (AF64a) into the striatum and PPTg. FIG. 4A and FIG. 4C depict open Field video tracks and their quantification 30 h post injection of 1 ul of increasing concentrations of AF64a into the right striatum (FIG. 4A) or right PPTg (FIG. 4B) of control animals revealing a dose dependent turning bias that is ipsilateral to the injection side for striatal and contra lateral for the PPTg injections. Turning bias was calculated for each animal as relative “meander” between −180/cm to +180/cm. Significance determined as p<0.05 by post hoc test after ANOVA, n=4/dose or genotype. FIG. 4B and FIG. 4D are graphs showing the quantification of Shh expression in the vMB using the 3′ “TAQman” quantitative expression assay with results expressed as fold change over the contra lateral control side. FIG. 4B represents the striatal injections of AF64a result in a dose dependent upregulation of Shh in the vMB. FIG. 4D represents the AF64a injections into the PPTg leads to upregulation of Shh in the vMB comparable to the upregulation of Shh seen after striatal injections. Note that vehicle injections (“0” drug) into the PPTg cause noticeable motoric asymmetry (FIG. 4C) and significant upregulation of Shh in the vMB (FIG. 4D) in contrast to striatal AF64a injections. These results appear consistent with the observation that stereo taxic injection of the PPTg causes physical damage to a large proportion of this small cholinergic nucleus whereas very few cholinergic neurons of the striatum are affected by the needle as such. Tissue for mRNA preparation was collected 36 hours post injection of AF64a. Significance determined as p<0.05 by post hoc test after ANOVA, n=4/dose or genotype.

FIGS. 4E-F are graphs showing the quantification of fold gene expression changes in the vMB between ipsi- (experimental) and contra lateral (control) vMB after AF64a injections into the striatum (FIG. 4E) or PPTg (FIG. 4F). Bars above X-axis: up-regulation; Bars below X-axis: down-regulation. Expression quantification for each gene based on quantitative PCR using “Taqman” expression assays. *: significance as p<0.05. T-test, n=5/treatment group and genotype.

FIG. 4G is a schematic summarizing the results and anatomic context. DA neurons of the vMB are in dark grey, ACh neurons are in grey. + and − indicate stimulatory or inhibitory neuromodulatory input.

FIG. 5 is a schematic representation of the neurogenic niche of the adult SVZ in mouse. Stem cells (“B”) are in blue, rapid amplifying cells (“C”) in green, and migrating neuroblasts (“A”) in red. Note that B cells elaborate a primary cilium into the lumen of the ventricle, which potentially renders them sensitive to Shh present in the cerebrospinal fluid. All cells of the niche elaborate cellular contacts with the microvasculature. A and C cells innervated by dopaminergic DA neurons of the substantia nigra, potentially exposing those cells to Shh produced by mesencephalic DA neurons. LV: lateral ventricle, vMB: ventral midbrain, Shh: sonic hedgehog, VTA: ventral tegmental area, RRF: retrorubral field, SNpc: substantia nigra pars compacta., E: ependymal cells.

FIG. 6 depicts the expression of GDNF in the striatum and skeletal muscle in the adult mouse. In the Striatum: Immunohistochemical chromogenic (FIG. 6B-C) and fluorescent (FIG. 6E-G) staining for ChAT and β-Gal and chromogenic mRNA in situ hybridization analysis with a GDNF cDNA probe (FIG. 6D) on coronal sections of a 6 weeks old male mouse with a lacZ gene integrated behind the mRNA cap site in the GDNF locus by homologous recombination ((A), Moore et al., 1996; Bizon, J Comp Neurol. 1999 May 31; 408(2):283-98)). Fluorescent staining was documented by confocal microscopy. All cholinergic neurons of the adult striatum express GDNF. In Muscle: Chromogenic staining for X-gal activity in the limb of a 6 week old male mouse harboring the GDNF gene Expression tracer allele depicted in (FIG. 6A). FIG. 6H is a lateral view of Gastrocnemius (superficial muscle) and Soleus (deep muscle). LacZ staining is visible in both muscles in muscle spindles. FIG. 6I is an enlargement of a section of Gastrocnemius muscle. LacZ staining in muscle spindles is prominent. Calf was skinned and muscles exposed prior to incubation in staining solution. Whole mounts were fixed after staining, and dehydrated.

FIG. 7 depicts immunofluorescent studies. FIG. 7A is a schematic of LoxP flanked Shh-nlacZ conditional ablation allele encoding a bicistronic mRNA for Shh and nuclear lacZ. FIG. 7B-E are photographic images showing Shh expression in dopaminergic cells of the SNpc that revealed by immunohistochemical colocalization of TH [(FIG. 7D) at high (FIG. 7C) and low (FIG. 7B) magnification] and β-Gal [(FIG. 7E) and FIG. 7B-C] in a 8 week old Shh-IRES-nlacZ mouse. All Th positive (dopaminergic) neurons of the mesencephalon express Shh in the adult. (FIG. 7F) Only dopaminergic neurons of the mesencephalon but not those of the diencephalon or those resident in the olfactory bulb express Shh at 8 weeks of age.

FIG. 8 depicts the conditional deletion of Shh from mesencephalic DA− neurons. FIG. 8A is a schematic representation of the conditional Shh allele and the Dat-cre driver used for the genetic ablation of Shh from dopaminergic neurons. FIG. 8B-C show immunohistochemical staining for β-Gal and TH on coronal sections of the SNpc and VTA of mice heterozygous for the conditional ShhIRESnlacZ allele (FIG. 6A) and either Dat-Cre− (B) or Dat-Cre+ (C). Expression of Shh as revealed by β-gal immunoreactivity is strongly reduced in DA neurons in Dat-Cre+ mice as compared to Dat-Cre− mice. FIG. 8D is a graph showing the quantification of TH and β-Gal double positive cells (left axis) in vMB as a whole, SNpc, VTA, Retrorubral Field (RRF) of Shh L/+, Dat-Cre+ (black bars) vs. Shh L/+, Dat-Cre− (white bars) mice. The number of βGal+ cells in the MeA is plotted in the same graph (right axis). The efficiency of Cre mediated ablation of Shh is about 80% and specific for DA neurons (p<0.05, t-test, averages±SEM are shown, 2 mice of each genotype, 5 sections spaced evenly encompassing the whole anterior-posterior extent of the mesencephalic DA nuclei, left and right hemispheres analyzed separately). FIG. 8E-F shows whole mount (“glass-brain”) preparations, ventral view, to assess qualitatively the tissue specificity of Cre recombination: The overall pattern of x-Gal stained nuclei remains unaltered with the exception of the absence of staining in the DA neurons of the vMB (right-hand side arrows). Left-hand side arrows point to the MeA.

FIG. 9 is a schematic showing Summary of results and anatomic context. DA neurons of the vMB are in dark grey, ACh neurons are in grey. + and − indicate stimulatory or inhibitory neuro-modulatory input. Shh upregulation in DA neurons inhibits expression of GDNF by cholinergic neurons of the striatum in adult mice.

FIG. 10 is a graph showing the quantification of fold gene expression changes in the vMB between ipsi- (experimental) and contra lateral (control) striatum after AF64a injections into PPTg of either animals with genetic ablation of Shh from DA neurons or control animals. While ChAT and vAChT gene expression is downregulated in the striatum by AF64a injection into the PPTg regardless of Shh expression by DA neurons, GDNF expression is significantly more repressed in animals with Shh expression by DA neurons. Tissue for mRNA preparation was collected 36 hours post injection of AF64a. Significance determined as p<0.05 by post hoc test after ANOVA, n=4/dose. cDNA synthesis and qtPCR was performed according to the manufacturer's recommendation (Applied Biosystems). Expression quantification for each gene based on quantitative PCR using “Taq-man” expression assays. *: significance as p<0.05. T-test, n=5/treatment group.

FIG. 11 depicts the summary of temporal and spatial expression pattern of Shh in the spinal cord of chick at stages 25, 28, 36. FIGS. 11A-F, G, I, K are photographs of chromogenic mRNA in situ hybridization. FIG. 11H and FIG. 11L are photographic images of triple and double confocal immunofluorescence analysis. FIG. 11M represents a pixel density quantification of the red channel (Shh) of FIG. 11L. All panels of each stage are serial sections 16 nm apart of each other. FP: floorplate MNC: medial motor column, LMCm: medial subdivision of the lateral motor column; LMCl: lateral subdivision of the lateral motor column.

FIG. 12 represents a summary of Shh expression in the spinal motor neuron system in mouse using a gene expression tracer allele for Shh expression. FIG. 12A is a schematic of a construct used in the mouse strain 15-60 to determine the expression pattern of Shh in the spinal cord of adult mice by visualizing the expression tracer nLacZ. In this mouse line the expression of Shh is strictly linked to the expression of nLacZ due to a germline modification by homologous recombination in ES cells that leads to the transcription of a bicistronic mRNA coding for both, Shh and nLacZ (FIG. 6A). This recombinant allele is a very useful experimental tool to reveal unambiguously those cells in a multi-cellular setting that express Shh (Machold et al. 2003, Jeong et al. 2004, Lewis et al. 2004). FIGS. 12B-E are photographic images of whole mount x-gal staining for lacZ activity revealing Shh expression. FIG. 12B is a photographic image of an E14.5 mouse embryo, oblique lateral dorsal view. Strong contiguous Shh expression is apparent in the floorplate (FP, red arrows) and notochord (NC, “chain of beds”, blue arrows). Flanking the FP bilaterally, Shh expression in MN (black arrows) of the brachial and lumbar enlargements can be recognized. The limb level restriction of Shh expression in MN is lost by E16.5. FIGS. 12C-E are photographic images of whole mount x-gal stainings indicating Shh expression at brachial and thoracic levels at P2 (FIG. 12C) and P30 (FIG. 12D) and at lumbar levels at P80 (FIG. 12E), post transcardial perfusion with 4% PFA and ventral lamelectomy to expose ventral aspects of the spinal cord. Red arrows: midline, black arrows: DRG, BE: brachial enlargement, T: thoracic, LMC: Lateral motor column, MMC: Medial motor column. Whole mounts were fixed after staining, dehydrated and cleared in a 50/50 mixture of Benzyl alcohol/Benzoate. FIG. 12F is a photographic image depicting C5 analysis of the pectoralis MN pool at E17.5. About 30% of MNs expressing the MN pool specific marker Pea3 also express Shh. FIG. 12G is a photographic image depicting LS4 analysis. About 20% of all MNs labeled green in a mouse double heterozygous for a ChAT GFP gene expression tracer allele and for the Shh gene expression tracer allele (FIG. 12A) coexpress Shh. C5: cervical 5; LS4: lumbar-sacral 4.

FIG. 13A-B depicts the analysis of Olig2Cre. FIG. 13A is a schematic representation of the conditional Shh allele and the Olig2-cre driver used for the genetic ablation of Shh from spinal cord motor neurons. FIG. 13B is a graph showing that Cre recombination removes exon 2 and 3 as well as the LacZ expression tracer cassette from the Shh locus. Quantifying LacZ expression therefore provides a means to determine the efficiency of Cre recombination. A better than 80% recombination frequency in spinal MN of all levels is observed.

FIG. 13C is a photographic image of Shh L/L olig 2 cre mice. These animals have a genetic ablation of Shh expression from Motor neurons (MN). Homozygous mutant mice are much smaller.

FIG. 13D-F are graphs characterizing Shh L/L mice. FIG. 13D shows that mutant animals die by 3 weeks of age. FIG. 13E shows that mutant animals are born with normal weight but gain weight at a much reduced rate. FIG. 13F shows that at 20 days of age the muscle mass of gastrocnemius and soleus in mutant animals is half the mass of those muscles in controls.

FIG. 14 demonstrates that GDNF expression is increased in Gastrocnemius and Soleus muscle in the absence of Shh expression by motor neurons. FIG. 14A-B represent a longitudinal, comparative analysis of GDNF expression in Gastrocnemius and Soleus muscle in animals with genetic ablation of Shh expression from motor neurons and controls. In the Gastrocnemius GDNF expression is 8 fold increased in the absence of Shh. In the Soleus GDNF expression is 4 fold increased in the absence of Shh. Muscle Tissue for mRNA preparation was collected at E16, p2 and p17. cDNA synthesis and qtPCR was performed according to the manufacturer's recommendation (Applied Biosystems). Expression quantification for GDNF based on quantitative PCR using “Taq-man” expression assays. *: significance as p<0.05. T-test, n=5/time point.

FIG. 15 is a schematic representation of the progressive phenotype development of the transgenic G93A SOD model of familial ALS. FF: fast fatigable fibers; FR: fast resistant fibers, MN: motor neurons, x-axis: age of animals in days.

FIG. 16 depicts that Shh expression in the spinal cord is increased in 125 day old G93A SOD animals. FIG. 16A is a graph showing that mRNA expression for Shh is increased and for ChAT decreased in G93A SOD animals compared to controls. Spinal cord tissue for mRNA preparation was collected at p125 from animals double heterozygous for the G93A SOD—and the Shh IRES nLacZ tracer allele (FIG. 12A; experimental) and from animals heterozygous for the Shh IRES nLacZ tracer allele only (FIG. 12A; control). cDNA synthesis and qtPCR was performed according to the manufacturer's recommendation (Applied Biosystems). Expression quantification for Shh and Chat based on quantitative PCR using “Taq-man” expression assays. *: significance as p<0.05. T-test, n=5. FIG. 16B represents enzymatic X-Gal assays in protein extracts derived from the ventral spinal cord. There is a significant increase in β-gal activity in extracts derived from experimental animals, consistent with increased Shh expression. *: significance as p<0.05. T-test, n=5.

FIG. 17 is a graph showing that GDNF and CNTF expression in the soleus is absent in endstage G93A SOD animals. Longitudinal, comparative analysis of GDNF and CNTF expression in the Soleus muscle in animals transgenic for the G93A SOD allele and in control littermates. While upregulated moderately at intermediate stages of the disease, the expression of GDNF and CNTF is completely turned off in animals that have reached disease endstage. Muscle tissue for mRNA preparation was collected at p30, p70, p90, and p125. cDNA synthesis and qtPCR was performed according to the manufacturer's recommendation (Applied Biosystems). Expression quantification for GDNF and CNTF based on quantitative PCR using “Taq-man” expression assays. *: significance as p<0.05. T-test, n=5.

FIG. 18 is a graph showing the pharmacological inhibition of the Shh pathway in peripheral muscle of endstage G93A SOD animals results in a dose dependent up-regulation of GDNF and CNTF. The right soleus of 125 day old G93A SOD transgenic animals were injected with 0.5, 1, or 2 μg of Cyclopamine in 50 μl saline (experimental). The left soleus of each animal was injected with 50 μl of Saline (control). 30 h post injection the soleus muscles were dissected and mRNA preparations, cDNA syntheses and qtPCR were performed according to the manufacturer's recommendation (Applied Biosystems). Expression levels for GDNF and CNTF are expressed as fold change in gene expression over control side. The maximal up regulation of GDNF is achieved with 1 mg of Cyclopamine (27 fold) and the maximal upregulation of CNTF is achieved with 2 mg of cyclopamine (20 fold). Expression quantification for GDNF and CNTF based on quantitative PCR using “Taq-man” expression assays. *: significance as p<0.05. T-test, n=5 per dose.

FIG. 19 shows Shh mRNA expression in motor neuron ontogeny in the chick embryo. FIG. 19A is a photograph showing that at stage 10 to 14, Shh expression is restricted to the floorplate (FP) and notochord (N) depicted schematically in FIG. 19B. From stage 15 onwards, Shh is also expressed in MNs which at that time have migrated laterally forming the ventral horns of the developing spinal cord (FIG. 19C). For a review of this process, see Yamada et al., Cell. 1993 May 21; 73(4):673-86; Roelink et al., Cell. 1994 Feb. 25; 76(4):761-75; Ericson et al., Cold Spring Harb Symp Quant Biol. 1997; 62:451-66; Gunhaga et al., Development. 2000 August; 127(15):3283-93; and Briscoe et al., Mol. Cell. 2001 June; 7(6):1279-91.

FIG. 20 are photomicrographs of Shh mRNA expression in a subset of somatic motor neurons. Constructuon of the ChAT-GFP fusion protein was based on Tallini et al., Physiol Genomics. 2006 Nov. 27; 27(3):391-7.

FIG. 21A shows Shh expression in MN is repressed at the transcriptional level by signals from the developing limb: (1) Limb bud ablation was performed at stage 17 in ovo and spinal cord gene expression was analyzed at stage 27 prior to the peak of programmed cell death of MNs. (2) Detailed comparative analysis of gene expression of Shh, Pea3, ER81, Raldh2, and Isl1 by RNA in situ hybridization. Black arrows point to MN pools in which Shh expression is upregulated upon limb ablation. In contrast, Pea 3 and ER81 expression is almost completely lost upon limb ablation (Lin et al., Cell. 1998 Oct. 30; 95(3):393-407). Note that the unchanged and symmetric expression patterns of Raldh2 and Isl1 indicate that limb bud-removal does not lead to gross changes in MN numbers, MNC organization or spinal cord symmetry. (3) Shh expression in MNs upon unilateral sciatic nerve axotomy. Injection of retrograde tracers into contralateral calf muscles shows that MN pools contributing to the sciatic nerve exhibit up to 50% more MNs that express Shh upon sciatic nerve axotomy on the ipsilateral side. (4) There is no change in Shh expression at Brachial or Thoracic levels ipsilateral to sciatic nerve axotomy. The grey bars depict controls, and the black bars depict the experimental results.

FIGS. 21B-C represents a summary of schiatic nerve axatomy in the adult mouse. FIG. 21B are photomicrographs which compare qualitatively the relative frequency of expression of Shh among all MN in the ventral horns at level lumbar sacral 4 (LS4) on the axotomized and contra lateral control side. Both expression frequency and level of expression in each MN is visibly increased. The images are taken from a 6-month old mouse subjected to sciatic nerve axotomy. FIG. 21C depicts quantification of results shown in FIG. 21B: Frequency of expression among all MN doubles from ˜45 to ˜90% (n=2, 20 sections counted each, p<0.05, students t-test). Expression levels in each MN that expresses Shh almost doubles from in average 30 to ˜58 (arbitrary expression units by pixel density counting). Analysis was performed from confocal microscope pictures using Zeiss LSM 450 software according to the manufacturers recommendation. (N=2, 50 cells analyzed each, p<0.05, students t-test. Compare results with analysis of the G93A SOD1 transgenic animals shown in FIG. 25E. Results are qualitatively highly similar indicating that in the SOD1 model of familial ALS as well as in the axotomy paradigm Shh expression is modulated by signals from the periphery). In FIG. 21C: The white bars depict controls, and the black bars depict the experimental results.

FIG. 22 summarizes schematically the inventor's results on modulating Shh expression in spinal motor neurons. Shh expression is highly dynamic and highly sensitive to peripheral manipulations. Axotomy as well as muscle damage induced by cardiotoxin, physically parsing apart muscle with dull instruments, freeze/pinching of muscle fibers and even single injection needle stabs into peripheral muscles will up-regulate Shh expression in those MN that contribute to the innervation of the manipulated muscle. Shh in the MN system, as well as in the basal ganglia in the brain, as demonstrated in the Examples herein, is a sensitive sentinel for the intergraty of the axonal projections and projection areas of neurons which express Shh (See also Description of Figures for FIGS. 35-36).

FIG. 23 is a schematic depicting the sequential development of the specific neuromuscular phenotype observed in the transgenic G93A SOD model of familial ALS and a scheme of timepoints for determining Shh expression levels in the course of phenotype development in this model. See Schaeffer et al., Psychopharmacology (Berl). 2005 September; 181(2):392-400; Pun et al., 2006; and Saxena et al., Nat Neurosci. 2009 May; 12(5):627-36.

FIG. 24A is a panel of one representative section of each cranial MN pool of each hemisphere derived from a 12 month old male mouse stained for ChAT (rabbit anti ChAT, revealed by CY3 conjugated secondary antibodies and LacZ (chicken anti LacZ, revealed by FITC conjugated secondary antibodies). For determining the percentage of Shh expressing MNs over all MNs of a given MN nucleus, only ChAT immunopositive cellular profiles were counted in which the cellular nucleus was visible. Quantification is depicted in FIG. 24C.

FIG. 24B are micrographs that show Shh expression in MNs of lumbar sacral levels of a mouse double heterozygous for ShhIRESnLacZ and ChAT:EGFP alleles. Endogenous EGFP staining is unemplified, LacZ is revealed immunohistochemically in red (Cy3 conjugated sec. antibodies). Ventral-lateral quadrants of the spinal cord are shown. Level assignments are based on combination of recognizing the start of the lateral MN column at transition from thoracic to lumbar levels, identification and counting of ventral roots and dorsal root ganglia, end of medial MN column, and overall specific spinal cord structure at thoracic, lumbar and sacral levels. Sections are spaced about 800 mm for LS1 to 5, and about 180 mm for LS6a-d. Distribution and pattern of MNs as revealed by endogenous ChAT::EGFP expression is highly similar to the description of MN localization in L6 of rat allowing tentative assignment of pool identity in the mouse (pools identified in panel LS6b using nomenclature depicted in panel “rat L6”. “rat L6” is a section of ventral horn of level L6 of rat stained for ChAT immunoreactivity revealing a distinct location of individual MN pools contributing to the nudeus of Onuf taken from Schroder et al., 1980. Nomenclature is adapted from Schroder et al., 1980 and Ogier et al., 2006. EUS: external urethral sphincter; IC: Ischiocavernosus; BC: Bulbocavernosus; LA Levator Ani; EAS: external Anal Sphincter, DM: dorso-medial-, DL: dorso-lateral-, RDL: retro dorsal-lateral MN group. Quantification is depicted in FIG. 24C.

FIG. 24C depicts the quantification of the ratio of Shh expressing MNs over all MNs in cranial and locus of Onuf MN nuclei. Black bars: MN nuclei innervating extraocular muscles, light grey bars: non-extra ocular, cranial MN nuclei, green bars: Locus of Onuf MN pools DM and DL. Quantification based on the analysis of 2-6 cross sections per motor nucleus of two animals with separate analysis of left and right hemisphere. There are more Shh expressing MNs found among all MNs in extraocular MN nuclei (ocular, trochlear, abducens grouped together: 88%+/−13) than in the trigeminal, facial and hypoglossal nuclei combined (57% (+/−11): p<0.0003; 1-way ANOVA; F(5,23)=40.6.30% (+1-20) of all MNs in the dorsal lateral MN group of the locus of Onuf express Shh, a significantly smaller fraction when compared to the hypoglossal MN pool where 53% (+/−15) of all MNs express Shh (p<0.01, student's t-test).

FIGS. 25A-B are graphs depicting longitudinal analysis of Shh expression in the G93A model of familial ALS. FIG. 25A shows the fold change in mRNA expression for Shh and Choline Acetyl transferase (ChAT) in G93A SOD mice vs. control. The results show that while the MN marker ChAT declines due to MN death, Shh which is expressed by MNs increases. Since Shh expression occurs in MN the remaining MNs in endstage animals dramatically increase their expression of Shh over controls. This conclusion is further corroborated in FIG. 25C and FIG. 25D. FIG. 25B corroborates the observed increase of Shh by measuring LacZ activity in animals double heterozygous for the Sod1 transgene and the Shh expression tracer allele (see FIG. 1A) longitudinally at brachial and lumbar spinal cord levels. Again, in endstage animals, at both spinal levels, a significant increase in Shh expression is revealed.

FIG. 25C are confocal laser photomicrographs demonstrating a upregulation of Shh expression in single MN using LacZ expression as a tool to recognize Shh expression in animals double heterozygous for the Sod1 transgene and the Shh expression tracer allele (see FIG. 1A). While in control animals only about 50% of all MN recognized by ChAT staining (green) express Shh (recognized by nuclear lacZ staining in red in the nucleus), in G93A Sod1 experimental animals all surviving MNs express Shh at levels significantly elevated over shh expression in Shh positive MN in the control animals.

FIG. 25D are graphs that quantitate the relative numbers of Shh expressing MNs in controls and in the G93A model of familial ALS and the expression levels of Shh in individual Shh expression positive MNs in controls and in the G93A model of familial ALS. The relative expression rate of Shh among all MN is significantly increased in the disease model (from ˜60 to ˜90%, n=5, p<0.01, student's t-test) and expression levels of Shh in Shh expressing MN is more than doubled (from ˜25% to 57%, n=6, p<0.05, student's t-test). Quantification was performed from confocal laser microscope images using Zeiss LSM 450 software following the manufacturers recommendation. The white bars depict controls, and the black bars depict the mutant mice.

FIG. 26 are photomicrographs showing that the MN specific ablation of Shh from motor neuron causes a muscle fiber phenotype. Staining muscle cross sections of the lateral Gastrocnemius for the expression of a slow twitch muscle fiber marker, slow myosin heavy chain (sHMC) reveals that in mutant animals (Shh L/L) the numbers of slow twitch fibers is dramatically reduced at postatal day 15. The analysis of migrating myoblasts during early muscle development at embryonal day E12.5 does not reveal any qualitative or quantitative alterations in mutant animals. This analysis supports the idea that Shh expression by MN which begins around embryonal day 13.5 affects only secondary myogenesis.

FIG. 27 is a schematic depicting that Shh expression is increased. 100% of motor neurons remaining at p125 express Shh at high levels. To examine cells/tissues that are responsive to motor neuron Shh, conditional Gli1 reporter mice can be used in order to assess the function of Shh expressed by notor neurons. To analyze motor neuron specific Shh loss of function, Olig2-cre mice as well as Hb9-creERT2 mice can be used. Various translational aspects in the context of motor neuron disease can be assessed: (1) whether Shh regulates trophic factor expression; (2) whether Shh modulates motor neuron excitation; (3) whether Shh takes part in the inflammation of the SC; and (4) whether Smo agonists or antagonists modify phenotype progression in the SOD model.

FIG. 28A-H show photomicrographs of unaltered numbers of granule cells but expansion of the ER81+ population of granule cells in the bulb of animals without expression of Shh in DA neurons. FIG. 28A and FIG. 28E show the distorted laminar structure of granule cell layer revealed by Niss1 staining FIGS. 28B-D and FIGS. 28F-H are images that show double immunofluorescent labeling of ER81 and NeuN expressing granule cells. Domain of ER81 expressing granule cells extends from lamina 1 to 5 in mutant animals (Shh L/L; Dat::cre) compared to a restriction of ER81+ cells to lamina 1 and 2 in wt animals (Shh L/+; Dat::cre).

FIG. 28I is a schematic representation summarizing the expansion of the ER81 expression domain in mutant animals.

FIGS. 28J-K are graphs that quantify the proportion of ER81+ granule cells among all granule cells as a function of Shh expression by DA neurons (FIG. 28J; Student's t-Test, * p<0.05) and quantify granule cell numbers as a function of Shh expression by DA neurons. (FIG. 28K). The grey bars depict controls, and the black bars depict the mutant mice.

FIG. 29 are photomicrographs and graphs that shows altered proportions of Pax6+ and Olig2+ precursor cells within the SVZ. The relative proportions of Pax6 and Olig2 expressing cells within the SVZ and RMS were quantified by immunofluorescent double labeling on coronal sections (FIG. 29A, low power over view; and FIG. 29B is an enlargement of section of the SVZ indicated in FIG. 29A). Pax6 expressing cells within the SVZ are identified by pink arrows, Olig2 expressing cells by green arrows in FIG. 29B. FIG. 29C shows the quantification of relative proportions of Pax6 or Olig2 expressing cells over all DAPI nuclei in the SVZ and RMS. Results are expressed as the mean+/−SEM for genotype. Cells were counted along the entire a/p extend of the SVZ on 20 um cryostat sections (20 sections with a 5-section interval n=4 per genotype, left and right hemisphere analyzed separately. Student's t-Test, * p<0.01.

FIG. 30 is a schematic for neurogenesis, providing the basis to ask how sensors of physiological cell stress (e.g, functional or structural damage) interface and produce instructive signals for neurogenesis. There was previously no evidence that qualitative outcome of neurogenesis is altered by physiological need since the genetic induction of apoptosis, the only approach so far tried in the literature, failed to alter neurogenesis in the adult brain.

FIG. 31 is a schematic of cell lineage determination in the developing spinal cord of mice and chicken.

FIG. 32 is a diagram of Shh regulation of gene expression in the subventricular zone (SVZ), rostral migratory stream (RMS), and the Olfactory Bulb (OB). GL, glomerular layer; MCL, mitral cell body layer; GCL, granule cell layer. As known from studies of spinal cord development (FIG. 31), Shh signaling inhibits the expression of Pax6. Consistent with its action during development, the absence of Shh signaling in the SVZ via the ablation of Shh from DA neurons results in increased production of Pax6 lineage derivatives i.e. ER81+ granule cells and dopaminergic, Th+ periglomerular neurons as demonstrated in FIGS. 3E-N.

FIG. 33 are graphs that depict Shh ablation from DA neurons leads to Olfactory Deficit at 8 weeks of age. FIG. 33A is a graph showing that control and mutant animals do not differ in overall locomotion activity or in time spend in the center or periphery of an open field arena. FIG. 33B is a graph showing that control and mutant animals habituate with indistinguashable kinetics to new environments like an open field arena. FIG. 33C is a graph showing the Rum-Almond Test. Mice were single caged and habituated to a neutral odor probe over night. The next day animals were exposed to a total of six consecutives rum-odor-probes for 20 seconds each over a 30 minute period followed by a final exposure to an almond odor probe. All exposure trials were video recorded. Motor activity was assessed from tapes by an observer blinded to genotype and test order. Control animals (squares) increased locomotor activity upon exposure to the new odor whereas animals with conditional ablation of Shh from dopamine neurons (diamonds) did not.

FIG. 34 is a schematic showing that Shh expression levels in neurons that project to the SVZ are influenced by the physiological state of neurons that are connected to the Shh expressing projection neuron. Hence Shh expression itself can be viewed as a “sentinel” for network function and structural integrity. Shh has morphogen activity i.e. it posesses as demonstrated for its function in development, i.e. in the differentiation of the spinal cord (FIG. 19 and FIG. 31) In the adult brain however, Shh can not act through a gradient that forms by the secretion of Shh from a fixed source and extending over a field of Shh responsive precursor cells. Instead Shh is transported via axons of neurons that project to the germinal niche (i.e. DA neurons). Hence, “organizer activity” of Shh expressed by DA neurons is linked to neuronal connectivity and activity. Organizer activity at a distance includes: (1) Axon bridges anatomical discontinuity of organizer with patterning field; (2) Network of Shh expressing nuclei in the adult CNS; (3) Shh expressing neurons project collaterals to germinal niches; (4) Shh expression is sensitive to physiological stress in the immediate circuits in which these neurons reside; and (5) Changes in Shh expression has a morphogen function for the neurogenic niche in the SVZ.

FIG. 35 is a schematic depicting the idea that the sentinel function of Shh expression is not restricted to dopaminergic projections to the SVZ. Without being bound by theory, Shh expressing projection neurons act on SVZ neurogenesis through the expression and delivery of Shh into the germinal niche. However, Shh expression in these different classes of SVZ projecting neurons is modulated by the physiological state of the neurons that make up the microcircuit in which the Shh expressing neuron resides in. Dysfunction in any of these connected neurons will alter the effective, overall concentration of Shh in the SVZ towards a concentration by which the production of that neuronal identity which is under physiological cell stress, is produced. Both up and down modulation of effective Shh concentrations in the SVZ will occur.

FIG. 36 is a schematic depicting that neuronally expressed, damage-induced, Shh regulates germinal niches both in the basal ganglia and in the spinal-muscular system at a distance in the adult organism.

FIG. 37 are graphs that show the quantification of the numbers of Th expressing dopaminergic neurons in the substantia nigra pars compacta in the MPTP paradigm with and without inhibition of Shh signaling by cyclopamine. FIGS. 37A-D are experimental flow charts. FIG. 37E is a graph that shows absolute numbers of surviving Th+ cells at day 33. Cell numbers were calculated by stereological quantification using a Steroinvestigator 4.34 (MicroBrightField, Colchester, Vt.) software running an automatic x-y stage on a Zeiss Axioplan2 microscope equipped with a planapochromat 100× oil objective, cells were counted on 40 μm floating sections encompassing the entire a/p extent of the SNpc (12 sections with a 4-section interval, left and right hemisphere analyzed separately. Student's t-Test, * p<0.05).

FIG. 38 are graphs demonstrating the number of TH+ and ChAT+ cells. FIG. 38A is a graph showing decreased cell numbers of Th expressing cells in the SNpc of conditional knockouts in phenotype phase II, III and IV but not at 1 month (phase I) of age. FIG. 38B is a graph showing a decreased number of choline-acetyl-transferase (ChAT) expressing cells, i.e. cholinergic neurons, in the striatum of conditional knockouts in phase II, III and IV but not in phase I (see FIG. 39A for definition of phenotype phases). Cell numbers were calculated by stereological quantification using a Steroinvestigator 4.34 (MicroBrightField, Colchester, Vt.) software running an automatic x-y stage on a Zeiss Axioplan2 microscope equipped with a planapochromat 100× oil objective, cells were counted on 40 μm floating sections encompassing the entire a/p extent of the SNpc (12 sections with a 4-section interval) and striatum (12 sections with a 4-section interval), 4 animals per genotype, left and right hemisphere analyzed separately. Student's t-Test, * p<0.01. The grey bars depict controls, and the black bars depict the experimental results.

FIGS. 39A-B are graphs that demonstrate behavioral changes in mice with Shh ablation in DA neurons revealed by open field analysis. FIG. 39A is a graph that shows progressive horizontal locomotion deficits in the absence of Shh from DA neurons. Animals with the Shh ablation show indistinguishable locomotion behavior to controls at 1 month of age (phase I), hypolocomotion between 2 and 5 months (phase II) and hyperlocomotion between 7 and 12 months (phase III). Phase IV is characterized by no alterations in locomotion activity but is unstable and followed rapidly by progressive neurological decline leading to pelvic dragging, partial hindlimb paralysis and premature death by about 18 months of age. FIG. 39B is a graph that shows progressive vertical locomotion deficits in the absence of Shh expression from DA neurons. Phenotype follows in fair agreement horizontal locomotion disturbances. Locomotion was quantified by an automated video tracking system (EthoVision-Noldus Information Technology) during a 10 min open field trial. Results based on sequential testing of 2 cohorts (control/knockouts, n=7-9 per genotype) each of them showed identical results for (FIGS. 39A-B). The grey bars depict controls, and the black bars depict the mutant mice.

FIGS. 39C-D are graphs that show gait dynamics and stride length. FIG. 39C is a bar graph showing the analysis of gait dynamics in the absence of DA neuron produced Shh at different ages. Stride length variability increases in front and hind limbs in phase IIIb (11-13 month old animals) compared to age matched litter controls but is unaltered in younger animals. Results are expressed as changes in Coefficient of Variability (CV; SD/average×100). Results are presented as mean±SEM of determinations from 10 measures (left, right limb), 5 mice/genotype/age. *=P<0.05 determined by Student's t test. FIG. 39D is a bar graph showing the effects of Levodopa (L-Dopa) and Trihexyphenidyl (THP) on increased variability of stride length in the absence of DA neuron produced Shh. The increased variability in stride length observed in experimental animals (CV, FIG. 39D) was normalized to control levels by L-Dopa (20 mg/kg SC) [Drug×Genotype, F(1,37)=3.5, p<0.05]. THP (3 mg/kg, IP) also normalized the increased CV observed in experimental animals to control levels [Drug×genotype (1,37)=4.2, p<0.04]. (*) indicates significant interaction relative to vehicle treated animals at p<0.05 determined by 2-Way ANOVA followed by Tokey HSD post-hoc test; n=10 measures (right and left hind limbs) from 5 animals of 12 months of age/genotype. The effects of these drugs were similar in forelimbs. The grey bars depict controls, and the black bars depict the mutant mice.

FIGS. 39E-I are graphs that demonstrate the analysis of the fluidity and complextity of spontaneous locomotion activity. FIGS. 39E-F show that there were no differences between contrrols and mutants for the accelaration and deceleration segments in phase II. FIGS. 39G-H show that mutant animals spend significantly more time relative at low speed levels and less time relative at high speed levels compared to controls in the acceleration segment and more time at high and low speed levels in the deceleration segment compared to controls in phase III. (n=12, p<0.05, students t-test). FIG. 39I shows the quantitation of Surges. In phase II, while mutant animals are hypoactive compared to controls (FIG. 39A), mutant animals switch more often between acceleration and deceleration than controls. In contrast in phase III, when mutant animals are hyperactive (FIG. 39A), mutant animals show a reduction in movement fluidity. The grey bars depict controls, and the black bars depict the mutant mice.

FIGS. 40A-C are graphs that demonstrate Brake to Stride ratios. Brake to Stride ratios are affected in both limbs at 12 months but not 7 months of age in mutant animals (FIG. 40A). Results are presented as mean±SEM of determinations from 10 measures (left, right limb), 5 mice/genotype/age. *=P<0.05 determined by Student's t test. Interestingly, L-Dopa did not correct the reduction in brake time observed in experimental animals but instead reduced Brake-Stride ratios in both experimental and control animals [Genotype×Drug, F(1,37)=0.01; not significant; FIG. 40B]. In contrast, THP normalized Brake-Stride ratios to control levels [genotype×Drug, F(1,37)=3.3; p<0.05; FIG. 40C]. (*) indicates significant interaction relative to vehicle treated animals at p<0.05 determined by 2-Way ANOVA followed by Tokey HSD post-hoc test; n=10 measures (right and left hind limbs) from 5 animals of 12 months of age/genotype. The effects of these drugs were similar in forelimbs. The grey bars depict controls, and the black bars depict the mutant mice.

FIGS. 40D-F are graphs showing spontaneous locomotion analysis. FIG. 40D shows the duration of locomotion bouts is slightly larger in mutant animals in phase II but unaltered in phase III. FIG. 40E shows maximal locomotion speed indistingushable between control and mutant animals in phase II and phase III. FIG. 40F is a schematic description of the “speed bin” analysis and quantitation of “surges” (see Example 10). The grey bars depict controls, and the black bars depict the mutant mice in FIGS. 40D-E.

FIGS. 40G-H are graphs that demonstrate the effects of Levodopa (L-Dopa) and Trihexyphenidyl (THP) on time spent at different speed levels in each locomotion bout. Both drugs ameliorate the deficits at low speeds but do not normalize the differences at high locomotion speeds. (n=12, p<0.05, students t-test).

FIGS. 41A-F are photomicropgraphs of confocal microscopy analysis showing that cholinergic neurons in the striatum express GDNF.

FIGS. 41G-I are graphs that depict biochemical confirmation for GDNF expression by cholinergic neurons. FIG. 41H shows that injection of AF64a, which kills cholinergic neurons, reduces GDNF tissue content in the striatum as measured by quantitative ELISA. FIG. 41G shows that in the animal model (loss of Shh from DA neurons which causes long term degeneration of cholinergic neurons), a reduction in GDNF tissue content correlates with relative loss of cholinergic neurons over 16 months as measured by quantitative ELISA. FIG. 41I shows quantitative PCR for GDNF and GDNF receptors in the striatum of mice with genetic ablation of Shh from DA neurons at 1 month (1st column) and 12 months (2nd column of each pair). GDNF expression is lost 6 fold and 70 fold respectively, but receptors for GDNF are robustly up-regulated. Cells that make GDNF die, hence the progressive reduction in GDNF. With reduced ligand expression, the system upregulates receptor expression in order to compensate for ligand loss. In FIGS. 41G-H, the grey bars depict controls, and the black bars depict the experimental results.

FIG. 42 represents the mesostriatal circuitry. Gabaergic (grey) medium spiny projection neurons (msP) of the striatum receive converging glutamatergic input from the cortex and thalamus (blue arrows). Glutamatergic drive of msPs is powerfully gated by striatal resident cholinergic—(green) and distinct populations of gabaergic—(Parv+, Som+, and Cal+, resp, all grey) interneurons. All striatal interneurons and msPs receive dopaminergic input from the mesencephalon. Cholinergic interneurons project to all striatal neuronal subtypes and regulate dopamine release via presynaptic signaling. (Glut: glutamine; GABA: γ-aminobutyric acid; ACh: acetylcholine; DA: dopamine; ChAT: Choline-Acetyl Transferase; Th: Tyrosine Hydroxylase; FS: “fast spiking”; Parv: Parvalbumin; Som: Somatostatin; Cal: Calretinin; vMB: ventral midbrain.)

FIGS. 43A-M show Shh expression by mesencephalic DA neurons is inhibited by signals emanating from TANS in the adult brain. FIG. 43A shows a coronal section of vMB of a Shh-nLZ^{c/+} mouse stained with x-Gal. FIG. 43B shows that 100+/−0% of Th⁺ dopaminergic neurons in the mesencephalon express Shh at p90 (683 cells, 2 mice). FIG. 43C shows a coronal section of striatum of a Ptc1-nLZ mouse stained with x-Gal. FIG. 43D shows that a subset of neurons and non neuronal cells express Ptc1 in the striatum. FIG. 43E shows that chat⁺ cholinergic interneurons (TANS) of the striatum express Ptc1. FIG. 43F shows that Parv1⁺ gabaergic interneurons (FS) of the striatum express Ptc1. FIG. 43G shows that 25+/−1.8% of Ptc1⁺ striatal cells are neurons (for all quantitation herein, data are represented as mean+/−s.e.m.; n=612 cells, 3 mice). FIG. 43H shows that 6+/−0.8% of striatal neurons express Ptc1 (n=620 cells, 3 mice); 100+/−0% of TANS express Ptc1 (n=140 cells, 3 mice); 98+/−0.2% of FS express Ptc1 (n=126 cells, 3 mice). FIG. 43I shows that unilateral striatal injection of the cholinotoxin AF64a results in increased motor activity contra-lateral and in ipsi-lateral turning. FIGS. 43K and L show that increased concentrations of AF64a (0-1 mM) injected unilaterally into the striatum cause a proportional increase in turning bias observed by open field video tracking (*p<0.05, **p<0.01, AF64a dose vs. vehicle (0 mM AF64a), ANOVA followed by Tuckey's post hoc test (n=5/dose)). The effect of AF64a was dose-related for 0-1 mM dose range (R²=0.77). FIG. 43M shows that AF64a injections into the striatum elicit an ipsilateral up-regulation of Shh transcription in the vMB quantified by qrtPCR (for all quantitative gene expression analysis herein data is expressed as relative fold change of experimental over control conditions with down-regulation shown as red bars pointing down and up-regulation shown as green bars pointing up; *p<0.05, AF64a dose vs. vehicle (0 mM AF64a), ANOVA followed by Tuckey's post hoc test (n=5/dose)). The effect of AF64a was dose-related for 0-1 mM dose range (R²=0.83).

FIGS. 44A-H show neuronal subtype specific and progressive degeneration of the striatum in the absence of Shh signaling from mesencephalic DA neurons. FIG. 44A shows that ablation of Shh from DA neurons causes a down regulation of the transcription of Shh signaling components in the striatum and a transcriptional activation of Shh loci in the vMB quantified by qrtPCR using an amplicon for exon 1 of the Shh locus which is not deleted be Cre recombination (FIG. 50A). FIG. 44B shows the analysis of nuclear heterochromatin pattern (FIG. 56A-B) and nuclear size (FIG. 56C) reveals a reduction in the numbers of FS, TANS and cells with nuclear circumference greater than 28 um at 6 months of age. (*p<0.05, **p<0.001, t-test (10 striatal slices/subject, n=7 mice per genotype). FIG. 44C shows adult onset, progressive reduction in numbers of ChAT⁺ neurons (unbiased stereological counting by optical fractionation; *p<0.05, unpaired t-test (n=6-7/group, 12 sections per subject with a 5 section interval). FIG. 44D shows adult onset, progressive reduction in numbers of Parv⁺ neurons (unbiased stereological counting by optical fractionation; *p<0.05, unpaired t-test (n=4/group, 12 sections per subject with a 5 section interval). FIGS. 44E-F show loss of TANS is most pronounced in lateral, equatorial areas of the dorsal striatum (quantified in FIGS. 57A-B)). FIG. 44G shows that extracellular ACh concentration in the striatum is reduced ** p<0.001, unpaired t-test (n=8/genotype, 4 samples/subject). FIG. 44H shows that transcription of cholinergic-, and trophic signaling-markers, and parvalbumin is altered prior to GABAergic and dopaminergic markers (* p<0.01, ** p<0.001, two tailed t-test; n=5/genotype).

FIGS. 45A-L show that Shh signaling inhibits GDNF expression by TANS. FIGS. 45A-F show that 100+/−0% of TANS throughout the striatum express GDNF (GDNF-LZ mice, 270 cells; n=2). FIG. 45G shows that striatal GDNF tissue content decreased 37+/−3% after unilateral, striatal administration of AF64a. (* p<0.05, unpaired t-test, n=12-14/group). FIG. 45H shows the progressive decline of GDNF in the striatum in the absence of Shh expression by DA neurons of the vMB (* p<0.01, unpaired t-test; n=10-11/group). n=5-9, mean+/−SEM calculated from quadrupled measurements. The reduction of striatal GDNF tissue content (%) is correlated with the reduction of TANs (FIG. 3: C; R2=0.89, T (5.9), p<0.02). FIG. 45I shows that unilateral injection of AF64a into the PPTg causes a contra-lateral turning bias in Shh-nLZ^{C/C}; Dat-Cre mice and Shh-nLZ^{C/+}; Dat-Cre control littermates (FIG. 60) consistent with reduced DA tone ipsi-lateral observed upon toxicological insult to PPTg neurons (Dunbar et al., 1996). FIG. 45K shows that injection of AF64a into the PPTg elicits ipsi-lateral up-regulation of the transcription of full length Shh mRNA in the vMB in control animals but not in Shh-nLZ^{C/C}; Dat-Cre mutant mice (*** p<0.0001, AF64a×genotype ANOVA followed by Tukey's post hoc Test (n=5/group/genotype). Dopaminergic markers are altered irrespective of genotype (*p<0.05; n=5/group/genotype). FIG. 45L shows that in control animals, but not in Shh-nLZ^{C/C}; Dat-Cre mutant mice, AF64a elicited ipsi-lateral down-regulation of GDNF transcription in the striatum (****P<0.00001, AF64a×genotype ANOVA followed by Tukey's post hoc Test (n=5/group/genotype). The cholinergic markers ChAT and VAChT were down regulated irrespective of genotype (*p<0.05; n=5/group/genotype).

FIGS. 46A-M show that ablation of Shh from DA neurons results in progressive cellular and physiological abnormalities in the vMB. FIGS. 46A-D show that Th staining of VTA and SNpc is inconspicuous at one month but diminished at 12 months of age in the absence of Shh expression by DA neurons. FIG. 46E shows adult onset, progressive reduction in numbers of Th⁺ neurons in the SNpc. (unbiased stereological counting by optical fractionation; *p<0.05, unpaired t-test (n=7-8/genotype/age, 12 sections per subject with a 5 section interval). No reduction in GAD67⁺ positive cells was observed in the SNpc at 12 months. FIG. 46F shows adult onset, progressive reduction in numbers of Th⁺ neurons in the VTA (unbiased stereological counting by optical fractionation;*p<0.05, unpaired t-test (n=5/group/age, 12 sections per subject with a 5 section interval). FIG. 46G shows decreased density NeuN⁺ and no increase in Th⁻ neurons in SNpc and VTA at 12 months of age (*p<0.05 unpaired t-test (n=6-7/group). FIG. 46H shows that striatal Th⁺ nerve fiber density is increased at 6 months but decreased at 14 months (*p<0.05 unpaired t-test, n=10-11/group). FIG. 46I shows that dopamine content in the vMB is increased early in phenotype progression but diminished in end stage mutants (*p<0.05 unpaired t-test, n=5-6/genotype/age). FIG. 46K shows that DA content of the striatum is highly dynamic with a decrease early in phenotype progression followed by an increase and then eventual diminishment in end-stage mutants (*p<0.05, unpaired t-test, n=5-8/group). FIG. 46L shows that a deficit in elicited DA mobilization manifests between 28 and 60 days of age test (**p<0.001 ANOVA with drug and genotype as independent factors followed by Tukey's post-hoc test, n=7-8/genotype/age/treatment). FIG. 46M shows that transcription of dopaminergic markers is down—and of physiological stress markers up regulated at 4 weeks of age but appears unaltered at 12 months of age in the vMB (* p<0.01, ** p<0.001 two tailed t-test, n=5/genotype).

FIGS. 47A-C show apparent cell autonomous protection of DA neurons by Shh. FIG. 47A shows Shh expressing DA neurons in Shh-nLZ^{C/C}; Dat-Cre mutant animals at 12 months of age. FIG. 47B shows an increase in the frequency of Shh expressing DA neurons among all DA neurons in the SNpc in Shh-nLZ^{C/C}; Dat-Cre mutant animals with phenotype progression (*p<0.05, 1 vs. 12 month of age, unpaired t-test, n=12/group, 100 cells/subject). FIG. 47C shows that Th⁺, Shh⁻ DA-neurons are smaller compared to Th⁺, Shh⁺ DA neurons in the SNpc of Shh-nLZ^{C/C}; Dat-Cre mutant animals (*p<0.05, single comparison by Mann-Whitney U test; box-whisker plots for median±95% CI bar, 25-75 percentiles box, n=4, 50 cells/subject).

FIGS. 48A-L show abnormalities in locomotion and Gait dynamics in the absence of Shh expression by DA neurons. FIG. 48A shows that quantification of horizontal activity in an “Open Field” defines 5 phases of phenotype progression with relative hypo-kinesis in phase 2 and relative hyper-activity in phase 3 (*p<0.05, repeated measures ANOVA follow by Tukey's post hoc test; n=10/group). FIG. 48B shows that cumulative data of 23 litters reared around the year reveals high temporal predictability of transition from phase II to phase III (n=83-100; p<0.000001, phase X genotype ANOVA). FIGS. 48C-E show that a ventral view footpad videography on translucent tread mill belt (DigiGait, Inc.) demonstrates an increase in stride length variability (CV) and absolute paw angle, and a reduction in brake/stride ratio during phase III (n=5; p<0.01; ANOVA followed by Tukey's test). FIG. 48F shows that a quantification of alternations between acceleration and deceleration reveals increased complexity of bouts of locomotion during phase II but a reduced complexity during phase III (n=5; 50 bouts; p<0.05; Mann Whitney test). FIG. 48G shows that increased stride length CV was reversed by L-D OPA (sc., 20 mg/kg) and THP (ip, SC 3 mg/kg; n=8/genotype; *p<0.05, genotype x time ANOVA for repeated measures followed by Tukey's post-hoc test. FIG. 48H shows that decreased time allocated to braking in each gait cycle was normalized by THP but not L-DOPA (n=8/genotype; *p<0.05, ANOVA for repeated measures followed by Tukey's post-hoc test. FIG. 48I shows that increased absolute paw angle was normalized by L-DOPA but not by THP (*p<0.05, ANOVA for repeated measures followed by Tukey's post-hoc test (n=8/genotype). FIGS. 48K-L show that delayed acceleration and deceleration (FIGS. 64C-D) in phase III was partially normalized by L-Dopa (K) and THP (L; n=5/group *p<0.05 drug vs. vehicle in Shh-nLZ^{C/C}; Dat-Cre mutant mice, repeated measures ANOVA followed by Tukey's post-hoc test). Neither drug effected the time spend locomoting at sub-maximal speeds.

FIGS. 49A-D show that Shh signaling from dopaminergic neurons controls structural and neurochemical homeostasis in the meso striatal circuit. FIG. 49A shows additional means of communication between dopaminergic and cholinergic neurons in the meso-striatal circuit: DA neurons communicate with TANS by Shh signaling in addition to dopamine, TANS communicate with DA neurons by GDNF signaling in addition to acetylcholine. FIG. 49B shows rheostat properties of trophic factor signaling: Shh signaling represses GNDF transcription by TANS which are a source of a signal “X” that in return inhibits Shh transcription by DA neurons. Regulation of expression by target derived repressive signals renders trophic signaling responsive to physiological cell stress in the target cell of trophic factor action and provides a mechanism for the homeostatic limitation of trophic signaling. FIG. 49C shows that Shh signaling from mesencephalic DA neurons regulates set-point of cholinergic signaling in the striatum: ambient Shh signaling in the normal brain maintains cholinergic tone through the concerted regulation of the expression of muscarinic autoreceptors and their coupling to Ca++ channels by RGS4. In the absence of Shb signaling the transcription of RGS 4 is down and M2 up-regulated resulting in increased efficacy in autoreceptor signaling and a corresponding decrease in extracellular ACh tone (red arrows). FIG. 49D shows that absence of Shh signaling exposes the meso striatal circuit to increased risk for structural and functional corruption: Control: homeostatic GDNF and Shh signaling results in structural stability and balanced DA and ACh tone. (phase II): chronic absence of Shh signaling causes a reduction in striatal GDNF production and dopaminergic tone leading to hypokinesia and dopaminergic and cholinergic neuro degeneration. (phase III): further neuronal decay in the striatum and compensatory production of DA causes a DA-ACh imbalance resulting in hyperactivity, bradykinesia and gait abnormalities. Phase IV: Compensatory capacity of DA neurons is reached and DA, ACh and GDNF levels in the striatum collapse resulting in more dramatic neurological signs. (dopamine nuclei in red, TANS in green, FS in blue. Red arrows denote reduction, green arrows increases).

FIGS. 50A-E show that Cre mediated ablation of the conditional Shh allele results in loss of function. FIG. 50A shows a homologous recombination strategy. Location of PCR primers for genotyping (1,2,3) and qrtPCR for monitoring transcription of exon 1 (x1, x2) and exon 2,3 (y1, y2) are indicated by red arrow. Dat-Cre mediated recombination deletes exon 2 and 3 and the intervening intron but leaves in place many of the cis-acting elements that control the transcriptional activity of the Shh locus (Lang et al., 2010). Therefore quantifying the concentration of exon 1 containing RNA in mutant animals allows to assess the transcriptional activity of the truncated Shh locus in mutant animals. FIG. 50B shows a Southernblot showing ES cell clone heterozygous for recombinant Shh allele. BamH1 digested genomic DNA was hybridized with the 5′ and 3′ probes indicated by red lines in FIG. 50A. FIG. 50C shows genotyping of mice heterozygous and homozygous for the conditional Shh allele by PCR using oligos #1 and #2 indicated in FIG. 50A. PCR fragment derived from the conditional allele is 79 by longer due to the insertion of an adaptor introducing an additional BamH1 site and a LoxP site. FIG. 50D shows that the homozygous ablation of the conditional Shh allele produced by Hsp70-cre in the germline results in embryonal lethality with morphological features phenocopying the unconditional, homozygous ablation of Shh (see Example 11). FIG. 50E shows that Dat-Cre mediated recombination of the conditional allele produces the ShhN allele present in genomic DNA of the vMB but not of tail (T) or olfactory bulb (B) revealed by PCR using oligos #1 and #3 indicated in FIG. 50A.

FIGS. 51A-C show that a small fraction of Th⁺ neurons lose Shh expression with aging. FIG. 51A shows rare Th⁺, dopaminergic neurons that have lost expression of Shh at 20 months of age (white arrows). FIG. 51B shows that the frequency of Shh expression among DA neurons falls from 100% at 1 months to 4+/−1% at 12 months and 10+/−0.8% at 20 months (* p<0.05, unpaired t-test, n=12/group, 100 cells/subject. FIG. 51C shows that the soma of Th⁺, Shh⁻ DA neurons is smaller compared to Th⁺, Shh⁺ DA neurons in the SNpc of Shh-nLZ^{C/C}; Dat-Cre mutant animals (* p<0.05, single comparison made by Mann-Whitney U test; box-whisker plots for median±95% CI bar, 25-75 percentiles box (n=4, 50 cells/subject)).

FIGS. 52A-D show that Ptc1 is not expressed by mesencephalic DA neurons. FIGS. 52A-B show the immunohistochemical staining for β-Gal (green) and Th (red) on coronal sections of the SNpc and VTA of Ptc1-nLZ tracer mice (Goodrich et al., 1999) at low (A) and higher (B) magnification reveals no Ptc1 expression in DA neurons. FIGS. 52C-D show single channel fluorescence for Th (C) and Ptc1-nLZ (D).

FIGS. 53A-C show unilateral 6-OH-DA injections into the medial forebrain bundle (mFB). FIG. 53A shows that unilateral 6 OH-DA challenge causes contra lateral turning bias. Heterozygous GDNF mutant mice (GDNF-nLZ; Moore et al., 1996) were more sensitive to toxin challenge (* p<0.05, ANOVA followed by post hoc test, n=5/genotype). FIG. 53B shows that unilateral 6 OH-DA injections elicit an ipsi-lateral up-regulation of Shh transcription in the vMB quantified by rtPCR. Heterozygous GDNF mutant mice up-regulated Shh to greater extent than wt animals. Expression of Th was not affected (*p<0.05, two tailed t-test; n=5/group). FIG. 53C shows that unilateral 6 OH-DA injections elicit an ipsi-lateral up-regulation of GDNF and ChAT transcription in the striatum quantified by rtPCR. No up regulation of GDNF transcription was detected in wt animals (*p<0.05, two tailed t-test; n=5/group).

FIGS. 54A-C show that Dat Cre mediated recombination results in tissue specific and efficient ablation of Shh expression from mesencephalic DA-neurons. FIG. 54A shows that quantification of TH⁺, β-Gal⁺ double positive cells in vMB as a whole, SNpc, VTA, Retrorubral Field (RRF) and Medial Amygdala (MeA) of Shh-nLacZ^{c/+}, Dat-Cre⁺ (black bars) vs. Shh-nLacZ^{c/+}; Dat-Cre⁻ (white bars) mice. The efficiency of Cre mediated ablation of Shh is about 80% and specific for DA neurons (* p<0.05, t-test, averages±SEM are shown, 2 mice of each genotype, 5 sections spaced evenly encompassing the entire anterior-posterior extent of the mesencephalic DA nuclei, left and right hemispheres analyzed separately). FIGS. 54B-C show whole mount (“glass-brain”) preparations, ventral view, to assess qualitatively the tissue specificity of Cre recombination: The overall pattern of x-Gal stained nuclei remains unaltered with the exception of the absence of staining in DA neurons of the vMB (red arrows). Green arrows point to the MeA.

FIGS. 55A-E show that striatal- and cortical-morphology, and cellularity is inconspicuous in Shh-nLZ^{C/C}, Dat-Cre mutant mice. FIGS. 55A-B show chromogenic immunohistochemical staining for Th counterstained with Niss1 in Shh-nLZ^{C/C}, Dat-Cre (A) vs. Shh-nLZ^{C/+}; Dat-Cre (B) mice. FIG. 55C shows the average (+/−SEM) transversal area of striatum and cortex (n=8/group, 10 sections/subject). FIG. 55D shows the sterological measurement of striatal volume (mm³) calculated by Cavalieri method (Gundersen and Jensen, 1987). No difference in striatal volume was detected between genotypes at either age (two-way ANOVA; genotype effect: p=0.12; 10 sections/subject, n=8/group). FIG. 55E shows the stereological counting of Toto3 stained nuclei and NeuN⁺ cells at 12 months of age. No difference in cell number was detected between genotypes at either age (two-way ANOVA; genotype effect: p=0.14; n=8/group, 10 sections/subject).

FIGS. 56A-C show that the nuclear size distinguishes ACh-neuron and FS from all other striatal cells. FIGS. 56A-B show that triple staining with anit ChAT- and anti Parv-antisera, and ToPro3 reveals distinct perinuclear staining patterns of ACh-neuron and FS neurons by ToPro3 (Matamales et al., 2009). FIG. 56C shows the quantification of nuclear circumference of striatal cell types identified by perinuclear staining patterns as defined by Matamales et al., 2009 (*p<0.05, single comparisons, Mann-Whitney U test; box-whisker plots, median±95% CI bar, 25-75 percentiles box).

FIGS. 57A-B show that degeneration of ACh-neuron is greatest in lateral aspects of the dorsal striatum in the absence of Shh signaling from DA neurons. FIG. 57A shows that coronal sections of striatum were operationally divided into 3 stripes further segmented at the equator roughly modeled upon somato-topographic projections from the cortex and thalamus and nuclear accumbens (Haber, 2010; Medial dorsal (Md), nucleus acumbens (NA), central dorsal (Cd), central ventral (Cv), lateral dorsal (Ld), lateral ventral (Lv). FIG. 57B shows the relative reduction in numbers of ACh-neuron in each striatal segment (*p<0.05, unpaired two-tailed T-test, n=5/genotype).

FIGS. 58A-D show the progressive activation of a physiological cell stress response in the striatum. FIGS. 58A-B show that mRNA in situ hybridization reveals selective up-regulation of Grp78 (BiP), an indicator for the activation of ER based physiological cell stress response (Lindholm et al., 2006, Zhao and Akerman, 2006), in the striatum of 5 week old animals in large bodied cells (arrow heads). FIG. 58C shows the quantification of in situ signal in cells larger than 20 μm in diameter in Shh-nLZ^{C/C}; Dat-Cre mutant animals (black bar) and Shh-nLZ^{C/+}; Dat-Cre litter controls (grey bar, * p<0.05, unpaired t-test, n=4/group, 10 section/subject). FIG. 58D shows that rtPCR analysis demonstrates activation of Grp78 transcription in striatal extracts at 12 months of age in Shh-nLZ^{C/C}; Dat-Cre mutant animals compared to Shh-nLZ^{C/+}; Dat-Cre litter controls (* p<0.05, unpaired t-test, n=4/genotype).

FIG. 59 shows that GDNF is selectively expressed by cholinergic neurons of striatum. Using the genetic gene expression tracer allele GDNF-LZ (Moore et al, 1996) reveals GDNF restricted expression in cholinergic neurons of the striatum only. MeSeptum: medial septum, d. Band vl: vertical limb of the diagonal band; D. band hl: horizontal limb of the diagonal band; Mg PO: magnocellular locus of the preoptic nucleus; PPTg/LDT: pendunculopontine tegmental nucleus/lateral-dorsal tegmental nucleus.

FIGS. 60A-B show that unilateral injection of AF64a into the PPTg results in contra lateral turning. FIG. 60A shows Open Field video traces 30 h post injection of 1 ul 0.5 mM AF64a into the right PPTg of Shh-nLacZ^{c/c}, Dat-Cre mutant and Shh-nLacZ^{c/+}, Dat-Cre control mice. Shh-nLacZ^{c/c}, Dat-Cre are more sensitive to the inhibition of cholinergic activity in the PPTg. FIG. 60B shows the quantification of turning bias (* p<0.05, post hoc test after ANOVA, n=4/genotype, injection of PPTg was verified by histological analysis post sectioning).

FIGS. 61(1)-(3) show a similar pattern of gene expression alterations upon cholinergic lesions in the striatum and in Shh-nLacZ^{c/c}, Dat-Cre+ mutant mice. FIGS. 61(1-2) show increased Shh transcription and reduced transcription of dopaminergic markers in the ipsi-lateral vMB upon unilateral injection of 1 μl of 0.2 mM (1) or 1 μl of 1 mM (2) AF64a into the striatum (5′ Shh: exon 1 amplicon: x1-x2, 3′ Shh: exon 2-3 amplicon: y1-y2, FIG. 50A. FIG. 61(3) shows that up-regulation of Shh expression and down-regulation of DA neuron marker expression in the vMB of Shh-nLZ^{CC+}; Dat-Cre mutant animals is qualitatively similar to FIG. 61(2). Reduction in 3′ Shh amplicon is reflective of Cre mediated deletion of exons 2 and 3 of the conditional Shh locus (FIG. 50A; n=5/genotype or treatment group * p<0.01, two tailed t-test).

FIG. 62 shows the longitudinal analysis of vertical activity in the “Open Field”: longitudinal pattern of rearing activity mirrors horizontal activity hypo-activity in phase II (2-5 months of age) and hyperactivity in phase III (7-12 months of age; * p<0.05 unpaired t-test; n=10-12/group).

FIGS. 63A-G show the kinetic analysis of spontaneous locomotion. FIG. 63A shows the velocity profile of an individual bout of locomotion from a Shh-nLZ^{C/C}; Dat-Cre mutant (red) and Shh-nLZ^{C/+}; Dat-Cre control (blue) mouse captured at 6 Hz in an open field. FIG. 63B shows the corresponding path of bouts displayed in FIG. 63A showing linear displacement in the open field. FIG. 63C shows that the average duration of locomotion bouts (10±1 cm/s amplitude) does not differ between Shh-nLZ^{C/C}; Dat-Cre mutant and Shh-nLZ^{C/+}; Dat-Cre control mice in phase II or III (10 min observation, * p>0.05, unpaired t-test, n=5/genotype). FIG. 63D shows that the average amplitude of locomotion bouts does not differ between Shh-nLZ^{C/C}; Dat-Cre mutant and Shh-nLZ^{C/+}; Dat-Cre control mice in phase II and III. (n=5/genotype, 10 min observation, p>0.05, unpaired t-test). FIG. 63E shows that the frequency of locomotion bouts of all amplitudes is decreased in Shh-nLZ^{C/C}; Dat-Cre mutant in phase II (n=5/genotype, 10 min observation, p>0.05, unpaired t-test). FIG. 63F shows that the frequency of locomotion bouts of all amplitudes is increased in Shh-nLZ^{C/C}; Dat-Cre mutant in phase II (n=5/genotype, 10 min observation, p>0.05, unpaired t-test). FIG. 63G shows a scheme detailing the analysis of time a mouse spends locomoting with different speeds within a given bout of locomotion by binning locomotion speeds. Analysis is performed separately for the acceleration phase defined as the part of the speed profile before the top speed within a given bout of locomotion is reached and the deceleration phase, defined as the part of the speed profile after the top speed was reached. 10 bins of speed are formed relative to max. speed reached in a given bout.

FIGS. 64A-B show the time profiles animals spend locomoting at different speeds during bouts of locomotion. FIG. 64A shows that in phase II Shh-nLZ^{C/C}; Dat-Cre mutant and Shh-nLZ^{C/+}; Dat-Cre control mice spend most time at initial and max speeds during acceleration and deceleration phases with no discernable differences between the genotypes (n=5/group; p>0.05, ANOVA followed by Tukey's post-hoc test). FIG. 64B shows that in phase III Shh-nLZ^{C/C}; Dat-Cre mutants spend more time at low speed levels and less time at sub maximal speed levels during acceleration phases and more time at sub maximal speed levels and at low speed levels during deceleration phases compared to Shh-nLZ^{C/+}; Dat-Cre control mice (* p<0.05, ANOVA followed by Tukey's post-hoc test, n=5/group).

FIGS. 64C-D are graphs that show the time profiles animals spend locomoting at different speeds during bouts of locomotion in Phase III.

### DETAILED DESCRIPTION OF THE INVENTION

Currently there are no treatments that will cause the replenishment of neurons lost in neurodegenerative diseases. There are also no treatments available that will halt, or even just slow the relentlessly progressive neurodegeneration observed in the clinic. Treatments that will simply slow the progressive neuronal demise of neurons are therefore the single most important unmet need in diseases like Parkinson's disease (PD), progressive supranuclear palsy (PSP), spinocerebellar ataxias (SCA), multiple system atrophy (MSA), corticobasal degeneration (CBD), or amyotrophic lateral sclerosis (ALS) (Olanow C W. Rationale for considering that propargylamines might be neuroprotective in Parkinson's disease. Neurology 2006; 66 (Suppl 4): S69-S79.).

Neurons affected in neurodegenerative diseases also die during aging in the normal brain, however at a much slower rate. Without being bound by theory, this observation demonstrates that there are mechanisms in place in the normal brain which maintain otherwise vulnerable neuronal populations and/or replenish lost neurons through neurogenesis during life. As discussed in the Examples herein, it was investigated as to how mesencephalic dopamine neurons (DA neurons) and spinal cord motor neurons (MN), those neuronal subtypes that degenerate in the above mentioned diseases, are maintained during adulthood. Without being bound by theory, knowledge of those mechanisms that impinge on the longterm maintenance of neurons and/or the regulation of neurogenesis will provide guidance to biochemical processes whose pharmacological manipulation can slow neurodegeneration and/or change the qualitative outcome of neurogenesis towards neurons that are needed for replacement in neurodegenerative conditions.

Using gene expression tracer, conditional gene ablation, and pharmacological strategies, the Examples herein demonstrate that the Sonic Hedgehog (Shh) cell signaling pathway is a crucial regulator of neuronal maintenance, neurogenesis and gene expression in the adult brain. Shh is a cell signaling molecule which is indispensable for early embryogenesis, later organogenesis and overall congruent tissue growth during development. Shh acts through Smoothened (Smo), a 7-transmembrane domain, G-protein coupled receptor protein (GPCR) for which pharmacology was developed previously (see Stanton B Z, Peng L F. Small-molecule modulators of the Sonic Hedgehog signaling pathway. Mol. Biosyst. 2010 January; 6(1):44-54). The Examples herein demonstrate that Shh is expressed in select neuronal populations of the adult CNS including mesencephalic DA neurons and spinal motor neurons. The functions of Shh expression in these adult neuronal cell populations that are disclosed in the Examples herein were previously unknown.

Consistent with the concentration dependent repertoire of Shh functions during development, Shh in the adult CNS has: (1) neurotrophic activity and maintains cholinergic neurons of the striatum; (2) regulates the expression of Shh target genes in the projection areas of DA and MN neurons; and (3) determines the acquisition of particular neuronal cell fates of newly formed neurons during neurogenesis.

Based on these results, the Examples herein show several new utilities for the pharmacological inhibition of Shh signaling in the adult: Reduced Shh signaling (a) leads to an up-regulation of the potent neurotrophic factor GDNF in the basal ganglia and peripheral muscle tissue; and (b) causes increased production of neurons with dopaminergic cell fate by neurogenesis.

Upregulation of Endogenous GDNF by Shh Inhibition

GDNF is a target-secreted neuroprotective, neurotrophic, and neuromodulatory factor. The Neuroprotective role of GDNF has been demonstrated in rodent models of Parkinson's Disease (PD), and ALS. Moreover, GDNF affects the mesolimbic dopaminergic system, making it relevant for drug addiction, as well as hyper-dopaminergic psychiatric conditions such as Schizophrenia, bipolar affective disorder, or Attention-Deficit Hyperactivity Disorder. Unfortunately, GDNF cannot cross the blood-brain barrier, and direct delivery of GDNF into target sites in the brain or spinal cord is not a feasible therapeutic approach due to its invasiveness and due to GDNF immunogenicity. Here, Shh is a signaling pathway that controls the production of the GDNF. This signaling pathway controls target GDNF production and is amenable to manipulation by small molecule compounds. A small molecule approach to selectively enhance GDNF production therefore holds a promise of becoming an effective treatment for ALS and PD.

Without being bound by theory, the partial, pharmacological inhibition of Shh signaling in the adult CNS will up-regulate GDNF expression and in turn help to protect DA neurons of the mesencephalon from neurodegeneration. The Examples presented herein demonstrate results obtained from the analysis of mice with either genetic, conditional Shh loss of function in mesencephalic DA neurons or in somatic spinal cord motor neurons and from mice with induced up-regulation of Shh in mesencephalic DA neurons.

The biological function of GDNF and Shh has been studied in detail during vertebrate development and, to a lesser extent, in the adult organism. Cell to cell signaling, mediated by either protein, take part in the regulation of cell fate determination and congruent tissue growth during early patterning of the embryo and during organogenesis. Expression of both proteins is also readily detected in select cell populations in the adult mouse including distinct neuronal and non neuronal identities of the adult CNS. Interestingly, both signaling pathways exhibit similar functional repertoires acting, however, on distinct target cell populations: both molecules (1) act as “dependence” ligands, leading to the engagement of apoptotic pathways by their receptors in the absence of ligand binding; (2) regulate the expression of distinct sets of target genes as a function of ligand concentration; (3) have neuromodulatory activity on dopaminergic and glutamatergic synapses. Although evidence was published recently for a functional cross talk in the development of the enteric nervous system during embryogenesis (Reichenbach et al., Dev Biol. 2008 Jun. 1; 318(1):52-64.), the Examples presented herein first reveal a regulatory interaction of both pathways in the adult CNS.

GDNF as a Neuroprotective, Neurotrophic, and Neuromodulatory Factor and Use in Medical Applications

GDNF is a potent neurotrophic factor for dopamine- and motor-neurons in the adult CNS. In rodent and primate models Parkinson's Disease (PD, reviewed in Deierborg et al., Prog Neurobiol. 2008 August; 85(4):407-32), GDNF has been shown to protect dopaminergic nigrostriatal neurons from neurotoxins and to induce fiber outgrowth when administered directly into the brain (Akerud et al. 2001, Choi-Lundberg et al., 1997, Gash et al., 1996, Kordower et al., 2000, Rosenblad et al., 1998, Tomac et al., 1995). Mesencephalic dopamine neurons express GDNF receptor-α, and c-Ret, the heterodimer receptor system of GDNF (Kramer et al., 2007). Likewise, the temporally controlled, genetic ablation of GDNF in the adult mouse cause progressive loss of mesencephalic DA neurons and noradrenergic cells in the locus coeruleus, which are affected in early stages of PD (Pascual et al., Nat. Neurosci. 2008 July; 11(7):755-61). GDNF also protects other neurons from neurotoxic damage, particularly noradrenergic cells in the locus coeruleus, which are affected in early stages of Parkinson's disease as well as in Alzheimer's disease and other brain disorders (Arenas et al., 1995). Two open-label clinical trials have evaluated the therapeutic effects of intrastriatal GDNF infusion by canula in patients with Parkinson's disease with encouraging clinical and neurochemical results (Gill et al., 2003; Slevin et al, 2005; Kirik et al., 2004).

GDNF also protects somatic spinal cord motor neurons (MNs) from neuro-degeneration in a number of different models (Henderson et al., 1994, Mohajeri et al., 1999, Acsadi et al., 2002, Wang et al., 2002) and is present in the embryonic limb and adult muscle (Wang et al., 2002, Keller-Peck et al., 2001), the projection areas of MNs. GDNF also increases neural sprouting and prevents cell death of motor neurons (Keller-Peck et al., 2001, Blesch et al., 2001, Deshpande et al., 2006). Healthy motor neurons express GDNF receptor-α and c-Ret, the heterodimer receptor system of GDNF, and can bind, internalize, and transport the protein in both antero- and retrograde directions in a receptor-dependent manner (Glazner et al. 1998, Leitner et al., 1999, von Bartheld et al, 2001). Muscle derived, but not centrally derived, transgenically expressed GDNF protects MNs from progressive degeneration otherwise observed in the transgenic G93A SOD1 model of familial amyotrophic lateral sclerosis in ALS (Li et al., 2007). Direct muscle delivery of GDNF with human mesenchymal stem cells improves motor neuron survival and function in the transgenic G93A SOD rat model of familial ALS (Suzuki et al., 2008).

GDNF reduces cocaine and ethanol self-administration in rats, a widely used animal paradigm to model stimulant addiction (Messer, et al., 2000, Green-Sadan et al, 2003, Green-Sadan et al., 2005, He, et al., 2005). Using methamphetamine self-administration and extinction-reinstatement models, the reduction in the expression of GDNF potentiates methamphetamine self-administration, enhances motivation to take methamphetamine, increases vulnerability to drug-primed reinstatement, and prolongs cue-induced reinstatement of extinguished methamphetamine-seeking behavior that had been previously extinguished (Yan et al., 2007). These findings demonstrate that the reduction in GDNF expression can be associated with enduring vulnerability to the reinstatement of methamphetamine-seeking behavior. GDNF is thus also a potential target for the development of therapies to control relapse (Yan et al., 2007) and provides a good candidate for a therapeutic agent against psycho-stimulants dependence (Niwa et al. 2007).

GDNF expression is up-regulated by tricyclic antidepressants (Hisaoka et al 2007). These experiments demonstrate that the regulation of GDNF production in the adult brain can be an important action of antidepressant that is independent of the modulation of monoamine availability. These findings further demonstrate a possible role for the regulation of GDNF in the pharmacological treatment of depression.

Before GDNF therapy for medical conditions in general and within the CNS in particular can become a reality several obstacles need to be overcome (Sherer et al., 2006, Hong et al., 2008): (a) The delivery of GDNF to the central nervous system (CNS) is challenging because GDNF is a large protein which is immunogenic and is unable to cross the blood-brain barrier; (b) Chronic canulation of the striatum is labor intensive, costly and requires long term maintenance, and can lead to wound infection. Re-canulation is needed in 1 out of 6 patients receiving canulae implants. Canulation destroys healthy CNS tissue; (c) GDNF is a large protein with low diffusion causing protein build up at the tip of the canula and vasogenic edema; (d) GDNF is immunogenic causing the production of antibodies against GDNF in 7 out 10 patients who received GDNF infusion; (e) Expression of GDNF from viral vectors raises concerns about tissue transformation, immunogenic response, and surgical damage during virus application (Hong et al., Neuron. 2008 Nov. 26; 60(4):610-24); (f) Expression of GDNF from transplanted cells raises concerns about histocompatibility and other immunological and surgical complications; and (g) The pharmacological activation of the GDNF receptor or the induction of the expression of GDNF itself in relevant tissues can overcome most of the problems associated with the delivery of GDNF protein into the CNS (Bespalov and Saarma, 2007).

The development of small molecules that specifically activate the GDNF receptor or induce the expression of GDNF itself in relevant tissues and can be administered systemically will overcome most of the problems associated with the delivery of GDNF protein into the brain, with GDNF expression from viral vectors, or with the use of encapsulated GDNF producing cells (Bespalov and Saarma, 2007). XIB4035, a non-peptidyl small molecule that acts as a GDNF family receptor (GFR)α1 agonist and mimics the neurotrophic effects of GDNF in Neuro-2A cells, might have beneficial effects for the treatment of PD (Tokugawa et al., 2003). The oral administration of PYM50028, a non-peptide neurotrophic factor inducer, to 1-methyl-4-phenyl-1,2,3,6-tetrahydropyridine (MPTP)-lesioned mice resulted in a significant elevation of striatal GDNF and attenuated the loss of dopaminergic neurons from the substantia nigra (Visanji et al., 2008).

Understanding the regulation of GDNF expression from relevant sources in the adult brain will give guidance to the development of more specific and efficacious pharmacological strategies to boost GDNF expression. As discussed in the Examples herein, the relevant source of GDNF was first identified in the adult basal ganglia and then the maintenance of the cells that produce GDNF and the regulation of the expression of GDNF in these cells was examined. The Examples herein demonstrate that GDNF is expressed by cholinergic (ACh-) neurons of the adult striatum and that continuous Shh signaling originating from mesencephalic DA neurons is necessary for the maintenance of these neurons. It is further demonstrated that GDNF expression in ACh neurons is inhibited by Shh signaling. Finally, it is demonstrated that injection of cyclopamine into limb muscle, an antagonist of the Shh co-receptor Smo, causes the up regulation of GDNF expression in adult muscles of wild type (wt) and G93A SOD1 mice. As discussed by Ulloa and Briscoe (Cell Cycle. 2007 Nov. 1; 6(21):2640-9), the physiological effects that Shh signaling exerts on target cells is strictly concentration dependent: low levels are needed for the maintenance of cells, medium concentrations regulate the expression of distincts sets of target genes, among which are cell fate determining factors and high levels of Shh have mitogenic effects. Interestingly, at medium concentration ranges, Shh regulates sets of genes, both stimulating or repressing gene expression dependent on the target gene, in such a way that about 1.8 fold changes in effective Shh concentrations causes the execution of distinct transcriptional gene expression programs.

Mechanisms of GDNF Dependent Neuronal Maintenance

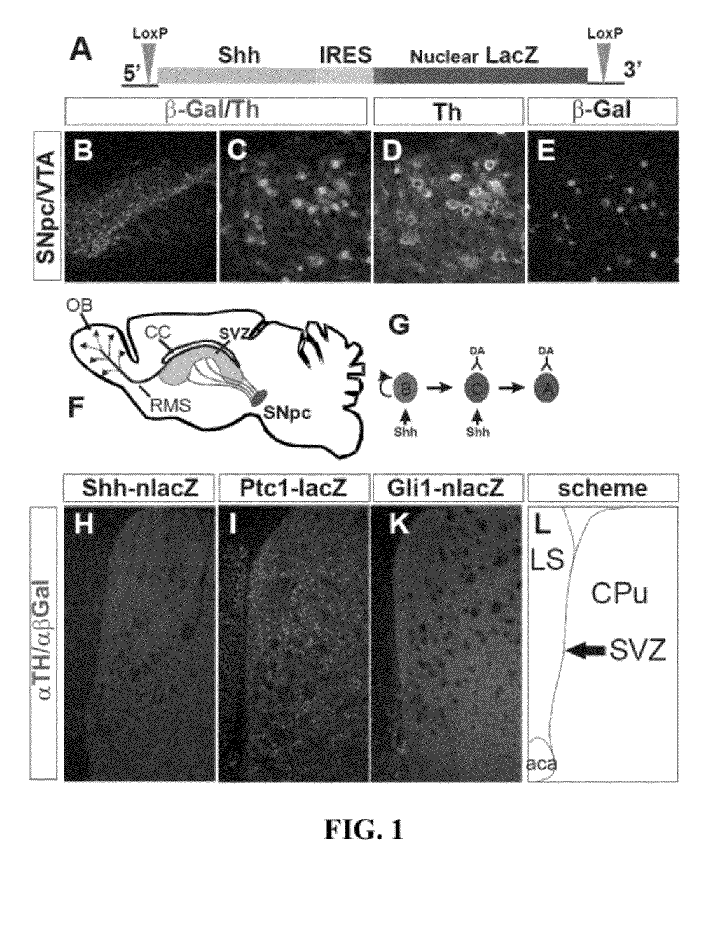

How GDNF withdrawal causes GDNF dependent neurons to die is not well understood. Without being bound by theory, GDNF receptors, like those for BDNF, NGF, Shh and others, act through a ligand “dependence” mechanism in which the ligand unoccupied receptors activate apoptosis through a caspase dependent exposure of a “death” signal in their cytoplasmic domains (FIG. 1; Chao, Sci STKE. 2003 Sep. 16; 2003(200):PE38). Consistent with such a mechanism, Yu et al., J. Neurosci. 2008 Jul. 23; 28(30):7467-75) demonstrated that death receptors and caspases, but not mitochondria, are activated in GDNF deprived dopaminergic neurons in vitro.

Neuromodulatory Function of GDNF

GDNF acutely potentiates the release of dopamine by regulating neuronal excitability via modulating A-type K+ channels and Ca2+ channels in mesencephalic DA neurons (Yang et al., 2001; Wang et al., Neurosignals. 2003 March-April; 12(2):78-88). GDNF also increases the quantal size of dopamine release (Phothos et al., 1998). It has been hypothesized that GDNF withdrawal forces DA neurons to increase dopamine production in order to maintain normal “dopaminergic tone” in the basal ganglia. Such increased metabolic demand has been suggested to contribute to neurodegeneration in disease settings (Calabresi et al., 2006). Pharmacological reduction in GDNF expression might therefore constitute an alternative strategy to achieve dampening of dopaminergic tone in setting of dopaminergic hyper function (i.e. in schizophrenia and other psychotic illnesses).

Parkinson's Disease Treatments Available

Parkinson's disease (PD) is a chronic, degenerative neurological disorder that affects 1% of the population over age 60. With the population aging, the prevalence of PD is projected to grow to 0.25% of the population by 2025. The average age at disease onset is 60. In about 10% of the patients the disease onset is at or below the age of 40. Total number of patients is estimated at 1 million in the USA and 6 million worldwide. There is no effective treatment for slowing or stopping disease progression. Present therapies for Parkinson's disease treat symptoms, by replacing dopamine lost when neurons producing this neurotransmitter are destroyed. There is consequently a tremendous unmet medical need for therapies that treat the etiology of PD.

Available PD Treatment. Levodopa (generic) and other dopamine agonists are commonly used drugs that activate dopamine receptors and reduce many of the symptoms of Parkinsonism. For example, Sinemet (Levodopa+Carbidopa) by Brystol Meyers Squibb, and Requip by GlaxosmithKline are treatments that are available.

Other medicines help prolong and balance the effect of Levodopa, such as COMT inhibitors. COMTAN by Novartis is one example of a COMT inhibitor. Selegiline, amantadine, and anticholinergic medications have also been useful in some patients.

In advanced or unresponsive patients, deep brain stimulation has proven effective for ameliorating some of the motor symptoms.

ALS Treatments Available

Based on U.S. population studies, a little over 5,600 people in the U.S. are diagnosed with ALS each year. It is estimated that as many as 30,000 Americans have the disease at any given time. Disease onset is usually between ages 40 and 70. The average age at diagnosis is 55. ALS is a devastating, incurable disease. The 3-yr. survival rate is about 50% and 10-yr survival rate is about 10%.

Current drug treatment for ALS consists of Riluzole (Sanofi Aventis). The benefits of Riluzole, although consistent, are modest. Riluzole prolongs survival in ALS patients for several months, but has not been shown to have significant effect on measures of function.

The invention is directed to methods of using inhibitors of Sonic Hedgehog signaling (e.g., cyclopamine and related compounds) to up-regulate the expression of endogenous GDNF and/or CNTF to treat subjects afflicted with neurodegenerative diseases. Examples of neurodegenerative diseases include, but are not limited to, Parkinson's Disease (PD), Amyotrophic Lateral Sclerosis (ALS), Alzheimer's, and Supra Nuclear Palsy.

The invention is further directed to methods of using agonists of Sonic Hedgehog signaling to down regulate the expression of endogenous GDNF and/or CNTF in settings of dopaminergic hyperactivity like psychoses (Schizophrenia and others).

The invention is also directed to methods of using antagonists of Sonic Hedgehog signaling to up-regulate the expression of endogenous GDNF and/or CNTF in settings of addiction (e.g., cocaine, alcohol and others).

The invention is directed to methods of using existing and newly discovered compounds that regulate the Shh pathway as adjuvants in settings where exogenous GDNF is given to a patient.

The invention is directed to methods of using existing and newly discovered compounds that regulate the Shh pathway as adjuvants in the preparations of neuronal extracts and cell suspensions for dopaminergic and cholinergic replacement therapies for neurodegenerative diseases like Parkinson's and Alzheimer's and other diseases.

Non-limiting examples of Shh antagonists include cyclopamine, KAAD-cyclopamine, KADAR-cyclopamaine, jervine, SANT 1, SANT 2, SANT 3, SANT 4, Cur-61414, IPI-926, GDC-0449, and robotnikinin (see Stanton B Z, Peng L F. Small-molecule modulators of the Sonic Hedgehog signaling pathway. Mol. Biosyst. 2010 January; 6(1):44-54, which is hereby incorporated by reference in its entirety). For example, GDC-0449 (developed by Curis Inc.) is in Phase II trials (in collaboratinon with Genentech), under evaluation for ovarian and colorectal cancer. For example, Cur-61414 (developed by Curis Inc.) is an aminoproline Hh antagonist and a topical small molecule that inhibits the Hedgehog signaling pathway. It was developed for the treatment of basal cell carcinoma. For example, IPI-926 (Infiniti Discovery Inc.), is an analog of cyclopamine It is in Phase I clinical trials, and was developed for cancer applications. For example, IPI-609 (also known as MEDI562; Infiniti Discovery Inc.) is a small molecule which acts through the inhibition of the hedgehog cell signaling pathway. It was under development as an oral formulation for the treatment of solid tumors. For example, R3616 (Roche) is a hedgehog systemic small molecule which blocks the Hedgehog signaling pathway and is being developed as an oral formulation for the treatment of medulloblastoma. For example, BMS833923 (Bristol-Myers Squibb Company) is a small molecule inhibitor of the hedgehog signaling pathway that inhibits cell proliferation and differentiation in normal development. BMS833923 is being developed for the treatment of advanced or metastatic cancer. For example, MEDI562 (AstraZeneca) is a small molecule targeted for cancer therapy, which acts through the inhibition of the hedgehog cell signaling pathway. For example, XL139 (Exelixis Inc.) is a small molecule inhibitor of the hedgehog signaling pathway that inhibits cell proliferation and differentiation in normal development. XL139 is being developed for the treatment of advanced or metastatic cancer. For example, Actar AB has generated Gli-specific inhibitors act by inactivating the hedgehog (Hh) signaling pathway.

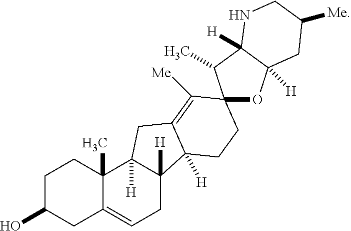

The structure of cyclopamine is:

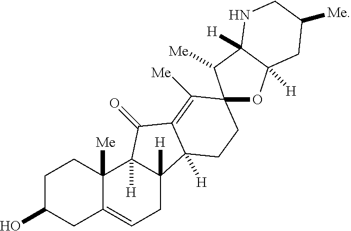

The structure of jervine is:

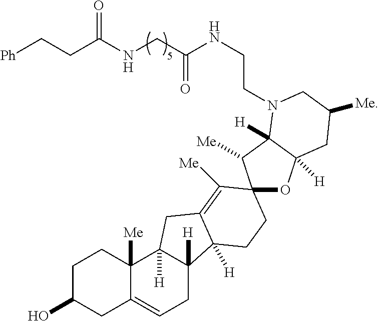

The structure of KAAD-cyclopamine is:

The structure of GDC-0449 is:

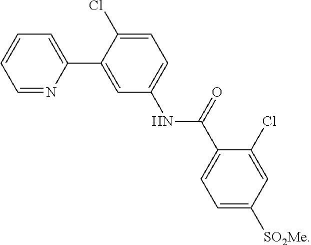

Further discussion of the characteristics of the GDC-0449 compound is found at Wong et al., Xenobiotica. 2009 November; 39(11):850-61; and Robarge et al., Bioorg Med Chem. Lett. 2009 Oct. 1; 19(19):5576-81, each of which are hereby incorporated by reference in their entireties.

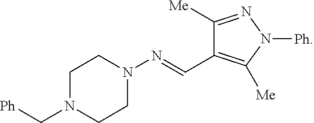

The structure of SANT1 is:

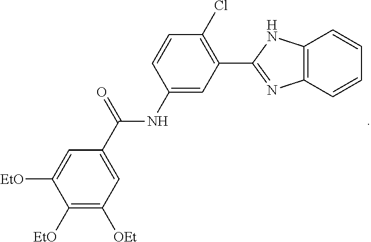

The structure of SANT2 is:

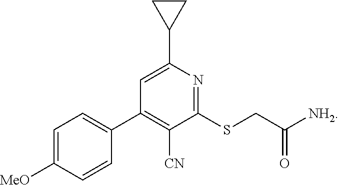

The structure of SANT3 is:

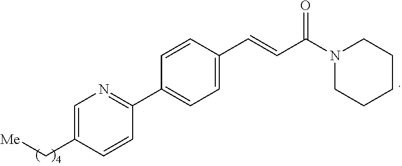

The structure of SANT4 is:

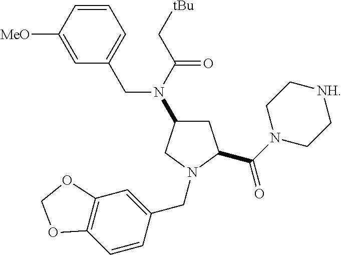

The structure of Cur-61414 is:

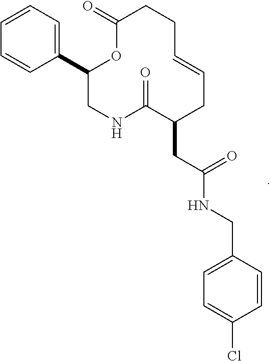

The structure of robotnikinin is:

Non-limiting examples of Shh agonists include purmorphamine or SAG (see Stanton B Z, Peng L F. Small-molecule modulators of the Sonic Hedgehog signaling pathway. Mol. Biosyst. 2010 January; 6(1):44-54, which is hereby incorporated by reference in its entirety). For example, Procter & Gamble Company has generated Hedgehog Small Molecule Agonist that activates the Hedgehog signaling pathway. Hedgehog Small Molecule Agonist was under development as a topical formulation. For example, Wyeth has generated Hedgehog small molecule agonists that are orally available compounds. However, in 2008, Wyeth decided that it would no longer pursue its development efforts on the Hedgehog agonist program.

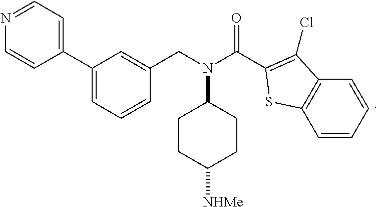

The structure of SAG is:

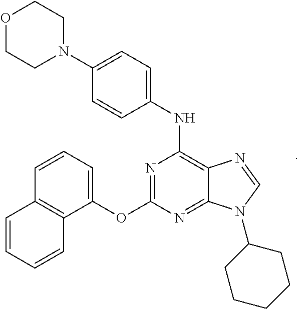

The structure of purmorphamine is:

In some embodiments, a Shh antagonist can be a small molecule that binds to the Smoothened receptor, the Gli effector protein, or Shh ligand. The small molecule can disrupt protein function and/or downstream signaling effects and/or effectors. In some embodiments, a Shh agonist can be a small molecule that binds to the Smoothened receptor, the Gli effector protein, or Shh ligand, enhancing the functions of the proteins. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that inhibit Shh can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries (Werner et al., (2006) Brief Funct. Genomic Proteomic 5(1):32-6).

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

In one embodiment, the Shh antagonist can be cyclopamine or KADAR-cyclopamaine. In another embodiment, the Shh antagonist can be any one of the cyclopamine analogues or hedgehog antagonist compounds disclosed in U.S. Pat. Nos. 7,230,004 and 6,545,005 (each of which is incorporated by reference in their entireties). For example, cyclopamine is a natural product that inhibits the Shh pathway by affecting the active and inactive forms of the Smoothened protein.

A Shh antagonist can also be a protein, such as an antibody (monoclonal, polyclonal, humanized, and the like), or a binding fragment thereof, directed against the smoothened receptor protein, Smo, or the Shh ligand. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (see Steinitz M. Hum Antibodies. 2009; 18(1-2):1-10; Grönwall C, Ståhl S. Engineered affinity proteins—generation and applications. J. Biotechnol. 2009 Mar. 25; 140(3-4):254-69; Jenkins N, Meleady P, Tyther R, Murphy L. Biotechnol Appl Biochem. 2009 May 6; 53(Pt 2):73-83; and Weisser N E, Hall J C. Biotechnol Adv. 2009 July-August; 27(4):502-20, each of which are hereby incorporated by reference in their entireties).

Production of Dopaminergic Neurons by Endogenous Neurogenesis

Adult neurogenesis in the sub ventricular zone (SVZ) of the undisturbed forebrain can produce a multitude of neuronal and non-neuronal cell identities in vivo which replenish various neuronal populations in the olfactory bulb and oligodendrocytes in the forebrain (Alvarez-Buylla and Lim, 2004; Hoeglinger et al., 2004; Imayoshi et al., 2008; reviewed in Zhou et al., 2008). These observations demonstrate the omnipotency of neuronal stem cells present in the adult brain and provide the basis for the hope that these stem cells can be coaxed into replenishing brain tissue(s) with functional neurons and glia that are lost in neurodegenerative diseases (Okano and Sawamoto, 2008). While earlier attempts to demonstrate specific tissue replenishment from SVZ neurogenesis upon pharmacologically or genetically induced cell ablation in the adult brain has met with little success (reviewed in Breunig et al., 2007), recent work demonstrates that dopamine depletion as well as ischemic brain injury can lead to the production of striatal neuroblasts and subsets of striatal interneuron populations (de Chevigny et al., 2008, Yang et al., 2008).

In translational stem cell research, particular interest has been devoted to neural precursor/stem cells resident in regions that display neurogenesis in adult mammals (Gage, 2000; Sohur et al., 2006). This is due to the promise that neuronal stem cells resident in the adult brain can be coaxed into replenishing brain tissue with functional neurons and glia that are lost in neurodegenerative disease (reviewed in Breunig et al., 2007), such as Alzheimer's Disease or Parkinson's Disease. Many neurodegenerative diseases lead to changes in the cytoarchitecture and qualitative outcome of SVZ neurogenesis, pointing to pathological as well as adaptive and corrective functional alterations in the SVZ dependent on the specific disease (reviewed in Curtis et al., 2007).

A physiological adaptation of neurogenic outcome to current physiological needs of the adult CNS requires at least two functions: a) the generation of a cell type specific signal for functional and/or structural deterioration b) a mechanism by which this signal is translated into appropriate alterations in cell fate determination in the SVZ. While there is excellent evidence that adult neurogenesis in the undisturbed brain can produce a multitude of neuronal and non-neuronal cell identities in vivo (Alvarez-Buylla and Lim, 2004; Hoglinger et al., 2004), it is not known by which mechanisms this diversity is generated (Merkle et al., 2007). Likewise, no dynamic signal, that can act as a “sentinel” for structural and functional corruption and that can interface with SVZ neurogenesis, has been identified. However, knowledge of the regulatory mechanisms that impinge on neurogenesis in the adult brain appear to provide the most straight forward guidance to those biochemical processes whose pharmacological manipulation can change the qualitative outcome of neurogenesis towards neurons that are needed for replacement in disease. Together with the observation that many neurodegenerative diseases lead to changes in the cyto-architecture and qualitative outcome of SVZ neurogenesis in the SVZ, pointing to pathological as well as adaptive and corrective functional alterations, dependent on the specific disease (reviewed in Curtis et al., 2007 and Thompson et al., 2008), the qualitative outcome of SVZ neurogenesis can be adapted to physiological need in vivo.

Sonic Hedgehog in Ontogeny

During vertebrate development, morphogens, emanating from localized sources, form gradients of extracellular signals that organize fields of cells and govern the specification of cell fate by inducing the expression of different target genes at different concentrations in responding cells (Wolpert, 1996; Gurdon and Bourillot 2001, Jaeger and Reinitz, 2006,). Sonic hedgehog (Shh) is such a morphogen and is required for multiple aspects of development in a wide range of tissue types (reviewed in McMahon et al., 2003; Ash and Briscoe, 2007, Ulloa and Briscoe, 2007). During the development of the CNS, distinct neuronal subtypes emerge in a precise spatial order from progenitor cells arrayed along the dorsal-ventral axis of the spinal cord (reviewed in Dessaud et al., 2008): Here, Shh acts as a long-range graded signal that controls the pattern of neuronal differentiation during embryogenesis. In vitro assays indicate that incremental two- to threefold changes in Shh concentration generate five distinct neuronal subtypes characteristic of the ventral neural tube (Ericson et al. 1997a). Shh acts by regulating the spatial pattern of expression of transcription factors that include members of the homeodomain protein (HD) and basic helix-loop-helix (bHLH) families (Ericson et al. 1997b; Briscoe et al. 2000; Muhr et al. 2001; Novitch et al. 2001; Pierani et al. 2001; Vallstedt et al. 2001). These transcription factors are subdivided into two groups, termed class I and II proteins, on the basis of their mode of regulation by Shh signaling (Briscoe et al. 2000). Class I proteins are repressed by Shh signaling, whereas neural expression of class II proteins requires exposure to Shh (Ericson et al. 1997b; Qiu et al. 1998; Briscoe et al. 1999, 2000; Pabst et al. 2000; Vallstedt et al. 2001). Cross-repressive interactions between pairs of class I and class II proteins define the spatial extent of individual progenitor domains and establish sharp boundaries between adjacent domains (Ericson et al. 1997b; Briscoe et al. 2000). Changing the progenitor homeodomain code alters neuronal subtype in a predictable manner, indicating that the profile of class I and class II protein expression within a progenitor cell determines the subtype identity of neurons generated (Briscoe et al. 2000).

Three zinc finger-containing transcription factors, Gli1, Gli2, and Gli3, mediate Shh-dependent gene expression (Lee et al. 1997; Sasaki et al. 1997; Ruiz i Altaba 1998; Ingham and Mc-Mahon 2001). Work by Stamtaki et al. (2005) revealed that the incremental changes in Shh concentration that are necessary to switch between alternative neuronal subtypes in the neural tube can be mimicked by similarly small changes in the level of Gli activity demonstrating that a particular extracellular concentration of Shh leads to a distinct level of Gli expression inside a responsive cell that is exposed to Shh.

Shh also plays a mitogenic role in the expansion of granule cell precursors during CNS development and when ectopically expressed in the developing spinal cord (Wechsler-Reya and Scott, 1999; Rowitch et al., 1999; Dahmane and Ruiz-1-Altaba, 1999; Wallace, 1999, Lewis et al., 2004). In Shh null mice, dorso-ventral patterning and the specification of ventral cell populations along the entire neuraxis, and general brain proliferation are all affected. In these mutants the spinal cord is dorsalized with absent ventral cell types, including floorplate cells and motor neurons (Chiang et al., 1996). The telencephalon is greatly dysmorphic, much reduced in size and appears as a single fused vesicle that is strongly dorsalized (Chiang et al., 1996; Rallu et al., 2002). Oligodendrocyte differentiation is completely blocked in Shh mutants (Lu et al., 2000). In general agreement with the loss-of-function phenotype, gain-of-function approaches have demonstrated that misexpression of Shh in the embryonic telencephalon results in the expression of ectopic ventral markers (Kohtz et al., 1998; Gaiano et al., 1999; Gunhaga et al., 2000), abnormal proliferation (Gaiano et al., 1999), and the appearance of supernumerary oligodendrocytes (Nery et al., 2001).

In CNS ontogeny, distinct neuronal subtypes emerge in a precise spatial order from progenitor cells arrayed along the dorsal-ventral axis of the neural tube (Wolpert 1996; Gurdon and Bourillot 2001; reviewed in Ulloa and Briscoe, 2007). Ventrally, Shh is secreted from the floorplate and notochord and acts as a long-range, graded, morphogenic signal by forming a concentration gradient from ventral to dorsal along the midline that controls cell fate determination. The genetic ablation of Shh causes a dorsalization of the spinal cord with ventral cell types missing and the complete a blockade of oligodendrocyte differentiation (Chiang et al., 1996, Lu et al, 2000). In vitro assays indicate that incremental two- to threefold changes in Shh concentration determine the identity of at least five distinct neuronal subtypes characteristic of the ventral neural tube (Ericson et al. 1997a). Shh acts by regulating the spatial pattern of the expression of transcription factors that include members of the homeodomain protein (HD) and basic helix-loop-helix (bHLH) families (Ericson et al. 1997b; Briscoe et al. 2000; Muhr et al. 2001; Novitch et al. 2001; Pierani et al. 2001; Vallstedt et al. 2001). These transcription factors are subdivided into two groups, termed class I and II proteins, on the basis of their mode of regulation by Shh signaling (Briscoe et al. 2000). Class I proteins, like Pax6 and Pax7, are repressed by Shh signaling, whereas neural expression of class II proteins, like Nkx, Olig2, requires exposure to Shh (Ericson et al. 1997b; Qiu et al. 1998; Briscoe et al. 1999, 2000; Pabst et al. 2000; Vallstedt et al. 2001). Changing homeodomain code in progenitors alters neuronal identity, indicating that the profile of class I and class II protein expression within a progenitor cell determines the identity of neurons generated (Briscoe et al. 2000).

Shh and Adult Neurogenesis

Throughout adult life, cells are born in the SVZ and most of them traverse a long distance anteriorly through the rostral migratory stream (RMS) to replenish olfactory bulb (OB) interneuron populations (reviewed in Alvarez-Buylla and Lim, 2004). At least three types of cells can be distinguished in the stem cell niche of the SVZ. Infrequently dividing GFAP+ astrocytes, with stem cell properties (type B cells), which in turn give rise to highly proliferative, EGF-receptor+ precursors (type C cells) forming clusters next to chains of PSA-NCAM+ neuroblasts (type A cells) most of which migrate through the RMS towards the olfactory bulb (Alvarez-Buylla and Garcia-Verdugo, 2002; Riquelme et al., 2008).

Cells in the adult SVZ express the Shh receptor patched (Ptc1) and the signal transduction components smoothened (Smo), Gli1, Gli2 and Gli3 (Charytoniuk et al., 2002a, Machold, et al., 2003, Palma, et al., 2005, Ahn and Joyner, 2005). Co-localization of Gli1 with mitotic markers and a reduction of mitotic activity in mice with Nestin-Cre mediated Smo ablation in the SVZ demonstrates that at least a subpopulation of actively proliferating cells in the SVZ are responsive to Shh (Machold et al., 2003). The number of apoptotic cells was also increased in the SVZ of these mice, indicating that in addition to a possible mitogenic function, Shh might also act as a trophic factor for the maintenance of progenitor cells. Neurosphere assays reveal that Shh cooperates with low doses of EGF to regulate the number of adult SVZ stem cells (Palma et al., 2005). Shh agonist administration increases the number of Gli1+, mitotic cells in the SVZ (Machold, et al., 2003), while inhibition of Shh signaling attenuates Gli1 expression and decreases SVZ cell proliferation in vivo (Palma et al., 2005). Ahn and Joyner (2005) utilized an in vivo, genetic, cell fate mapping strategy based on Cre activity which is co-dependent on pharmacologically induced translocation of the protein into the nucleus and the Shh dependent transcriptional activation of the Gli1 locus (Gli1-CreERt2) to mark Shh responsive cells in the SVZ and their progeny. Their data demonstrate that both quiescent stem cells (“B”-cells) and transit amplifying cells (“C-cells”) are Shh responsive and that these cells give rise to a multitude of cell types in the adult animal.

Despite the evidence for an involvement of Shh in SVZ neurogenesis it remains unclear which cells in vivo act as the relevant source(s) of Shh (Palma et al., 2005, Charytoniuk et al., 2002a, for review see Riquelme et al, 2008). Interestingly, Shh can be transported through, and released from, axons preserving its biological activity: In the fly, hedgehog (Hh) is transported through axons from the soma of photoreceptor neurons into the medulla. Upon its release from axon terminals Hh takes part in the medulla in the temporal restricted formation of topographically organized “cartridges” of 1^{st} order relay neurons (Huang and Kunes, 1996). More recently the Kunes lab has identified a conserved amino acid motif (G*HWY) in the c-terminal half of the unprocessed Hh, which targets Hh into axons. This sequence is also present in Shh (Chu et al., 2006).

Specification of Neuronal Subtype Identities in Adult Neurogenesis

At least 5 distinct populations of olfactory bulb interneurons at fixed relative numbers are produced continuously through SVZ neurogenesis: GABAergic-granular interneurons, Pax6+, TH+ periglomerular interneurons, calretinin+-periglomerular interneurons and calbindin+-periglomerular interneurons, and ER81+ granular interneurons of the outer layers (Altman, 1969, Luskin, 1993; Lois and Alvarez-Bualla, 1994; Kosaka et al., 1985; Kosaka et al., 1998; Saghatelyan et al. 2004; Kohwi et al., 2005). It is not known how this diversity of neurons is generated and whether the concept of regional specification of neuronal subtype identities through morphogen gradients that is prominent in embryogenesis also applies to adult neurogenesis. The physical size of the SVZ makes it unlikely that morphogen gradients emanating from specific tissues with “organizer” activity within the SVZ can operate across the entire neurogenic niche (Guerrero and Chiang, 2007). Nevertheless, Hack et al., (2005) demonstrated that cell fate decisions in the SVZ occur in a hierarchical organized fashion and result from mechanisms similar to those operating in the specification of neurons during development (Lee et al., 2005; Ericson et al., 1997): The expression of Pax6 marks a neuronal precursor lineage many of which further differentiate into various interneuron populations of the OB, whereas Olig2 defines a lineage that almost exclusively will form mature oligodendrocytes. Interestingly, Merkle et al., (2007) provided evidence through heterotopic grafting of SVZ stem cells for a “prepattern” of the SVZ by stem cells of distinct differentiation potential which are distributed within the SVZ in a mosaic arrangement. In summary, reports are consistent with a scenario in which Shh is provided to the SVZ by distinct neuronal nuclei of the adult brain, which provide distinct, topographically organized innervation of the SVZ through axon-colaterals. Thus, Shh signaling is critical for the modulation of the number of cells with stem cell properties, for the proliferation of early precursors and consequently for the production of new neurons.

The invention is directed to methods of modulating the Sonic Hedgehog (Shh) signal transduction pathway which can be used to alter the qualitative outcome of neurogenesis in the adult brain. The invention is also directed to compounds that regulate the Shh signal transduction pathway that can be used to alter the qualitative outcome of neurogenesis in the adult brain. The invention further provides methods that allow regulation of expression of Shh, a potent maintenance- and differentiation-factor of stem cells, in vivo in the adult brain, thus giving rise to specific cells that need to be replaced in neurodegenerative diseases

The invention is directed to methods of regulating Shh production and delivery by DA neurons of the mesencephalon to the SVZ via axonal projection. The invention is also directed to methods of influencing cell fate decisions in SVZ neurogenesis and interfaces between the detection of physiological stress in neurons and the alteration of the qualitative outcome of SVZ neurogenesis. Resident neuronal stem cells can be coaxed into replenishing neurons and glia for which a physiological need exists, serving as a mode of neuronal replacement for CNS repair.

The invention further provides methods of replacing neurons, for example, dopamine neurons in Parkinson's Disease, and cholinergic neurons in Alzheimer's Disease and Supra Nuclear Palsy through alterations in the qualitative outcome of SVZ neurogenesis. In one embodiment, the production of a particular neuronal cell type (such as a dopamine neuron or a cholinergic neuron) can be induced with a compound. For example, injection of AF64a (a cholinotoxin) results in up-regulation of Shh by dopaminergic (DA) neurons. Without being bound by theory, this increase in Shh expression in turn directs the production of more cholinergic neurons by neurogenesis, correlating with the loss of Shh from dopamine cells causing the production of more dopamine cells by neurogenesis. A switch in cell fate determination is, thus, a function of the levels of Shh expression by mesencephalic DA neurons.

In one embodiment, the invention demonstrates that Shh expressed by adult dopaminergic (DA) neurons of the mesencephalon and delivered to the subventricular zone (SVZ) by axonal projection, is a key regulator of adult neurogenesis. In another embodiment, tissue-specific, genetic ablation of Shh from DA neurons alters neurogenic activity, cell fate determination in the SVZ and the olfactory bulb. In a further embodiment, Shh expression by DA neurons is up-regulated dynamically in correlation with the severity of cell physiological stress and neuronal dysfunction in connected neurons. In some embodiments, newly formed DA neurons migrate into the substantia nigra, where up-regulation of Shh expression in mesencephalic DA neurons causes the production of DA neurons.

In one embodiment, the invention provides for therapeutic replacement of neurons lost in neurodegenerative diseases, such as dopamine neurons in Parkinson's Disease, and cholinergic neurons in Alzheimer's Disease and Supra Nuclear Palsy. In another embodiment, the invention provides for therapeutic use for neurological conditions such as stroke, Huntington's Disease, spinal cord repair and regeneration. The invention provides mechanistic insights that can be used for other stem cell therapies targeted at cancer, cardiovascular diseases, diabetes and tissue engineering.

As discussed previously herein, non-limiting examples of Shh antagonists include cyclopamine, KAAD-cyclopamine, KADAR-cyclopamaine, jervine, SANT 1, SANT 2, SANT 3, SANT 4, Cur-61414, IPI-926, GDC-0449, and robotnikinin (see Stanton B Z, Peng L F. Small-molecule modulators of the Sonic Hedgehog signaling pathway. Mol. Biosyst. 2010 January; 6(1):44-54, which is hereby incorporated by reference in its entirety).

As discussed previously herein, non-limiting examples of Shh agonists include purmorphamine or SAG (see Stanton B Z, Peng L F. Small-molecule modulators of the Sonic Hedgehog signaling pathway. Mol. Biosyst. 2010 January; 6(1):44-54, which is hereby incorporated by reference in its entirety).

Pharmaceutical Compositions and Administration for Therapy

The pharmaceutical composition is provided in an amount effective to treat the disorder in a subject to whom the composition is administered, to protect neurons in a subject afflicted with or is at risk of developing a neurodegenerative disorder, or to regenerate neurons in the subventricular zone (SVZ) of a subject afflicted with a neurodegenerative disorder. As used herein, “effective amount” means effective to ameliorate or minimize the clinical impairment or symptoms resulting from a neurodegenerative disorder, effective to regenerate neurons in the SVZ of a subject afflicted with a neurodegenerative disorder, or effective to protect neurons from neuronal death. For example, the clinical impairment or symptoms of ALS or PD can be ameliorated or minimized by reducing/diminishing any pain or discomfort suffered by the subject; by extending the survival of the subject beyond that which will otherwise be expected in the absence of such treatment; or by inhibiting or preventing the development of the disorder.

The amount of pharmaceutical composition that is effective to treat a neurodegenerative disorder in a subject will vary depending on the particular factors of each case including, for example, the type or stage of the neurodegenerative disorder, the subject's weight, the severity of the subject's condition and the method of administration. These amounts can be readily determined by a skilled artisan.

As discussed in the Examples herein, the Shh antagonist cyclopamine was administered at 8 mg/kg/day, 20 mg/kg/day, and 50 mg/kg/day in mice. One of ordinary skill in the art will appreciate that the dosing range of Shh antagonists or agonists administrated to humans should be at a much lower side. Furthermore, dosages for oncology clinical trials directed at Shh antagonists are high (e.g, 150 mg/kg/day) since cell death of transformed cells is the objective. According to the methods of the invention, cell death is not the goal, but rather upregulation of endogenous GDNF or regeneration of dopaminergic neurons. For example, dosages of GDC-0449 used by Von Hoff et. al. (N Engl J. Med. 2009 Sep. 17; 361(12):1164-72.) in patients were 150 mg/day and 270 mg/day. In one embodiment, the dosing range used according to the invention is at least 100× less than what is used in clinical oncology trials. In some embodiments, the effective amount of the administered Shh antagonist or agonist is at least about 0.01 μg/kg body weight, at least about 0.025 μg/kg body weight, at least about 0.05 μg/kg body weight, at least about 0.075 μg/kg body weight, at least about 0.1 μg/kg body weight, at least about 0.25 μg/kg body weight, at least about 0.5 μg/kg body weight, at least about 0.75 μg/kg body weight, at least about 1 μg/kg body weight, at least about 5 μg/kg body weight, at least about 10 μg/kg body weight, at least about 25 μg/kg body weight, at least about 50 μg/kg body weight, at least about 75 μg/kg body weight, at least about 100 μg/kg body weight, at least about 150 μg/kg body weight, at least about 200 μg/kg body weight, at least about 250 μg/kg body weight, at least about 300 μg/kg body weight, at least about 350 μg/kg body weight, at least about 400 μg/kg body weight, at least about 450 μg/kg body weight, at least about 500 μg/kg body weight, at least about 550 μg/kg body weight, at least about 600 μg/kg body weight, at least about 650 μg/kg body weight, at least about 700 μg/kg body weight, at least about 750 μg/kg body weight, at least about 800 μg/kg body weight, at least about 850 μg/kg body weight, at least about 900 μg/kg body weight, at least about 950 μg/kg body weight, or at least about 1000 μg/kg body weight.

In other embodiments, the Shh antagonist or agonist is administered at least once daily for up to 5 days, up to 7 days, up to 15 days, up to 18 days, up to 19 days, up to 20 days, up to 21 days, up to 22 days, up to 23 days, up to 24 days, or up to 25 days. As a provilactive treatment, it is envisoned that Shh antogonists are administered intermittantly once weekly or biweekly over prolonged times (e.g., several years, such as 1 year, 2 years, 3 years, 4 years, 5 years, 7 years, 10 years, 15 years). The rational here is that intermittant boosts of GDNF has trophic benefits that extend the period of GDNF upregulation. Such a dosing strategy might also not interfere with other concentration dependent functions of endogenous GDNF.

In the methods of the present invention, the pharmaceutical composition can be administered to a human or animal subject by known procedures including, without limitation, oral administration, parenteral administration (e.g., epifascial, intracapsular, intracutaneous, intradermal, intramuscular, intraorbital, intraperitoneal, intraspinal, intrasternal, intravascular, intravenous, parenchymatous or subcutaneous administration), transdermal administration and administration by osmotic pump. One method of administration is parenteral administration, by intravenous or subcutaneous injection.

Shh antagonists or agonists to be used according to the invention can be incorporated into pharmaceutical compositions suitable for administration. Such compositions can comprise a Shh antagonist or and a pharmaceutically acceptable carrier.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition containing a Shh antagonist or Shh agonist can be administered in conjunction with a pharmaceutically acceptable carrier, for any of the therapeutic effects discussed herein. Such pharmaceutical compositions can comprise, for example antibodies directed to polypeptides comprising the Shh signaling cascade (see, for example, FIG. 1 of Stanton B Z, Peng L F. Small-molecule modulators of the Sonic Hedgehog signaling pathway. Mol. Biosyst. 2010 January; 6(1):44-54, which is hereby incorporated by reference in its entirety). The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethelene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the Shh antagonist or Shh agonist in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art In some embodiments, the Shh antagonist or agonist can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Methods for the Regulation of GDNF Expression in the Adult Organism by Small Molecular Weight Drugs

The development of systemically administered small molecules that specifically activate or antagonize the GDNF receptor, induce or repress the expression of GDNF itself in relevant tissues will overcome most of the problems associated with the delivery of GDNF protein into the brain.

One must determine the following: (1) whether there are relevant sources of GDNF in the adult organism; (2) how GDNF expression is regulated in these tissues; (3) lead compounds that can regulate the expression of Shh in these tissues in the adult organism; and (4) whether such a compound will lead to the upregulation of GDNF expression in relevant tissues in a validated model of a neurodegenerative disease whose disease course can be modified by GNDF application.

This Example illustrates that (a) cholinergic neurons of the dorsal and ventral striatum express GDNF throughout life, potentially exposing all dopamine neurons of the mesencephalon to GDNF in the adult brain; (b) up-regulation of Shh causes an inhibition of GDNF expression in the striatum; and (c) injection of the cholinotoxin AF64a into the penduncolo pontine nucleus (PPTg) causes an up-regulation of Shh expression in dopamine neurons of the mesencephalon. Furthermore, the following observations as to the spinal somatic motor neuron system were made: subsets of all somatic spinal motor neurons in the adult spinal cord express Shh; GDNF is expressed in adult skeletal muscle; the genetic ablation of Shh expression from motor neurons increases GDNF and CNTF expression in the muscle; there is a loss of GDNF expression in the muscle and a concomittant up regulation of Shh in motor neurons in the G93A SOD transgenic model of ALS; the injection of the Shh pathway antagonist Cyclopamine into calf muscles of control animals causes a 20 fold up regulation of GDNF expression in the muscle; and injection of the Shh pathway antagonist Cyclopamine into the calf muscles of end stage G93A SOD mice causes a 2,000 fold upregulation of GDNF.

These genetic and pharmacological experiments demonstrate that the manipulation of Shh mediated cell signaling, causes alterations in GDNF expression in the adult animal. Existing as well as forthcoming pharmacology targeting the Shh signaling pathway can be utilized to either induce or inhibit endogenous expression of GDNF.

Pharmacological stimulation of endogenous GDNF production using low-molecular weight drugs that specifically activate the GDNF receptor or induce the expression of GDNF itself in relevant tissues can be administered systemically. To test this, it will be (a) determined whether there are relevant sources of GDNF in the adult organism; and (b) determined how GDNF expression is regulated in these tissues. Lead compounds will be identified that can regulate the expression of Shh in these tissues in the adult organism. To demonstrate that such a compound will lead to the upregulation of GDNF expression in relevant tissues, a validated model of a neurodegenerative disease whose disease course can be modified by GDNF application will be used.

The ascending, mesencephalic dopamine system and the cholinergic system of the basal forebrain, in aggregation, provide part of the anatomic substrate for a wide variety of neurodegenerative diseases (i.e. Parkinson's Disease, Alzheimer's, Huntington's, supra nuclear palsy and others), addiction, and psychosis (Schizophrenia). It was therefore first sought to clarify whether there is an endogenous source of GDNF in the adult brain which can expose these neuronal nuclei to GDNF. The regulation of the expression of GDNF in these tissues was then studied. The following was found: (a) cholinergic neurons of the dorsal and ventral striatum express GDNF throughout life, potentially exposing all dopamine neurons of the mesencephalon to GDNF in the adult brain; (b) injection of the cholinotoxin AF64a into the penduncolo pontine nucleus (PPTg) causes an up-regulation of Shh expression in dopamine neurons of the mesencephalon; and (c) the up-regulation of Shh causes an inhibition of GDNF expression in the striatum.

These observations were then extended to the spinal somatic motor neuron system. It was shown that (a) subsets of all somatic spinal motor neurons in the adult spinal cord express Shh; (b) GDNF is expressed in adult skeletal muscle; (c) the genetic ablation of Shh expression from motor neurons increases GDNF and CNTF expression in the muscle; (d) there is a profound loss of GDNF expression in the muscle and a concomitant up regulation of Shh in motor neurons in the G93A SOD transgenic model of ALS; and (e) that the injection of the Shh pathway antagonist Cyclopamine into the calf muscles of end stage G93A SOD mice causes a 27 fold up-regulation of GDNF and a 20 fold up-regulation of CNTF. Genetic and pharmacological experiments demonstrate that the manipulation of Shh mediated cell signaling causes alterations in GDNF expression in the adult animal. Pharmacology targeting the Shh signaling pathway can be utilized to either induce or inhibit endogenous expression of GDNF.

The transgenic G93A SOD model of familial ALS is a well established model for progressive motor neuron degeneration. Elevating GDNF content in peripheral muscles of G93A SOD transgenic rats and mice either by the expression of GDNF from transplanted cells or from muscle specific transgenic expression vectors protects motor neurons from apoptotic death and extends the life span of these animals (Suzuki et al., 2008, Li et al., 2006). GDNF expression in peripheral muscles of G93A SOD transgenic animals was shown to be reduced compared to control animals. It was further shown that the injection of the Shh pathway antagonist Cyclopamine into the calf muscles of end stage G93A SOD mice causes a 27 fold up-regulation of GDNF and a 20 fold up-regulation of CNTF.

GDNF Expression Pattern

GDNF is a target derived neurotrophic factor for developing DA neurons (Oo et al., 2003) and a postnatal survival factor for midbrain DA neurons (reviewed in Krieglstein, 2004 and Sariola & Saarma, 2003). GDNF protects DA neurons from the effects of neurotoxins such as MPTP (Airaksinen and Saarma, 2002; Kordower et al., 2003). The tissue specific ablation of the GDNF receptor Ret from DA neurons (Kramer et al., 2007) or the conditional ablation of GDNF in the adult animal (Pascual et al., 2008) cause progressive and late degeneration of the nigrostriatal system demonstrating the relevance of GDNF signaling for the survival of SNpc neurons in vivo. The relevant source of GDNF in the adult brain, however, has not been identified.

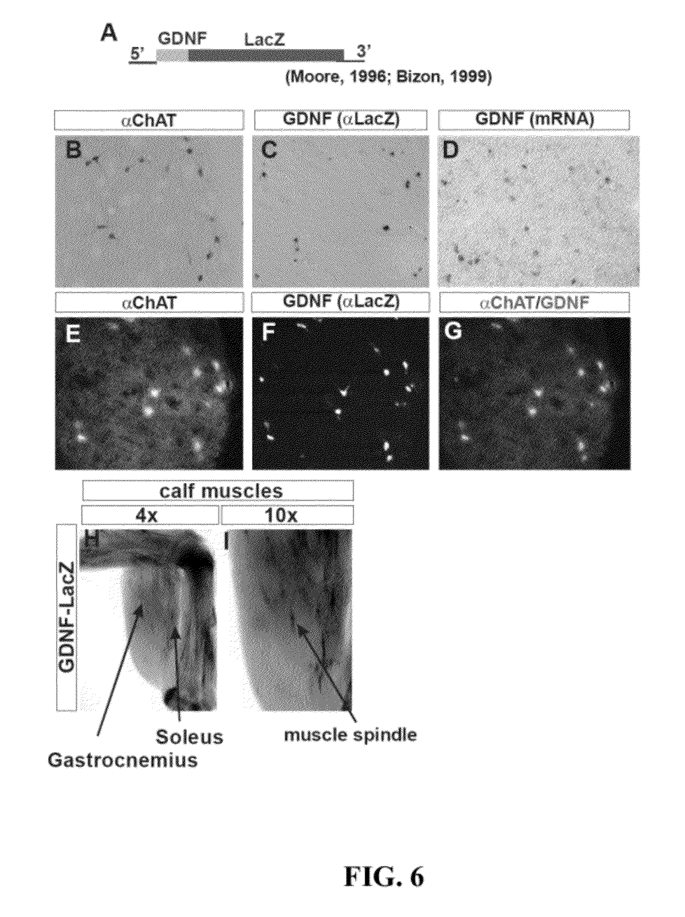

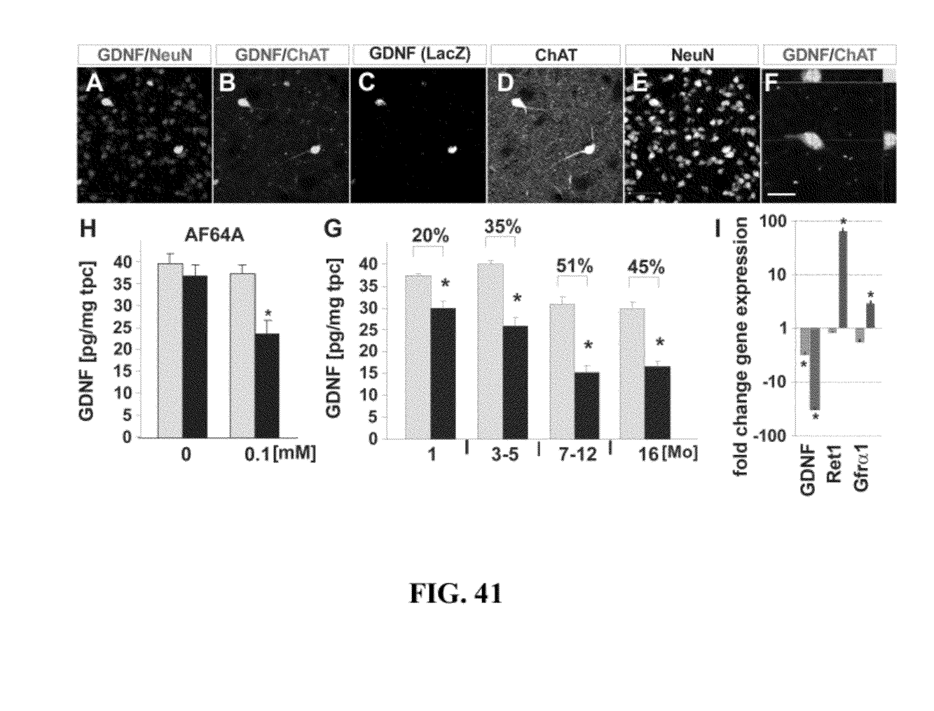

A heterozygous LacZ based indicator mouse was utilized for GDNF expression, in which the β-Gal gene is inserted 3′ to the mRNA cap site in the endogenous GDNF locus via homologous recombination (Moore et al., 1996; FIG. 6A). This methodology sidesteps possible confounding technical difficulties arising from either immuno-histochemical detection of a secreted factor like GDNF or from the detection of the mRNA coding for GDNF in combination with the determination of cell identity. As shown in FIG. 6, within the striatum the pattern of cells which are immuno-positive for ChAT is qualitatively and quantitatively highly similar to the pattern of cells that express LacZ in the GDNF-lacZ expression tracer mouse line and of cells that express GDNF mRNA (FIGS. 6B-D). Confocal double fluorescent immunohistochemistry for ChAT and LacZ expression then reveals that GDNF and ChAT is co-expressed in all striatal cholinergic neurons of the adult brain. Since DA neurons of the mesencephalon project massively to the striatum where they form monosynaptic connections with cholinergic neurons (Pisani et al., 2007), all DA neurons are exposed to GDNF produced by striatal cholinergic neurons (FIGS. 41A-F).

That cholinergic neurons of the adult striatum are a source of GDNF pharmacologically in wild-type adult mice was confirmed via the injection of the cholinotoxin AF64a (Leventer S M, et al., Neuropharmacology. 1987 April; 26(4):361-5; Sandberg K, et al., Brain Res. 1984 Feb. 13; 293(1):49-55; Fan Q I, et al. Neurochem Res. 1999 January; 24(1):15-24; and Hanin I. Life Sci. 1996; 58(22):1955-64). Unilateral injection of 1 μl of a 0.1 mM solution of AF64a into the striatum results into 35% reduction in GDNF tissue content when analyzed by quantitative ELISA (FIG. 41H). These results were further corroborated by analyzing GDNF protein and RNA expression in an animals model with progressive cholinergic neuron loss in the striatum. In these animals, a progressive reduction in the striatal tissue content (FIG. 41G) and mRNA expression (FIG. 41I) is found.

The same genetic gene expression tracer strategy was utilized to investigate the potential expression of GDNF in skeletal muscles (FIG. 6H-I). Chromogenic staining for LacZ activity in whole mount preparations of entire, skinned limbs, revealed that muscle spindles of all muscles express GDNF. In addition, certain muscles reveal LacZ expression in a subset of extrafusal fibers. The data herein confirm previous results (Vrieseling and Arber, 2006).

Shh Expression by Dopaminergic Neurons of the Mesencephalon

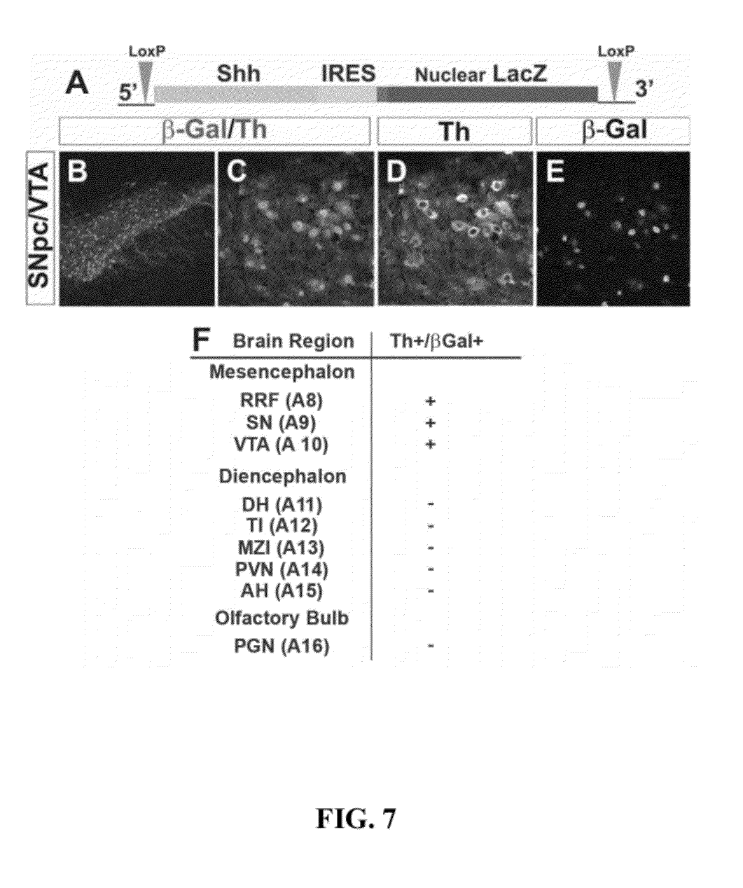

A recombinant allele of Shh from which a bicistronic RNA is transcribed that encodes both Shh and nuclear localized βGal, an expression tracer by homologous recombination in embryonic stem cells, was produced (FIG. 7A). This recombinant allele is a very useful experimental tool to reveal and identify unambiguously those cells in a multi-cellular setting that express Shh. In agreement with and extending on previously published studies of Shh in the adult brain by RNA in situ hybridization (Traiffort et al., 1999), expression of Shh is found in many brain nuclei, including motor neuron populations of the brain stem, the Purkinje cell layer of the cerebellum, and select neuronal populations in the hypothalamus, thalamus, cortex, hippocampus and olfactory bulb. In the mesencephalon, Shh expression is observed in virtually all Th+ cells in the substantia nigra pars compacta (SNpc), cell groups classified by Dahlstroem and Fuxe (1964) as “A9”, (FIG. 7B-E), the ventral tegmental area (VTA, “A10”, FIG. 7B) and the retro rubral field (RRF, “A8”) along the entire anterior posterior axis of these nuclei. Expression of Shh in dopaminergic neurons of the diencephalon (cell groups “A11”, “A12”, “A13”, and “A14”) and olfactory bulb (cell group “A16”; FIG. 7F) was not observed.

Tissue Specific Ablation of Shh from DA Neurons of the Mesencephalon

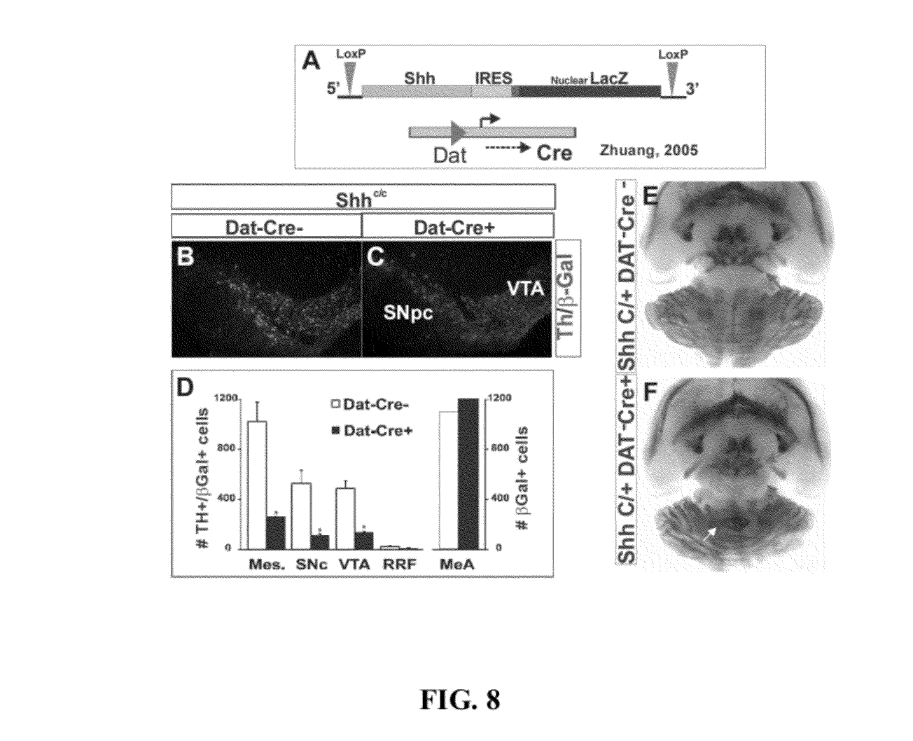

To begin to test the function of DA neuron produced Shh, animals with tissue specific, homozygous Shh ablations mediated by Cre activity expressed from the dopamine transporter locus (Dat-cre, Zuang et al., 2005, FIG. 8A) were produced. It was previously shown that the Dat-Cre allele leads to highly efficient (>95%) activation of a cre dependent lacZ reporter allele (Rosa26R, Soriano, 1999) in a dopamine neuron restricted manner from late embryonic stages to aged mice (Zhuang et al., 2005, Kramer et al., 2007). The efficiency and tissue specificity of Dat-Cre mediated recombination of the conditional Shh allele (ShhL) were assessed by quantifying the numbers of cells that had lost the expression of LacZ in mesencephalic dopaminergic neurons and in the medial amygdala (MeA) of 6 week old animals (FIG. 8B-D). An overall 80% reduction (with respect to Dat-Cre negative, ShhL mice, n=3, each hemisphere counted separately, p<0.01) in the number of lacZ/Th double positive neurons in the ventral midbrain is found (FIG. 8D). Recombination frequency was comparable among the dopaminergic neurons of the SN, VTA and the retro rubral field. LacZ expression in the MeA was not effected (FIG. 8D, FIG. 8F). To assess the tissue specificity of the recombination of the Shh conditional allele more globally in the adult brain, X-gal was used as enzymatic substrate for b-Gal activity in combination with “glass brain” whole mount preparations. Comparative analysis of optically flattened images of translucent, X-gal stained entire brains derived from (ShhL) and (Dat-Cre, ShhL) mice reveal overall highly similar patterns of β-gal activity with the exception of a pronounced absence of staining in ventral midbrain regions corresponding to the SN, VTA and retro rubral field which comprise a single continuous constellation of dopaminergic neurons approximating the form of an ellipsoid encircling the medial lemniscus, in Dat-Cre, ShhL mice (right-hand side arrows in FIG. 8E-F). Animals without Shh expression in DA neurons are produced with mendelian frequency and are mobile and active. These animals show no overt phenotype as young adults at 6 weeks of age.

Unilateral Injection of the Cholinotoxin Ethylcholine Mustard Aziridium (AF64a) into the Striatum and PPTg Upregulates Shh Expression in DA Neurons

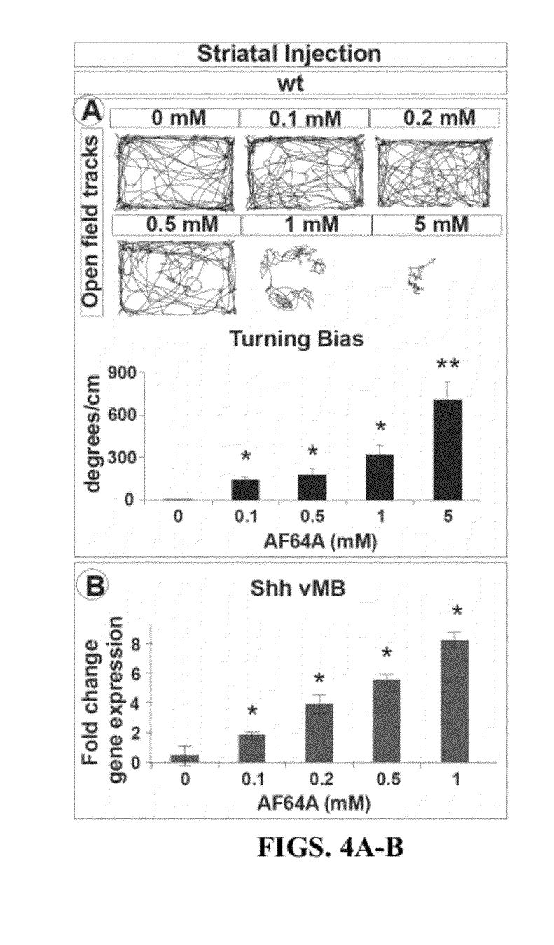

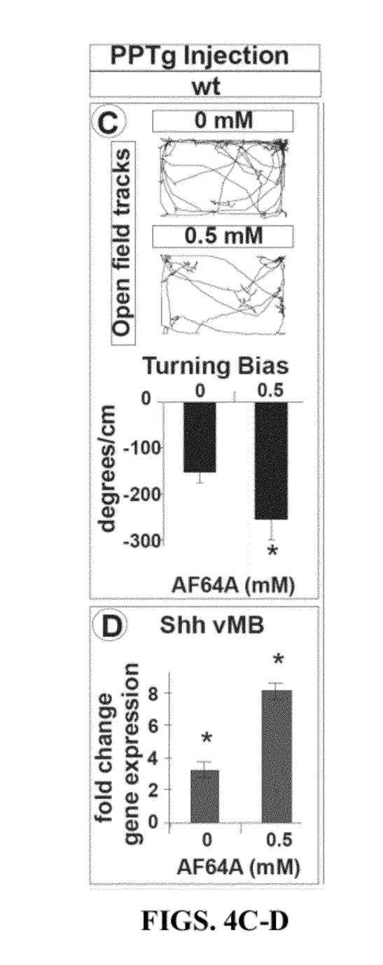

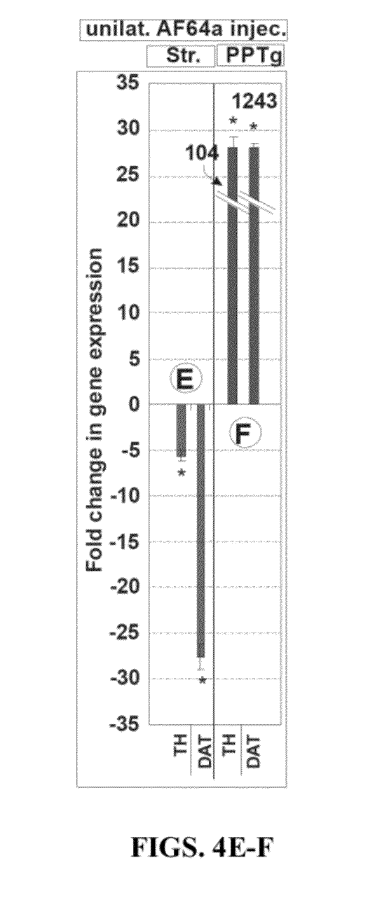

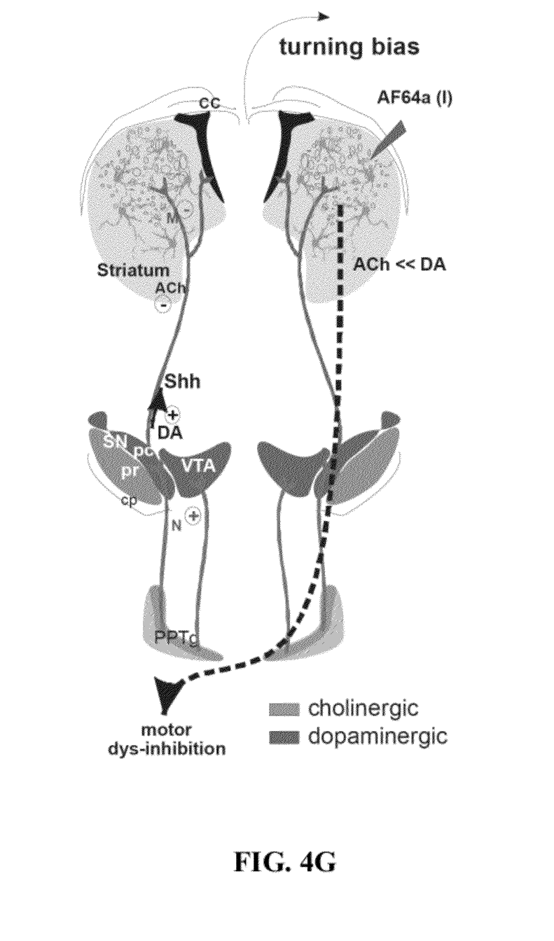

AF64a is a compound with structural similarities to choline, which acts as a competitive and reversible inhibitor of both Choline Transporter and Choline Acetyl Transferase (ChAT; Dudas et al., 2003; Amir et al., 1988; Leventer et al., 1987; Sandberg et al., 1984; Fan and Hanin, 1999). AF64a application causes an acute inhibition of—and physiological stress response in-cholinergic neurons (Hanin, 1996). A functional dose response for unilateral, striatal AF64a injection was first established by measuring the asymmetry of locomotor output 30 hours post injection of 8 week old wt C57Bl/6 male mice. An ipsilateral turning bias is observed, which increases from 0.1 mM to 5 mM AF64a. The observation of ipsilateral turning behavior is consistent with the muscarinic receptor mediated, inhibitory neuromodulatory role of acetylcholine in the striatum: A reduction in acetylcholine tone will lead to ipsilateral disinhibition of striatal motor output and contralateral increased spinal cord motor activity (FIG. 4G, FIG. 4A). Shh expression was quantified in the ventral midbrain (vMB) by quantitative rtPCR using “TAQman”-type expression assays for Shh (Applied Biosystems). A dose dependent, stepwise, 2 to 8 fold up-regulation of Shh expression in the ipsilateral vMB 36 h post striatal AF64 injection is found (FIG. 4B).

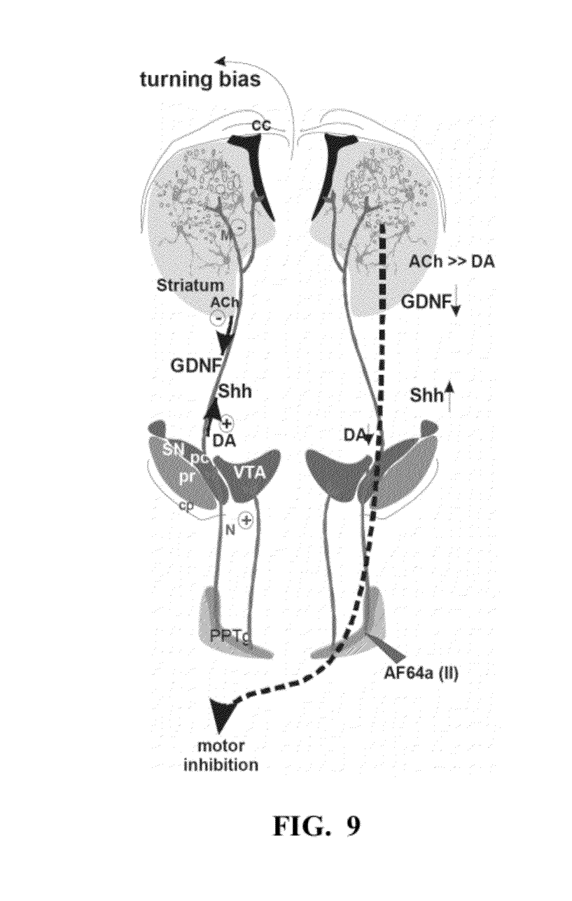

The PPTg provides monosynaptic, stimulatory, nicotinic receptor mediated cholinergic input to the SNpc (Futami et al., 1995; FIG. 9). Cholinotoxin injection into the PPTg elicits a contra lateral turning bias (negative values in FIG. 4C) consistent with a lower dopaminergic tone in the ipsilateral striatum due to reduced nicotinic receptor mediated cholinergic stimulation of the SNpc (FIG. 9). In these animals Shh expression in the ipsilateral vMB is 8 fold over expressed compared to the contra lateral control vMB (FIG. 4D).

Shh Up-Regulation in the Ventral Midbrain Down-Regulates GDNF Expression in the Striatum

The experiments described above established that Shh up-regulation is a common response to the injection of the cholinotoxin AF64a into the striatum and the PPTg. Hence AF64a injection into the PPTg of mice with genetic ablation of Shh from DA neurons allows one to investigate which genes, if any, in the experimentally uncompromised striatum are functionally regulated by Shh expression in the ventral midbrain.

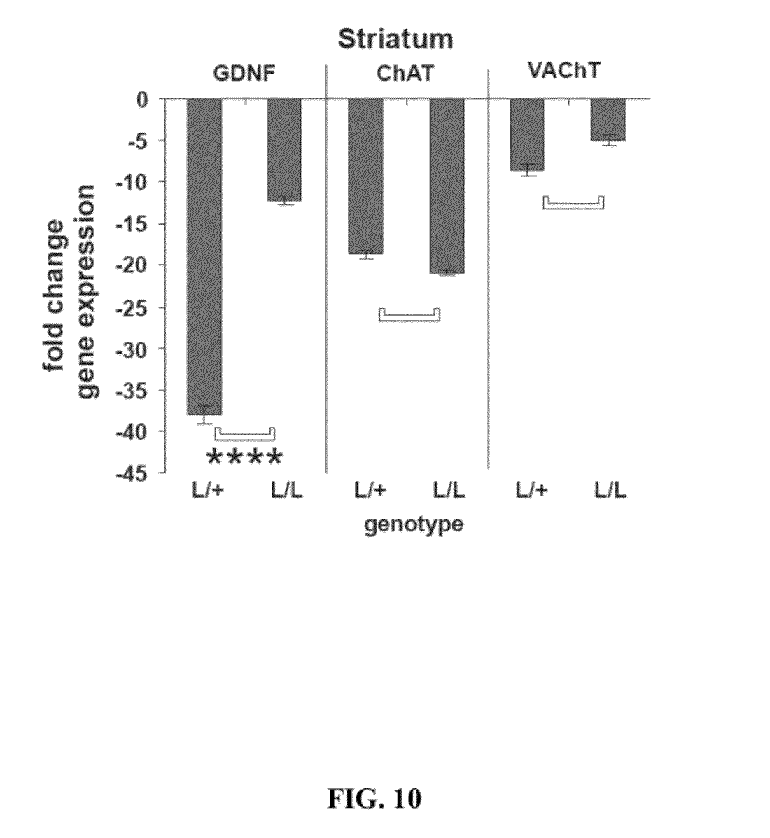

Using “TAQman”-type quantitative PCR expression assays for cholinergic markers on cDNA derived from striatal mRNA preparations, the expression of ChAT and vAChT in the striatum are repressed regardless of Shh expression by DA neurons (FIG. 10). However, there is a 35 fold down-regulation of GDNF expression in the striatum upon AF64a injection into the PPTg of control mice, i.e. mice that can express Shh in DA neurons, but only a 12 fold down-regulation in animals with genetic ablation of Shh from DA neurons (FIG. 10). These experiments demonstrate that GDNF expression in the striatum is functionally under negative transcriptional control through Shh signaling originating from mesencephalic DA neurons.

Shh Expression by Spinal Motor Neurons

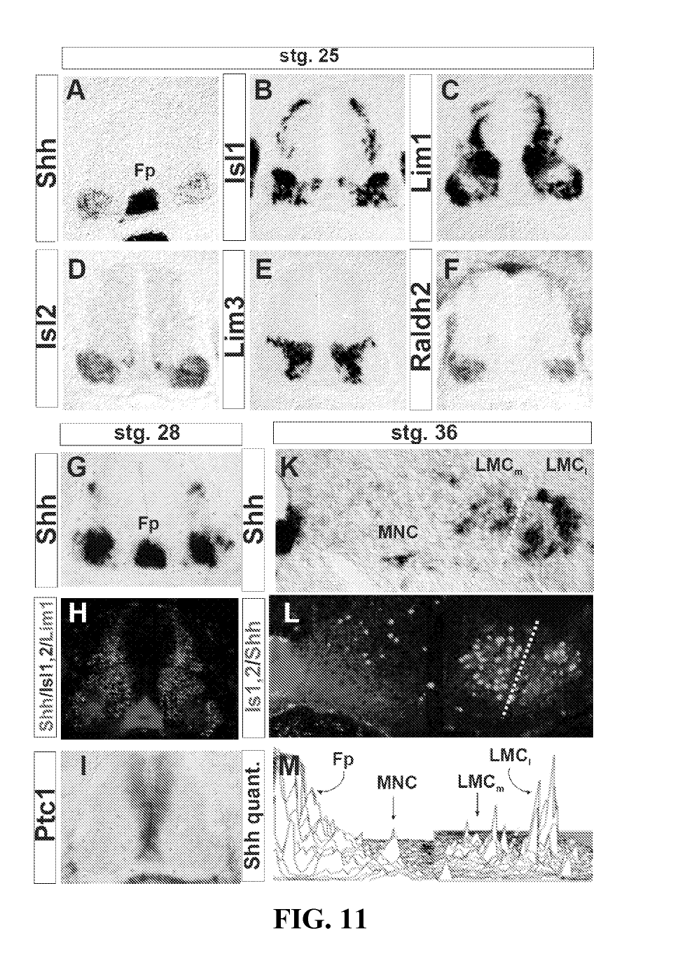

The mature Shh expression pattern in MN develops in chick and mouse in a stereotypic and conserved manner over a period of several days during MN ontogeny. In both species Shh becomes first expressed in brachial MNs once these MN have migrated to the extreme lateral margins of the ventral spinal cord (FIG. 11A).

Using markers for the temporal and spatial development of the columnar organization of the spinal MN system in chicken at all stages analyzed (Raldh2, Lim1, Isl1 Lim3, Isl2, ChAT), it was determined that Shh is expressed by MNs of all motor neuron columns (MNC, mMNC, medial and lateral halves of the lateral MNC, FIG. 11A-F).

At stage 28 in chick, Shh is expressed in MNs and floorplate (FP) at comparable levels (FIG. 11G). However, very little Shh protein is detectable in the ventral horns (FIG. 11H). There is also little to no expression of Ptc1, whose up-regulation is a sensitive, biomarker for the reception of a productive Shh signal, in ventral horns (FIG. 11I). Without being bound by theory, Shh produced by MN is mainly transported through MN neurites away from the MN soma at these developmental stages. At stg. 36, MN pool patterns are fully established in chick Immunohistochemical analysis of Shh expression reveals that many, but not all MNs of the MNC and LMCs express Shh (FIG. 11K and FIG. 11L). At these stages Shh protein is readily detectable in and around MNs in the ventral horns leaving open the possibility of a function for MN produced Shh locally within the spinal cord. Interestingly, pixel density quantification of Shh immuno-reactivity demonstrates that Shh expressing MNs of different pools express distinct levels of Shh (FIG. 11M).

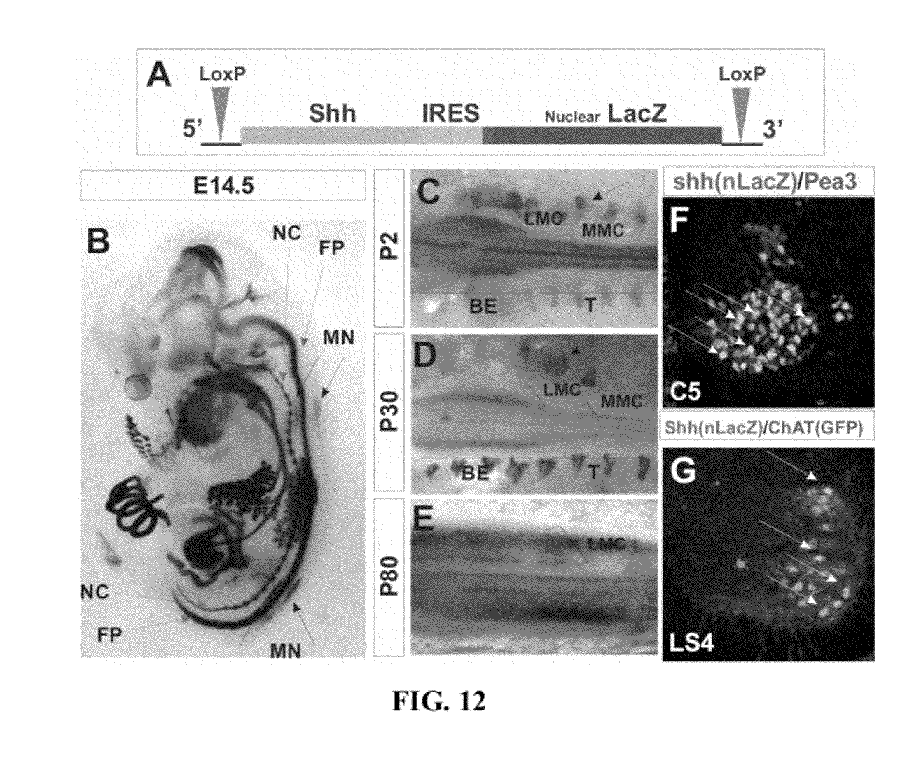

The pattern of Shh expression in MN in the mouse, as revealed by the nuclear LacZ expression tracer allele for Shh (FIG. 12A) appears fully mature by P2 and is then stable throughout life (FIG. 12C-E). Analysis of the large, readily identifiable Pectoralis MN pool at brachial levels utilizing the nLacZ expression tracer for Shh by triple fluorescent immunohistochemistry, confirms that only a subset of all Pea3 expressing Pectoralis MNs coexpress Shh (FIG. 12F). The restricted expression of Shh among MNs of all MNCs is maintained throughout life in mouse and it is estimated that, dependent on the MN pool, 20 to 50% of all MNs express Shh (FIG. 12G).

Tissue Specific Ablation of Shh from Motor Neurons

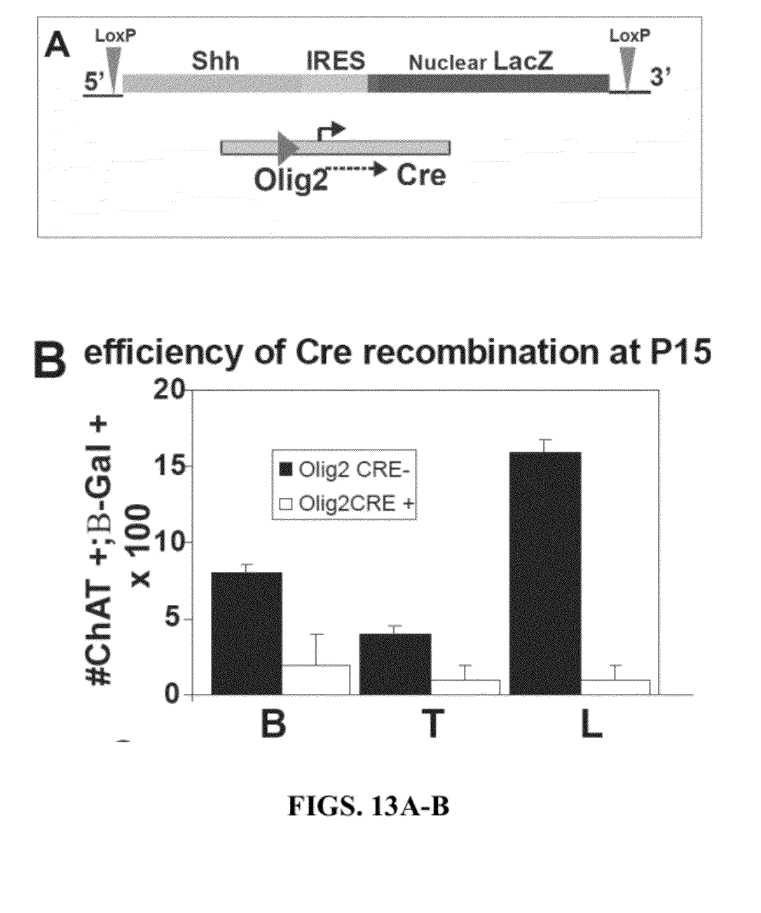

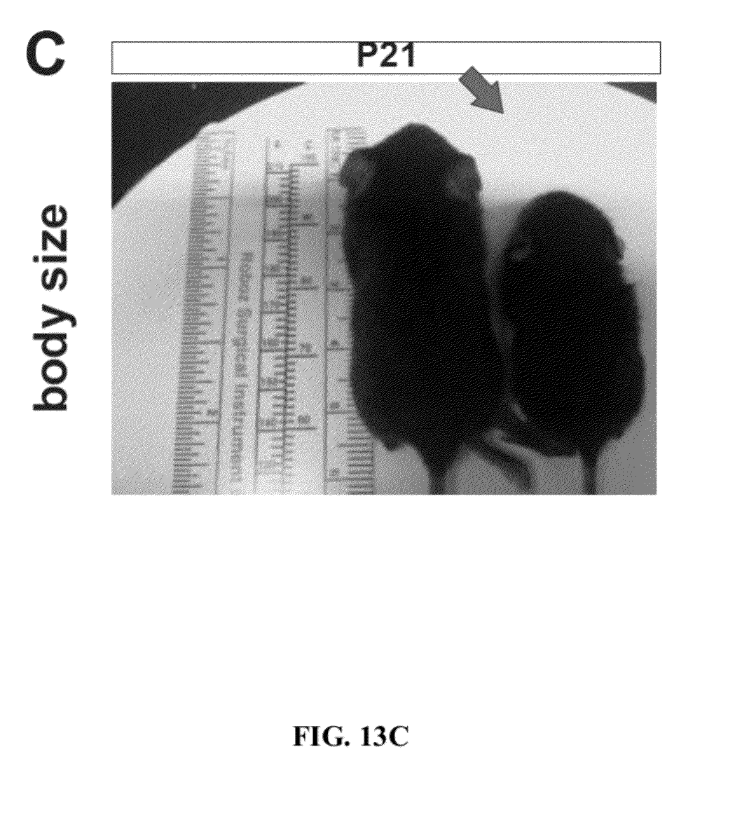

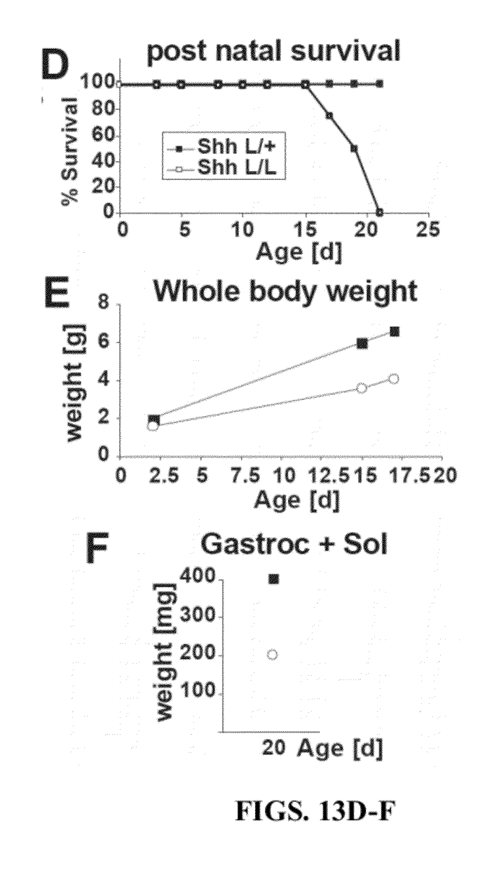

As a first step towards the functional characterization of MN expressed Shh, a conditional genetic ablation approach based on Cre activity expressed from the Olig2 Cre locus was used. The transcription factor Olig 2 is expressed selectively by MN precursors in the developing spinal cord (Novitch et al., 2001). In mice double heterozygous for the Olig2-cre and the conditional Shh allele (FIG. 13A), a better than 80% recombination efficiency in MNs along the entire spinal cord at E15 is observed (FIG. 13B). Animals heterozygous for Olig2Cre and homozygous for the conditional Shh allele (Olig2Cre, Shh L/L) are born alive and mobile with similar birth weight (FIG. 13E) but fail to thrive (FIG. 13C, FIG. 13E-F) despite active nursing evidenced by milk filled stomachs and die around 3 weeks of age (FIG. 13D).

Up-Regulation of GDNF in Skeletal Muscles in the Absence of Shh Expression by Spinal Motor Neurons

Based on previous work in the nigro striatal system, Shh expression in MNs can inhibit the expression of GDNF in the muscle. GDNF is expressed at high levels in the embryonic limb (Wang et al., 2002), but is down regulated post-natal and becomes restricted to muscle spindles (FIG. 6 Gould et al., 2008, Vrieseling and Arber, 2006).

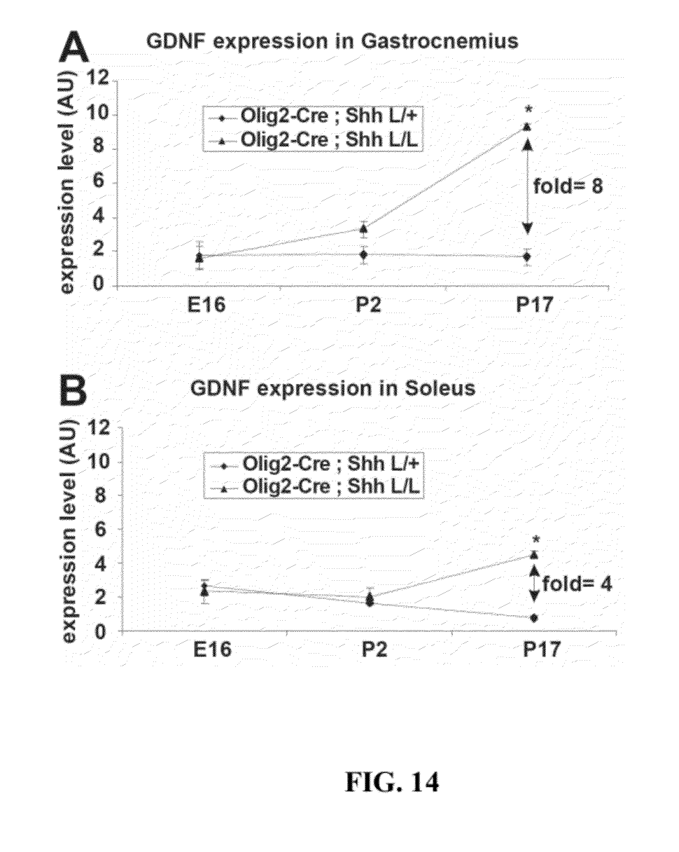

The expression of GDNF in two calf muscles, Gastrocnemius, a predominantly fast twitch muscle and Soleus, a predominantly slow twitch muscle, was therefore analyzed longitudinally using quantitative, rtPCR (“TaqMan” type expression assays). A 6 and respectively 5 fold up-regulation of GDNF expression in the Gastrocnemius and Soleus at p17 in the absence of MN expressed Shh was found (FIG. 14). These results demonstrate that the expression of GDNF in the limb is functionally under negative control through Shh signaling originating from MN. These results are consistent with findings in the nigro striatal system, where Shh expression by DA neurons inhibits GDNF expression by cholinergic neurons of the striatum.

The Transgenic G93A SOD Model of ALS

In the SOD G93A transgenic model of familial ALS temporally defined selective vulnerabilities of distinct MN synapses and axons precede the premature degeneration and death of lower MNs. Here, MNs innervating fast muscle fibers are affected at symptom-onset whereas MNs innervating slow muscle fibers are resistant initially and re-innervate vacated neuromuscular junctions (NMJs) on fast muscle fibers through terminal sprouting. Eventually, also MNs innervating slow muscle fibers succumb to the degenerative process. These studies demonstrate that physiological differences among related MN subtypes are critical determinants of disease progression (Frey et al., 2000, Pun et al., 2006).

In G93A SOD1 mice many peripheral synapses between MN and muscles (neuromuscular junctions, NMJs) are lost from P50 on, before detectable losses of motor axons in ventral roots exiting the spinal cord and long before any clinical sign of disease (Frey et al. 2000; Fisher et al. 2004). Muscle denervation occurs in a muscle specific temporal order and with stereotypic and specific topographic patterns within individual hindlimb muscles. These observations pointed to the possibility that the predictable patterns of denervation and eventual MN loss might reflect differences among motoneurons and/or muscle fibers. The soma of motoneurons innervating skeletal muscles are clustered in muscle specific “pools” along the anterior-posterior axis within the ventral horns of the spinal cord. Each pool consists of a muscle specific mixture of functionally distinct MN subtypes: fast-fatigable (FF), fast fatigue-resistant (FR) and slow (S), which show distinct excitability and recruitment properties and establish motor units with markedly different fatigue and force properties (Burke et al. 1994). The distinct and characteristic motoneuron subtype compositions of each pool of MN innervating different muscles, determines the functional properties of each muscle (Burke et al. 1994). The characteristic patterns of selective denervation in FALS might thus reflect selective vulnerabilities of subtypes of motoneurons, muscles and/or motor units.

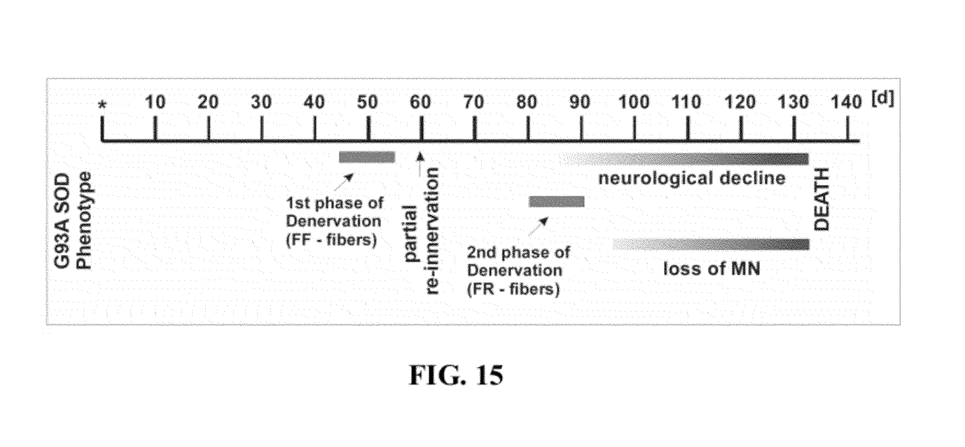

Previous work has provided strong evidence that different MN subtypes exhibit selective vulnerabilities towards degeneration in the G93A model of ALS. Here, Pun et al. (2006) exploited a combination of Thy1-transgenic mice expressing green fluorescent protein (GFP) fusion proteins in only a few neurons (De Paola et al. 2003) to establish a quantitative map of the innervation of hindlimb muscle compartments by motoneurons and their functional subtypes in the mouse. They then applied these maps to elucidate principles of early disease progression in FALS mice. Their results identify a stereotypical sequence of denervation with axons innervating fast-fatigable fibers degenerating first, followed by fast fatigue-resistant fiber innervating axons. In contrast, motoneuron axons innervating slow muscle fibers resist the disease and compensate through sprouting and reinnervation (FIG. 15). The axonal vulnerability process was alleviated by peripheral applications of CNTF (Pun et al., 2006).

In summary, the current data demonstrate the existence of factor(s) expressed in a “subpool” specific pattern in motor neurons. Such factors will take part in the determination of the different physiological properties of MN and can influence the degree of vulnerability of MN towards mutant SOD function.

Up Regulation of Shh in MNs and Down-Regulation of GDNF and CNTF in Skeletal Muscles in the Course of the SOD Phenotype

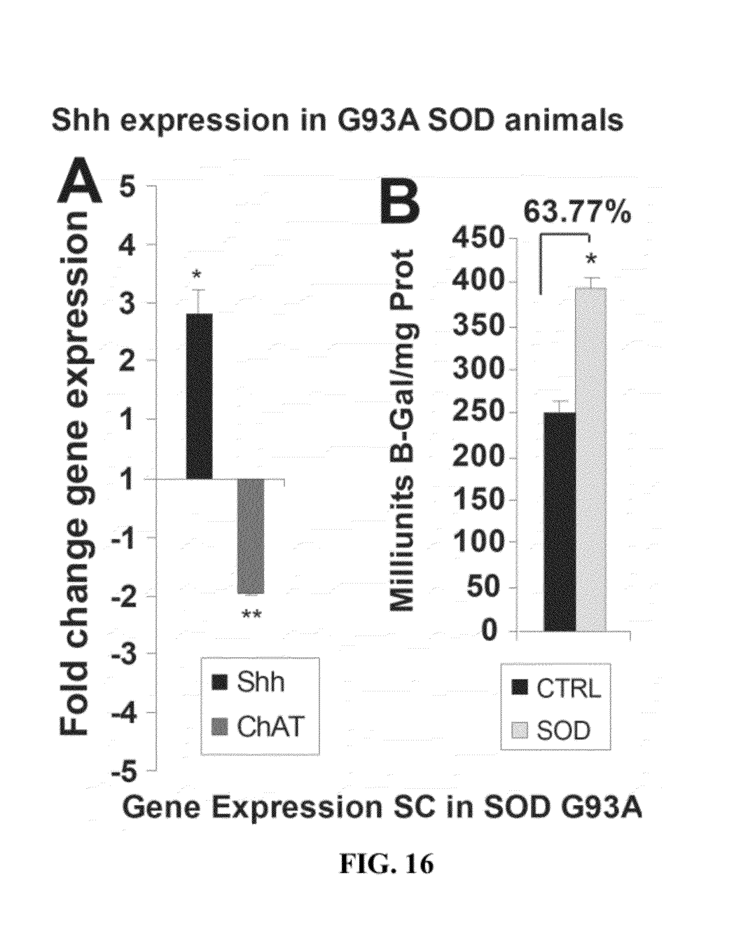

Based on the expression- and genetic loss of function-studies herein, Shh can be a factor whose dynamic expression in MN can modify the disease progression in ALS. To test this, the expression of Shh in the ventral spinal cord was quantified by quantitative rtPCR (“TAQman” type expression assays) and by measuring β-gal activity in animals that were double heterozygous for the G93A SOD transgene and the conditional Shh IRES lacZ gene expression allele (FIG. 12A). As shown in FIG. 16, an increase in Shh mRNA, but a decrease in ChAT expression in 125 day old double heterozygous animals compared to heterozygous Shh IRES lacZ controls, were found. At this age MN death is rampant in experimental animals (FIG. 15) causing the reduction in ChAT expression. Hence, the up regulation of Shh in the MNs that are still alive at this time is much higher than the measured 2.5 fold. Consistent with the mRNA data, β-gal activity is also increased (FIG. 16B).

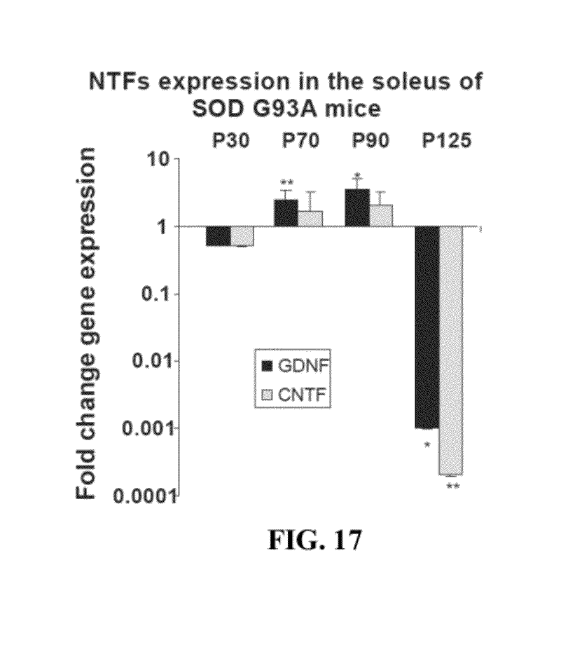

The expression of GDNF and CNTF in the soleus muscle as a function of age of the animal was analyzed in a longitudinal study design through the course of phenotype development in the transgenic G93A SOD model by quantitative rtPCR (“TaQMan type expression assays). As shown in FIG. 17, GDNF and CNTF expression is decreased about 1000 fold and 5000 fold (resp.; i.e. trophic factor expression is completely switched off) in the Soleus muscle of 125 day old G93A SOD transgenic animals.

Pharmacological Inhibition of Shh Signaling in Soleus Muscle of Endstage G93a SOD Transgenic Animals Up-Regulates GDNF and CNTF Expression Above Control Levels

The inverse correlation of Shh and GDNF expression and increase in peripheral GDNF expression in mice with genetic ablation of Shh expression by motor neurons is consistent with a scenario in which Shh signaling inhibits GDNF expression in a direct fashion. It follows that application of Shh antagonists to the muscle should relieve Shh mediated repression of GDNF and CNTF expression. This was tested through injection of cyclopamine, a widely used, generic antagonist of Shh signaling, into the soleus of endstage G93A SOD transgenic animals.

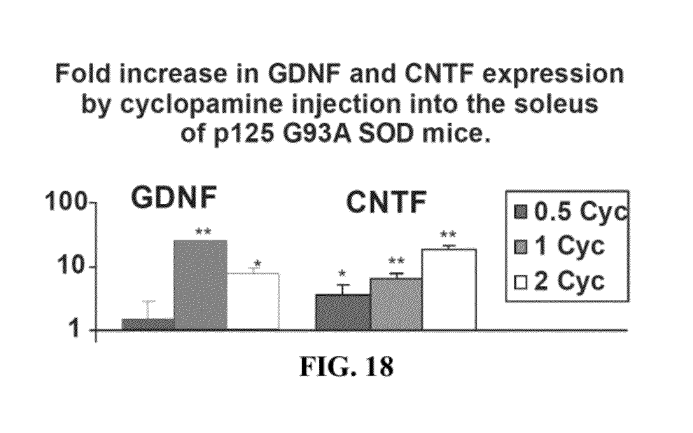

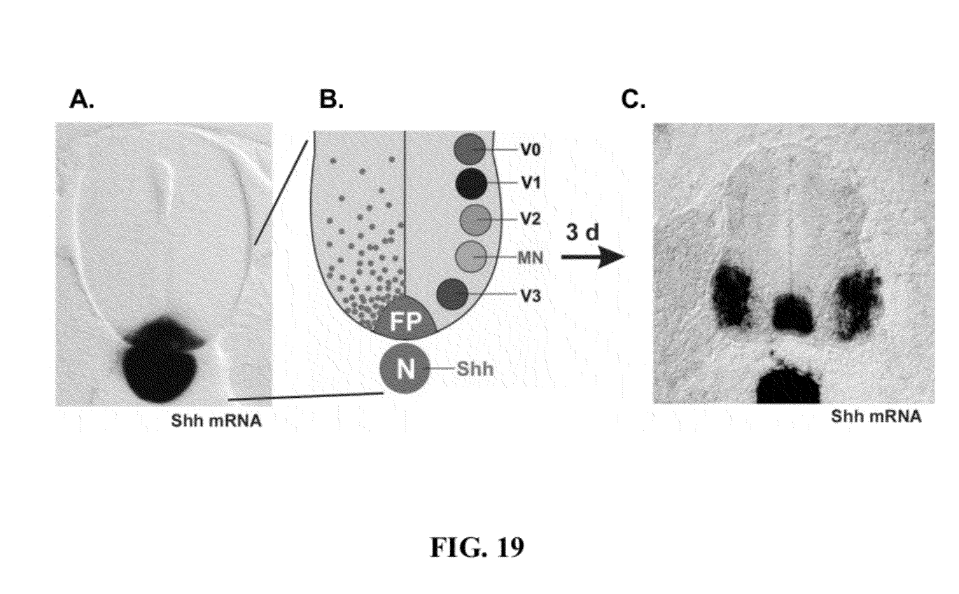

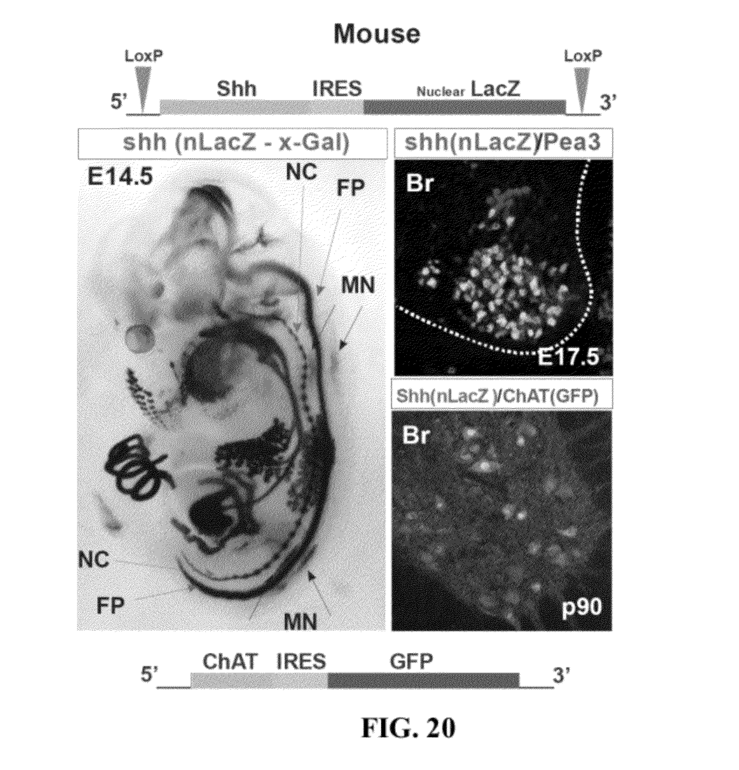

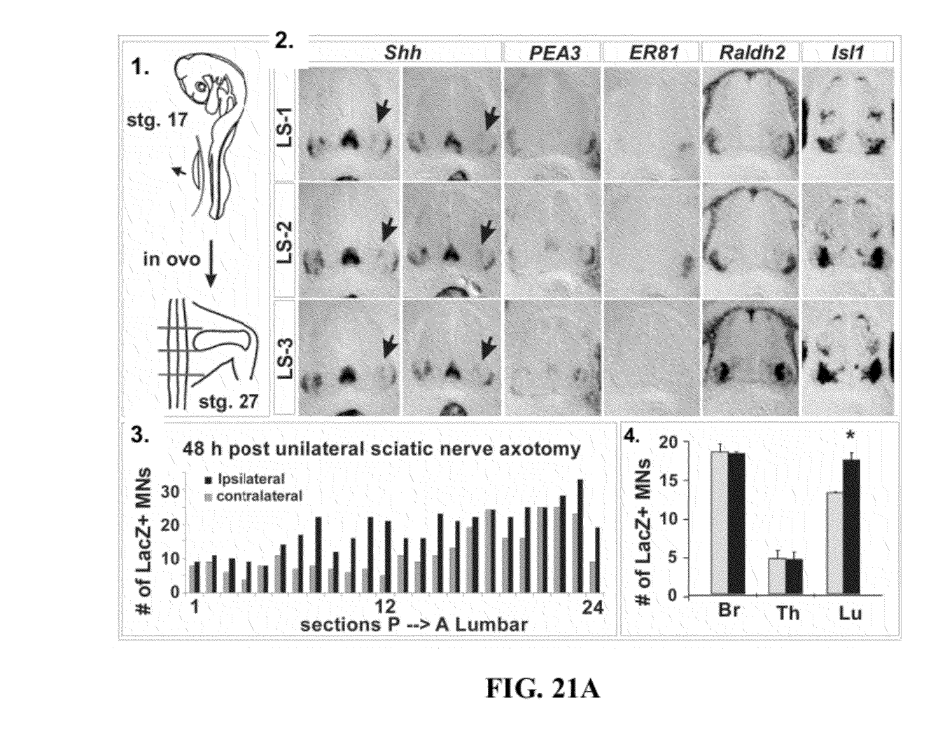

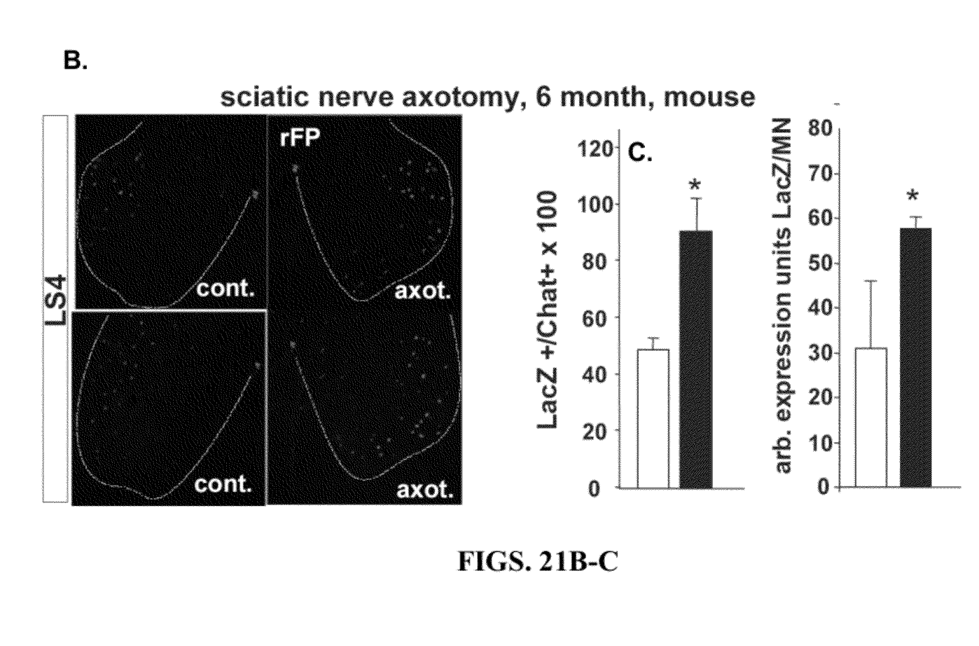

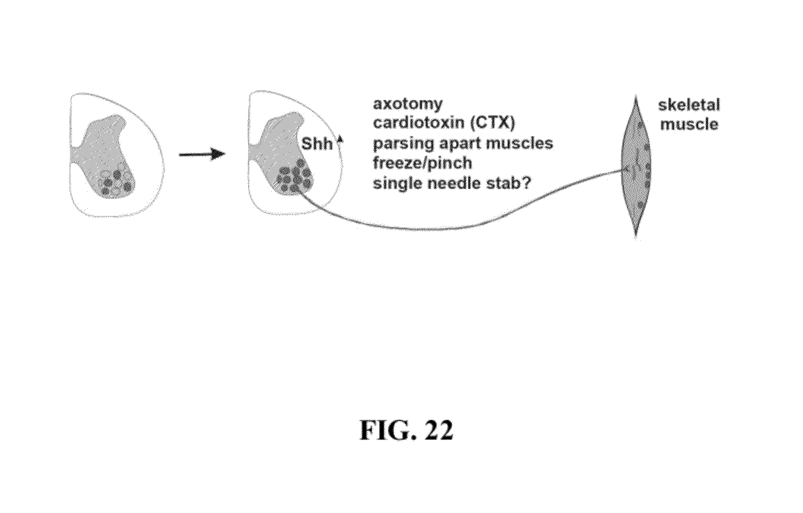

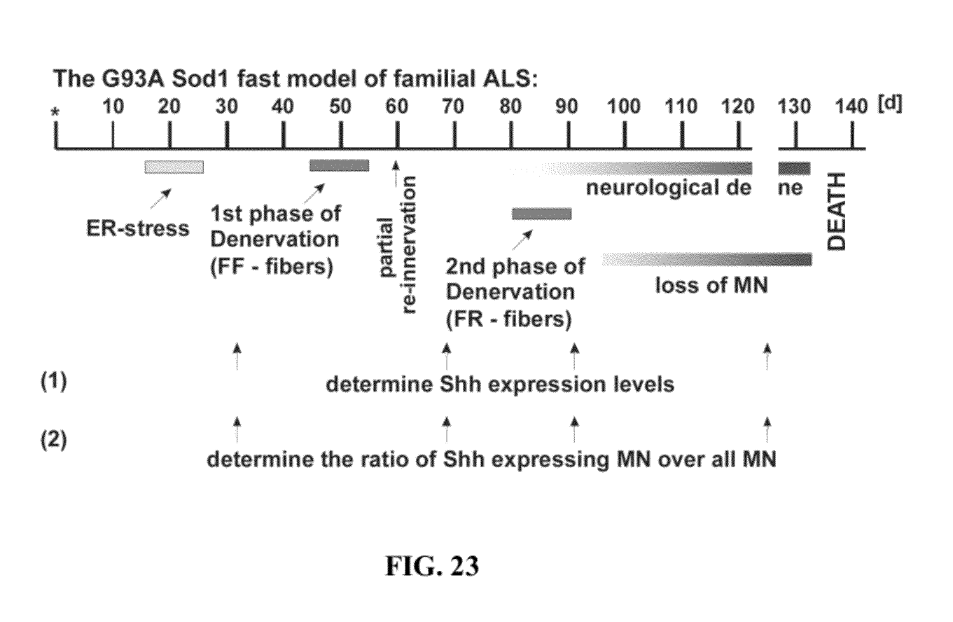

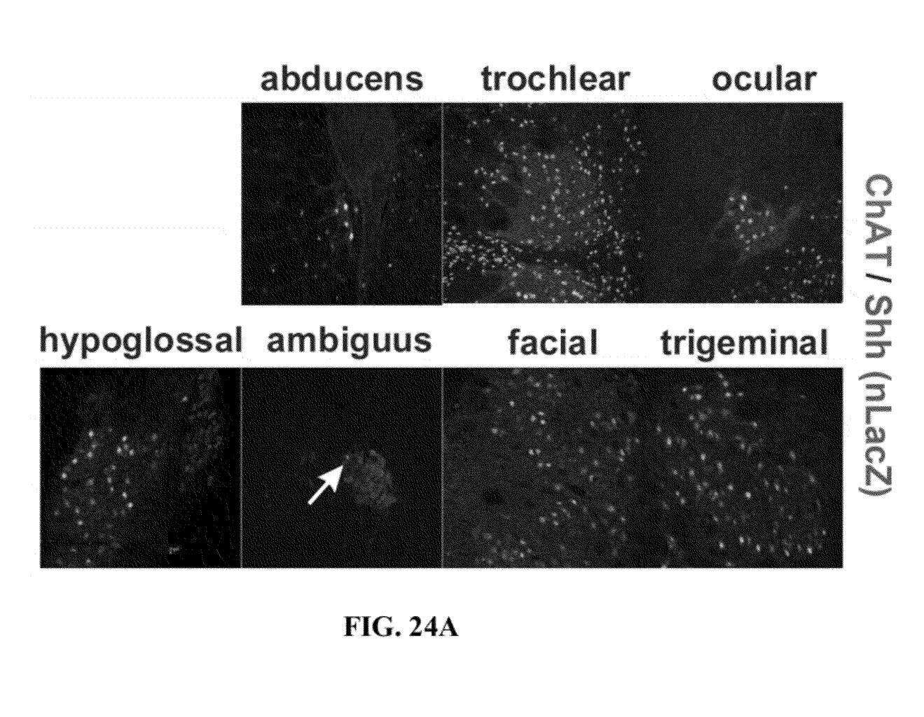

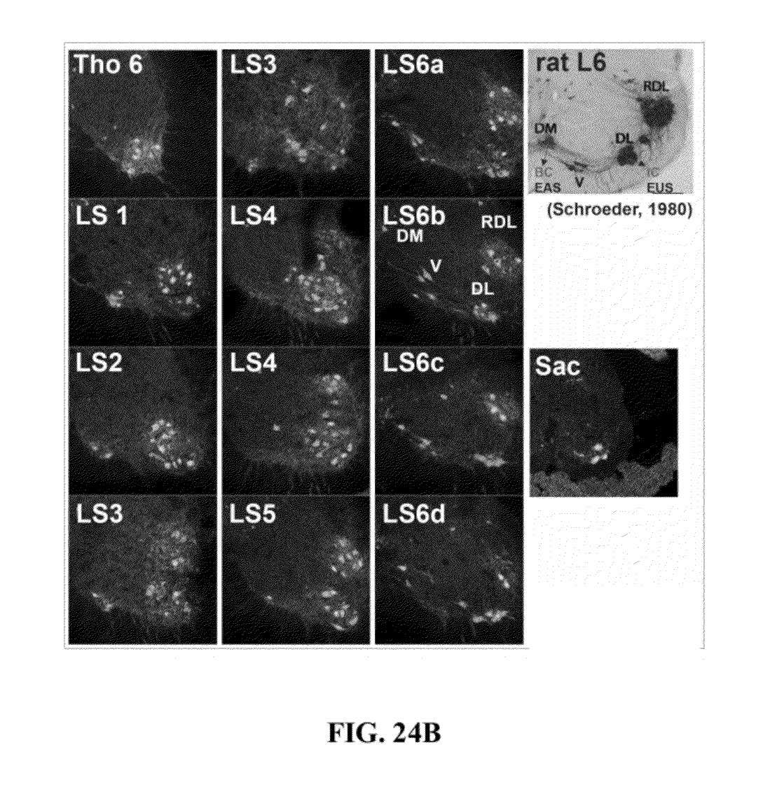

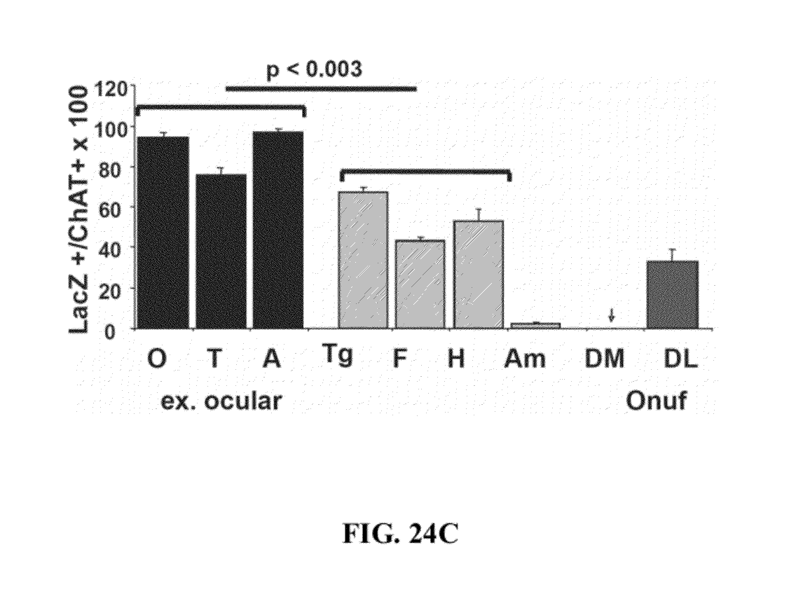

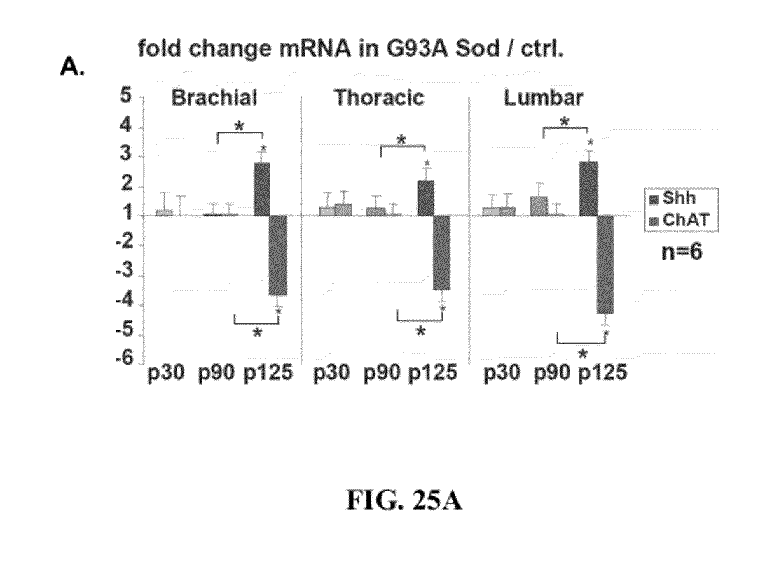

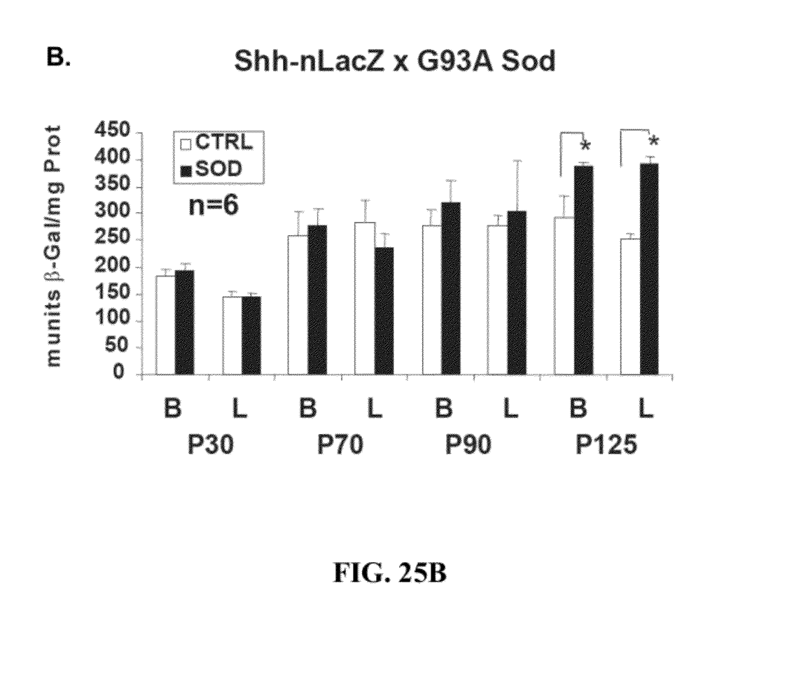

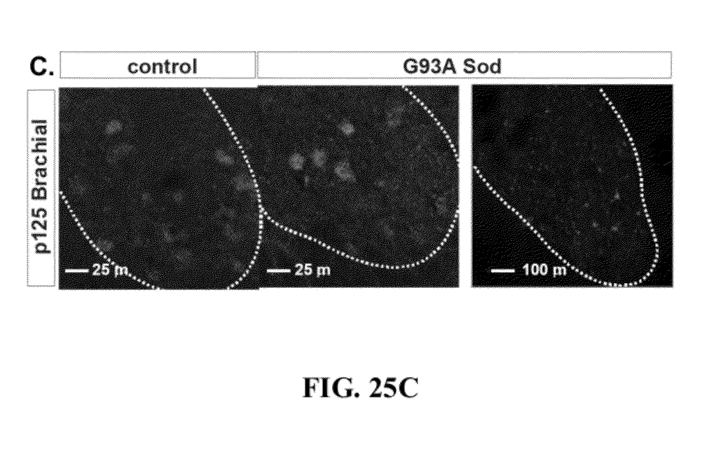

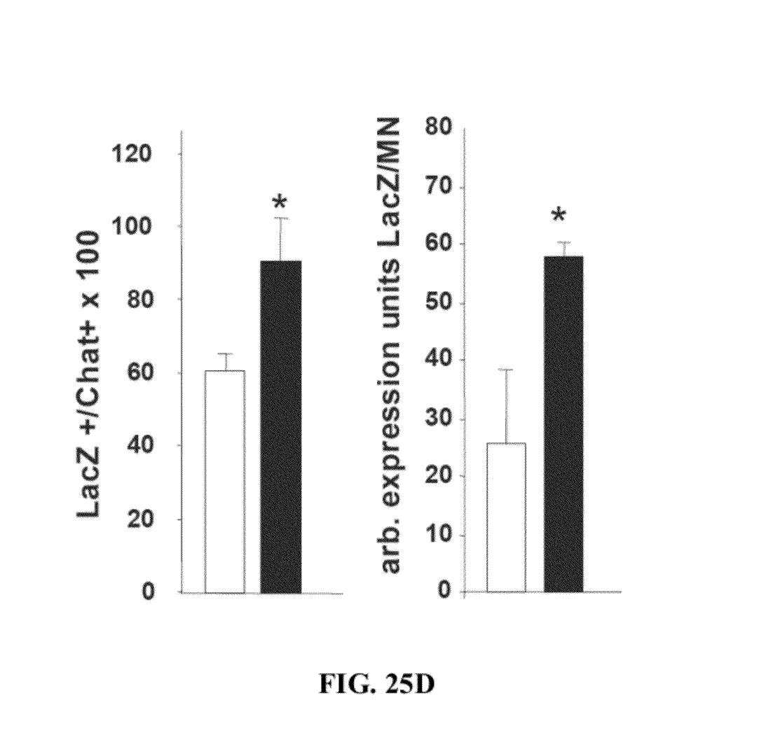

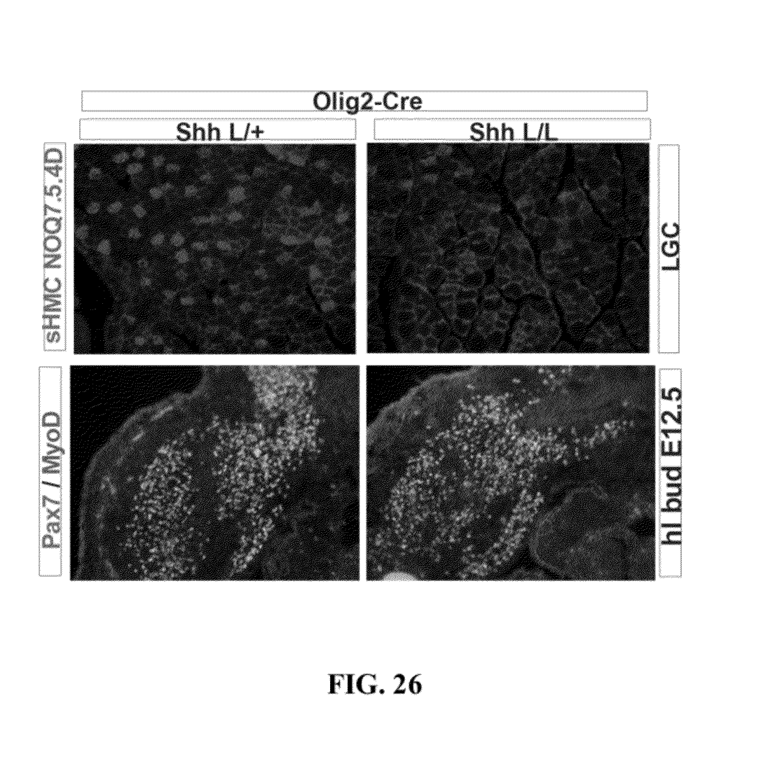

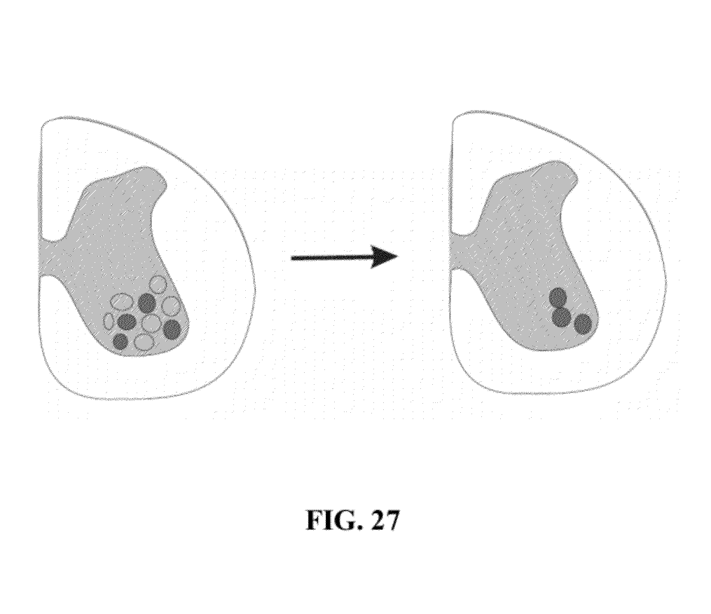

As shown in FIG. 18A-B, the unilateral injection of cyclopamine into the soleus of 125 day old G93A SOD transgenic animals leads to a dose dependent up-regulation of neurotrophic factor expression resulting in a 12 fold increase in GDNF—and a 8 fold increase in CNTF-expression over the saline injected contra lateral control soleus.

These experiments demonstrate that the pharmacological inhibition of the Shh pathway leads to an up-regulation of GDNF in the face of increased Shh production centrally and demonstrates that even in end stage animals the muscle remains sensitive to Shh signaling and competent to express GDNF.

### REFERENCES

Acsadi, G, Anguelov, RA, Yang, H, Toth, G, Thomas, R, Jani, A et al. (2002). Increased survival and function of SOD1 mice after glial cell-derived neurotrophic factor gene therapy. Hum Gene Ther 13: 1047-1059.
Airaksinen M S, Saarma M. (2002) The GDNF family: signalling, biological functions and therapeutic value. Nat Rev Neurosci. 2002 May; 3(5):383-94.
Akerud, P., Canals, J. M., Snyder, E. Y. & Arenas, E. Neuroprotection through delivery of glial cell line-derived neurotrophic factor by neural stem cells in a mouse model of Parkinson's disease. J. Neurosci. 21, 8108-8118 (2001).
Amir A, Pittel Z, Shahar A, Fisher A, Heldman E. Cholinotoxicity of the ethylcholine aziridinium ion in primary cultures from rat central nervous system. Brain Res. 1988 Jun. 28; 454(1-2):298-307.
Arenas, E., Trupp, M., Akerud, P. & Ibanez, C. F. GDNF prevents degeneration and promotes the phenotype of brain noradrenergic neurons in vivo. Neuron 15, 1465-1473 (1995).
Bespalov M M, Saarma M (2007) GDNF family receptor complexes are emerging drug targets. Trends Pharmacol Sci. 2007 February; 28(2):68-74. Epub 2007 January 10
Bezard E, Baufreton J, Owens G, Crossman A R, Dudek H, Taupignon A, Brotchie J M. (2003) Sonic hedgehog is a neuromodulator in the adult subthalamic nucleus. FASEB J. 17: 2337-2338.
Blesch, A and Tuszynski, M H (2001). GDNF gene delivery to injured adult CNS motor neurons promotes axonal growth, expression of the trophic neuropeptide CGRP, and cellular protection. J Comp Neurol 436: 399-410.
Burke R E. (1994) Physiology of motor units. In Myology. 464-484
Chiang C, Litingtung Y, Lee E, Young K E, Corden J L, Westphal H, Beachy P A. (1996) Cyclopia and defective axial patterning in mice lacking Sonic hedgehog gene function. Nature. 383(6599):407-13.
Choi-Lundberg, D. L. et al. Dopaminergic neurons protected from degeneration by GDNF gene therapy. Science 275, 838-841 (1997).
DAHLSTROEM A, FUXE K. (1964) EVIDENCE FOR THE EXISTENCE OF MONOAMINE-CONTAINING NEURONS IN THE CENTRAL NERVOUS SYSTEM. I. DEMONSTRATION OF MONOAMINES IN THE CELL BODIES OF BRAIN STEM NEURONS. Acta Physiol Scand Suppl. 1964:SUPPL 232:1-55.
De Paola V, Arber S, Caroni P. (2003) AMPA receptors regulate dynamic equilibrium of presynaptic terminals in mature hippocampal networks. Nat. Neurosci. 6:491-500.
Deshpande, DM, Kim, YS, Martinez, T, Carmen, J, Dike, S, Shats, I et al. (2006). Recovery from paralysis in adult rats using embryonic stem cells. Ann Neurol 60: 32-44.|
Dudas B, Rose M, Hanin I. Dose-dependent effect of cholinotoxin AF64A on the cholinergic elements of the cingulum bundle in rat. Brain Res. 2003 Mar. 7; 965(1-2):295-8.
Fan Q I, Hanin I. Effects of AF64A on gene expression of choline acetyltransferase (ChAT) in the septo-hippocampal pathway and striatum in vivo. Neurochem Res. 1999 January; 24(1):15-24.
Fisher L R et al. (2004) Amyotrophic lateral sclerosis is a distal axonopathy: evidence in mice and man. Exp. Neurol. 185:232-240.
Frey D et al. (2000) Early and selective loss of neuromuscular synapse subtypes with low sprouting competence in motoneurons diseases. J. Neurosci. 20:2534-2542
Futami T, Takakusaki K, Kitai S T (1995) Glutamatergic and cholinergic inputs from the pedunculopontine tegmental nucleus to dopamine neurons in the substantia nigra pars compacta. Neurosci Res. 1995 February; 21(4):331-42
Gash, D. M. et al. Functional recovery in parkinsonian monkeys treated with GDNF. Nature 380, 252-255 (1996).
Gill, S. S. et al. Direct brain infusion of glial cell line-derived neurotrophic factor in Parkinson disease. Nat. Med. 9, 589-595 (2003).
Glazner, G W, Mu, X and Springer, J E (1998). Localization of glial cell line-derived neurotrophic factor receptor alpha and c-ret mRNA in rat central nervous system. J Comp Neurol 391: 42-49.
Gould T W, Yonemura S, Oppenheim R W, Ohmori S, Enomoto H. (2008) The neurotrophic effects of glial cell line-derived neurotrophic factor on spinal motoneurons are restricted to fusimotor subtypes. J. Neurosci. 2008 Feb. 27; 28(9):2131-46.
Green-Sadan, T., N. Kinor, I. Roth-Deri, et al. 2003. Transplantation of glial cell line-derived neurotrophic factor-expressing cells into the striatum and nucleus accumbens attenuates acquisition of cocaine self-administration in rats. Eur. J. Neurosci. 18: 2093-2098.
Green-Sadan, T., Y. Kuttner, T. Lublin-Tennenbaum, et al. 2005. Glial cell line-derived neurotrophic factor-conjugated nanoparticles suppress acquisition of cocaine self-administration in rats. Exp. Neurol. 194: 97-105.
Hanin I. The AF64A model of cholinergic hypofunction, Life Sci. 1996; 58(22):1955-64.
He, D. Y., N. N. McGough, A. Ravindranathan, et al. 2005. Glial cell line-derived neurotrophic factor mediates the desirable actions of the anti-addiction drug ibogaine against alcohol consumption. J. Neurosci. 25: 619-628.
Henderson, C E, Phillips, H S, Pollock, R A, Davies, A M, Lemeulle, C, Armanini, M et al. (1994). GDNF: a potent survival factor for motoneurons present in peripheral nerve and muscle. Science 266: 1062-1064.
Hisaoka K, Takebayashi M. (2007) [Glia as targets for antidepressants: an involvement in glial cell line-derived neurotrophic factor [Article in Japanese], Nihon Shinkei Seishin Yakurigaku Zasshi. 2007 November; 27(5-6):173-9.
Hong M, Mukhida K, Mendez I. (2008) GDNF therapy for Parkinson's Disease. Expert Reviews in Neurotherapy 8: 1125-1139.
Keller-Peck, C R, Feng, G, Sanes, J R, Yan, Q, Lichtman, J W and Snider, W D (2001). Glial cell line-derived neurotrophic factor administration in postnatal life results in motor unit enlargement and continuous synaptic remodeling at the neuromuscular junction. J Neurosci 21: 6136-6146.
Kirik D, Georgievska B, Björklund A. Localized striatal delivery of GDNF as a treatment for Parkinson disease. Nat. Neurosci. 2004 February; 7(2):105-10. Epub 2004 January 27. Review.
Kordower J H. (2003) In vivo gene delivery of glial cell line—derived neurotrophic factor for Parkinson's disease. Ann Neurol. 2003; 53 Suppl 3:S120-32; discussion S132-4
Kordower, J. H. et al. Neurodegeneration prevented by lentiviral vector delivery of GDNF in primate models of Parkinson's disease. Science 290, 767-773 (2000).
Kramer E R, Aron L, Ramakers G M, Seitz S, Zhuang X, Beyer K, Smidt M P, Klein R. Absence of Ret signaling in mice causes progressive and late degeneration of the nigrostriatal system. PLoS Biol. 2007 March; 5(3):e39.
Krieglstein K. (2004) Factors promoting survival of mesencephalic dopaminergic neurons. Cell Tissue Res. 2004 October; 318(1):73-80. Epub 2004 August 6
Leitner, ML, Molliver, D C, Osborne, P A, Vejsada, R, Golden, JP, Lampe, P A et al. (1999). Analysis of the retrograde transport of glial cell line-derived neurotrophic factor (GDNF), neurturin, and persephin suggests that in vivo signaling for the GDNF family is GFRalpha coreceptor-specific. J Neurosci 19: 9322-9331.
Leventer S M, Wulfert E, Hanin I. Time course of ethylcholine aziridinium ion (AF64A)-induced cholinotoxicity in vivo. Neuropharmacology. 1987 April; 26(4):361-5.
Lewis P M, Gritli-Linde A, Smeyne R, Kottmann A, McMahon A P. (2004) Sonic hedgehog signaling is required for expansion of granule neuron precursors and patterning of the mouse cerebellum. Dev Biol. 270(2):393-410.
Li W, Brakefield D, Pan Y, Hunter D, Myckatyn T M, Parsadanian A. (2007) Muscle-derived but not centrally derived transgene GDNF is neuroprotective in G93A SOD1 mouse model of ALS. Exp Neurol. 2007 February; 203(2):457-71. Epub 2006
Machold R, Hayashi S, Rutlin M, Muzumdar M D, Nery S, Corbin J G, Gritli-Linde A, Dellovade T, Porter J A, Rubin L L, Dudek H, McMahon A P, Fishell G. (2003) Sonic hedgehog is required for progenitor cell maintenance in telencephalic stem cell niches. Neuron. 39: 937-950
Messer, C. J., A. J. Eisch, W. A. Carlezon Jr., et al. 2000. Role for GDNF in biochemical and behavioral adaptations to drugs of abuse. Neuron 26: 247-257.
Mohajeri, M H, Figlewicz, D A and Bohn, M C (1999). Intramuscular grafts of myoblasts genetically modified to secrete glial cell line-derived neurotrophic factor prevent motoneuron loss and disease progression in a mouse model of familial amyotrophic lateral sclerosis. Hum Gene Ther 10: 1853-1866.
Moore M W, Klein R D, Fariñas I, Sauer H, Armanini M, Phillips H, Reichardt L F, Ryan A M, Carver-Moore K, Rosenthal A. (1996) Renal and neuronal abnormalities in mice lacking GDNF. Nature. 1996 Jul. 4; 382(6586):76-9
Niwa M, Yan Y, Nabeshima T. (2008) Genes and molecules that can potentiate or attenuate psychostimulant dependence: relevance of data from animal models to human addiction. Ann N Y Acad. Sci. 2008 October; 1141:76-95.
Niwa, M., A. Nitta, K. Yamada, et al. 2007. The roles of glial cell line-derived neurotrophic factor, tumor necrosis factor-a, and an inducer of these factors in drug dependence. J. Pharmacol. Sci. 104: 116-121
Novitch B G, Chen A I, Jessell T M. (2001) Neuron. 31(5):773-89.
Oo T F, Ries V. Cho J. Kholodilov, N Burke R E (2005) Anatomical basis of glial cell line derived neurotprhic factor expression in the striatum and related basal ganglia during postnatal development of the rat. J. Comp. Neurology 484:57-67.
Pascual A, Hidalgo-Figueroa M, Piruat J I, Pintado C O, Gómez-Diaz R, López-Barneo J. (2008) Absolute requirement of GDNF for adult catecholaminergic neuron survival. Nat. Neurosci. 2008 July; 11(7):755-61. Epub 2008 June 8
Pisani A, Bernardi G, Ding J, Surmeier D J. (2007) Re-emergence of striatal cholinergic interneurons in movement disorders. Trends Neurosci. 2007 October; 30(10):545-53.
Pothos E N, Davila V, Sulzer D. (1998) J. Neurosci. 1998 Jun. 1; 18(11):4106-18.
Pun, S., A. F. Santos, et al. (2006). Selective vulnerability and pruning of phasic motoneuron axons in motoneuron disease alleviated by CNTF. Nat Neurosci 9(3): 408-19.
PYM50028, a novel, orally active, nonpeptide neurotrophic factor inducer, prevents and reverses neuronal damage induced by MPP+ in mesencephalic neurons and by MPTP in a mouse model of Parkinson's disease.
Rosenblad, C., Martinez-Serrano, A. & Bjorklund, A. Neuroscience 82, 129-137 (1998)
Saavedra A, Baltazar G, Duarte E P. (2008) Driving GDNF expression: The green and the red traffic lights. Prog Neurobiol. 2008 November; 86(3):186-215. Epub 2008 September 7
Sandberg K, Hanin I, Fisher A, Coyle J T. Selective cholinergic neurotoxin: AF64A's effects in rat striatum. Brain Res. 1984 Feb. 13; 293(1):49-55.
Sariola H, Saarma M. (2003) Novel functions and signalling pathways for GDNF. J Cell Sci. 2003 Oct. 1; 116(Pt 19):3855-62.
Slevin, J. T. et al. Improvement of bilateral motor functions in patients with Parkinson disease through the unilateral intraputaminal infusion of glial cell line-derived neurotrophic factor. J. Neurosurg. 102, 216-222 (2005).
Soriano P (1999) Generalized lacZ expression with the ROSA26 Cre reporter strain. Nat. Genet. 1999 January; 21(1):70-1.
Suzuki M, McHugh J, Tork C, Shelley B, Hayes A, Bellantuono I, Aebischer P, Svendsen C N. (2008) Direct muscle delivery of GDNF with human mesenchymal stem cells improves motor neuron survival and function in a rat model of familial ALS. Mol. Ther. 2008 December; 16(12):2002-10. Epub 2008 September 16
Tokugawa K, Yamamoto K, Nishiguchi M, Sekine T, Sakai M, Ueki T, Chaki S, Okuyama S. (2003) Neurochem Int. 2003 January; 42(1):81-6.
Tomac A, Lindqvist E, Lin L F, Ogren S O, Young D, Hoffer B J, Olson L. Protection and repair of the nigrostriatal dopaminergic system by GDNF in vivo. Nature. 1995 Jan. 26; 373(6512):335-9.
Traiffort E, Charytoniuk D A, Faure H, Ruat M. (1998) Regional distribution of Sonic Hedgehog, patched, and smoothened mRNA in the adult rat brain. J. Neurochem. 70: 1327-1330.
Visanji N P, et al. PYM50028, a novel, orally active, nonpeptide neurotrophic factor inducer, prevents and reverses neuronal damage induced by MPP+ in mesencephalic neurons and by MPTP in a mouse model of Parkinson's disease FASEB J. 2008 July; 22(7):2488-97. Epub 2008 March 25
von Bartheld, CS, Wang, X and Butowt, R (2001). Anterograde axonal transport, transcytosis, and recycling of neurotrophic factors: the concept of trophic currencies in neural networks. Mol Neurobiol 24: 1-28.
Vrieseling E, Arber S. (2006) Target-induced transcriptional control of dendritic patterning and connectivity in motor neurons by the ETS gene Pea3. Cell. 2006 Dec. 29; 127(7):1439-52.
Wang J, Chen G, Lu B, Wu C P GDNF acutely potentiates Ca2+ channels and excitatory synaptic transmission in midbrain dopaminergic neurons. (2003). Neurosignals. 2003 March-April; 12(2):78-88.
Wang, C Y, Yang, F, He, X P, Je, H S, Zhou, J Z, Eckermann, K et al. (2002). Regulation of neuromuscular synapse development by glial cell line-derived neurotrophic factor and neurturin. J Biol Chem 277: 10614-10625.
Yan, Y., K. Yamada, M. Niwa, et al. 2007. Enduring vulnerability to reinstatement of methamphetamine-seeking behavior in glial-cell-line-derived neurotrophic factor mutant mice. FASEB J. 21: 1994-2004
Yang F, et al. GDNF acutely modulates excitability and A-type K(+) channels in midbrain dopaminergic neurons. (2001) Nat. Neurosci. 2001 November; 4(11):1071-8.
Yang, S. C., Markey, S. P., Bankiewicz, K. S., London, W. T., and Lunn, G. (1988). Recommended safe practices for using the neurotoxin MPTP in animal experiments. Lab. Anim. Sci. 38, 563-567.
Zhuang X, et al. (2005) Targeted gene expression in dopamine and serotonin neurons of the mouse brain. J Neurosci Methods. 143(1):27-32.

### Example 2

### Regulating of GDNF Expression in the Adult Organism by GDC-0449

The genetic and pharmacological experiments described herein will demonstrate that manipulating Shh mediated cell signaling with the Shh antagonist GDC-0449 will cause alterations in GDNF expression in the adult animal, e.g, inhibit endogenous expression of GDNF.

Pharmacological stimulation of endogenous GDNF production using low-molecular weight drugs that specifically activate the GDNF receptor or induce the expression of GDNF itself in relevant tissues can be administered systemically. To test this, it will be (a) determined whether there are relevant sources of GDNF in the adult organism; and (b) determined how GDNF expression is regulated in these tissues. Lead compounds will be identified that can regulate the expression of Shh in these tissues in the adult organism. To demonstrate that such a compound will lead to the upregulation of GDNF expression in relevant tissues, a validated model of a neurodegenerative disease whose disease course can be modified by GDNF application will be used.

The ascending, mesencephalic dopamine system and the cholinergic system of the basal forebrain, in aggregation, provide part of the anatomic substrate for a wide variety of neurodegenerative diseases (i.e. Parkinson's Disease, Alzheimer's, Huntington's, supra nuclear palsy and others), addiction, and psychosis (Schizophrenia). As discussed in Example 1, whether an endogenous source of GDNF in the adult brain exposes these neuronal nuclei to GDNF will be confirmed. The regulation of the expression of GDNF in these tissues was then studied.

The transgenic G93A SOD model of familial ALS is a well established model for progressive motor neuron degeneration. Elevating GDNF content in peripheral muscles of G93A SOD transgenic rats and mice either by the expression of GDNF from transplanted cells or from muscle specific transgenic expression vectors protects motor neurons from apoptotic death and extends the life span of these animals (Suzuki et al., 2008, Li et al., 2006). GDNF expression in peripheral muscles of G93A SOD transgenic animals was shown to be reduced compared to control animals. The Shh pathway antagonist GDC-0449 will be injected into the calf muscles of end stage G93A SOD mice to see its effect on GDNF and CTNF expression and regulation.

GDNF Expression Pattern

A heterozygous LacZ based indicator mouse in which the β-Gal gene is inserted 3′ to the mRNA cap site in the endogenous GDNF locus via homologous recombination, will be utilized for GDNF expression (Moore et al., 1996). This methodology sidesteps possible confounding technical difficulties arising from either immuno-histochemical detection of a secreted factor like GDNF or from the detection of the mRNA coding for GDNF in combination with the determination of cell identity. The pattern of cells which are immuno-positive for ChAT within the striatum will be examined as to whether they are qualitatively and quantitatively highly similar to the pattern of cells that express LacZ in the GDNF-lacZ expression tracer mouse line and of cells that express GDNF mRNA. Confocal double fluorescent immunohistochemistry will be used for ChAT and LacZ expression to examine whether GDNF and ChAT is co-expressed in all striatal cholinergic neurons of the adult brain.

The same genetic gene expression tracer strategy will be used to investigate the potential expression of GDNF in skeletal muscles. Chromogenic staining for LacZ activity in whole mount preparations of entire, skinned limbs will be performed.

Shh Expression by Dopaminergic Neurons of the Mesencephalon

The recombinant allele of Shh described in Example 1 will be used to reveal and identify those cells in a multi-cellular setting that express Shh. The expression of Shh in brain nuclei, including motor neuron populations of the brain stem, the Purkinje cell layer of the cerebellum, and select neuronal populations in the hypothalamus, thalamus, cortex, hippocampus and olfactory bulb, will be examined.

Tissue Specific Ablation of Shh from DA Neurons of the Mesencephalon

The function of DA neuron-produced Shh in animals with tissue specific, homozygous Shh ablations that is mediated by Cre activity expressed from the dopamine transporter locus, will be examined (Dat-cre, Zuang et al., 2005). To globally assess the tissue specificity of the recombination of the Shh conditional allele in the adult brain, X-gal will be used as enzymatic substrate for b-Gal activity in combination with “glass brain” whole mount preparations.

Unilateral Injection of the Cholinotoxin Ethylcholine Mustard Aziridium (AF64a) into the Striatum and PPTg

AF64a is a compound with structural similarities to choline, which acts as a competitive and reversible inhibitor of both Choline Transporter and Choline Acetyl Transferase (ChAT; Dudas et al., 2003; Amir et al., 1988; Leventer et al., 1987; Sandberg et al., 1984; Fan and Hanin, 1999). AF64a application causes an acute inhibition of—and physiological stress response in—cholinergic neurons (Hanin, 1996). A functional dose response for unilateral, striatal AF64a injection will be established by measuring the asymmetry of locomotor output 30 hours post injection of 8 week old wt C57Bl/6 male mice. Shh expression in the ventral midbrain (vMB) will be subsequently quantified by quantitative rtPCR using “TAQman”-type expression assays for Shh (Applied Biosystems).

Shh Up-Regulation in the Ventral Midbrain Effect on GDNF Expression in the Striatum

As discussed in Example 1, AF64a injection into the PPTg of mice with genetic ablation of Shh from DA neurons will allow one to investigate which genes, if any, in the experimentally uncompromised striatum are functionally regulated by Shh expression in the ventral midbrain.

Using “TAQman”-type quantitative PCR expression assays for cholinergic markers on cDNA derived from striatal mRNA preparations, the expression of ChAT and vAChT in the striatum will be examined.

Shh Expression by Spinal Motor Neurons

Using markers for the temporal and spatial development of the columnar organization of the spinal MN system in chicken at all stages analyzed (Raldh2, Lim1, Isl1 Lim3, Isl2, ChAT), whether Shh is expressed by MNs of all motor neuron columns will be examined. The pattern of Shh expression in MN in the mouse using the nuclear LacZ expression tracer allele for Shh will also be examined.

Tissue Specific Ablation of Shh from Motor Neurons

To functionally characterize MN expressed Shh, a conditional genetic ablation approach based on Cre activity expressed from the Olig2 Cre locus (as discussed in Example 1) will be used.

Up-regulation of GDNF in skeletal muscles in the absence of Shh expression by spinal motor neurons

The work in the nigro striatal system discussed in Example 1 demonstrated that Shh expression in MNs inhibit expression of GDNF in the muscle. GDNF is expressed at high levels in the embryonic limb (Wang et al., 2002), but is down regulated post-natal and becomes restricted to muscle spindles. The expression of GDNF will be therefore analyzed longitudinally in two calf muscles: the Gastrocnemius, a predominantly fast twitch muscle, and the Soleus, a predominantly slow twitch muscle using quantitative, using rtPCR (“TaqMan” type expression assays).

The Transgenic G93A SOD Model of ALS

In the G93A SOD transgenic model of familial ALS, temporally defined selective vulnerabilities of distinct MN synapses and axons precede the premature degeneration and death of lower MNs. Previous work has provided evidence that different MN subtypes exhibit selective vulnerabilities towards degeneration in the G93A model of ALS (Pun et al. (2006)). The G93A SOD1 mice have been described in Example 1.

Up Regulation of Shh in MNs and Down-Regulation of GDNF and CNTF in Skeletal Muscles in the Course of the SOD Phenotype

Shh can be a factor whose dynamic expression in MN can modify the disease progression in ALS. To test this, the expression of Shh in the ventral spinal cord will be quantified by quantitative rtPCR (“TAQman” type expression assays) and 13-gal activity will be measured in animals that were double heterozygous for the G93A SOD transgene and the conditional Shh IRES lacZ gene expression allele. The expression of GDNF and CNTF in the soleus muscle will then be analyzed as a function of age of the animal in a longitudinal study design through the course of phenotype development in the transgenic G93A SOD model by quantitative rtPCR (“TaQMan type expression assays).

Pharmacological Inhibition of Shh Signaling in Soleus Muscle of Endstage G93A SOD Transgenic Animals

Without being bound by theory, the inverse correlation of Shh and GDNF expression and increase in peripheral GDNF expression in mice with genetic ablation of Shh expression by motor neurons is consistent with a scenario in which Shh signaling inhibits GDNF expression in a direct fashion. Thus, application of Shh antagonists to the muscle should relieve Shh mediated repression of GDNF and CNTF expression. This will be tested through injection of GDC-0449, an antagonist of Shh signaling, into the soleus of endstage G93A SOD transgenic animals.

GDC-0449 will be injected unilaterally into the soleus of 125 day old G93A SOD transgenic animals and whether there is a dose dependent up-regulation of neurotrophic factor expression (e.g., in GDNF and/or in CNTF expression) over the saline injected contra lateral control soleus will be examined.

The experiments described herein will be carried out with additional Shh antagonist compounds in order to demonstrate whether pharmacological inhibition of the Shh pathway leads to an up-regulation of GDNF in the face of increased Shh production centrally and whether the muscles in end-stage animals remain sensitive to Shh signaling and competent to express GDNF.

### Example 3

### Smo Antagonists Boost Endogenous GDNF Expression in the Adult Striatum

GDNF protects DA neurons of the mesencephalon and noradrenergic neurons of the locus coeruleus from neurotoxins when administered directly into the brain. Genetic ablation of either c-Ret, the GDNF co-receptor, from DA neurons or GDNF in the adult mouse, causes an adult onset, progressive loss of mesencephalic DA neurons. Compounds that will boost the production of GDNF from relevant endogenous sources in the adult brain can overcome many of the side effects and inefficiencies associated with infusion of exogenous GDNF.

Shh signaling is best known for its concentration dependent function on target cells: While basal and high concentrations regulate cellular survival and proliferation respectively, intermediate concentrations regulate differential gene expression during the development of the CNS. The experiments herein in adult mice reveal also concentration dependent, multiple functional roles of Shh signaling in the adult nigro-striatal system. Using conditional gene ablation and gene expression tracer strategies in mice, it was demonstrated that DA neurons of the mesencephalon express Shh, and cholinergic neurons of the striatum GDNF throughout life. DA neuron produced Shh is necessary for the long term maintenance of ACh neurons of the striatum (Gonzalez et al., 2007). However, acute over-expression of Shh by DA neurons inhibits GDNF expression by striatal ACh neurons (Gonzalez and Kottmann, 2008). The data herein demonstrate that reciprocal signaling by Shh and GDNF between DA neurons of the mesencephalon and ACh neurons of the striatum is essential for the coordinated trophic maintenance of both neuronal populations and for homeostatic control of DA- and ACh-“tone” in the basal ganglia.

Without being bound by theory, developed Shh antagonists already in clinical use as anticancer treatments can be utilized to boost GDNF expression in diseased brains with potentially beneficial effects for the maintenance of DA neurons. This will be tested in 3 steps using commercially available agonists (SAG) and antagonists (cyclopamine, KADAR-cyclopamine):

1) Determination of ACh neuron survival, and kinetics of cholinergic marker- and GDNF expression as a function of Smo agonist and antagonist concentration and application regime in primary neuronal cell culture. This cell culture model has been previously established based on mice double heterozygous for ChAT-EGFP and GDNF-lacZ gene expression tracer alleles. The EGFP marker allows for purification of striatal ACh neurons derived from neonates by FACS and easy visualization of ACh neuron morphology in culture whereas the LacZ marker allows the selective quantification of GDNF expression by ACh neurons not confounded by GDNF expression by feeder cells derived from non-transgenic littermates.

2) Measurement of cholinergic marker and GDNF expression in adult C57Bl/6 wt animals after acute or chronic exposure to antagonist laced drinking water or unilateral striatal injection or chronic perfusion by use of osmotic micropumps.

3) Assessment of a neuroprotective and restorative effect of Smo specific antagonist treatment on nigral DA neurons by stereological quantification of numbers of DA neurons in the nigra and dopamine fiber density in the striatum as endpoint measures (a) after systemic MPTP intoxication; (b) in a relevant genetic model of PD with progressive, adult onset loss of nigral DA neurons. This model should not be based on the manipulation of either GDNF or Shh expression or involve ACh neurons of the striatum.

### Example 4

### Protection of Dopaminergic Neurons from Neurotoxicological Insult of MPTP In Vivo

The ablation of mesencephalic dopaminergic neurons through the injection of the neurotoxin MPTP produces a well established murine model of Parkinson's disease (Meridith et al., Parkinsonism Relat Disord. 2008; 14 Suppl 2:S112-5). The primary toxicity of MPTP occurs in DA neurons with SNpc DA neurons being especially sensitive and the quality and degree of cellular damage being a function of toxin concentration and application regime: Interestingly, chronic intoxication leads to the loss of DA neurons by apoptosis (Tatton and Kish, Neuroscience. 1997 April; 77(4):1037-48), whereas acute, moderate doses lead to striatal denervation and subsequent renervation (Jackson-Lewis et al., Neurodegeneration. 1995 4(3):257-69; Hoglinger et al., Nat. Neurosci. 2004 July; 7(7):726-35).

Guided by previous studies (Vila et al., J. Neurochem. 2000 February; 74(2):721-9), a semi chronic injection schedule of 30 mg/kg/day for 4 days of MPTP aiming to achieve a 40% reduction in the numbers of DA neurons in the ventral midbrain was chosen.

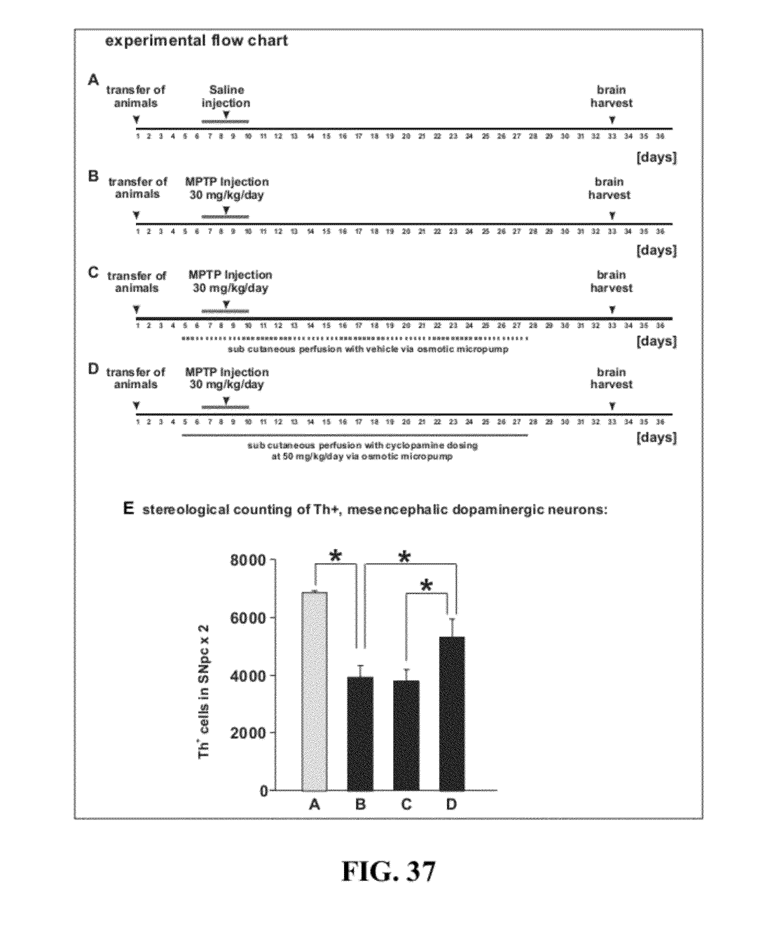

In this model, whether the co-injection of the Shh antagonist cyclopamine will increase the resilience of DA neurons to the neurotoxin MPTP was assessed. Four groups of 8 week old C57bl/6J male mice were analyzed: (1) vehicle (for MPTP) control, (2) MPTP (3) MPTP+Cyclopamine, (4) MPTP+ vehicle (for cyclopamine) control (FIG. 37). As schematized in the experimental flow chart in FIG. 37A-D, animals were habituated to the MPTP mice holding room upon delivery and fitted with “28 day” osmotic micropumps (Alzet) on day 4. Pumps were loaded the day before with either cyclopamine at a concentration to achieve the delivery of 50 mg/kg/day, or vehicle alone. Animals were then either injected with cyclopamine or carrier once a day on day 7, day 8, day 9, day 10 with 30 mg/kg of MPTP or vehicle. On day 33, animals were sacrificed by perfusion, brain extracted and prepared for cryostat sectioning. Floating sections of the ventral midbrain were immunohistochemically stained for tyrosine hydroxylase (Th) immunoreactivity. Th+ cells were quantified using a stereological cell counting techniques (see FIG. 37: Brief Description of the Figures discussed herein).

A 39% reduction (n=5, p<0.01; student's t-test) is found in the numbers Th+ cells at 33 days in animals injected with MPTP compared to vehicle controls (FIG. 37E). An MPTP injected animal which also received chronic vehicle injection for 28 days via osmotic micropump exhibited a similar reduction in Th+ cells (n=1, p<0.05; student's t-test). In contrast, the injection of MPTP in an animal which was perfused chronically by cyclopamine at 50 mg/kg/day for 33 days resulted in a statistically significant reduction in neurodegeneration: only a 21% reduction (n=1; p<0.05; student's t-test) is observed in Th+ cells in the ventral midbrain compared to control animals.

### Example 5

### Shh Expression in DA Neurons and Neurogenesis

This example discusses that Shh expressed by DA neurons can be both, a cell type specific sentinel for neuronal dysfunction and a morphogen whose expression at different levels can skew the qualitative outcome of SVZ neurogenesis towards cell identities of physiological need. Without being bound by theory, Shh produced by DA neurons of the mesencephalon and delivered to the SVZ by axonal projection, influences cell fate decisions in SVZ neurogenesis and interfaces between the detection of physiological stress in neurons and the alteration of the qualitative outcome of SVZ neurogenesis. This will be examined by quantization of the size and relative proportions of SVZ progenitor domains and interneuron populations of the olfactory bulb in animals that express various levels of Shh in DA neurons. Graded up-regulation of Shh in DA neurons will be evoked by inducing physiological cell stress in cholinergic neurons of the striatum and the Pendunculo Pontine Tegmental nucleus (PPTg). By varying the target cells for the induction of physiological cell stress and by performing these experiments in animals with tissue specific genetic ablation of Shh from DA neurons and controls, Shh effects will be further differentiated from other possible dopaminergic signals on SVZ neurogenesis.

The following will be determined: a) the mitotic index and size of the SVZ A-, B- and C-cell compartments in mice with Shh ablation in DA neurons; b) the numbers of Pax6 and Olig2 expressing precursors in the SVZ and the rostral migratory stream (RMS) as a function of Shh expression in DA neurons; and c) the relative proportions of 5 distinguishable olfactory bulb interneuron populations, which are replenished by neurogenesis as a function of Shh expression in DA neurons.

It will also be determined whether: (a) Shh expression in DA neurons is regulated by signals emerging from other neuronal nuclei and cellular structures besides mono-synaptically connected cholinergic cell populations; (b) established, genetic cell fate tracing techniques for the identification of neuronal identities produced in response to induced Shh expression in DA neurons in the adult mouse brain can be adapted; and (c) cholinotoxin induced up-regulation of Shh in DA neurons cause alterations in the relative size of SVZ precursor populations and changes in the cytoarchitecture of the olfactory bulb.

The SVZ neurogenic niche of the adult brain has been chosen as a model system to address two fundamental questions: a) is the qualitative outcome of neurogenesis static or dynamic? b) what are the signals that interface between sensing the need for neuronal replacement and the regulation of cell fate during neurogenesis?

The neuronal stem cells and their differentiation potential in the SVZ of intact, adult animals will be analyzed as a function of the expression of the cell signaling molecule sonic hedgehog (Shh) in dopaminergic neurons. Genetically altered mice that are either rendered unable to express Shh or that over-express Shh as a result of inducing neuronal dysfunction in cholinergic neurons will be used.

Neuronal stem cells will be analyzed in their physiological environment in the adult brain. Data derived from these studies will be of direct physiological relevance for devising methods that can alter the differentiation path of new neurons produced in the adult brain. Hence, these studies can contribute to finding approaches to stimulate in vivo resident stem cells to give rise to particular cells that need to be replaced in neuron degenerative diseases.

Methods that allow the alteration of the expression of a potent maintenance- and differentiation-factor, Shh, for neuronal stem cells in vivo in the adult brain have been devised. These methods are based on the induction of physiologically relevant neuronal dysfunction. One therefore can ask, whether different levels of Shh expression determines the production of particular neurons by SVZ neurogenesis. These experiments will help to assess the dynamic range of potential outcomes of neurogenesis in vivo in the adult brain.

The results demonstrate that the morphogen Sonic Hedgehog (Shh), expressed outside of the germinal niche by adult dopaminergic (DA) neurons of the mesencephalon, is a key regulator of adult neurogenesis. Genetic ablation of Shh from DA neurons causes an overall reduction of neurogenic activity, but an increase in the numbers of dopaminergic, periglomerular neurons of the olfactory bulb, and olfactory dysfunction. For example, Shh expression by DA neurons was shown to be up-regulated dynamically in correlation with the severity of cell physiological stress and neuronal dysfunction in connected neurons. Thus the data show that Shh expressed by DA neurons can be both, a cell type specific sentinel for neuronal dysfunction and a morphogen whose expression at different levels can skew the qualitative outcome of SVZ neurogenesis towards cell identities of physiological need.

Shh Expression in the Adult Brain

A genetic gene expression tracer allele for Shh, in which the expression of Shh is strictly linked to the expression of nLacZ, was produced (Kottmann and Jessell, FIG. 1A). This recombinant allele is a very useful experimental tool to reveal faithfully and with high sensitivity the cellular identity of those cells in a multi-cellular setting that express Shh and has been used for this purpose (Machold et al., 2003, Jeong et al., 2004, Lewis et al., 2004).

Using double fluorescent immuno-histochemistry and confocal microscopy, Shh expression was observed in virtually all tyrosine hydroxylase (TH) positive cells in the subtantia nigra pars compacta (SNpc, cell groups classified by Dahlstroem and Fuxe as “A9”, FIG. 1B-E), the ventral tegmental area (VTA, “A10”, FIG. 1B) and the retro rubral field (RRF, “A8”). No expression of Shh in dopaminergic neurons of the diencephalon and olfactory bulb was observed.

Within the SVZ, the resident progenitor B and C cell types are Shh responsive (Ahn and Joyner, 2005, Palma et al., 2005. FIG. 1G) whereas the C and A cell types express dopamine receptors (Hoglinger et al., 2004, Freundlieb et al., 2006; FIG. 1G). Surprisingly, utilizing the Shh gene expression tracer allele, one was unable to find Shh expressing cells within the SVZ proper or within a 20 cell diameter wide area extending from the subependymal cell layer (FIG. 1H). The analysis of Gli1::lacZ gene expression tracer mice reveals that 25% of all B cells, 57% of all C cells, and 18% of all A cells receive a productive Shh signal in the normal SVZ at a given moment in time. Transcriptional up-regulation of Ptc (the Shh receptor) and Gli1 (a mediator of Shh signaling) expression is a sensitive marker for those cells that receive a productive Shh signal. Utilizing indicator mouse lines in which LacZ expression is either linked to Ptc1 (Goodrich et al., 1997) or Gli1 (Bai et al., 2002), Ptc1 and Gli1 expression was readily found in the SVZ (FIG. 1 I, K). Based on the inability to detect expression of the ligand, Shh, locally within or in diffusion reach of the SVZ, despite the overwhelming functional and cytohistochemical evidence for active Shh signaling occurring in the SVZ in vivo, Shh can be provided by cells situated outside of the SVZ. One reasoned that neurons, like DA neurons of the mesencephalon, which elaborate axonal projections to the SVZ (Freundlieb et al., 2006; FIG. 1F), will be good candidates for providing Shh to the SVZ.

Using a genetic gene expression tracer allele for Shh, no evidence for Shh expression within or in the immediate vicinity of the SVZ was further found, but it was discovered that all dopaminergic (DA) neurons of the mesencephalon express Shh. These neurons elaborate topographically organized innervation of the SVZ demonstrating the possibility that DA produced Shh can reach the neurogenic niche of the SVZ through axons.

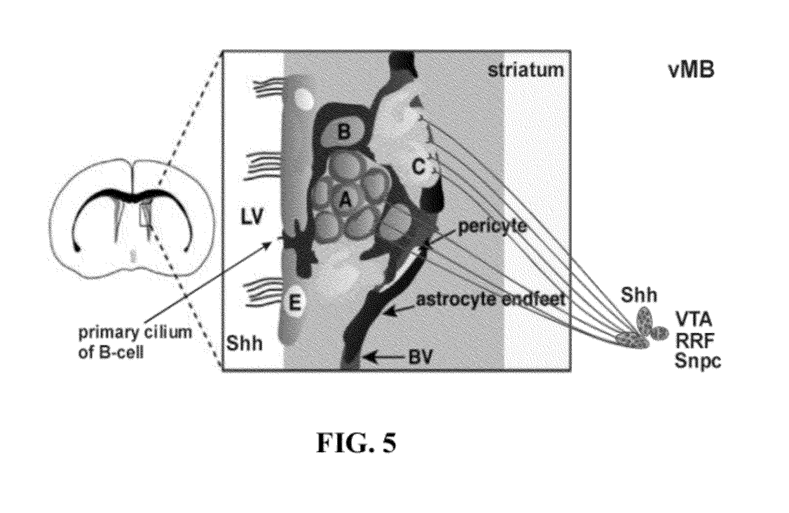

In the absence of evidence for the expression of Shh by resident SVZ cells, Shh can be provided by sources outside of the SVZ in the adult brain. Recent analysis of histological and morphological aspects of the neurogenic niche in the SVZ demonstrates 3 potential sources of Shh: (1) micro vasculature, (2) the lumen of the ventricle, and (3) neuronal innervation. B, C, and A cells are in contact with a rich plexus of microvessels that can expose all 3 cell types to Shh carried in blood serum (Tavazoie et al., 2008, Shen et al., 2008; FIG. 5). B-cells elaborate a primary cilium in between ependymal cells into the lumen of the ventricle potentially exposing it to Shh which is thought to be present in cerebrospinal fluid (Mirzadeh et al., 2008). C and A cells are innervated by dopaminergic ollaterals of mesencephalic dopaminergic neurons, which express Shh throughout life.

Ectopic Production of Shh

It was shown that Shh ectopically produced by dorsal root ganglion cells, transported through the dorsal root and subsequently released from axons in the dorsal spinal cord, can induce the appearance of ectopic ventral neuronal identities in the dorsal spinal cord in the chick embryo.

Protein components of the primary cilium, which is found on most vertebrate cells, are required for Shh signaling (Huangfu et al., 2003, reviewed in Rohatgi and Scott, 2007). Since only B cells, but not C and A cells elaborate primary cilia into the lumen of the ventricle it is possible that B cells receive Shh signaling from the lumen of the ventricle while A and C cells can receive Shh from other sources like dopaminergic innervation or the micro vasculature. Hence, the current morphological description of the germinal niche in the SVZ allows the interesting speculation that the resident constituent cell types of the SVZ, although in close proximity and intermingled, receive their respective Shh signal from different anatomic sources. Such a spatially segregated, cell type specific sensitivity towards Shh produced by different sources can allow the maintenance of the stem cell pool (B cells) by stable Shh signaling independently of dynamic alterations in cell fate determination through changes in Shh signal strengths acting on C and A cells (FIG. 5). Without being bound by theory, changes in Shh expression in mesencephalic DA neurons alters the qualitative outcome of SVZ neurogenesis by influencing cell fate decisions in the neurogenic niche of the SVZ.

Tissue Specific Ablation of Shh from DA Neurons

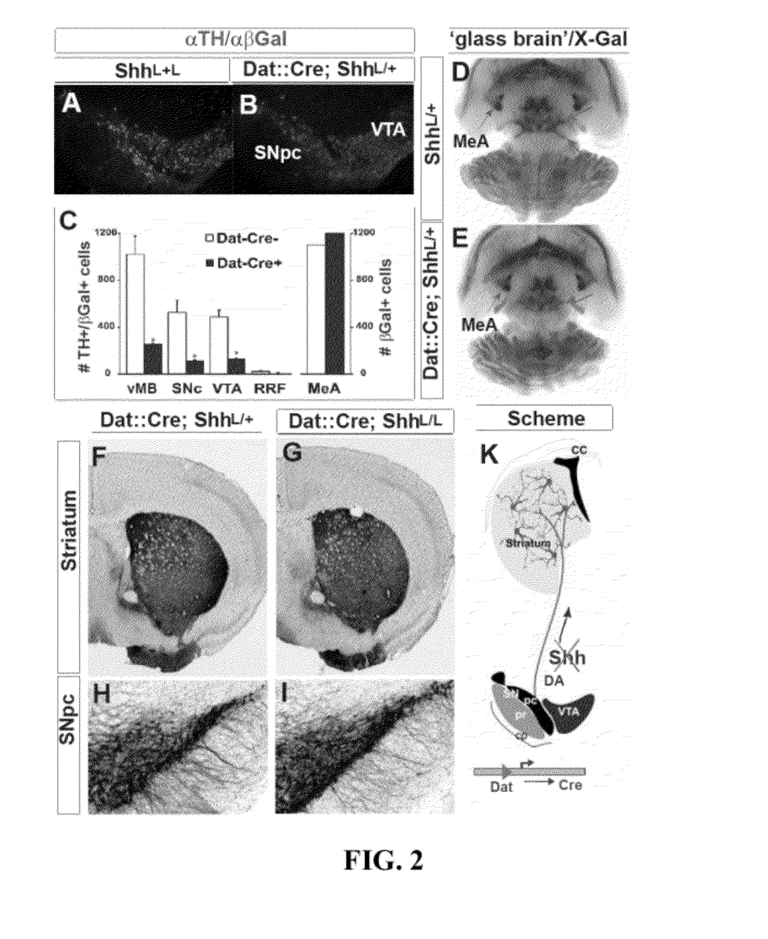

To begin to test whether DA neuron produced Shh regulates neurogenesis in the SVZ, animals with tissue specific, homozygous Shh ablations mediated by Cre activity expressed from the dopamine transporter locus were produced (DAT::cre, Zuang et al., 2005, FIG. 2K). Cre mediated ablation of Shh also deletes the nlacZ tracer from the Shh locus in these animals providing a means of quantifying the efficiency of locus recombination and assessing its tissue specificity (FIG. 2C-E). At 6 weeks of age, it is observed that 80% of DA neurons in the mesencephalon have lost the expression of Shh (FIG. 2A-C). There were no alterations in the expression of Shh in other brain areas as exemplified by the quantification of Shh expressing cells in the medial amygdala (MeA, FIG. 2C) and qualitatively assessed by “glass brain” preparations post whole mount staining for β-gal activity in the entire brain (FIG. 2D, FIG. 2E right-hand side arrows point to mesencephalic DA nuclei which are not stained after Cre mediated recombination, left-hand side arrows point to the medial amygdala).

Shh expressed by SNpc neurons can play a role in the maintenance and function of the nigro-striatal system. However, no qualitative difference was found in dopaminergic fiber density in the striatum or in the morphological appearance of the SNpc by immunostaining for Th (FIG. 2F-I). The quantification of the immunohistochemical preparations by stereology and of the locomotor activity in the Open Field paradigm, a sensitive measure of dopamine “tone” in the basal ganglia, did not reveal a phenotype in the absence of Shh from DA neurons.

Altered SVZ Neurogenesis in the Absence of DA Produced Shh

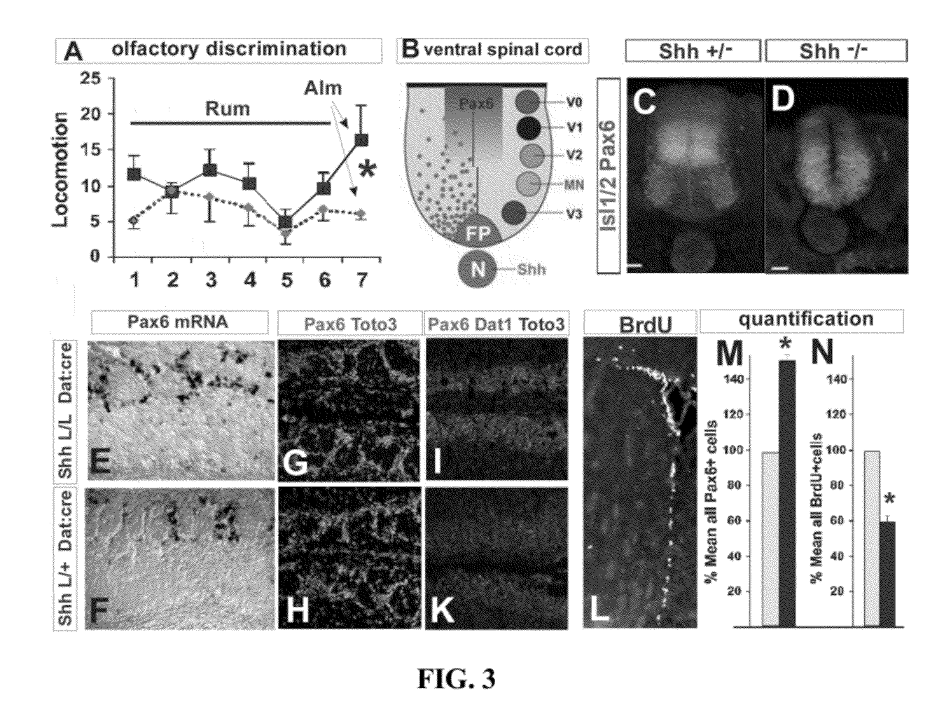

Whether DA neuron produced Shh influences the qualitative outcome of SVZ neurogenesis was tested. Without being bound by theory, subtle changes in SVZ neurogenesis caused by chronic absence of Shh from DA neurons can lead to a functional phenotype in olfaction due to the accumulative effect of qualitative and/or quantitative alterations in the replenishment of OB interneurons. The animals were therefore tested in an olfactory discrimination assay. Here, animals are habituated to a particular scent, Rum, through repeated exposure to a scented jar. After the 6^{th} trial the scent of the jar is switched to Almond. While wt animals react to the new scent through increased locomotor and explorative behavior, animals with Shh ablation from DA neurons apparently fail to detect the new scent (FIG. 3A). Olfactory discrimination deficits are a preclinical risk factor for Parkinson's Disease (Herting et al., 2008). Post mortem studies reveal increased numbers of periglomerular, DA neurons in the bulb (Huisman et al., 2004), a finding recapitulated in neurotoxicological models of PD (Winner et al., Exp Neurol. 2006 January; 197(1):113-21), demonstrating that increased DA tone in the bulb, a negative modulator of odorant perception (Huisman et al., 2004), can be responsible for causing the observed olfactory deficits. Periglomerular DA neurons arise from the Pax6 expressing cell lineage produced in the SVZ (Hoglinger et al., 2004).

Pax6 is a “class 1” transcription factor which is repressed by Shh signaling during spinal cord development (schematically depicted in FIG. 3B) and its expression domain extends into the ventral neural tube preventing the differentiation of ventral cell types, like motor neurons, in the absence of Shh signaling from the floorplate and notochord (FIG. 3C-D; Ericson et al., 1997b). The cytoarchitecture of the olfactory bulbs of animals with and without Shh expression was comparatively analyzed in mesencephalic DA neurons. A 40% increase of Pax6 expressing, DA neurons in the glomerular layer in the absence of Shh expression by mesencephalic DA neurons was also found (FIG. 3E-K and quantified in FIG. 3M), a finding similar to the results obtained by Winner et al. (2006) after the unilateral, neurotoxicological ablation of mesencephalic DA neurons. A much higher immunoreactivity for Dat within the glomerular layer consistent with a higher DA tone was noticed (FIG. 3I, FIG. 3K). The overall mitotic activity in the SVZ 24 hours post labeling of dividing cells was then analyzed by BrdU incorporation (FIG. 3L). A 40% reduction in overall mitotic activity (FIG. 3N), a reduction also seen after the neurotoxicological ablation of mesencephalic DA neurons was found (Winner et al., 2006, Hoglinger et al., 2004) and consistent with a mitogenic function of Shh in SVZ neurogenesis as demonstrated by Palma et al. (2005). Without being bound by theory, the reduction in overall mitotic activity in the SVZ in conjunction with an increase of Pax6, a cell fate marker repressed by Shh signaling, indicates a switch in cell fate in SVZ neurogenesis in the absence of DA neuron produced Shh and the increase in the numbers of Pax6 expressing cells must come at the expense of other cell identities produced by SVZ neurogenesis that has not yet been identified.

From these results a question arises for the function of DA neuron produced Shh in SVZ neurogenesis: Is Shh expression by DA neurons static or dynamic? Without being bound by theory, if its expression can be altered, a Shh signal provided from DA neurons can be involved in regulating different outcomes of SVZ neurogenesis. Guided by the finding that Shh expression in adult facial motor neurons can be upregulated by axotomy (Akazawa, 2004), whether physiological cell stress in the striatum can modulate Shh expression in mesencephalic DA neurons was explored. Cholinergic neurons of the striatum and of the Pendunculo Pontine Tegmental nucleus (PPTG), both of which are monosynaptically connected with mesencephalic DA neurons (FIG. 4G) and express the Shh receptor Ptc1, are a source of signals that modulate Shh expression in the ventral midbrain.

Unilateral Injection of the Cholinotoxin Ethylcholine Mustard Aziridium (AF64a) into the Striatum and PPTG

AF64a is a compound with structural similarities to choline, which acts as a competitive and reversible inhibitor of both Choline Transporter and Choline Acetyl Transferase (ChAT; Dudas et al., 2003; Amir et al., 1988; Leventer et al., 1987; Sandberg et al., 1984; Fan and Hanin, 1999). AF64a application causes an acute inhibition of—and physiological stress response in—cholinergic neurons (Hanin, 1996). A functional dose response for unilateral, striatal AF64a injection was established by measuring ipsilateral turning behavior 30 hours post injection in 6 week old wt C57B/6 male mice. The turning bias increases from 0.1 mM to 5 mM AF64a, consistent with the muscarinic receptor mediated, inhibitory neuromodulatory role of ACh in the striatum leading to ipsilateral disinhibition of striatal motor output and contralateral increased spinal cord motor activity (FIG. 4A). Interestingly, a dose dependent, step wise, up-regulation of Shh expression was found in the ipsilateral ventral midbrain (vMB) 36 h post striatal AF64 injection by real time quantitative PCR using “TaqMan”-type expression assays (rtqPCR; Applied Biosystems, FIG. 4B).

The PPTg provides monosynaptic, cholinergic input to the SNpc (Futami et al., 1995). Cholinotoxin injection into the PPTg elicits a contra lateral turning bias (negative values in FIG. 4C, FIG. 4D) consistent with a reduction of dopaminergic activity in the ipsilateral striatum due to an inhibition of nicotinic receptor mediated cholinergic stimulation of the SNpc (FIG. 4G). In these animals Shh expression in the ipsilateral vMB is 8 fold over expressed compared to the contra lateral control vMB.

Additionally, pharmacological insults to cholinergic neurons that are connected monosynaptically with DA neurons up-regulate Shh expression in DA neurons. Furthermore, tissue specific ablation of Shh from DA neurons causes an increase in the numbers of dopaminergic, Pax6+ periglomerular neurons in the olfactory bulb and olfactory dysfunction.

The qualitative opposite behavioral response to AF64a injections into the striatum and the PPTg demonstrated that the physiological response of DA neurons in these two models is different despite the similar upregulation of Shh. The expression of dopaminergic markers in the vMB by “TAQman” type rtqPCR 36 h after cholinotoxin injection into either the striatum or PPTg was therefore quantified. A down-regulation of Th and Dat upon striatal AF64a injections (FIG. 4E) but an up-regulation of Th and Dat upon AF64a injection into the PPTg (FIG. 4F) was found. Thus, DA neurons of the mesencephalon adjust their physiology to balance the inhibitory, cholinergic “tone” in the striatum. Upon the induction of cholinergic dysfunction in the striatum the production of DA is reduced whereas the lack of nicotinic receptor mediated stimulation of DA neurons by PPTg neurons leads to an up-regulation of DA production (also compare FIG. 4G). The independence of Shh regulation from the physiological adjustments of DA neurons in combination with the genetic ablation of Shh from DA neurons provides an experimental inroad into distinguishing Shh mediated effects from other DA neuron mediated affects on SVZ neurogenesis. Without being bound by reason, Shh expression in DA neurons of the mesencephalon is a sensitive sentinel for the functional and structural integrity of basal ganglia circuitry and a key regulator of SVZ neurogenesis.

Disease Relevance

Olfactory dysfunction is a premonitory symptom in many neurological and psychiatric diseases like Parkinson (PD), Huntington, Alzheimer's, schizophrenia, dementia, depression and others (Doty et al., 2003). The work discussed herein (as well as EXAMPLES below) can further define a mechanistic link between the integrity of the mesencephalic dopaminergic system and basal forebrain cholinergic cell populations, which are structurally and/or functionally corrupted in many neurological and psychiatric conditions like PD, Alzheimer's and schizophrenia, and the replenishment of olfactory bulb neurons. Hence this work can help identify preclinical disease markers.

### Example 6

### Experimental Designs for Role of Shh as Sentinel in SVZ Neurogenesis

The data presented in Example 3 show that DA neuron produced Shh is a “sentinel” for the structural integrity of neurons functionally connected to DA neurons and is a key regulator of SVZ neurogenesis. The Shh loss of function studies are consistent with a scenario in which a reduction of Shh expression by mesencephalic DA neurons signifies dopaminergic cell stress. Under these conditions, it is shown that SVZ neurogenesis is skewed towards increased production of Pax6 expressing precursor cell fates as evidenced by an increase in Pax6+, dopaminergic periglomerular cells in the olfactory bulb (FIG. 3). Based on the repression of Pax6- and the induction of Olig2 expression by Shh during spinal cord development (Ericson et al., 1997a; Ligon et al., 2006), increased Shh expression by DA neurons can lead to a reduction in the size of the Pax6, but an enlargement of the Olig2 expressing precursor populations. Without being bound by theory, changes in the relative sizes of SVZ precursor population caused by alterations in Shh expression in DA neurons lead to a reduction in pax6 lineage dependent OB interneurons like periglomerular, dopaminergic neurons, a result which will constitute a corollary to the finding of increased numbers of periglomerular DA neurons in the absence of DA neuron produced Shh (FIG. 2). This will be tested through qualitative and quantitative analysis of the precursor cell populations of the SVZ and the replenishing interneuron populations of the olfactory bulb in animals that express different levels of Shh in mesencephalic DA neurons.

As an experimental approach, a combination of the unilateral, physiological stress induced up-regulation of Shh in DA neurons (FIG. 4) with the tissue specific genetic ablation of Shh from DA neurons (FIG. 2), was chosen. As shown in Example 3, unilateral injection of the cholinotoxin AF64a into the striatum (FIG. 4A) or PPTg (FIG. 4C) causes a dose dependent, ipsilateral up-regulation of Shh in DA neurons. The striatum and PPTg, and the route of the stereotactic injection to reach these loci, are spatially segregated from the DA neurons of the mesencephalon, the SVZ and the OB and do not involve the DA projections through the midbrain bundle to the SVZ or the RMS. Hence, the induction of cholinergic dysfunction by AF64a application allows the up-regulation of Shh and the read out of its function in brain areas whose structure and connectivity have not been affected by the injection of the cholinotoxin. Moreover, the induction of reversible cholinergic dysfunction for evoking up-regulation of Shh in connected DA neurons appears milder and of greater physiological relevance than the induction of neuronal cell loss by exitotoxins or the genetic induction of apoptosis.

Evidence is provided that the functionality of DA neurons is altered in opposite ways upon induction of cholinergic dysfunction in the striatum and the PPTg: injections into the striatum lead to a down-regulation of DA markers, while PPTg injections lead to an up-regulation of DA markers consistent with an ipsilateral turning bias upon striatal injections but a contra lateral turning bias after PPTg injections (FIG. 4E-F). Shh up-regulation is, in contrast, a common response to cholinotoxin injection into either locus. Hence, consistent, dose dependent changes in SVZ physiology and qualitative outcome of SVZ neurogenesis after induced expression of Shh by AF64a injection into either the striatum or PPTg will therefore correlate with induced Shh expression and will exclude common dopaminergic functions like DA itself as the causative agent. Unilateral changes in SVZ physiology and qualitative outcome of neurogenesis that manifest after both AF64a injection into the striatum and the PPTg, and are not detected upon the genetic ablation of Shh from DA neurons can then be attributed to Shh upregulation in DA neurons.

As detailed further below, unilateral AF64a injection will be combined with labeling of mitotically active cells by systemic injections of the nucleotide analog BrdU. Based on the time course of Shh up-regulation in motor neurons upon axotomy (Akazawa et al., 2004) and in DA neurons post AF64a injection, BrdU will be injected 6 times spaced over 48 h beginning 24 hours post cholinotoxin application (see Methods section below). The experimental results will be expressed as relative changes between the ipsilateral and contralateral hemispheres in cell populations identified by coexpression of specific cell fate or neuronal identity markers and BrdU.

### Experiment 1

### Mitotic Index and Size of the Svz A-, B- and C-Cell Compartments in Mice with Shh Ablation in DA Neurons

“A” cells, which are innervated by dopaminergic terminals can be recognized by the marker PSA-NCAM. “C” cells, transit amplifying cells, are heavily innervated by DA neurons and can be recognized by the expression of EGF receptor. The number of BrdU labeled cells, which coexpress either PSA-NCAM or EGF-receptor and are located within 5 cell diameters next to the ependymal cell layer of the lateral wall of the ventricles, will be determined. The entire SVZ in its rostro-caudal extend will be sampled on 16 μm cryostat sections, spaced by 58 μm. Each cross section through the SVZ will be analyzed in its entirety. The rate of proliferation will be expressed as the number of A or C cells co-stained with BrdU over the total number of A or C cells as a function of rostro-caudal position.

The relative proportions of the different cell populations within the SVZ along the rostral-caudal axis exhibit a specific pattern. While “B” cells are found at all levels at fairly similar numbers, the most “C” cells are found in the middle third of the rostral caudal extend of the SVZ and the numbers of “A” cells gradually increase towards the rostral end of the SVZ (Garcia Verdugo et al., 1998). To visualize a potential difference in the distribution of A and C cells along the rostro caudal extend of the SVZ, one can perform immunohistochemical stainings on whole mount preparations of the SVZ (Doetsch and Alvarez-Buylla, 1996).

“B”-cells can be identified in situ through their expression of GFAP and Sox 2 (Brazel et al., 2005, Deotsch et al., 1997). “B” cells are not innervated by dopaminergic neurons (Hoglinger et al., 2004). However, changes in the proliferative index of “C”—and potentially “A”—cells can feed back onto the stem cell compartment. In fact, Palma et al (2005) showed that endogenous Shh signaling is necessary for the maintenance of the stem cell compartment. Likewise, Ahn and Joyner (2005) demonstrated that “B” cells are competent to receive a Shh signal in vivo. Since “B” cells appear to be spatially closely associated with “C” cells, which are heavily innervated by DA neurons, it is believed that Shh released from these terminals can have an effect onto nearby “B” cells.

### Experiment 2

### Localization and Quantification of Pax6 and Olig2 Expressing Precursor Cells in the SVZ and RMS

Pax6 is a “class 1” transcription factor which is excluded from ventral domains in the developing spinal cord by Shh signaling, whereas Olig2 is a “class 2” transcription factor, which is induced in ventral spinal cord domains by Shh signaling. In analogy to the situation in spinal cord development, it is believed that the exclusion of Pax6 expressing cells from the SVZ is due to the local action of Shh. Without being bound by theory, the numbers of Pax6 expressing cells among all “A” cells in the SVZ can increase in mice with Shh ablation in dopaminergic neurons. Likewise, the expression of Olig2 within the SVZ can be in part due to Shh signaling. Hence, in the absence of Shh produced in dopaminergic neurons a relative reduction in the numbers of Olig2 expressing precursors within the SVZ will be observed. The relative increase of Pax6 expressing cells and the relative reduction in the numbers of Olig2 expressing cells among all precursors will correspond to the “dorsalization” of the ventral spinal cord observed in the absence of Shh signaling (Ericson et al., 1997a). Correspondingly, the graded increase of Shh upon AF64a injections can lead to a decrease in Pax6 and an increase in Olig2 expressing precursor cells in the SVZ.

In normal animals Pax6 and Olig2 expressing precursor cells are spatially segregated into two distinct domains. Only 3% of all precursor cells within the SVZ express Pax6 whereas just outside of the SVZ, in the caudal end of the RMS 40% of all migrating precursor cells are Pax6 expressing cells. Olig2 exhibits the opposite gradient of expression in adult SVZ born precursor cells: 18% of all “A” cells in the SVZ are immunopositive for Olig2, whereas in the central RMS only 2% of all migrating precursors express Olig2 (Hack et al., 2005).

The percentage of Pax6 and Olig2 expressing precursors among all migrating, committed “A” cells at three rostro-caudal levels within the SVZ and within the caudal end of the RMS will be determined as a function of 5 different levels of Shh expressed by mesencephalic, DA neurons (no Shh expression [post genetic ablation of Shh], wt levels, and levels induced by either 0.1; 0.5; and 1 mM striatal AF64a injections, and levels induced by 0.5 mM AF64a injection into the PPTg).

### Experiment 3

### Effect of Shh Produced in DA Neurons on the Differentiation of Distinct Neuronal Identities in the Adult Brain

Whether Shh produced by DA neurons has an effect on the relative sizes of the end-differentiated populations of neurons in the olfactory bulb that are replenished through SVZ neurogenesis will be analyzed. There are at least 5 such populations of neurons in the bulb, which can be distinguished by anatomic location and marker expression (Hack et al., 2005, Kohwi et al., 2005). SVZ precursors will be pulse-labeled with BrdU. 21 days later, the relative proportions of the following populations among all BrdU labeled cells in the bulb will be determined as a function of Shh expression in DA neurons: (1) GABAergic granular cells, (2) GABAergic, ER81+ granular cells of the outer granule cell layers (3) Pax6 and TH expressing periglomerular neurons, (4) Pax6 and calretinin expressing neurons in the glomerular layer and (5) Pax6 and calbindin expressing neurons in the glomerular layer. The detection of relative differences in these populations will demonstrate that Shh expressed by DA neurons influences cell fate decisions in the SVZ that percolate through the ontogeny of the produced cells and manifest as cyto-architectural alterations in the OB.

This work will further define the regulatory and trophic environment in which adult neuronal stem cells reside. Data derived from these studies in this example will inform on in vivo mechanisms that, if engaged in vitro, can contribute to realize the full differentiation potential of neuronal stem cells for the production of distinct neuronal populations for neuronal replacement. These studies will also be a guide in approaches to stimulate in vivo resident stem cells to give rise to particular cells that need to be replaced due to neuron degeneration. The cell signaling pathway studied in this example, the Shh mediated signaling, has already attracted the interest of the pharmaceutical industry and a well defined pharmacology for the manipulation of this pathway has been developed. Without being bound by theory, graded Shh signaling in vivo can determine which neuronal cell types are produced during neurogenesis that can be used as either agonists or antagonists of Shh signaling in vivo to manipulate the qualitative outcome of SVZ neurogenesis.

### Methods

Husbandry and power of statistics considerations: In all studies, Dat-cre expressing male mice that are heterozygous for the Shh conditional allele [Dat-cre, Shh C/+] will be compared with Dat cre expressing male mice that are homozygous for the Shh conditional allele [Dat-cre; Shh C/C] and that are injected unilaterally with either AF64a or vehicle (artificial spinal fluid) as described in FIG. 4. These animals will be produced from crosses of [Dat-cre, Shh C/+] males with [Shh C/C] females. Each desired genotype will make up one quarter in the offspring. Empirical husbandry results demonstrate that on average 5 males of each desired genotype are obtained from 4 litters. It was determined empirically that 5 animals per group reveal reproducibly, statistically significant differences as a function of Shh gene dosage in the histological measures (see Example 3).

BrdU labeling: The DNA synthesis marker thymidine analog 5-bromo-2′-deoxyuridine (BrdU, Sigma, dissolved in 0.9% NaCl, 1.75% NaOH) will be injected intraperitoneally (100 mg/Kg of body weight) in a single dose 2 h before killing the mouse to assess proliferation in the SVZ and RMS or four injections repeated every 2 h, 21 days before killing to analyze the neuronal identities of BrdU labeled cells in the olfactory bulb. For BrdU double histochemical analysis non BrdU antigen will be detected first and signal fixed by Tyramide amplification prior to revealing the BrdU epitope by HCl treatment.

Immunohistochemistry: Mice will be deeply anesthetized with an overdose of pentobarbital (Sigma, 100 mg/Kg of body weight, i.p.) and perfused transcardially with 0.1 M sodium phosphate buffer (PBS) followed by 4% paraformaldehyde in 0.1 M PBS. The brains will be dissected out, postfixed and embedded for cryostat sectioning as described (Hack 2005, Hoglinger 2004); Primary antibodies: anti-GFAP (Sigma, mouse, 1:200 and DAKO, rabbit, 1:1:1000) anti-Pax6 (BABCO, rabbit, 1:500); anti PSA-NCAM (Chemicon, mouse, 1:400); anti-TH (Pel-Freez, rabbit, 1:500); anti rodent DAT (Chemicon, rabbit, 1:100); anti-synaptophysin (Upstate Biotech, mouse, 1:200, 1:200); anti-EGFR (Upstate Biotech, sheep, 1:50); anti-BrdU (ImmunologicalsDirect, rat, 1:200); anti-TuJ1 (Chemicon, rabbit, 1:5000); Anti-NeuN (Chemicon, mouse, 1:5000) anti Nestin (gift, Dr. Rene Hen, rabbit, 1:1000). Primary antibodies will be detected by subclass-specific secondary FITC-labeled antibodies, Cy3 and Cy5, or enhanced with tyramide amplification kit (Roche) or by diaminobenzidine methods (Vectastain).

Image analysis: Images will be captured using a digital camera coupled to a Nikon fluorescence microscope or a BioRad scanning confocal microscope. Three-dimensional reconstruction will be used to verify colocalization.

### LITERATURE CITED

Ahn S, Joyner A L (2005) In vivo analysis of quiescent adult neural stem cells responding to Sonic Hedgehog. Nature 437: 894-897.
Akazawa C, Tsuzuki H, Nakamura Y, Sasaki Y, Ohsaki K, Nakamura S, Arakawa Y, Kohsaka S. The upregulated expression of sonic hedgehog in motor neurons after rat facial nerve axotomy. J. Neurosci. 2004 Sep. 8; 24(36):7923-30.
Altman J (1969) Autoradiographic and histological studies of postnatal neurogenesis. IV. Cell proliferation and migration in the anterior forebrain, with special reference to persisting neurogenesis in the olfactory bulb. J Comp Neurol. 137(4):433-57.
Alvarez-Buylla A, Garcia-Verdugo J M (2002) Neurogenesis in adult subventricular zone. J. Neurosci. 22(3):629-34.
Alvarez-Buylla and Lim For the long run: maintaining germinal niches in the adult brain. Neuron. 2004 Mar. 4; 41(5):683-6. 2004
Amir A, Pittel Z, Shahar A, Fisher A, Heldman E. Cholinotoxicity of the ethylcholine aziridinium ion in primary cultures from rat central nervous system. Brain Res. 1988 Jun. 28; 454(1-2):298-307.
Bai C B, Auerbach W, Lee J S, Stephen D, Joyner A L. (2002) Gli2, but not Gli1, is required for initial Shh signaling and ectopic activation of the Shh pathway. Development. 129: 4753-4761.
Brazel C Y, Limke T L, Osborne J K, Miura T, Cai J, Pevny L, Rao M S. (2005) Sox2 expression defines a heterogeneous population of neurosphere-forming cellsn in the adult murine brain. Aging Cell. 4(4):197-207.
Breunig J J, Arellano J I, Macklis J D, Rakic P. (2007) Everything that glitters isn't gold: a critical review of postnatal neural precursor analyses. Cell Stem Cell. 2007, 1: 612-627.
Briscoe J, Pierani A, Jessell T M, Ericson J. (2000) A homeodomain protein code specifies progenitor cell identity and neuronal fate in the ventral neural tube. Cell. 101(4):435-45.
Charytoniuk D, Porcel B, Rodriguez Gomez J, Faure H, Ruat M, Traiffort E. (2002a) Sonic Hedgehog signalling in the developing and adult brain. J Physiol Paris. 2002 January-March; 96(1-2):9-16.
Charytoniuk D, Traiffort E, Hantraye P, Hermel J M, Galdes A, Ruat M. (2002b) Intrastriatal sonic hedgehog injection increases Patched transcript levels in the adult rat subventricular zone. Eur J. Neurosci. 16(12):2351-7.
Chiang C, Litingtung Y, Lee E, Young K E, Corden J L, Westphal H, Beachy P A. (1996) Cyclopia and defective axial patterning in mice lacking Sonic hedgehog gene function. Nature. 383(6599):407-13.
Chu T, Chiu M, Zhang E, Kunes S. (2006) A C-terminal motif targets Hedgehog to axons, coordinating assembly of the Drosophila eye and brain. Dev Cell. 10: 635-646.
Curtis M A, Faull R L, Eriksson P S. (2007) The effect of neurodegenerative diseases on the subventricular zone. Nat Rev Neurosci. 8: 712-723.
Curtis M A, Eriksson P S, Faull R L. (2007) Progenitor cells and adult neurogenesis in neurodegenerative diseases and injuries of the basal ganglia.
Clin Exp Pharmacol Physiol. 34: 528-532.
Doetsch F, Alvarez-Buylla A. (1996) Network of tangential pathways for neuronal migration in adult mammalian brain. Proc Natl Acad Sci USA. 93(25):14895-900.
Doetsch F, Garcia-Verdugo J M, Alvarez-Buylla A. (1997) Cellular composition and three-dimensional organization of the subventricular germinal zone in the adult mammalian brain. J. Neurosci. 17(13):5046-61.
Dudas B, Rose M, Hanin I. Dose-dependent effect of cholinotoxin AF64A on the cholinergic elements of the cingulum bundle in rat. Brain Res. 2003 Mar. 7; 965(1-2):295-8.
Ericson J, Rashbass P, Schedl A, Brenner-Morton S, Kawakami A, van Heyningen V, Jessell T M, Briscoe J. (1997a) Pax6 controls progenitor cell identity and neuronal fate in response to graded. Cell. 1997 90(1):169-80
Ericson J, Briscoe J, Rashbass P, van Heyningen V, Jessell T M. (1997b) Graded sonic hedgehog signaling and the specification of cell fate in the ventral neural tube. Cold Spring Harb Symp Quant Biol. 62: 451-466.
Fan Q I, Hanin I. Effects of AF64A on gene expression of choline acetyltransferase (ChAT) in the septo-hippocampal pathway and striatum in vivo. Neurochem Res. 1999 January; 24(1):15-24.
Freundlieb N, Francois C, Tande D, Oertel W H, Hirsch E C, Hoeglinger, GU (2006) Dopaminergic Substantia Nigra Neurons Project Topographically Organized to the Subventricular Zone and Stimulate Precursor Cell Proliferation in Aged Primates. J. Neuroscience 26(8): 2321-2325.
Futami T, Takakusaki K, Kitai S T (1995) Glutamatergic and cholinergic inputs from the pedunculopontine tegmental nucleus to dopamine neurons in the substantia nigra pars compacta. Neurosci Res 21: 331-342
Gage F H (2000) Mammalian neural stem cells. Science. 287(5457):1433-8.
Garcia-Verdugo J M, Doetsch F, Wichterle H, Lim D A, Alvarez-Buylla A. (1998) Architecture and cell types of the adult subventricular zone: in search of the stem cells. J. Neurobiol. 36(2):234-48.
Goodrich L V, Milenković L, Higgins K M, Scott M P. (1997) Altered neural cell fates and medulloblastoma in mouse patched mutants. Science. 277: 1109-1113.
Guerrero I and Chin Chiang. A conserved mechanism of Hedgehog gradient formation by lipid modifications. Trends in Cell Biology 2007 17: 1-5
Gurdon J B, Bourillot P Y. (2001) Morphogen gradient interpretation. Nature. 413(6858):797-803.
Hack M A, Saghatelyan A, de Chevigny A, Pfeifer A, Ashery-Padan R, Lledo P M, Gotz M. (2005) Neuronal fate determinants of adult olfactory bulb neurogenesis. Nat Neurosci 8(7):865-72.
Hanin I. The AF64A model of cholinergic hypofunction: an update. Life Sci. 1996; 58(22):1955-64.
Herting B, Schulze S, Reichmann H, Haehner A, Hummel T. (2008) A longitudinal study of olfactory function in patients with idiopathic Parkinson's disease. J. Neurol. 255: 367-370.
Hoglinger G U, Rizk P, Muriel M P, Duyckaerts C, Oertel W H, Caille I, Hirsch E C. (2004) Dopamine depletion impairs precursor cell proliferation in Parkinson disease. Nat Neurosci7(7):726-35.
Huang Z, Kunes S. (1996) Hedgehog, transmitted along retinal axons, triggers neurogenesis in the developing visual centers of the Drosophila brain. Cell. 86(3):411-22.
Huisman E, Uylings H B, Hoogland P V. (2004) A 100% increase of dopaminergic cells in the olfactory bulb may explain hyposmia in Parkinson's disease. Mov Disord. 19: 687-692.
Jeong J, Mao J, Tenzen T, Kottmann A H, McMahon A P. (2004) Hedgehog signaling in the neural crest cells regulates the patterning and growth of facial primordia. Genes Dev. 2004 18(8):937-51.
Kosaka T, Hataguchi Y, Hama K, Nagatsu I, WU J Y. (1985) Coexistence of immunoreactivities for glutamate decarboxylase and tyrosine hydroxylase in some neurons in the periglomerular region of the rat main olfactory bulb: possible coexistence of gamma-aminobutyric acid (GABA) and dopamine. Brain Res. 343(1):166-71.
Kosaka K, Toida K, Aika Y, Kosaka T. (1998) Neurosci Res. 30(2):101-10.
Kohwi M, Osumi N, Rubenstein J R, Alvarez-Buylla (2005) pax6 is required for making specific subpopulations of granule and periglomerular neurons in the olfactory bulb. J Neuroscience 25(30): 6997-7003.
Lee J, Platt K A, Censullo P, Ruiz i Altaba A. (1997) Gli1 is a target of Sonic hedgehog that induces ventral neural tube development. Development. 124(13):2537-52.
Lee S K, Lee B, Ruiz E C, Pfaff S L. (2005) Olig2 and Ngn2 function in opposition to modulate gene expression in motor neuron progenitor cells. Genes Dev. 2005 19(2):282-94.
Leventer S M, Wulfert E, Hanin I. Time course of ethylcholine aziridinium ion (AF64A)-induced cholinotoxicity in vivo. Neuropharmacology. 1987 April; 26(4):361-5.
Lewis P M, Gritli-Linde A, Smeyne R, Kottmann A, McMahon A P. (2004) Sonic hedgehog signaling is required for expansion of granule neuron precursors and patterning of the mouse cerebellum. Dev Biol. 270(2):393-410.
Ligon K L, Fancy S P, Franklin R J, Rowitch D H. Olig Gene Function in CNS Development and Disease GLIA 2006 54:1-10
Lois C, Alvarez-Buylla A. (1994) Long-distance neuronal migration in the adult mammalian brain. Science. 264: 1145-1148.
Lu Q R, Yuk D, Alberta J A, Zhu Z, Pawlitzky I, Chan J, McMahon A P, Stiles C D, Rowitch D H. (2000) Sonic hedgehog-regulated oligodendrocyte lineage genes encoding bHLH proteins in the mammalian central nervous system. Neuron. 25(2):317-29.
Luskin M B (1993) Restricted proliferation and migration of postnatally generated neurons derived from the forebrain subventricular zone. Neuron. 11(1):173-89
Machold R, Hayashi S, Rutlin M, Muzumdar M D, Nery S, Corbin J G, Gritli-Linde A, Dellovade T, Porter J A, Rubin L L, Dudek H, McMahon A P, Fishell G. (2003) Neuron. 39: 937-950
Machold R, Hayashi S, Rutlin M, Muzumdar M D, Nery S, Corbin J G, Gritli-Linde A, Dellovade T, Porter J A, Rubin L L, Dudek H, McMahon A P, Fishell G. (2003) Sonic hedgehog is required for progenitor cell maintenance in telencephalic stem cell niches. (Erratum) Neuron. 40(1):189
Merkle F T, Mirzadeh Z, Alvarez-Buylla A. (2007) Mosaic organization of neural stem cells in the adult brain. Science. 317: 381-384.
Muhr J, Andersson E, Persson M, Jessell T M, Ericson J. (2001) Groucho-mediated transcriptional repression establishes progenitor cell pattern and neuronal fate in the ventral neural tube. Cell. 104(6):861-73.
Novitch B G, Chen A I, Jessell T M. (2001) Coordinate regulation of motor neuron subtype identity and pan-neuronal properties by the bHLH repressor Olig2. Neuron. 31(5):773-89.
Pabst O, Herbrand H, Takuma N, Arnold H H. (2000) NKX2 gene expression in neuroectoderm but not in mesendodermally derived structures depends on sonic hedgehog in mouse embryos. Dev Genes Evol. 210(1):47-50.
Palma V, Lim D A, Dahmane N, Sanchez P, Brionne T C, Herzberg C D, Gitton Y, Carleton A, Alvarez-Buylla A, Ruiz i Altaba A. (2005) Sonic hedgehog controls stem cell behavior in the postnatal and adult brain. Development. 132: 335-344.
Paxinos, G., Franklin, K. B. J. The Mouse Brain in Stereotaxic Coordinates: Compact Second Edition. New York, Elsevier, 2003
Qiu M, Shimamura K, Sussel L, Chen S, Rubenstein J L. (1998) Control of anteroposterior and dorsoventral domains of Nkx-6.1 gene expression relative to other Nkx genes during vertebrate CNS development. Mech Dev. 72(1-2):77-88.
Riquelme P A, Drapeau E, Doetsch F. (2008) Brain micro-ecologies: neural stem cell niches in the adult mammalian brain. Philos Trans R Soc Lond B Biol Sci. 363: 123-137.
Saghatelyan A, de Chevigny A, Schachner M, Lledo P M. (2004) Nat. Neurosci. 7(4):347-56.
Sandberg K, Hanin I, Fisher A, Coyle J T. (1984) Selective cholinergic neurotoxin: AF64A's effects in rat striatum. Brain Res. 293: 49-55.
Sohur U S, Emsley J G, Mitchell B D, Macklis J D. (2006) Adult neurogenesis and cellular brain repair with neural progenitors, precursors and stem cells. Philos Trans R Soc Lond B Biol Sci. 361: 1477-1497
Ulloa F, Briscoe J. (2007) Morphogens and the control of cell proliferation and patterning in the spinal cord. Cell Cycle. 6: 2640-26409.
Vallstedt A, Muhr J, Pattyn A, Pierani A, Mendelsohn M, Sander M, Jessell T M, Ericson J. (2001) Different levels of repressor activity assign redundant and specific roles to Nkx6 genes in motor neuron and interneuron specification. Neuron. 31(5):743-55.
Wiiner B, Geyer M, Couillared-Despres S, Aigner R, Bogdahn U, Aigner L, Kuhn G, Winkler J. (2006) Experimental Neurology 66(7): 1044-1048.
Wolpert L. (1996) One hundred years of positional information. Trends Genet. 12(9):359-64.
Zhuang X, Masson J, Gingrich J A, Rayport S, Hen R. (2005) Targeted gene expression in dopamine and serotonin neurons of the mouse brain. J Neurosci Methods. 143(1):27-32

### Example 7

### Dopamine in Adult Neurogenesis

The importance of dopaminergic innervation and of dopamine itself in SVZ physiology has been clearly established: Hoeglinger et al. (2004) recognized that dopaminergic, TH+ afferents make contacts with “A” and “C” cells. “A” and “C” cells, but not “B” cells express D1 like (D1L) and D2 like (D2L) dopamine receptors. Functionally, dopaminergic transmission in the SVZ stimulates the proliferation of EGFR+ cells since dopamine and the D2L agonist bromocriptine increased the proliferation of SVZ derived EGFR+ cells grown as neurospheres in a dose dependent manner. In vivo, the systemic intoxication with the neurotoxin MPTP, which causes bilateral loss of DA neurons or the unilateral destruction of substantia nigra neurons through the unilateral injection of the neurotoxin 6-OHDA into the nigro-striatal pathway, leads to a 40% reduction in SVZ proliferation overall and a 50% reduction in proliferation in the C cell compartment as measured by the proliferation marker PCNA. A single dose of the D2L agonist Ropinirole injected systemically 1 hour prior to brain harvest restores the mitotic activity of the SVZ as measured by PCNA+ cells on the lesioned side and increases the proliferative index on the unlesioned side. Interestingly, the “B”-cell compartment appears not affected by dopaminergic denervation consistent with the finding that “B” cells do not express dopamine receptors.

Winner et al. (2006) reported that unilateral striatal deafferentation mediated by 6-OHDA leads to a 40% reduction in proliferation within the SVZ which is accompanied by a threefold increase in the production of newly born Pax6+, TH+ periglomerular DA neurons in the olfactory bulb ipsilateral to the lesion.

The studies herein demonstrate that dopaminergic innervation regulates C-cell proliferation within the stem cell niche of the SVZ and influences the production of specific neuronal subtype populations at defined relative sizes (Hoeglinger et al, 2004; Winner et al., Exp Neurol. 2006 January; 197(1):113-21). The acute pharmacological complementation of dopaminergic deafferentiation with the dopamine receptor agonist Ropinirole clearly establishes the mitogenic role of dopamine in the SVZ. However, these experiments did not address whether dopamine receptor stimulation also reverts the observed alterations in cell fate determination (i.e. the increase in the numbers of Pax6, Th+ periglomerular DA neurons) and cytoarchitecture of the olfactory bulb (OB). The current body of work does not indicate that other factors besides dopamine are provided to the SVZ by dopaminergic innervation that could be involved in cell fate determination and other aspects of SVZ physiology.

### Example 8

### Sonic Hedgehog (Shh) Expression in Adult Dopaminergic Neurons is Sensitive to Acute and Chronic Cell Physiological Stress in Cholinergic Neurons of the Striatum and Peduncolopontine Tegmental Nucleus (PPTg)

Compromised trophic support of neurons in the nigro-striatal system is thought to contribute to the progressive demise of neuronal populations observed in Parkinson's (PD) and other neurodegenerative diseases involving the basal ganglia. The in vivo regulation of sonic hedgehog (Shh) expressed by dopaminergic (DA) neurons of the mesencephalon in the adult mouse and its function in the expression of glial cell line-derived neurotrophic factor (GDNF) was investigated herein using a combination of conditional, genetic gene ablation studies and acute induction of cell physiological stress by the application of the cholinotoxin ethylcholine mustard aziridium (AF64a). It is found that Shh expression by adult DA neurons is repressed by signals originating from cholinergic (ACh) neurons of the striatum and the pedunculopontine tegmental nucleus (PPTg), rendering Shh expression sensitive to cell physiological stress in, or structural damage of, ACh neurons. In turn, Shh expression in DA neurons represses GDNF expression by ACh neurons of the striatum. The regulation of Shh in DA neurons is uncoupled from the regulation of DA neuron marker gene expression and from any particular cell stress response in DA neurons. Chronic cholinergic stress, as well as acute cholinergic dysfunction in the striatum or the PPTg leads to graded up-regulation of Shh in DA neurons. However, chronic cholinergic stress leads to oxidative stress and down-regulation of DA markers, while acute AF64a injection into the striatum causes a down-regulation of DA markers and an induction of ER stress pathways and acute AF64a injection into the PPTg causes an up-regulation of DA markers and the induction of ER stress pathways. Animals with genetic ablation of Shh expression in DA neurons reveal a heightened sensitivity to the cholinotoxin. The data herein reveal a neuroprotective function of Shh in the adult basal ganglia and demonstrate that physiological stress induced up-regulation of Shh in DA neurons causes the down-regulation of GDNF, a trophic factor for DA neurons. The cross repressive action of Shh and GDNF in this reciprocal trophic support loop can add to the list of vulnerabilities towards neurodegeneration of the adult nigro striatal system.

### Example 9

### Sonic Hedgehog (Shh) Expression in Adult Dopaminergic Neurons

Without being bound by theory, dynamic expression of the secreted cell signaling factor Sonic Hedgehog (Shh) in mesencephalic dopamine (DA) neurons acts as a sentinel for neuronal dysfunction, and, at the same time, as a morphogen in forebrain (SVZ) neurogenesis. In this scenario altered Shh expression by DA neurons of the mesencephalon will function as an instructive signal that is able to skew the qualitative outcome of neurogenesis towards cells of current physiological need.

Physiological cell stress in the projection areas of DA neurons induces graded up-regulation of Shh in DA neurons (FIG. 4) consistent with a “sentinel” function of DA produced Shh. Without being bound by theory, DA-neuron-produced Shh also acts as a morphogen in SVZ neurogenesis. Conditional ablation of Shh from DA neurons results in increased numbers of dopaminergic, periglomerular neurons in the olfactory bulb (OB), but decreased proliferative activity in the SVZ. Thus, the increase in tyrosine dopaminergic, periglomerular neurons must have occurred at the concomitant expense of the production of another, so far unidentified, population of cells normally generated by SVZ neurogenesis (FIG. 3).

To demonstrate a morphogen function for DA-neuron-produced Shh is crucial, the 3 goals below, which are geared towards establishing a morphogenic role of DA neuron produced Shh in SVZ neurogenesis in vivo, were formulated:

1. Determine the mitotic index and size of the SVZ A-, B- and C— cell compartments in mice with Shh ablation in DA neurons;

2. Determine the numbers of Pax6 and Olig2 expressing precursors in the SVZ and the rostral migratory stream (RMS) as a function of Shh expression in DA neurons; and

3. Determine the relative proportions of 5 distinguishable olfactory bulb interneuron populations, which are replenished by neurogenesis as a function of Shh expression in DA neurons.

The size of precursor cell populations and differentiated neuronal populations of the olfactory bulb in the presence of 5 different concentrations of Shh produced by DA neurons will be quantitated: no Shh, wt levels, and 3 distinct levels of increased Shh expression.

Results: In summary the results demonstrate that DA-neuron-produced Shh acts as a morphogen in SVZ neurogenesis.

Relative proportions of distinguishable olfactory bulb interneuron populations. The finding of increased numbers of Pax6+, periglomerular cells and of reduced proliferation in the SVZ indicated that there are other neuronal populations in the bulb that will be replenished less frequently in the absence of Shh expression by DA neurons. Therefore, additional neuronal subtype populations that are altered in animals with conditional ablation of Shh from DA neurons were sought to be identified.

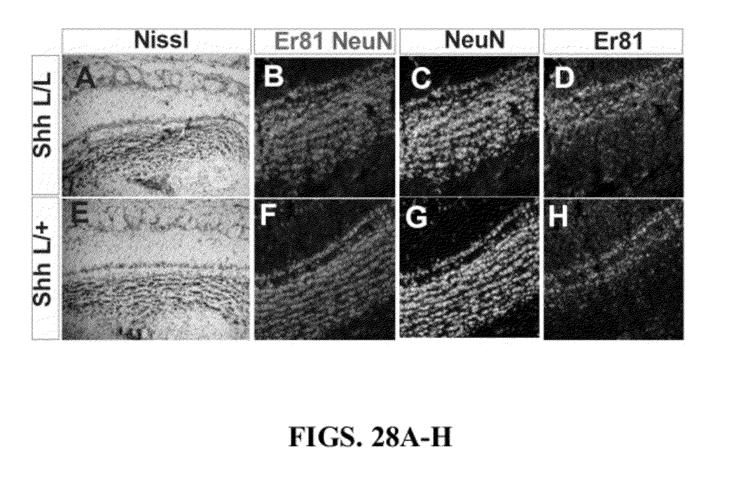

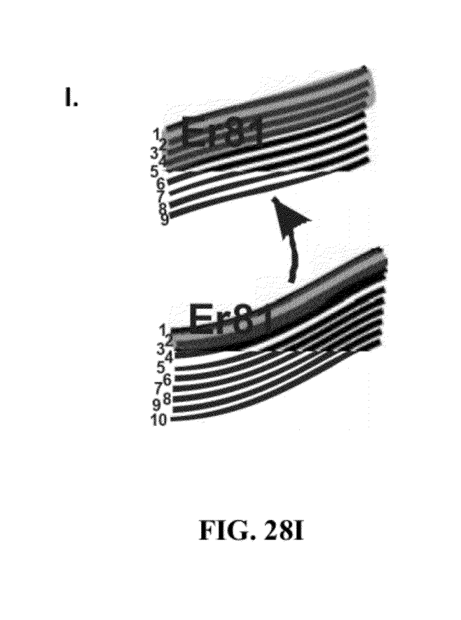

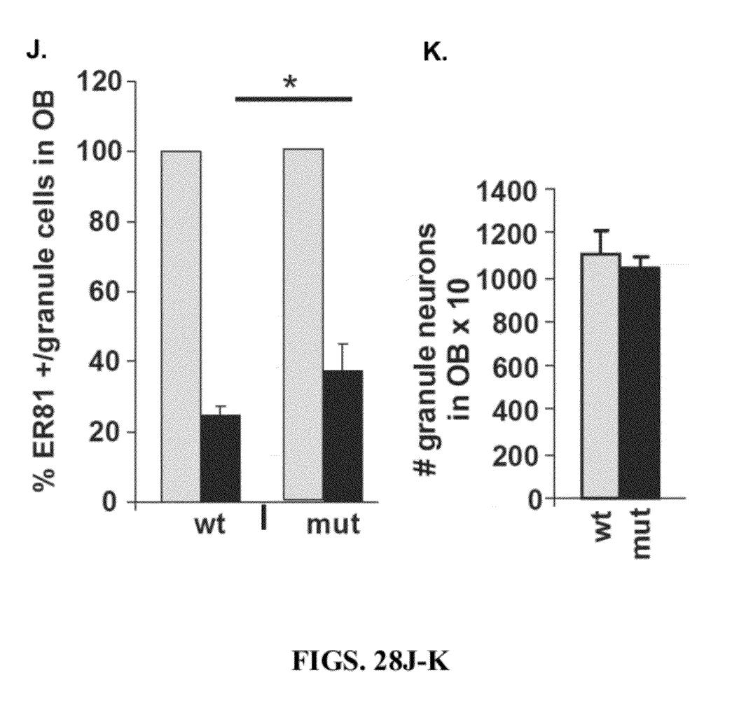

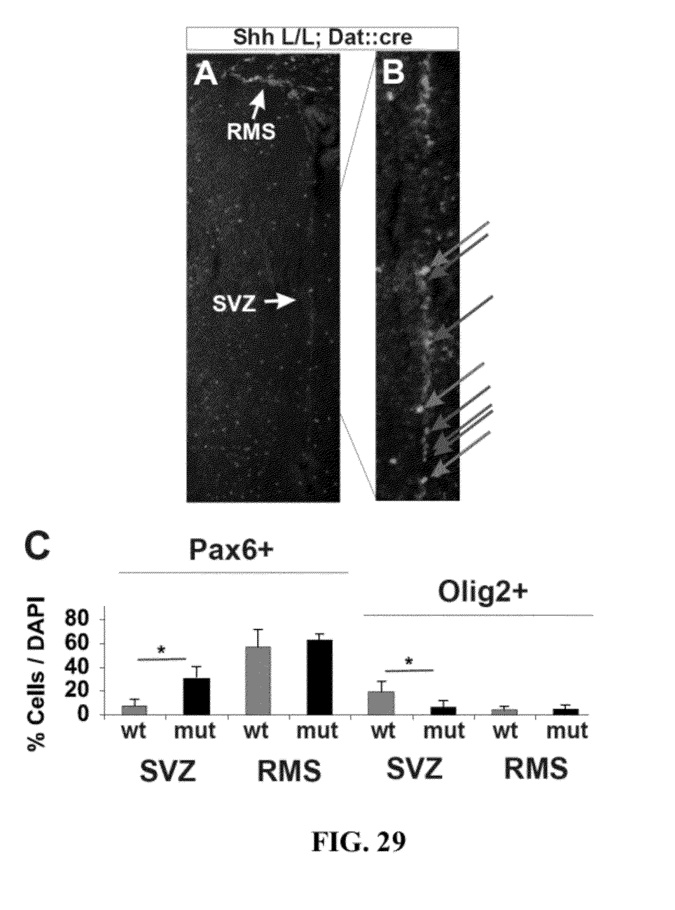

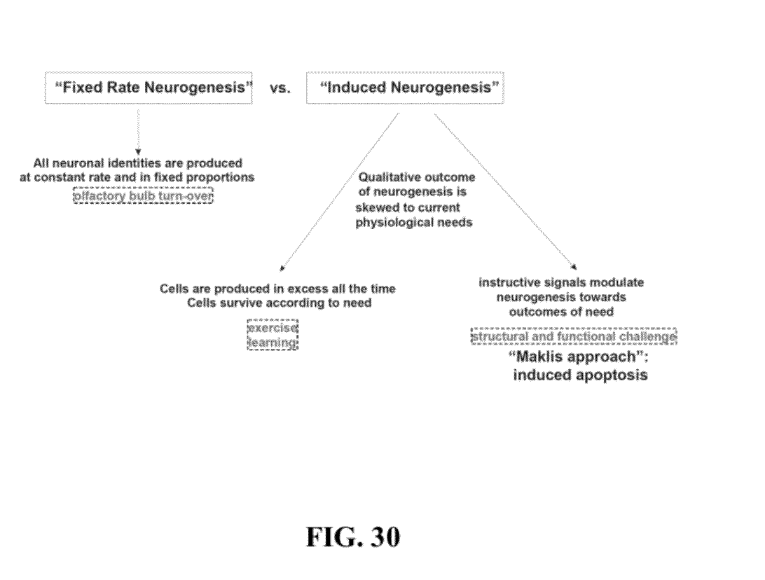

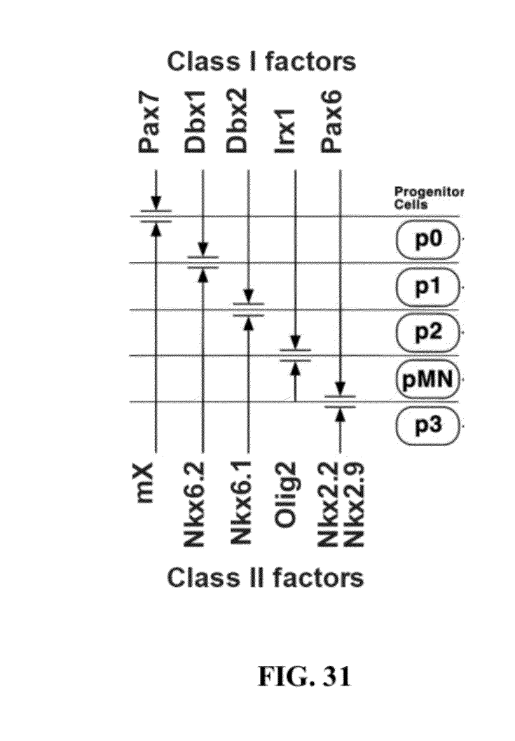

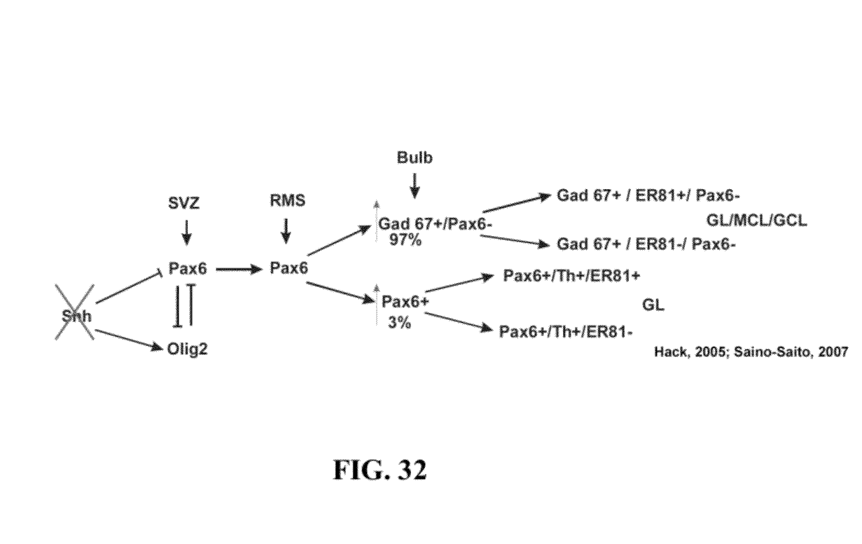

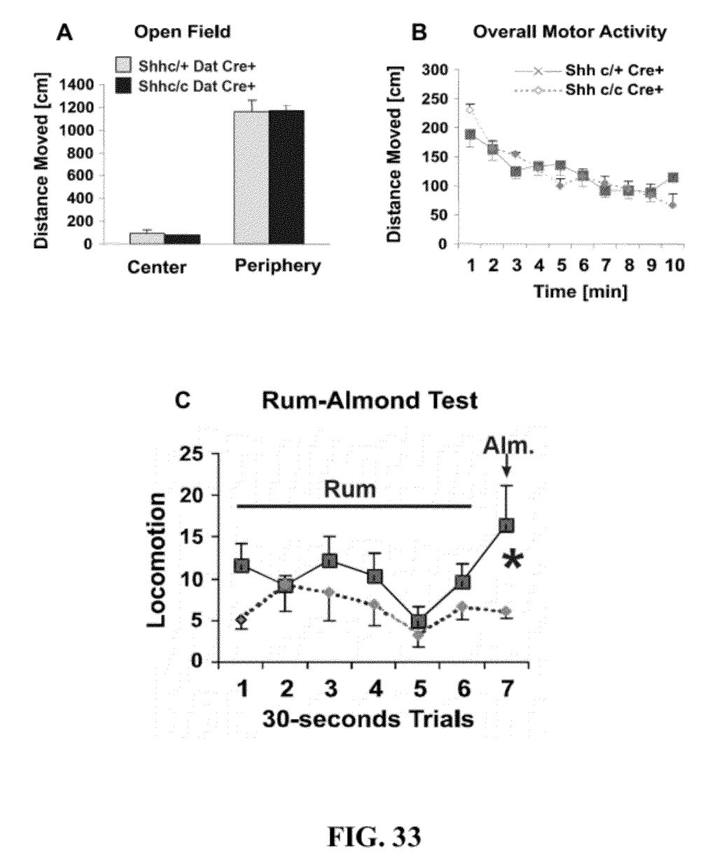

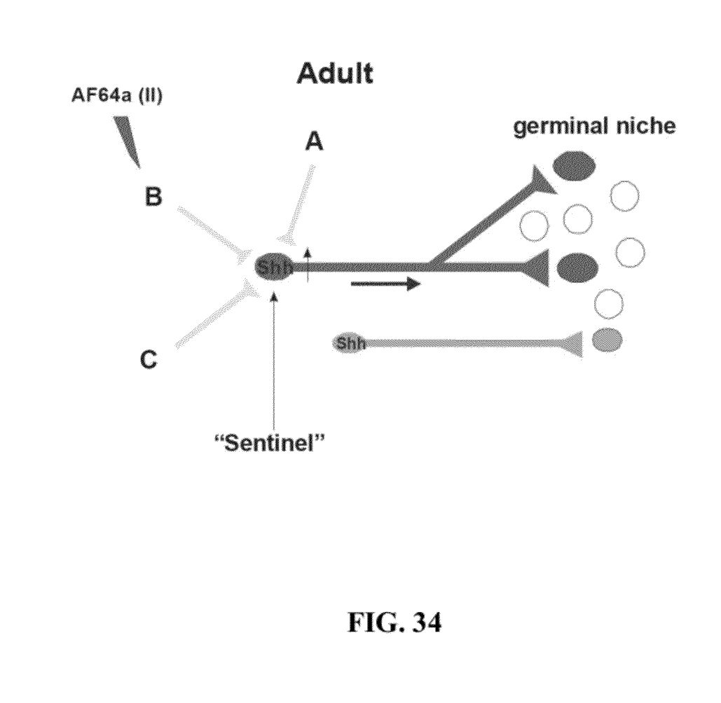

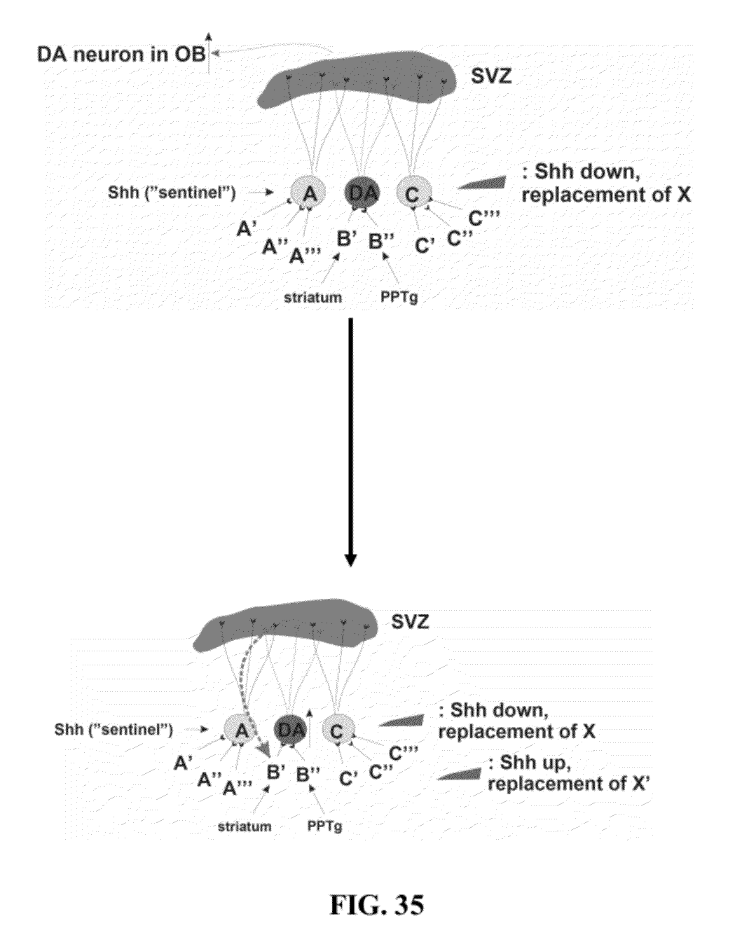

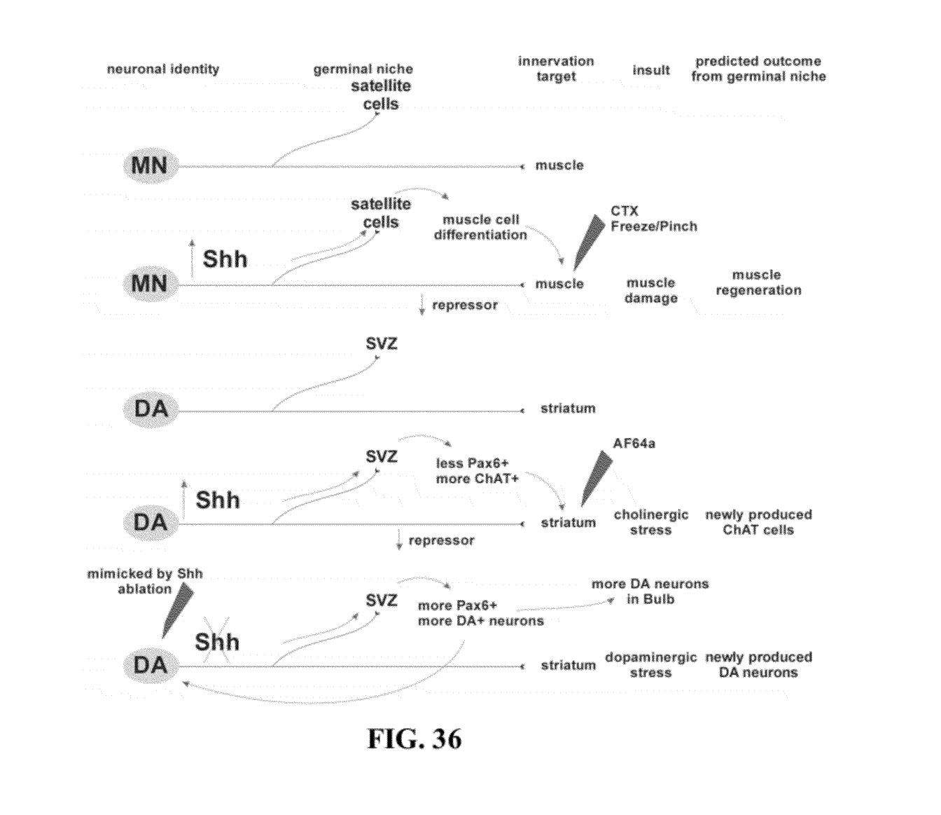

Closer inspection of Niss1 stained coronal sections of olfactory bulbs pointed to a distorted layering of granule cell cartridges in mutant animals (FIG. 28A, FIG. 28E). The transcription factor ER81 is expressed by a subset of granule cells (Saino-Saito S, et al., 2007). In analyzing ER81 marker expression in mutant and control animals, it was recognized that the expression domain of ER81 is extended from the outermost 2 layers of granule cells into layer 4 to 5 in mutant animals (FIG. 28B, FIG. 28D, FIG. 28F, FIG. 28G). Sterological counting of granule cells reveals that the total number of granule cells remains unaltered (FIG. 28H). Thus the expansion of the ER81+ domain occurs at the expense of the ER81− domain among granule cells of the olfactory bulb.

The proportion of ER81+ granule cells among all granule cells as a function of Shh expression by DA neurons was quantified. In wt animals, 24±4% of all granule cells express ER81 as compared to 38±10% (FIG. 28J). Results are expressed as the mean±SEM, cells were counted on 40 μm floating, coronal sections encompassing the entire a/p extent of the bulb (12 sections with a 4-section interval), 3 animals per genotype, left and right hemisphere analyzed separately.

Granule cell numbers as a function of Shh expression by DA neurons was quantified. There was no statistically significant difference in the numbers of granule cells between genotypes (FIG. 28K). Cell numbers were calculated by stereological quantification using a Stereoinvestigator 4.34 (Colchester, Vt.) software running an automatic x-y stage on a Zeiss Axioplan2 microscope. Cells were counted on 40 μm floating sections encompassing the entire a/p extent of the bulb (12 sections with a 4-section interval). n=3 animals per genotype/age, and left and right hemispheres were analyzed separately.

In subsequent experiments, the relative proportions ER81+ and ER81− granule cells in animals with induced up-regulation of Shh expression by DA neurons will be quantitated.

Pax6 and Olig2 expressing precursors in the SVZ and the rostral migratory stream (RMS). Alterations in the cyto-architecture of the bulb can result from changes in cell fate determination in precursor domains or from altered selection or survival of mature neurons. Thus, the relative proportions of precursor cell populations as a function of Shh expression levels were examined Olig2 and Pax6 expressing precursor cell populations were chosen for the study since class I type transcription factors like Pax6 and Pax7, are repressed by Shh signaling, whereas expression of class II proteins, like Nkx and Olig2, requires exposure to Shh (Ericson et al. 1997b; Qiu et al. 1998; Briscoe et al. 1999, 2000; Pabst et al. 2000; Vallstedt et al. 2001). These transcription factors are expressed in the adult SVZ and RMS in wt animals (Hack et al., 2005). Within the SVZ and RMS, the relative size of the cell populations that express these markers follow opposite gradients (Hack et al., 2005). While there are few Pax6 expressing cells in the SVZ proper, the majority of all cells in the RMS are Pax6+. In contrast, Olig2 is expressed relatively more abundantly in the SVZ and much more sparsely in the RMS.

In animals with Shh ablation from DA neurons compared to control litter mates, an enlargement of the Pax6 expressing precursor population was found in the SVZ from 7±5%) to 31±12% (p<0.05; students T-test). There was a slight, but not significant increase in the proportions of the Pax6+ cells in the RMS. Conversely, a decrease in the frequency of Olig2 expression in the SVZ from 20±12% to 8±6% (p<0.01) is found. There was no significant difference in the relative size of the Olig2 expressing population in caudal RMS.

From these experiments, it is concluded that Shh expressed by DA neurons influences cell fate determination in the SVZ following predictable rules similar to those that govern neuronal differentiation of the ventral CNS during embryogenesis. The differences in relative size of precursor populations within the SVZ do not translate into readily observable changes within the RMS.

Without being bound by theory, the failure to detect alterations in the relative sizes of migrating cell populations in the RMS can have several reasons: (1) Ceiling and flooring effects. The predicted changes will further increase the size of the Pax6+- and further reduce the size of the Olig2+-cell populations making it difficult to recognize these differences against the control situation; and/or (2) The mechanisms that act on cells emigrating from the SVZ into the RMS and “sculpt” the relative proportions of cell populations in the RMS can counteract the disturbances in cell fate determination in the SVZ. For example, Olig2+ cells can be subjected to a reduced frequency of apoptosis while Pax6++ cells might suffer apoptosis more frequently within the RMS. However, the results from FIG. 28 indicate that these compensatory mechanisms cannot balance out the alterations in cell fate determination completely since mature descendants of the Pax6 lineage, i.e. periglomerular dopaminergic- and ER81+ neurons, do accumulate in the olfactory bulb.

The same pair of transcription factors in animals with induced up-regulation of Shh expression by DA neurons will be examined.

### LITERATURE CITED

Briscoe J, Pierani A, Jessell T M, Ericson J. (2000) A homeodomain protein code specifies progenitor cell identity and neuronal fate in the ventral neural tube. Cell. 101(4):435-45.
Ericson J, Briscoe J, Rashbass P, van Heyningen V, Jessell T M. (1997b) Graded sonic hedgehog signaling and the specification of cell fate in the ventral neural tube. Cold Spring Harb Symp Quant Biol. 62: 451-466.
Hack M A, Saghatelyan A, de Chevigny A, Pfeifer A, Ashery-Padan R, Lledo P M, Gotz M. (2005) Neuronal fate determinants of adult olfactory bulb neurogenesis. Nat Neurosci 8(7):865-72.
Pabst O, Herbrand H, Takuma N, Arnold H H. (2000) NKX2 gene expression in neuroectoderm but not in mesendodermally derived structures depends on sonic hedgehog in mouse embryos. Dev Genes Evol. 210(1):47-50.
Qiu M, Shimamura K, Sussel L, Chen S, Rubenstein J L. (1998) Control of anteroposterior and dorsoventral domains of Nkx-6.1 gene expression relative to other Nkx genes during vertebrate CNS development. Mech Dev. 72(1-2):77-88.
Saino-Saito S, Cave J W, Akiba Y, Sasaki H, Goto K, Kobayashi K, Berlin R, Baker H. (2007) ER81 and CaMKIV identify anatomically and phenotypically defined subsets of mouse olfactory bulb interneurons. J Comp Neurol. 2007 Jun. 1; 502(4):485-96.
Vallstedt A, Muhr J, Pattyn A, Pierani A, Mendelsohn M, Sander M, Jessell T M, Ericson J. (2001) Different levels of repressor activity assign redundant and specific roles to NRx6 genes in motor neuron and interneuron specification. Neuron. 31(5):743-55.

### Example 10

### Mice with a Genetic Ablation of Shh from Mesencephalic DA Neurons Constitute a Model of Pd with Construct, Predictive and Face Validity

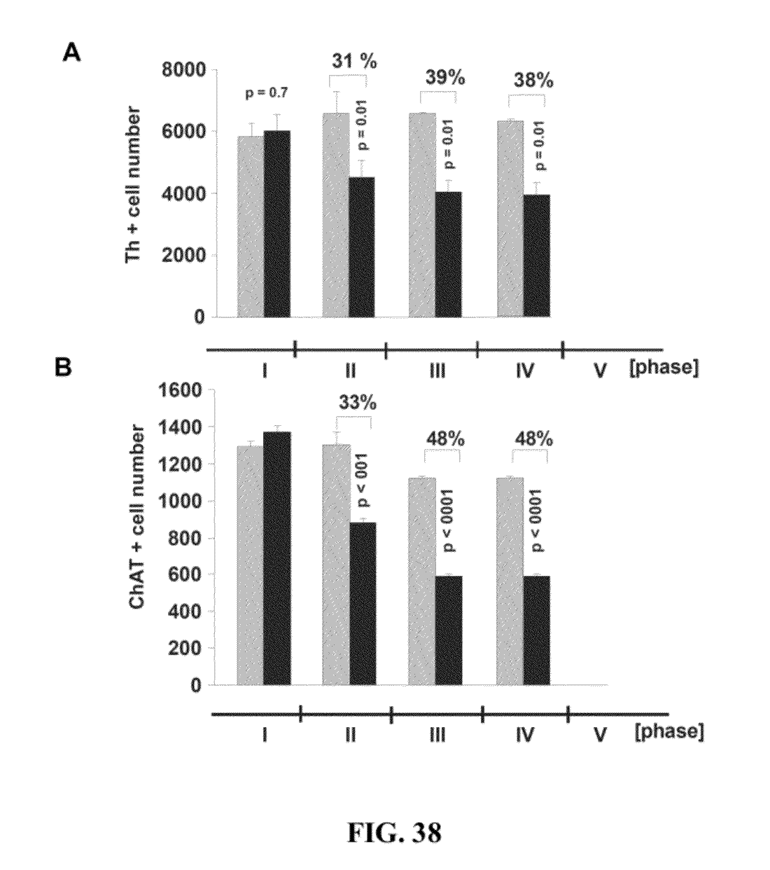

Animals with a genetic ablation of Shh from dopaminergic neurons as described in FIG. 8 exhibit adult onset, progressive loss of cholinergic neurons of the striatum (FIG. 38A), adult onset, progressively reduced production of striatal GDNF production (FIGS. 6G and 6I) and adult onset, progressive loss of mesencephalic DA neurons including Substantia Nigra pars compacta (SNpc) and ventral tegmental area (VTA) dopaminergic neurons (FIG. 38B).

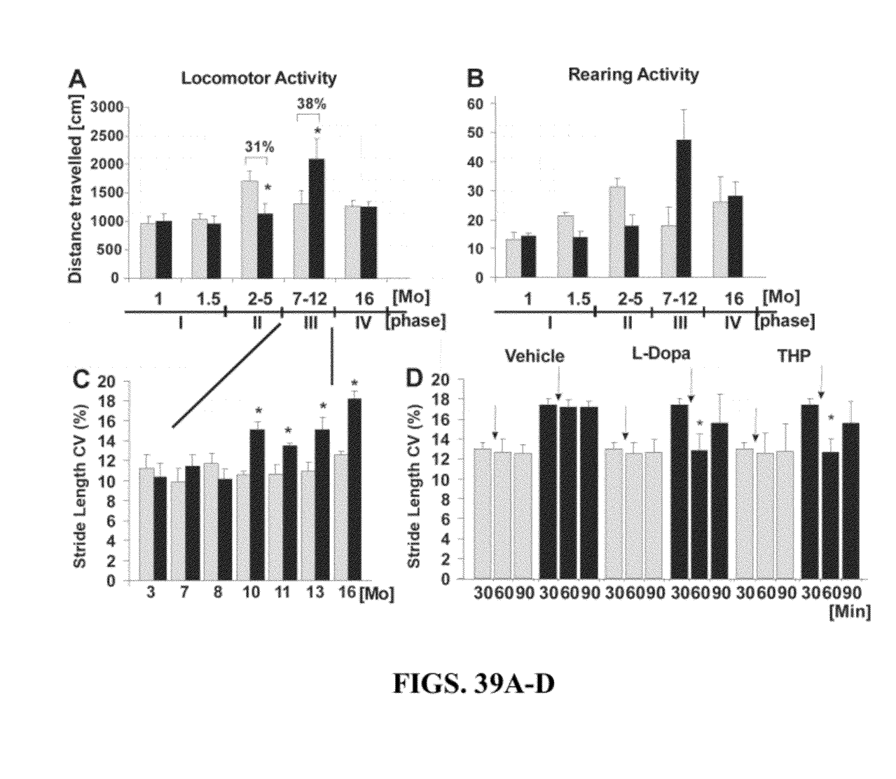

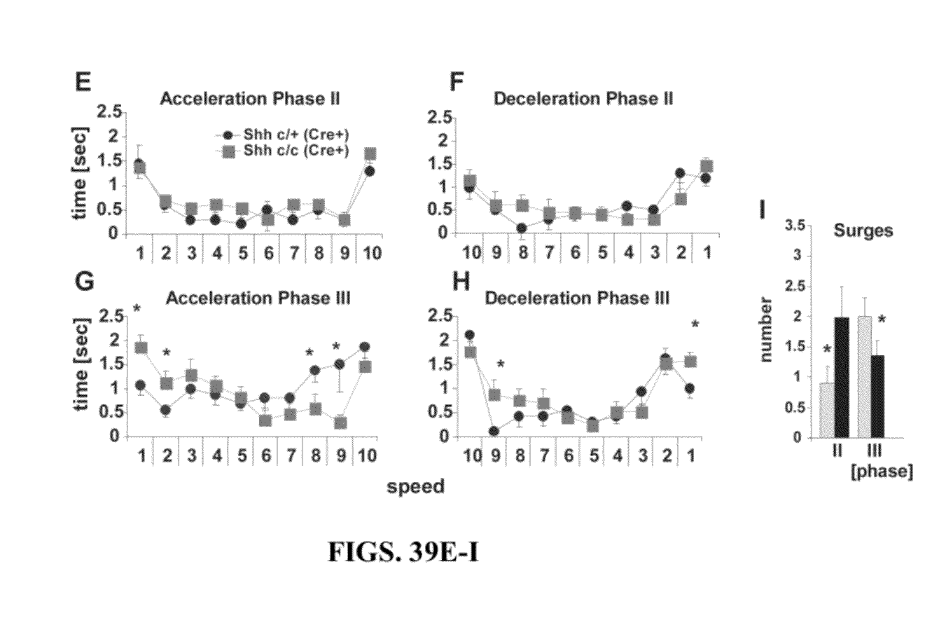

Longitudinal behavioral analysis provides endpoint measures for the functional changes associated with the tissue specific ablation of Shh from DA neurons. To assess the motor performance of experimental animals, qualitative home cage observations were first used and then spontaneous locomotion in the Open Field paradigm, a behavioral test used frequently to characterize animals with deficit in the dopaminergic nigrostriatal system, was quantified (Fleming et al., Behav Brain Res. 2005 Jan. 30; 156(2):201-13; Meredith et al, Mov Disord. 2006 October; 21(10):1595-606; Sedelis et al., Behav Brain Res. 2001 Nov. 1; 125(1-2):109-25; and Zhou and Palmiter, Cell. 1995 Dec. 29; 83(7):1197-209). Mutant animals appear inconspicuous in their home cage up to about 15 months of age at which point pelvic dragging becomes apparent. By 17 months animals exhibit partial hind limb paralysis and most animals die prematurely by about 18 months. However, automatic video tracking of locomotion in an “open field” arena reveals a multiphasic phenotype that let us define discreet phases: In fair agreement with the histological data, no difference in locomotion activity in juveniles and young adults (phase I) was observed between experimental and control animals. Between 3 to 5 months of age, however, mutant animals first exhibit hypokinesis (phase II, 30% reduction in activity, n=36 per genotype, several litters reared around the year, p<0.01, ANOVA) followed by hyperkinesis with a 100% increase in locomotion activity compared to phase II and a 38% increase compared to control animals between 7-12 months of age (phase III; n=37 per genotype, p<0.01, ANOVA). By 16 months of age (phase IV) locomotion activity has returned to control levels in mutant animals which then progress to a phase (V) of rapid neurological decline and premature death at 18 months of age (FIG. 39A). In fair agreement with the horizontal movement described above, rearing activity is also altered qualitatively with a similar multiphasic dynamics (FIG. 39B).

Given the involvement of the basal ganglia in the production of gait patterns, gait dynamics by ventral plane videography of mice walking on a translucent treadmill was investigated (Digigait system, Mouse specifics, Inc.), from which comparative temporal, spatial and force indices of gait were derived (Hampton et al., Physiol Behav. 2004 Sep. 15; 82(2-3):381-9; Amende et al., J Neuroeng Rehabil. 2005 Jul. 25; 2:20) of experimental and control animals from 3 to 16 months. Among all the measures (in total 41 indices obtained form the DigiGait system), gait length variability was indistinguishable until 8 months of age but increased significantly in front and hind limbs (n=5, p<0.046, p<0.001; resp., student's t-test) at 10 months of age but not at 2 and 7 months (FIG. 39C) and the time allocated to braking the swing phase was shortened at 12 months but not at 2 and 7 months in hindlimbs (n=5, p<0.003, student's t-test), FIG. 39A).

Dopamine substitution and anticholinergic pharmacology normalize gait disturbances in animals with genetic ablation of Shh from mesencephalic DA neurons. Levodopa therapy (Cotzias et al., Science. 1977 Apr. 29; 196(4289):549-51; Tolosa et al., Neurology. 1998 June; 50(6 Suppl 6):52-10; discussion S44-8) is the “gold standard” treatment for dopaminergic deficiency. Levodopa normalizes many of the locomotion deficits observed in PD, like reduction in gait length and increases in gait variability, within minutes of oral dosing in patients with early PD who were started on Levodopa recently (Singh et al., J Clin Neurosci. 2007 December; 14(12):1178-81; Moore et al., Neurobiol Dis. 2008 March; 29(3):381-90). L-DOPA also reverses motor impairments in mice with a loss of nigrostriatal DA neurons (Hwang et al., J. Neurosci. 2005 Feb. 23; 25(8):2132-7; Fleming et al., Behav Brain Res. 2005 January 30; 156(2):201-13; and Lindner et al., Brain Res Bull. 1996; 39(6):367-72). Anticholinergic drugs, like trihexiphenidyl (THP), were the first drugs available to the symptomatic treatment of the locomotion deficits in PD and are thought to be particularly efficacious in reducing rigidity and the frequency and duration of gait freezing (Brumlik et al, J Nerv Ment Dis. 1964 May; 138:424-31; Parmar et al., J Postgrad Med. 2000 January-March; 46(1):29-30; and Rezak, Dis Mon. 2007 April; 53(4):214-22).

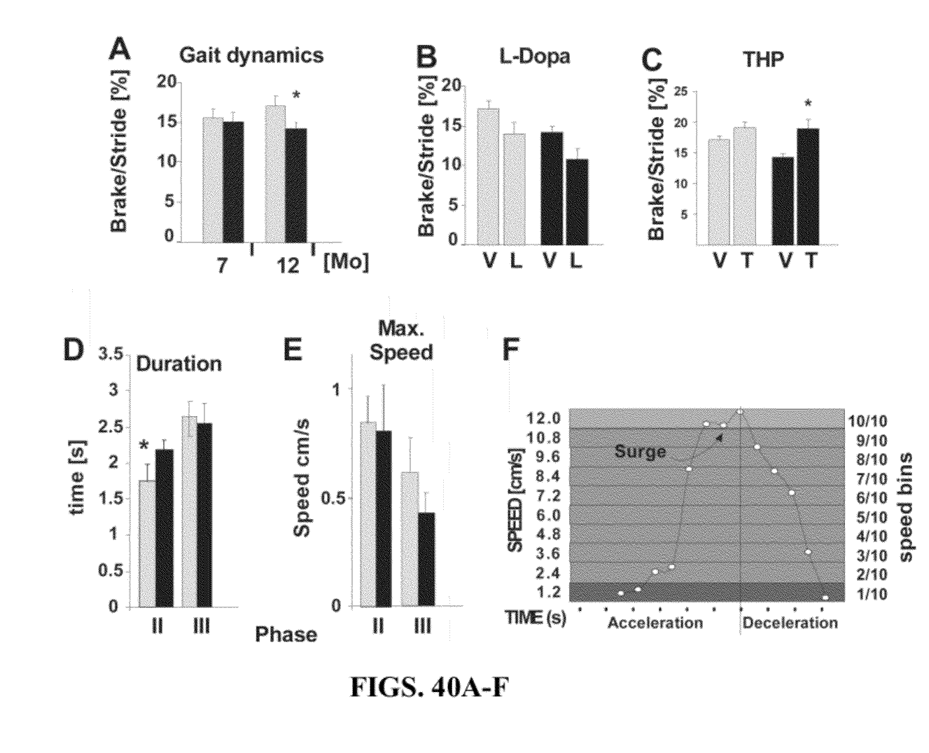

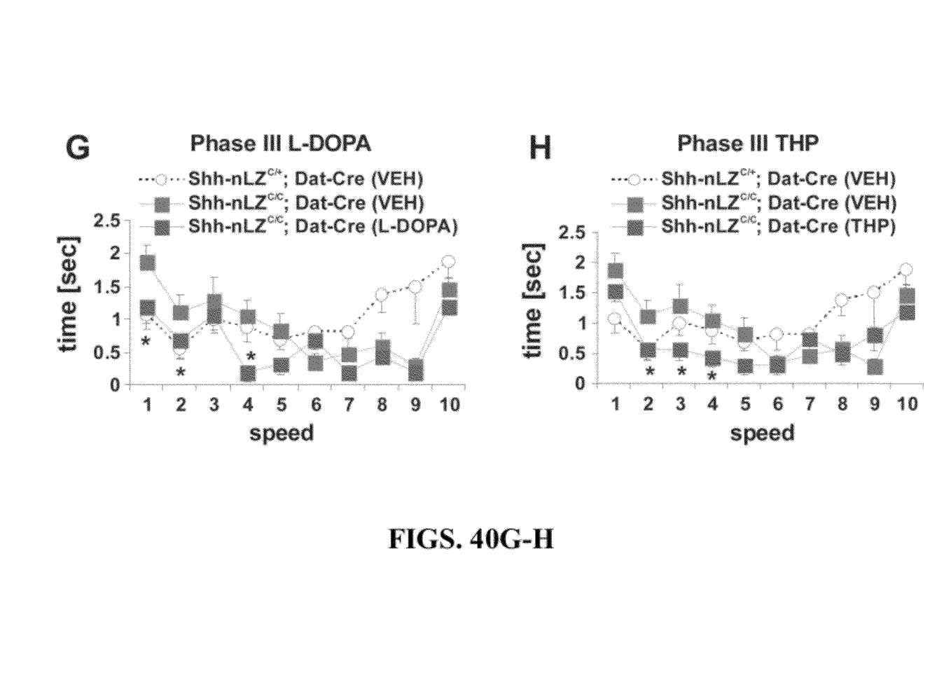

Whether Levodopa (20 mg/kg, SC, Fredriksson et al., Pharmacol Toxicol. 1990 October; 67(4):295-301) and/or THP (3 mg/kg, IP, Goldschmidt et al., Prog Neuropsychopharmacol Biol Psychiatry. 1984; 8(2):257-61) administration will acutely effect gait variability and the length of the brake phase in the absence of DA produced Shh was investigated. Either drug or vehicle control 30 minutes prior to the analysis of gait dynamics was injected in 12 month old animals. The increased variability in stride length observed in experimental animals (CV, FIG. 39C) was normalized to control levels by L-Dopa (20 mg/kg SC) [Drug×Genotype, F(1,37)=3.5, p<0.05] and THP (3 mg/kg, IP) [Drug×genotype (1,37)=4.2, p<0.04; 2-Way ANOVA followed by Tokey HSD post-hoc test; n=10 measures of right and left hind limbs derived from 5 animals of 12 months of age/genotype each] (FIG. 39D). L-Dopa did not correct the reduction in brake time observed in experimental animals but instead reduced Brake-Stride ratios in both experimental and control animals [Genotype×Drug, F(1,37)=0.01; not significant] (FIG. 39B). In contrast THP normalized Brake Stride ratios to control levels [genotype×Drug, F(1,37)=3.3; p<0.05; 2-Way ANOVA followed by Tokey HSD post-hoc test; n=10 measures of right and left hind limbs from 5 animals of 12 months of age/genotype each] (FIG. 40C).

Patients with neurological diseases of the basal ganglia and in particular PD exhibit a specific set of deficits in the realm of locomotion initiation and fluidity of movement. In particular, Bradykinesia is observed in PD. Bradykinesia is the slowed ability to start and continue movements, and impaired ability to adjust the body's position. Spontaneous locomotion aided by automatic video tracking in an “open Field” setting was therefore examined Slightly increased lengths of individual locomotion bouts in phase II but not differences in phase III were observed (FIG. 40D). There were no significant differences in the maximal speed that control and mutant mice can travel at (FIG. 40E). The relative time each animals spends at different speeds during acceleration and deceleration in individual locomotion bouts was analyzed. The numbers of “surges”, that are the reversals from acceleration to deceleration and back within a single locomotion bout, were quantitated. The “speed bin” analysis and quantitation of surges type is schematized in FIG. 40F.

Applying this model to the study of spontaneous locomotion in animals without Shh expression by DA neurons and controls, no difference among all animals in phase II for either the acceleration or deceleration segment in each locomotion bout (FIGS. 39E-F) is found. However, in phase III, this analysis reveals that mutant animals spend significant more time in low speed bins and less time in medium to high speed bins, although they reach the same maximal speed, than control animals during the acceleration segment (FIG. 39G). Hence, mutant animals take longer to start a locomotion bout, i.e., they spend relative more time at low speeds during locomotion initiation. For the deceleration segment, a complementary distribution of times spent at different speed levels is found: Mutant animals spend more time at the second highest and at the lowest speed levels (FIG. 39H). Hence, once mutant animals are traveling at top speed, it takes them longer to initiate deceleration. The phenotype discovered here corresponds to a classic recapitulation of Bradykinesia observed in PD.

L-Dopa and THP, drugs used in the management of PD, normalize the deficit during locomotion initiation but not those observed at higher speeds during the acceleration segment (FIGS. 40G-H).

Mutant animals also show reduced “fluidity of movement” as seen by a reduction of “surges” in phase III (FIG. 39I). Again, this deficit is similar to what is observed in PD.

Bradykinesia and “reduction in fluidity” of movement have so far not been reported in models of PD. In mutant mice, this phenotype is seen to develop in a temporal specific manner in that it only develops in the 2nd half of adult life The late onset correlates with the advance cellular deficits described in FIG. 38.

As discussed herein, a progressive genetic model of PD (or other progressive genetic models of neurodegenerative diseases) with face and predictive validity can be used for the purposes of drug screening, validation of already existing drugs marketed for other indications, as well as validation of other animals models for neurodegenerative diseases.

### Example 11

### Reciprocal Trophic Signaling Involving Sonic Hedgehog Maintains the Adult Nigrostriatal Circuit

Mesencephalic dopaminergic neurons, and cholinergic and gabaergic inter-neurons of the striatum form the mesostriatal circuit, which gates the glutamatergic drive onto striatal medium spiny projection neurons. Dopaminergic neurons produce sonic hedgehog (Shh) throughout life and transport it to the striatum, where it is necessary for the survival of cholinergic- and a subset of gabaergic-neurons and the regulation of extracellular actylcholine concentration. Moreover, acute Shh signaling inhibits expression of GDNF, a dopaminergic survival factor, in striatal cholinergic neurons. Reciprocally, signals emanating from cholinergic neurons repress Shh expression by dopaminergic neurons. Thus, mesencephalic dopaminergic neurons and striatal cholinergic neurons can promote each other's long-term survival through the relief of reciprocal negative feedback on trophic factor signaling. It is shown herein that loss of trophic signaling leads to progressive, late-onset, neuronal loss, alterations in the balance of striatal acetylcholine and dopamine, and functional deficits, thus defining a new mechanism for the homeostatic maintenance of the mesostriatal circuit.

A principal form of circuit design is the filtering of information propagated by long range excitatory pathways by neuromodulatory networks and local inhibitory interneurons which are organized in repetitive, functional units (Silberberg et al., 2005). In each unit several interneuronal subtypes are interconnected in seemingly optimized relative numbers that match the particular circuit function in subserving normal behavior (Grillner et al, 2005). The combinatory apposition of neuromodulatory, inhibitory and excitatory neuronal subtypes greatly expands the repertoire of strategies available to the CNS for information processing as well as for homeostatic regulation of plastic changes in circuit performance in the service of learning and memory (Maffei and Fontanini, 2009). It also raises questions about how the circuit type specific relative proportions of the constituent neuronal subtypes, which presumably have very different survival needs and risks, is maintained for many decades.

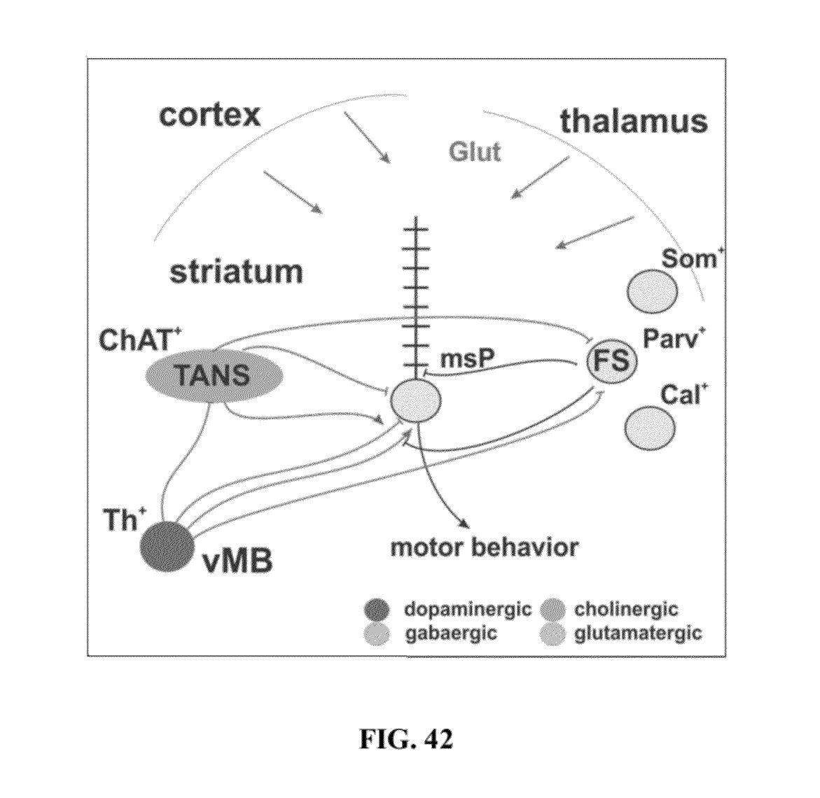

The basal ganglia have served since long as a model system for the study of mechanisms involved in the stabilization of complex neuronal circuitry throughout the lifespan of an organism in part motivated by the crucial involvement of the basal ganglia in many prominent, progressive neurodegenerative diseases. The striatum, the input nucleus of the basal ganglia, processes information of sensory and motor states and thereby facilitates the translation of thought into appropriate behavioral actions that lead to desired outcomes and the avoidance of undesirable ones (Yin et al., 2009; Jin and Costa, 2010; Ding et al., 2010). Striatal computations rely on neurotransmitter mediated correlated changes in the activity of meso striatal dopamine (DA-) neurons of the ventral midbrain and tonically active, cholinergic (ACh-) neurons and fast spiking (FS-), gabaergic neurons of the striatum that involve reciprocal presynaptic regulation of neurotransmitter release, and postsynaptic interactions (Threlfell et al., 2010; Bonsi et al., 2011; Tepper et al., 2010). The concerted actions of DA-neurons and the locally projecting ACh- and FS-neurons gate powerfully the glutamatergic input onto medium spiny projection neurons (msPs) of the striatum which results from massive and convergent projections from the cortex and thalamus (FIG. 42; Ding et al., 2010). msPs make up 85 to 95% of all striatal neurons while the populations of DA-, ACh-, and FS-neurons are 10 to 20 times smaller (Oorschot, 2010). These neuronal subtypes form cartridges of a repetitive meso-striatal circuit in which each msP contributes to few units, each DA-, ACh- and FS-neuron, however, participates in several 100 meso-striatal cartridges (Bolam et al., 2006).

The phylogenetic conservation of circuit architecture (Reiner, 2010) indicates that the relative proportions of the constituent cell types that make up the mesostriatal circuit are important for proper circuit function. This view is supported by the histopathological findings of the select corruption of individual classes of constituent neurons in prominent movement disorders: Huntington Disease is caused by the loss of msPs and Parkinson's Disease (PD) by the loss of DA-neurons while decreased numbers of ACh-neurons are associated with Supra nuclear palsy and of FS-neurons with Tourette Syndrome (Vonsattel and DiFiglia, 1998, Albin et al., 1989, Ruberg, et al., 1985 Kataoka et al, 2010). Many of the hyper- and hypo-locomotive states in these diseases are responsive to pharmacological treatment strategies revealing that the net effect of dopaminergic and cholinergic signaling has opposing effects on the generation of motor output signals from the striatum (Lester et al., 2010). These observations indicate the existence of powerful mechanisms that ensure homeostatic and coordinated regulation of dopaminergic and cholinergic signaling in the healthy striatum that might go beyond neurotransmitter mediated signaling.

The mechanisms maintaining cellular and neurochemical homeostasis in the mature meso-striatal system in the healthy brain have not been fully elucidated. However, neurotrophic factors have emerged as therapeutic tools for neurodegenerative diseases, owing to their effects on the promotion of survival, differentiation and phenotype of neurons (Manfredsson and Mandel, 2010) and indicating that signaling by target derived neurotrophic factors among the neuronal constituents can contribute to the maintenance of circuit architecture and function in the adult basal ganglia.

Among those agents that can play a role in the stabilization and function of meso striatal circuitry, the dopaminotrophic glial cell line-derived neurotophic factor (GDNF) has received special attention because of its potential utility in the treatment of PD (Airaksinen and Saarma, 2002). GDNF is a potent neurotrophic factor that protects catecholaminergic neurons from toxic damage, induces fiber outgrowth and is absolutely required for catecholaminergic neuron survival in the adult (Lin et al., 1993; Pascual et al., 2008). GDNF signaling can also act as a neuromodulator of dopaminergic signaling through the regulation of the quantal size of DA release and neuronal excitability (Pothos et al., 1998; Wang et al., 2001). Despite the implication of GDNF dependent signaling in DA neuron maintenance and function, which motivated several clinical trials to test the utility of GDNF based therapies in PD with controversial outcomes (Gill et al., 2003; Slevin et al, 2005; Lang et al., 2006), further studies can better define the relevant source of GDNF and the regulation of its expression in the adult brain.

Without being bound by theory, the secreted glycoprotein Sonic Hedgehog (Shh), a morphogen which takes part in the differentiation of mesencephalic DA neurons (Joksimovic et al, 2009; Hammond et al., 2009), is involved in the maintenance of mesostriatal circuitry in the adult brain. Supranigral administration of Shh into 1-methyl-4-phenyl-1,2,3,6-tetrahydropyridine (MPTP) treated marmosets, a neurotoxicological model of DA neuron degeneration induced Parkinsonism, is more potent than GDNF in reversing motor disabilities in this experimental model (Dass et al., 2002). Shh administered into the striatum of rats also reduces dopaminergic cell death upon intoxication with 6-hydroxy-dopamine (6-OHDA), another neurotoxicological model of DA neuron loss mediated parkinsonism, and reduces amphetamine-induced rotation when administered before and after striatal toxin treatment (Tsuboi et al., 2002). Expression of Shh from adenovirus associated viruses (AAV) in the striatum protects mesencephalic DA neurons from 6-OHDA toxicity (Dass, 2005). Shh can also act as a neuromodulator altering the frequency of neuronal discharges in slice preparations of the subthalamic nucleus and the tractus solitarius (Bezard, et al., 2003; Pascual et al., 2005). In vitro experiments indicate that ACh-neurons might be a potential target of Shh signaling within the mesostriatal circuit since neural growth factor (NGF) synergizes with Shh in providing trophic support to basal forebrain derived, post natal cholinergic neurons in vitro (Reilly et al, 2002). Without being bound by theory, there is a functionally relevant source of Shh that can act in the mature meso-striatal system and if so, which cell types will communicate by Shh signaling.

The studies described herein show a means by which mature mesencephalic DA neurons communicate selectively with striatal ACh- and FS-neurons, namely by Shh signaling. Based on results from genetic gene expression-tracer and conditional ablation strategies in combination with acute pharmacological and neurotoxicological perturbations, evidence that the mature meso-striatal circuit embodies a reciprocal, trophic factor support loop with homeostatic properties that is required for the interdependent maintenance of ACh- and FS-interneurons in the striatum on one side and DA-neurons of the ventral midbrain on the other is presented herein. One arm of this loop is provided by the expression of the axonaly transported cell signaling factor Shh by mesencephalic DA-neurons, which signals to ACh- and FS-neurons and whose expression is repressed by signals emanating from ACh-neurons. The other arm is provided by GDNF, which is expressed by all ACh-neurons throughout life and whose expression is repressed by DA-neuron produced Shh. Evidence that Shh signaling modulates the extracellular tone of ACh in the striatum through the regulation of muscarinic autoreceptor expression and efficacy of autoreceptor signaling is also presented herein. The chronic, genetic interruption of Shh signaling causes late life onset, progressive alterations in locomotion activity and gait dynamics that can be ameliorated by DA substitution and anti cholinergic pharmacology of proven efficacy in the management of PD. In wt animals, spontaneous loss of Shh expression by select DA neurons in late adulthood is observed, indicating an unexpected mechanism for the spread of increased risk of neuronal demise throughout the population of mesencephalic DA neurons with possible relevance to idiopathic neurodegenerative diseases involving the meso-striatal circuit.

### Results

Shh and Ptc1 Expression by Neurons of the Adult Meso-Striatal Circuit.

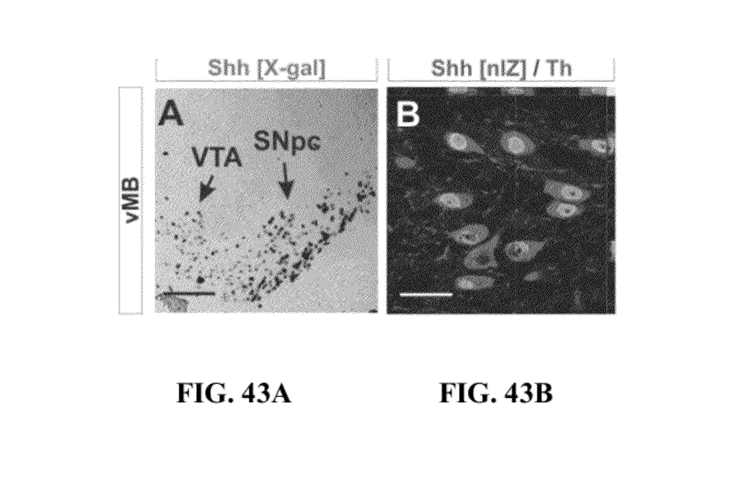

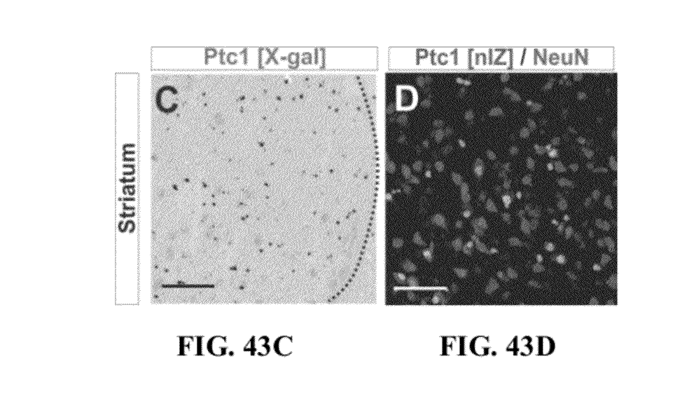

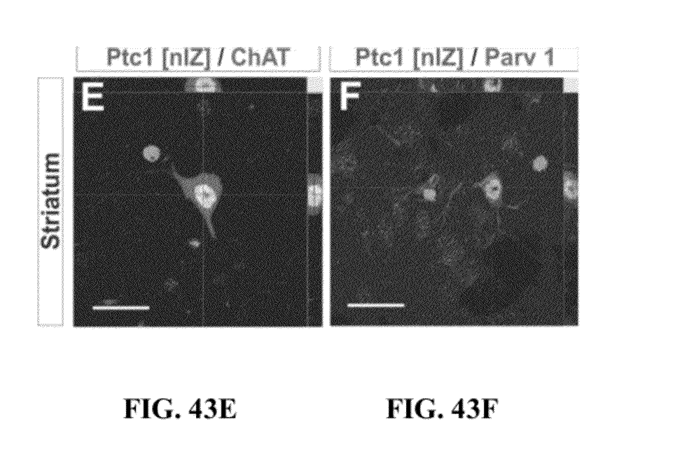

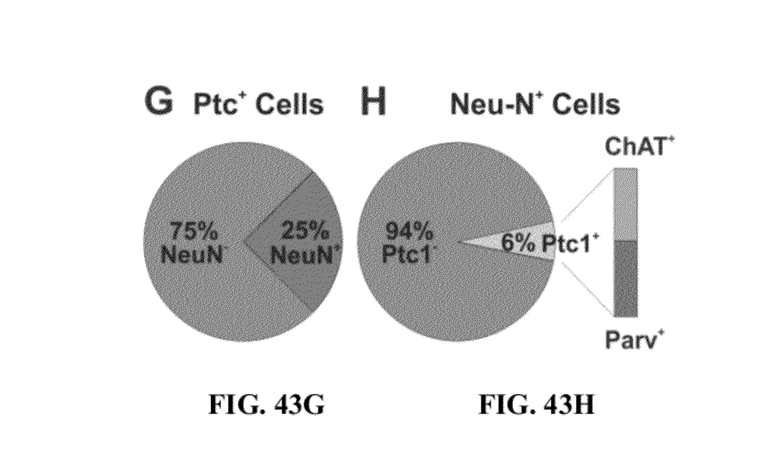

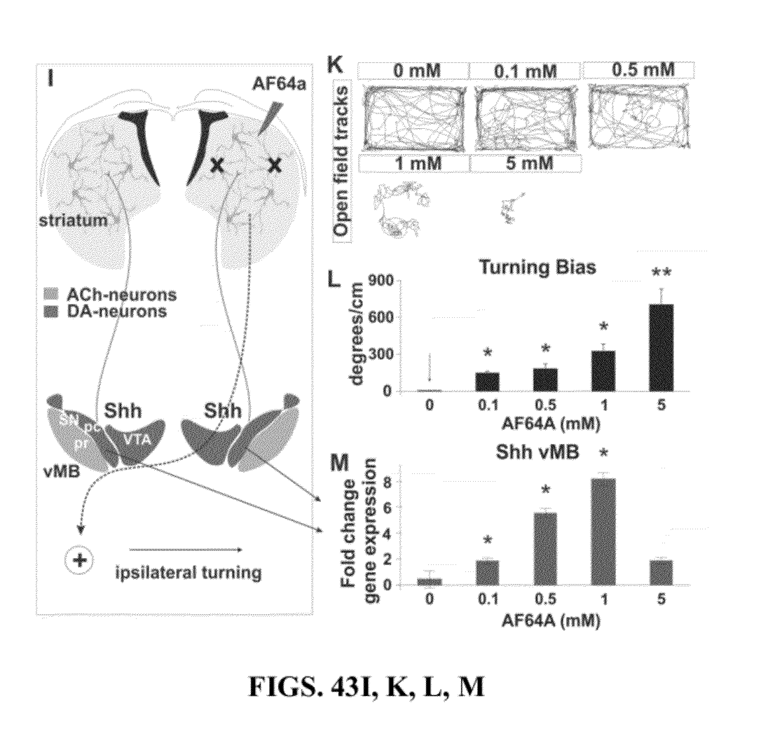

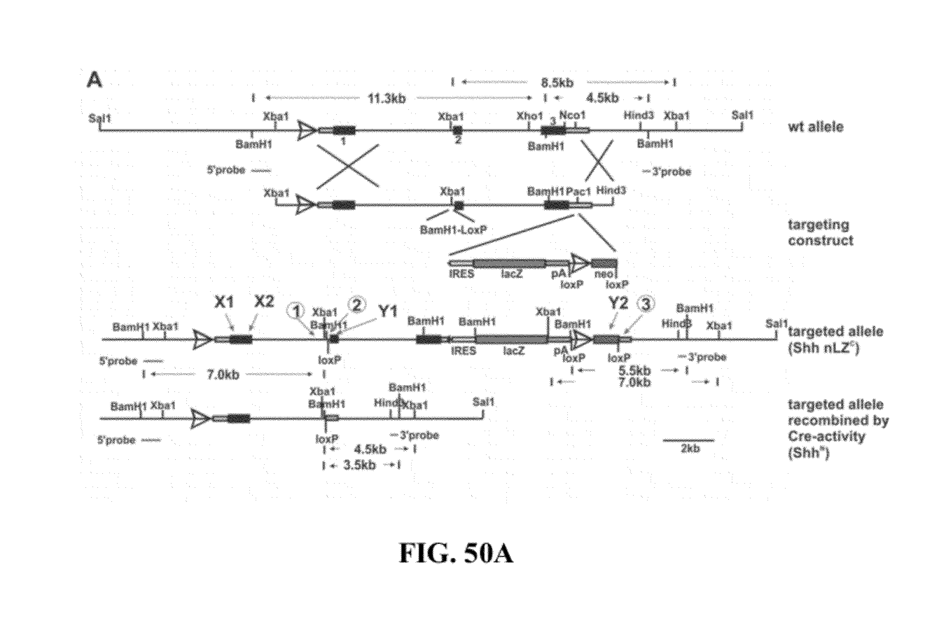

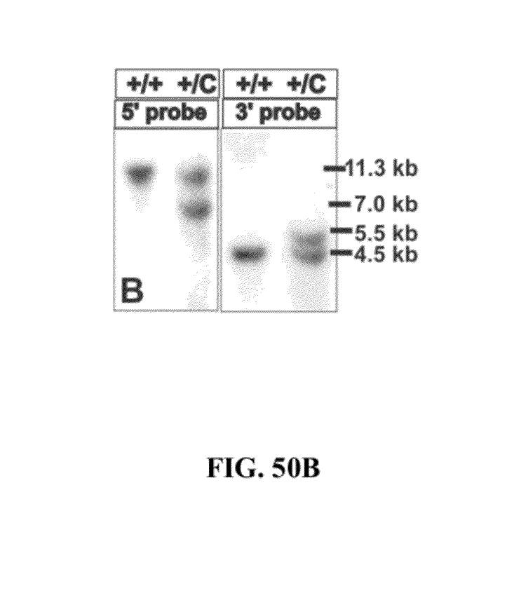

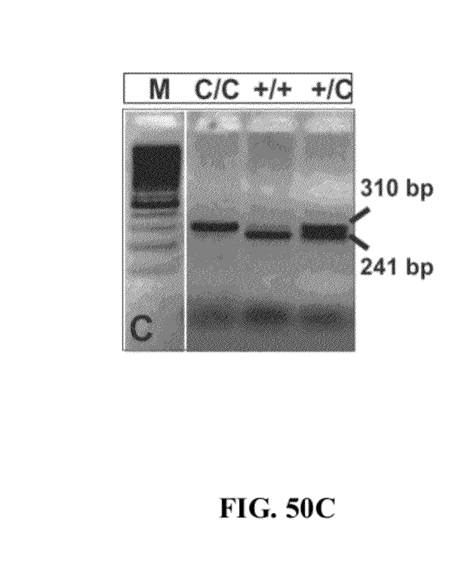

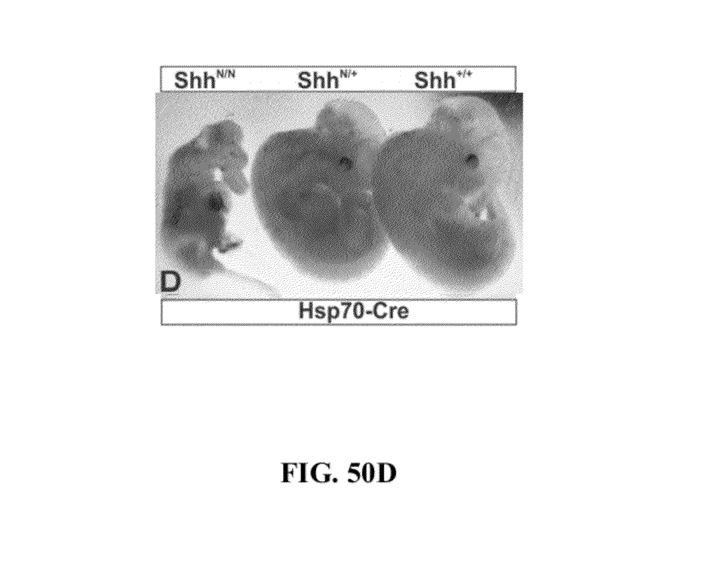

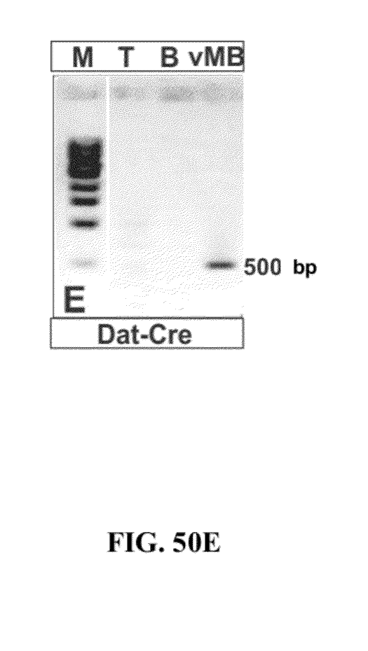

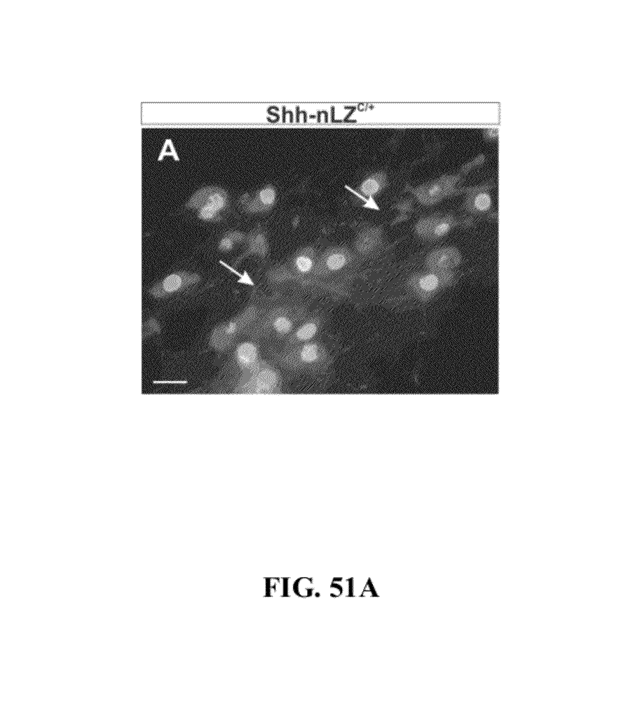

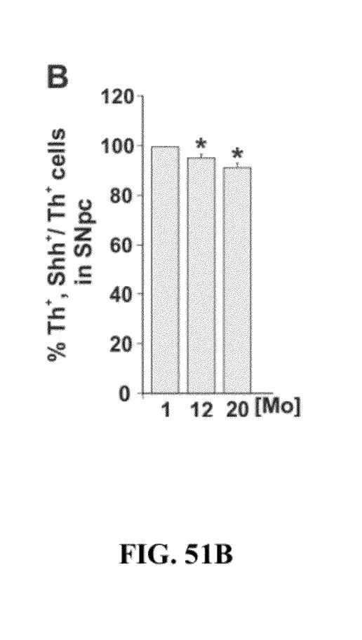

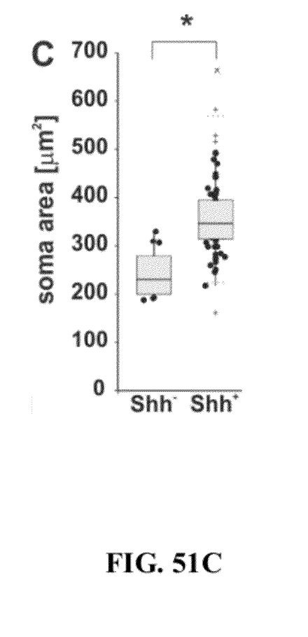

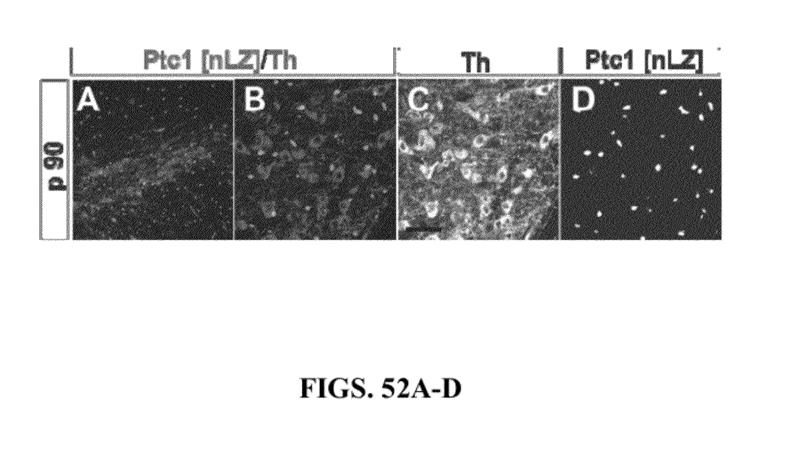

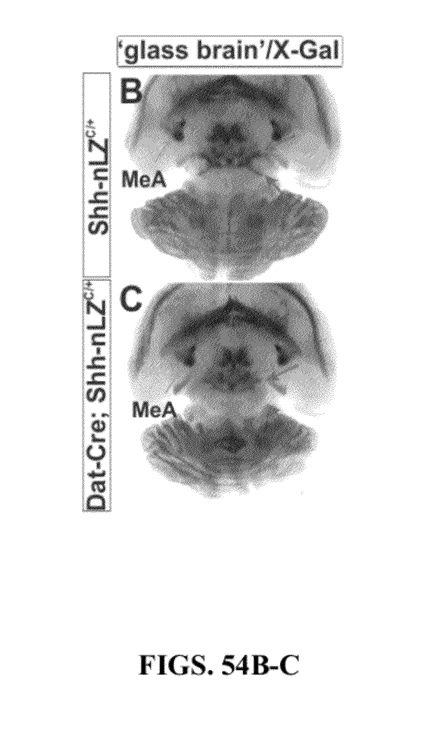

To examine whether Shh mediated cell signaling occurs among neurons of the meso-striatal circuit, expression of Shh and its receptors Patched (Ptc1 and Ptc2) was visualized in the adult brain. Using mice heterozygous for a conditional, gene expression tracer allele of Shh (Shh-nLZ^{C/+}; FIG. 50A), Shh expression was observed in all tyrosine hydroxylase positive (Th⁺), DA neurons in the substantia nigra (SN; cell group classified by Dahlstroem and Fuxe, (1964) as “A9”, (FIGS. 43A-B and FIG. 54B), the ventral tegmental area (VTA, “A10”, FIG. 43A) and the retro rubral field (RRF, “A8”) along the entire anterior posterior axis of these nuclei at p90 (100+/−0%, 683 cells, n=2) but not in DA neurons of the diencephalon and olfactory bulb. Shh is expressed throughout life in most mesencephalic DA neurons but a loss of Shh expression is observed in 4 and 10% of Th⁺ neurons at 12 and 20 months of age, respectively (FIGS. 51A-B). Consistent with published expression data based on RNA in situ hybridization (Traiffort et al., 2010), evidence for Shh expression was not found in the striatum, but expression of Ptc1 in the striatum and ventral midbrain (vMB) is readily revealed using a gene expression tracer mouse line (Ptc 1-nLZ, Goodrich et al., 1999; FIGS. 43C-F) and by mRNA in situ hybridization. No evidence for the expression of Ptc2 was found in either brain structure by mRNA in situ hybridization consistent with public gene expression data information (Gensat, http://www.gensat.org). Ptc1 expression in the vMB is largely non neuronal and does not occur in Th+ cells (FIGS. 52A-D). Within the striatum 25% of Ptc1 expressing cells are NeuN⁺ (FIGS. 43D and G) and 6% of all NeuN⁺ cells co-express Ptc1 (FIG. 43H). Striatal NeuN⁺, Ptc1⁺ neurons are found to fall exclusively into two classes with 100% of ChAT⁺ ACh-neurons (FIGS. 43E and H) and 98% of all Parv⁺ FS interneurons (FIGS. 43F and H) expressing Ptc1. Taken together with the presence of an evolutionary conserved asonal transport signal in Shh, which can result in the physiologically relevant release of Shh in the projections areas of Shh expressing neurons (Chu et al., 2006, Wallace and Raff, 1999) the expression data indicated that mesencephalic DA neurons can selectively communicate with ACh- and FS-neurons in the adult striatum through Shh signaling.

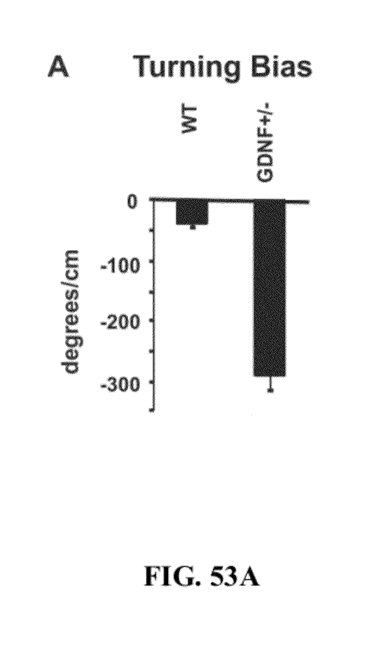

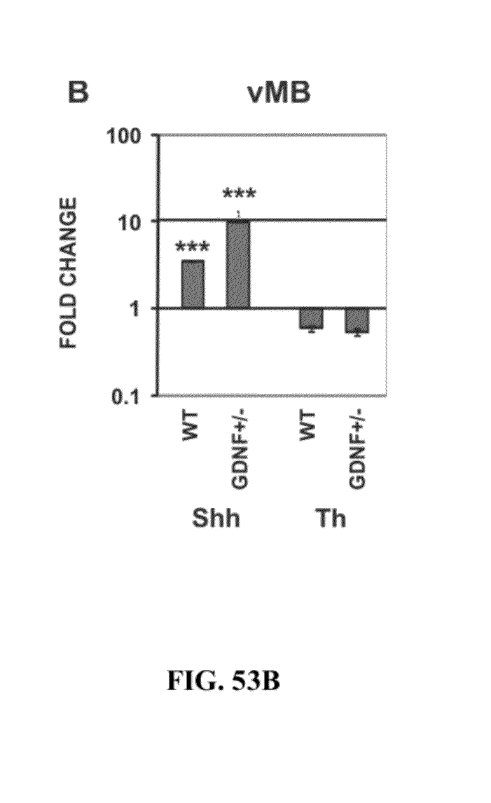

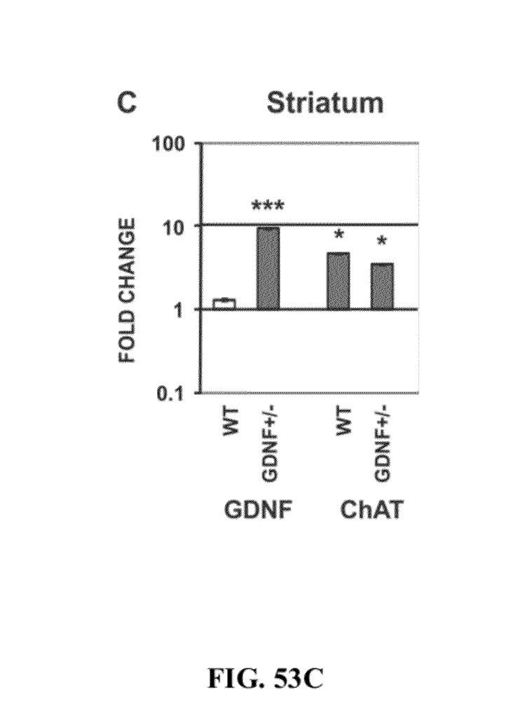

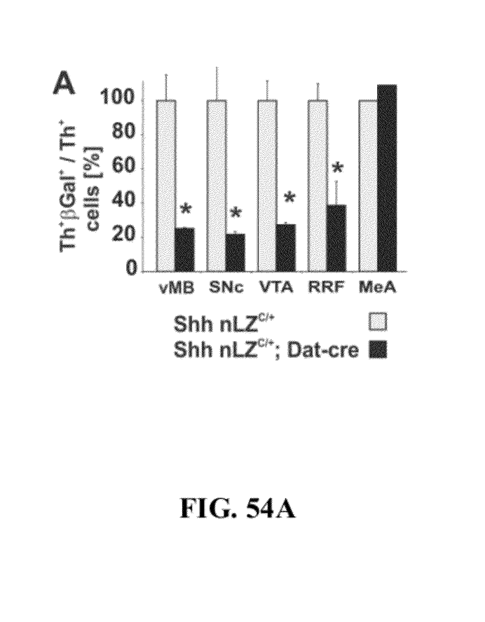

Based on results demonstrating that the expression of Shh by spinal motorneurons is repressed by signals originating in the periphery (Akazawa et al., 2004), whether Shh expression in mesencephalic DA neurons is controlled by signals emanating from the striatum was studied. 6-OHDA injection into the median forebrain bundle (mFB) is a well established neurotoxicological model for the interruption of mesostriatal communication. Unilateral 6-OHDA injections into wt BL/6 as well as heterozygous GDNF-nLacZ animals (Moore et al., 1996), which display heightened sensitivity to toxicological insults of the basal ganglia (FIG. 53A; Boger et al., 2006) results in the up-regulation of Shh transcription in the ipsilateral ventral midbrain FIG. 53B). To test whether cholinergic neurons of the striatum can be a source of a repressive signal that inhibits Shh transcription by DA neurons, the cholinotoxin AF64a, a compound with structural similarities to choline, which causes inhibition of activity of cholinergic neurons at low doses and death of cholinergic neurons at high doses, was utilized (Sandberg et al., 1984). Consistent with the observation that a relative decrease of cholinergic signaling over dopaminergic signaling facilitates motor output from the striatum (Lester et al., 2010), a correlation between a graded increase in ipsilateral turning bias caused by a concomitant increase in spinal cord activity on the contralateral side and the concentration of injected AF64a into wt C57B/6 male mice was observed (FIGS. 43I, K and L). In the ipsilateral ventral midbrain of these animals, a dose dependent, step wise, up-regulation of Shh transcription at moderate levels of AF64a which is abrogated under conditions for severe cholinergic damage is found (FIG. 43M). Together, these studies indicate that Shh expression by DA neurons is regulated by target derived signals similar to the inhibition of Shh expression in mature spinal cord motor neurons (Akazawa et al., 2004). To test whether Shh signaling within the meso striatal circuit is of physiological relevance, Shh expression from DA neurons was selectively ablated.

Ablation of Shh from Dopaminergic Neurons Causes Progressive Cellular and Functional Corruption of the Adult Striatum

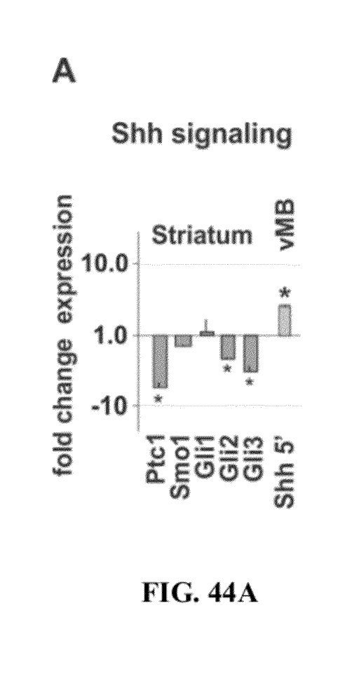

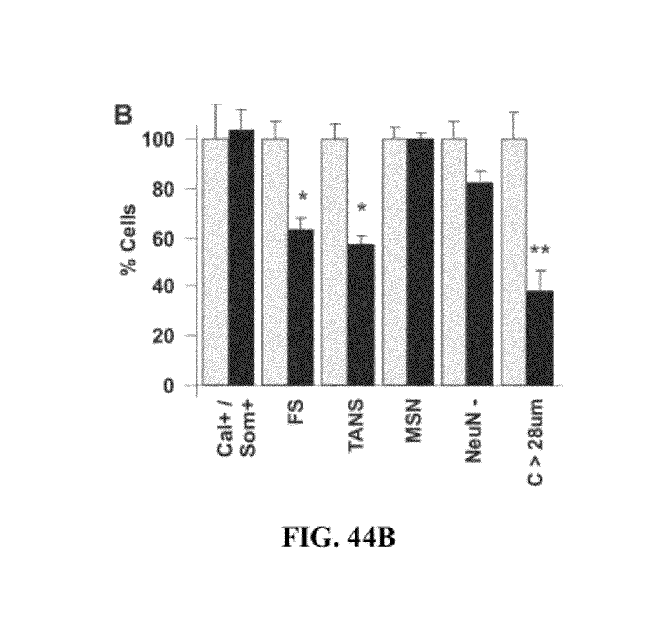

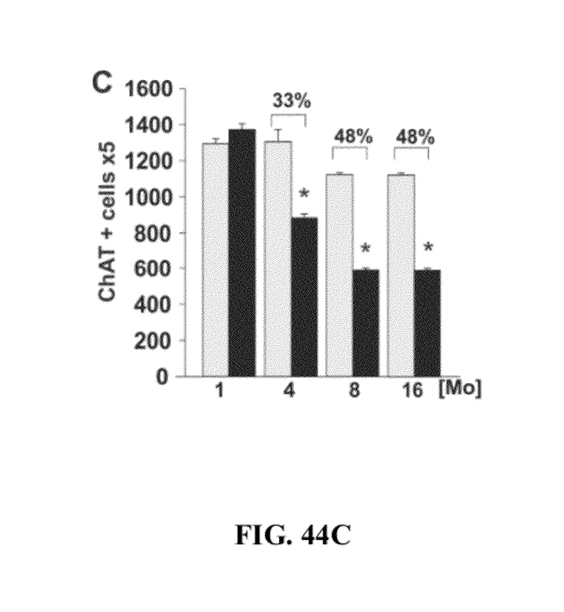

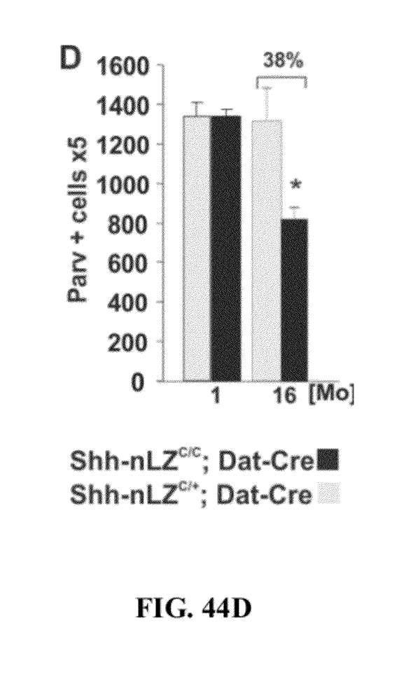

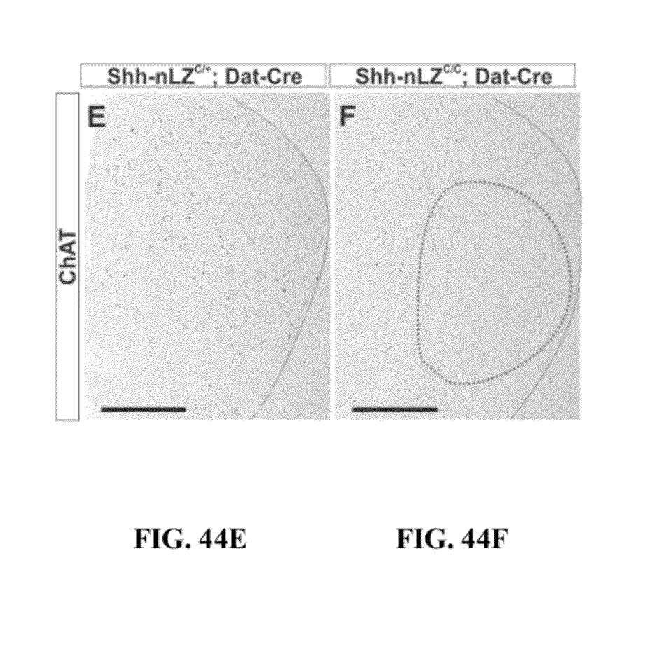

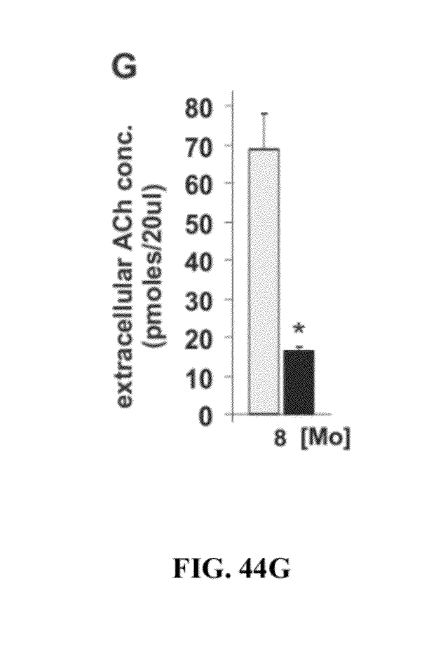

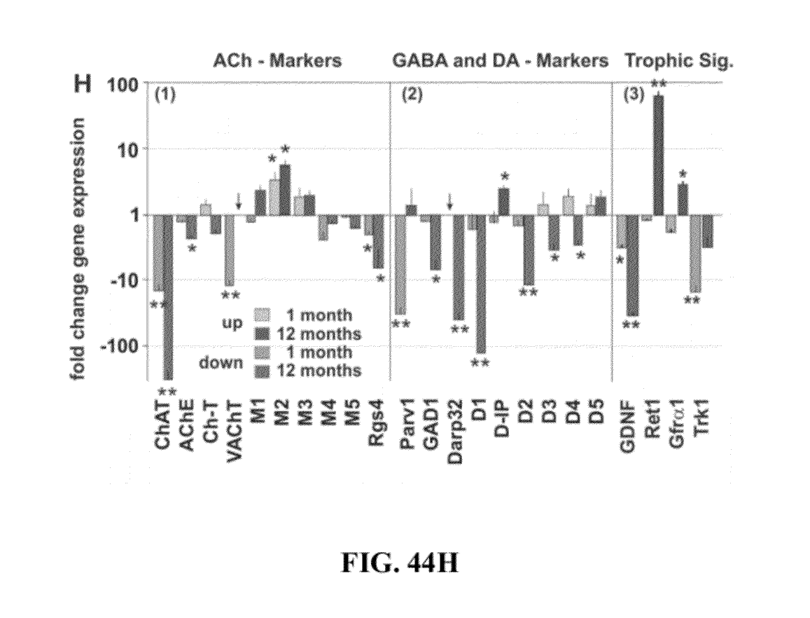

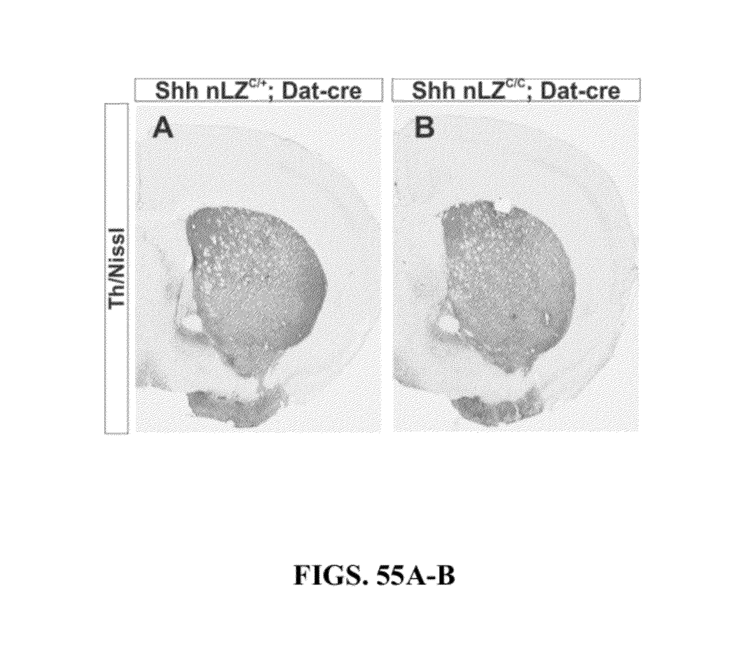

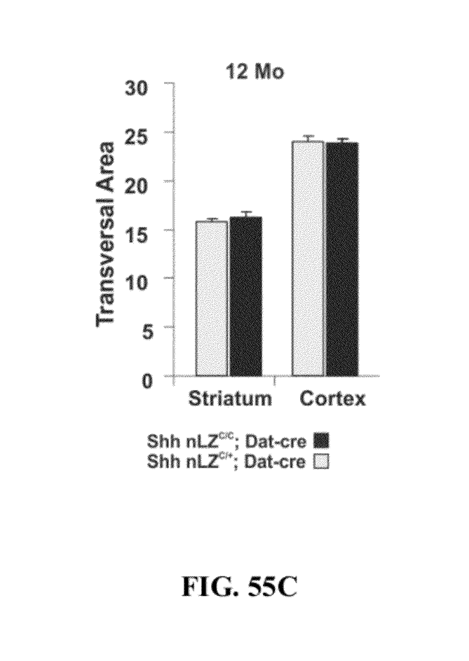

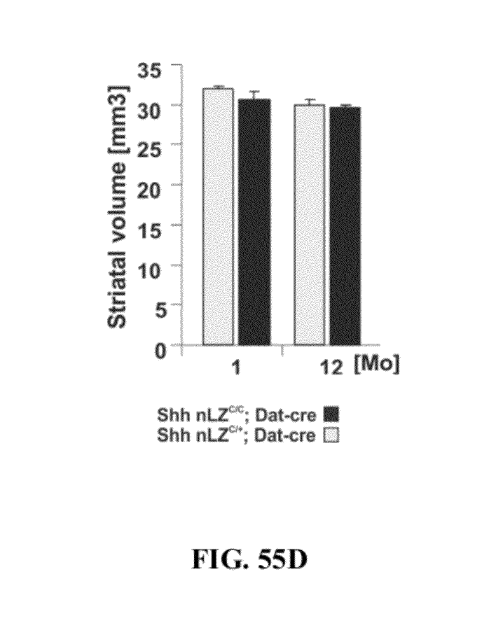

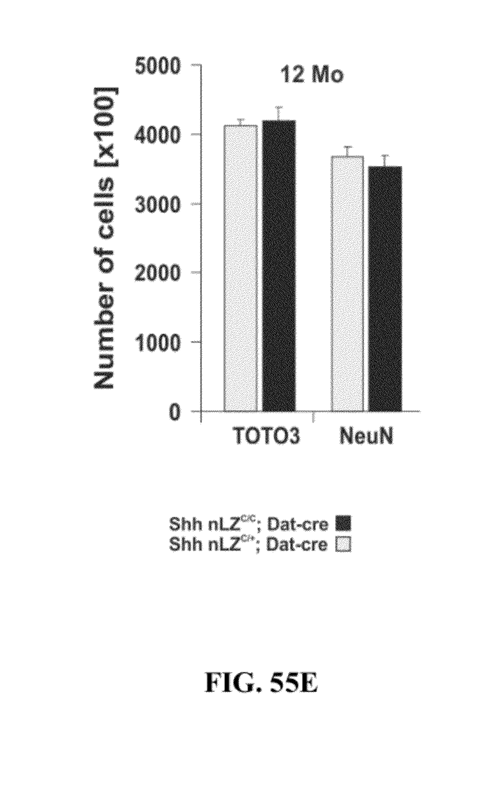

To achieve the tissue restricted ablation of Shh expression from DA neurons females homozygous for the conditional Shh allele (Shh-nLZ^{C/C}) were crossed with males double heterozygous for the conditional Shh allele (Shh-nLZ^{C/+}) and a recombinant allele of the dopamine transporter locus (Dat) in which a Cre expression cassette was inserted into the 5′ untranslated region (UTR) of the Dat gene (Dat-Cre; Zhuang et al., 2005; see Other Results and Discussion). Shh-nLZ^{C/C}; Dat-Cre mutant animals are born alive and mobile with expected mendelian frequency and no overt structural or motor signs at the end of postnatal development when compared to Shh-nLZ^{C/+}; Dat-Cre control littermates (FIG. 55A-E, Table 1; for all comparative analyses herein Shh-nLZ^{C/+}; Dat-Cre litter mates serve as controls). However, expression of the Shh signaling dependent genes Ptc1, Gli2 and Gli3, whose transcription is induced by productive Shh signaling (Hooper and Scott, 2005), is reduced in the striatum in Shh-nLZ^{C/C}; Dat-Cre mutant animals compared to litter controls at 1 month of age (FIG. 44A), indicating that DA neuron produced Shh acts in the striatum. The transcription of Shh loci in the vMB of Shh-nLZ^{C/C}; Dat-Cre mutant animals is increased (see Other Results and Discussion and FIG. 44A and FIG. 50A), consistent with striatal targets of Shh signaling ceasing to produce retrogradely acting signals in Shh-nLZ^{C/C}; Dat-Cre animals that otherwise inhibit Shh expression by mesencephalic DA neurons in the undisturbed brain.

TABLE 1
Summary of comparative single limb indices of forced locomotion in Shh-
nLZ^{CC+}; Dat-Cre mutant mice vs. Shh-nLZ^{C/+}; Dat-Cre control mice mice.

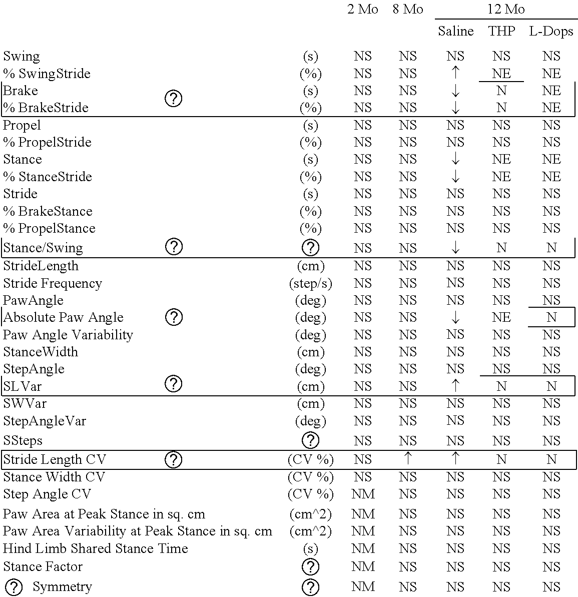

Locomotion performance was analyzed during forced treadmill walking (30 cm/s) in a DigiGait apparatus at 2, 8 and 12 months of age. At 12 months animals were also analyzed after acute treatment with L-Dopa or THP. Indices altered in mutant animals are boxed, those effected by dopamine substitution or anti cholinergic treatment are shaded, (arrows denote significant deviation from control indicating direction of change; *p <0.05, ANOVA for repeated measures followed by Tukey's post-hoc test (n = 8/genotype).
NS = not significant
NM = not measured
N = normalized by drugs
NE = drugs have no effect

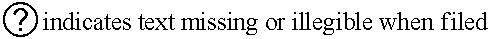

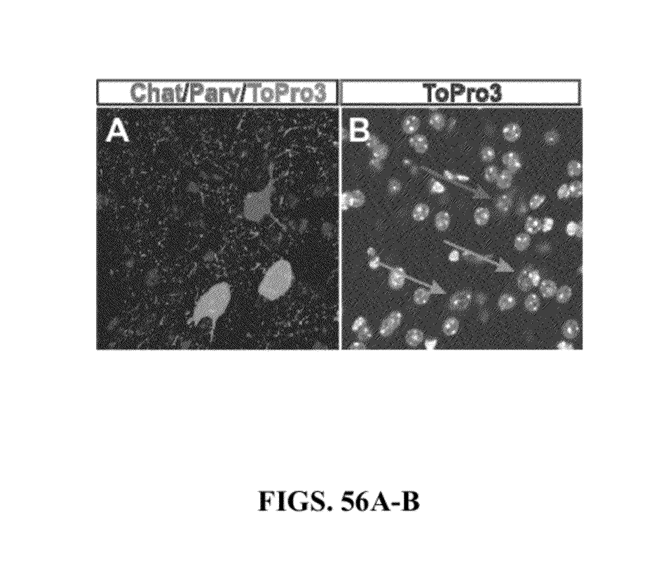

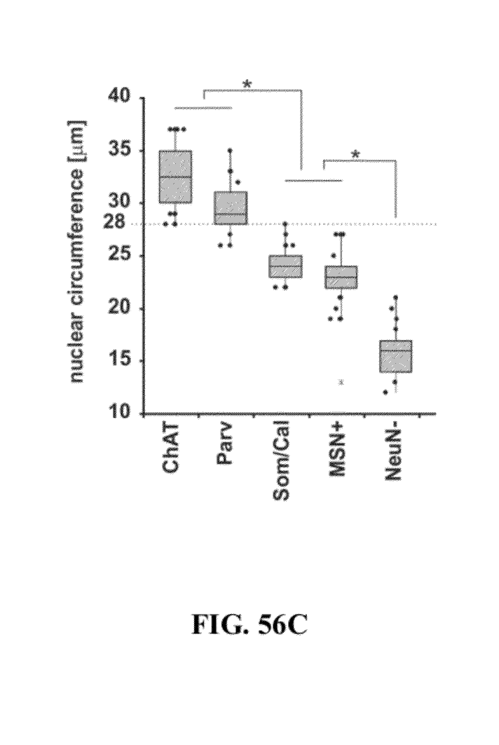

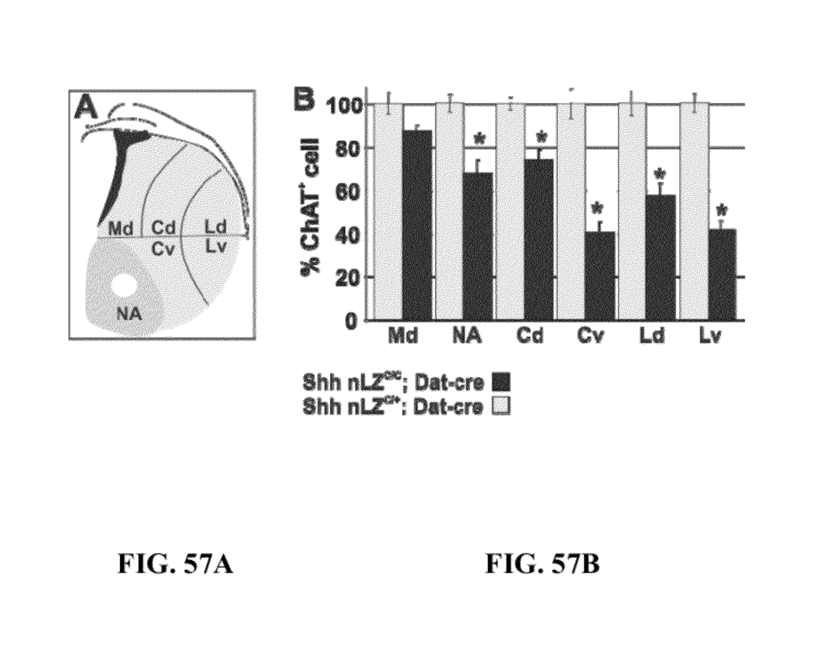

The main striatal cell populations can be distinguished independently of phenotypic marker expression based upon cell type specific perinuclear staining patterns visualized by the DNA intercalating dye ToPro3 (Matamales et al., 2009, FIGS. 56A-C). The quantitation of the relative size of striatal cell populations by perinuclear staining patterns at 3 months of age revealed a ˜40% reduction in the numbers of ACh- and FS-interneurons with no alterations in the relative size of calretinin⁺ and somatostatin⁺ interneurons, msPs, and non-neuronal cell populations in Shh-nLZ^{C/C}; Dat-Cre mutant animals compared to controls (FIG. 44B). In agreement, longitudinal, stereological quantitation of ACh- and FS-neurons based on the immuno-histochemical staining of ChAT and Parv, resp. reveals an adult onset, progressive reduction in the numbers of ChAT⁺ and Parv⁺ cells which plateaus at 8 months of age at about 50% and 40% resp. (FIGS. 44C-D). The reduction in ChAT⁺ neurons is most pronounced in lateral aspects of the dorsal striatum (FIGS. 44E-F; FIGS. 57A-B). ACh- and FS interneurons make up together only about 6% of total striatal neurons (FIG. 43H) and are locally projecting. These attributes make it difficult to distinguish neuronal degradation from a mere down-regulation of phenotypic marker expression by the quantitation of the total number of neurons or visualization of specific projection patterns. However, ACh- and FS-neurons as a group exhibit the largest nuclei and can be distinguished from all other striatal neurons by a nuclear circumference larger than 28 μm (FIG. 56C). Quantitation of the relative numbers of striatal cells with nuclear circumference larger than 28 μm reveals a 62+/−8% reduction in Shh-nLZ^{C/C}; Dat-Cre mutant animals compared to controls (FIG. 44B). Hence, in aggregate, the analysis of perinuclear staining pattern, nuclear size, and cell type specific marker gene expression, all reveal a cell type selective, adult onset, progressive, but incomplete degeneration of ACh- and FS-neurons in the absence of Shh expression by mesencephalic DA neurons.

Surviving ACh-neurons do not functionally compensate for the reduction in their numbers. Instead a much larger, 6 fold, reduction in basal levels of extracellular ACh is found, beyond of what will be expected from the mere loss of ˜50% ACh-neurons, in 8 month old Shh-nLZ^{C/C}; Dat-Cre mice compared to age matched controls by in vivo dialysis (FIG. 44G).

To explore the molecular underpinnings of the physiology of surviving ACh-neurons, potential alterations in the expression of candidate genes were investigated, which can inform about the neurophysiological status of the striatum before and after the manifestation of neuronal loss. Comparative quantitative rtPCR analysis of striatal derived mRNA was used for cholinergic-, gabaergic-, and dopaminergic-marker gene and trophic factor expression in 5 and 52 week old Shh-nLZ^{C/C}; Dat-Cre mutant animals and controls (all genes probed are listed in Table 2).

TABLE 2
List of all amplicons used for quantitative gene expression measurements. ABI
code identified gene probes for rtPCR (TaqMan ® Gene Expression Assays).
Oligonucleotides designed to amplified specific exon regions (amplicon length provided)
were synthesized by ABI based on a NCBI reference sequence (RefSeq).
(https://products.appliedbiosystems.com) For monitoring transcription of Shh exon 1 (x1,
x2), a custom amplicon was designed (Shh 5′; as described in FIG. 50A).

| Group | Gene name | Symbol | Assay ID ABI | RefSeq | Length |
|---|---|---|---|---|---|
| Shh- | sonic hedgehog | Shh-3′ | Mm00436527_m1 | NM_009170.3 | 105 |
| pathway | sonic hedgehog | Shh-5′ | Custom | NA | ? |
| | smoothened | Smo | Mm01162710_m1 | NM_176996.4 | 58 |
| | GLI-Kruppel family member | Gli1 | Mm00494645_m1 | NM_010296.2 | 68 |
| | GLI1 | | | | |
| | GLI-Kruppel family member | Gli2 | Mm01293116_m1 | NM_001081125.1 | 80 |
| | GLI1 | | | | |
| | GLI-Kruppel family member | Gli3 | Mm00492345_m1 | NM_008130.2 | 62 |
| | GLI1 | | | | |
| | patched homolog 1 | Ptc1 | Mm00436026_m1 | NM_008957.2 | 69 |
| | tyrosine hydroxylase | TH | Mm00447546_m1 | NM_009377.1 | 65 |
| Dopamine | dopamine receptor D1A | D1A | Mm02620146_s1 | NM_010076.3 | 148 |
| Markers | dopamine receptor 2 | Drd2 | Mm00438541_m1 | NM_010077.2 | 71 |
| | dopamine receptor 3 | Drd3 | Mm00432887_m1 | NM_007877.1 | 71 |
| | dopamine receptor 4 | Drd4 | Mm00432893_m1 | NM_007878.2 | 83 |
| | dopamine receptor 5 | Drd5 | Mm00658653_s1 | NM_013503.2 | 152 |
| | solute carrier family 6 | DAT | Mm00438388_m1 | NM_010020.3 | 74 |
| | solute carrier family 18 | VMAT2 | Mm00553058_m1 | NM_172523.2 | 58 |
| | calcyon neuron-specific vesicular | Drd1ip | Mm00503414_m1 | NM_026769.4 | 103 |
| | protein | | | | |
| | protein phosphatase 1, regulatory | DARPP- | Mm00454892-m1 | NM_144828.1 | 57 |
| | | 32 | | | |
| GABA | glutamic acid decarboxylase 1 | GAD1 | Mm00725661--s1 | NM_008077.4 | 66 |
| Markers | parvalbumin | Pvalb | Mm00443100--m1 | NM_013645.3 | 77 |
| Cholinergic | cholinergic receptor, muscarinic 5 | M5 | Mm01701855_s1 | NM_203491.1 | 70 |
| Markers | cholinergic receptor, muscarinic 3 | M3 | Mm00446300_s1 | NM_033269.4 | 64 |
| | cholinergic receptor, muscarinic 5 | M5 | Mm01701883_s1 | BC120615.1 | 87 |
| | cholinergic receptor, muscarinic 4 | M4 | Mm00432514_s1 | NM_007699.2 | 53 |
| | cholinergic receptor, muscarinic | M1 | Mm00432509_s1 | NM_001112697.1 | 90 |
| | 1, CNS | | | | |
| | choline acetyltransferase | ChAT | Mm01221880_m1 | NM_009891.2 | 70 |
| | solute carrier family 18 | VAChT | Mm00491465_s1 | NM_021712.2 | 53 |
| | solute carrier family 44, member 1 | CDWG2 | Mm00460214_m1 | NM_133891.3 | 80 |
| | regulator of G-protein signaling 4 | RGS4 | Mm00501389_m1 | NM_009062.3 | 116 |
| | acetylcholinesterase | AChE | Mm00477275_m1 | NM_009599.3 | 106 |
| | glyceraldehyde-3-phosphate | GAPDH | Mm99999915_g1 | NM_008084.2 | 107 |
| | dehydrogenase | | | | |
| Stress | heat shock protein 5 | GRP78 | Mm00517691_m1 | AL022860 | 75 |
| Markers | X-box binding protein 1 | Xbp1 | Mm00457357_m1 | NM_013842.2 | 56 |
| | glutathione peroxidase 1 | Gpx1 | Mm00656767_g1 | NM_008160.5 | 134 |
| | leucine-rich repeat kinase 2 | Lrrk2 | Mm00481934_m1 | NM_025730.3 | 88 |
| | RIKEN cDNA 5730590G19 | Lrrk1 | Mm00612895_m1 | NM_029835.1 | 76 |
| | gene | | | | |
| Parkinson | PTEN induced putative kinase 1 | Pink1 | Mm00550827_m1 | NM_026880.2 | 91 |
| Markers | PD (autosomal recessive, early | Park7 | Mm00498538_m1 | NM_020569.3 | 92 |
| | onset) 7 | | | | |
| | PD (autosomal recessive, | Park2 | Mm450186 m1 | NM_016694.3 | 115 |
| | juvenile) 2, parkin | | | | |
| | nuclear receptor subfamily 4 | Nr4a2 | Mm00443056_m1 | NM_013613.2 | 98 |
| | group A, member 2 | | | | |
| Trophic | ubiquitin carboxy-terminal | Uchl1 | Mm00495900_m1 | NM_011670.2 | 78 |
| Factors | hydrolase L1 | | | | |
| | synuclein, alpha | Synuclein | Mm01188700_m1 | NM_001042451.1 | 67 |
| | glial cell line derived | GDNF | Mm00599849_m1 | NM_010275.2 | 101 |
| | neurotrophic factor | | | | |
| | ret proto-oncogene | Ret | Mm00436304_m1 | NM_001080780.1 | 67 |
| | GDNF factor family receptor | GFRa1 | Mm00833897_m1 | NM_010279.2 | 153 |
| | alpha 1 | | | | |
| | neurotrophic tyrosine kinase, | Trk B | Mm00435422_m1 | NM_001025074.1 | 92 |
| | receptor, type 2 | | | | |

The expression of the striatal cholinergic markers ChAT, vesicular acetylcholine transporter (vAChT), NGF receptor TrkA and GTPase regulator RGS4 are down-regulated while the muscarinic autoreceptor M2 is up-regulated at 5 weeks of age (FIG. 44H(1)). The expression of ChAT, M2, RGS4 is further distorted at 12 months of age while the expression of vAChT and TrkA becomes normalized at that age suggesting a compensatory up regulation of the latter genes in surviving ACh-neurons (FIG. 44H(1)). Acetylcholine esterase (AChE) expression is unaltered at 5 weeks but reduced at 12 months of age consistent with the observed diminishment of striatal ACh levels at later stages (FIG. 44G-H(1)). ACh tone in the striatum is in part regulated by muscarinic autoreceptors M2 and M4 whose functions in turn are negatively modulated by the GTPase accelerator RGS4 (Ding et al., 2006). Hence the observed up-regulation of M2 and down-regulation of RGS4 gene expression indicates an enhancement of cholinergic auto-receptor function in the striatum of Shh-nLZ^{C/C}; Dat-Cre mutant animals.

Parvalbumin gene expression is strongly reduced at 5 weeks of age, but reaches normal levels at 12 months, indicating a compensatory up regulation by surviving FS (FIG. 44H(2)). General gabaergic marker- and dopamine receptor-gene expression are not affected at 5 weeks but dopamine receptors D1-D4 (but not D5), DARP32, and Gad1 are down- and dopamine receptor interacting protein (D-IP) up-regulated at 12 months of age, indicating that only subsequently to Ach- and FS-neurons other striatal cell populations become involved (FIG. 44H(2)).

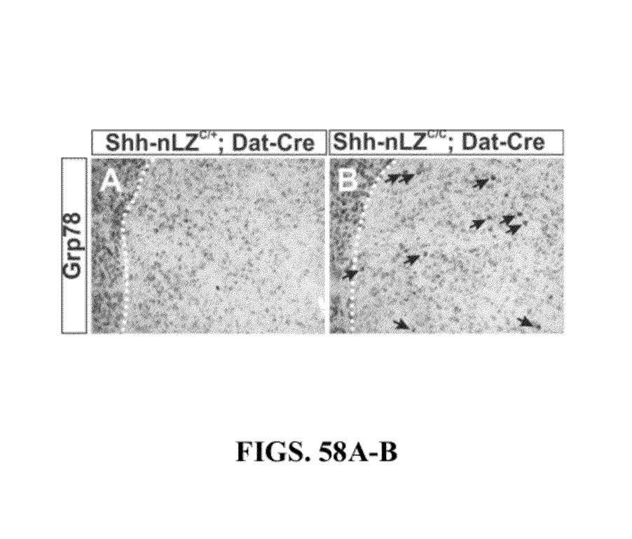

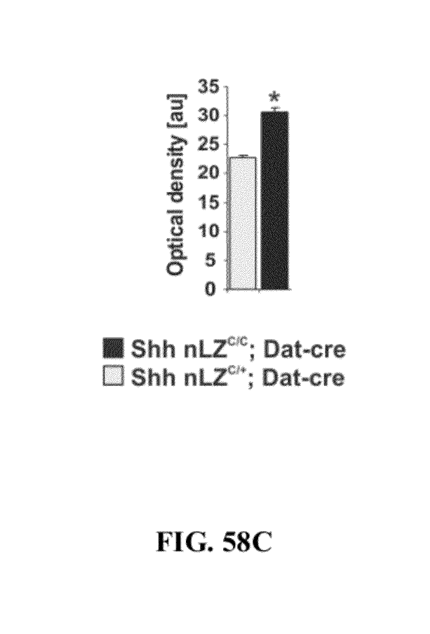

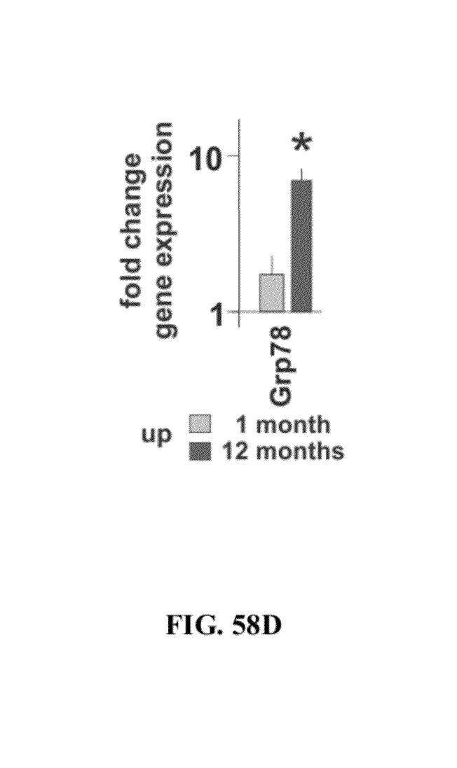

Consistent with the activation of physiological cell stress response pathways in ACh- and FS-neurons prior to neuro-degeneration, increased expression of the luminal endoplasmic reticulum (ER) protein BiP (Grp78) by large bodied cells in the striatum is found by mRNA in situ hybridization at 5 weeks of age (FIG. 58A-D). The gene expression studies also revealed an early and progressive down regulation of GDNF while the expression of the GDNF co receptors Ret1 and Gfra1 is strongly up-regulated at 12 months of age (FIG. 44H(3)). Thus, the data herein indicate that the absence of Shh signaling from DA neurons elicits a sequential structural and functional corruption of the striatum which begins with cell physiological alterations in ACh- and FS-neuron that in turn precludes functional adaptations by surviving ACh-neurons to the progressive distortions in cellular compositions of the striatum.

Shh Signaling Originating from DA Neurons Represses GDNF Transcription in the Striatum

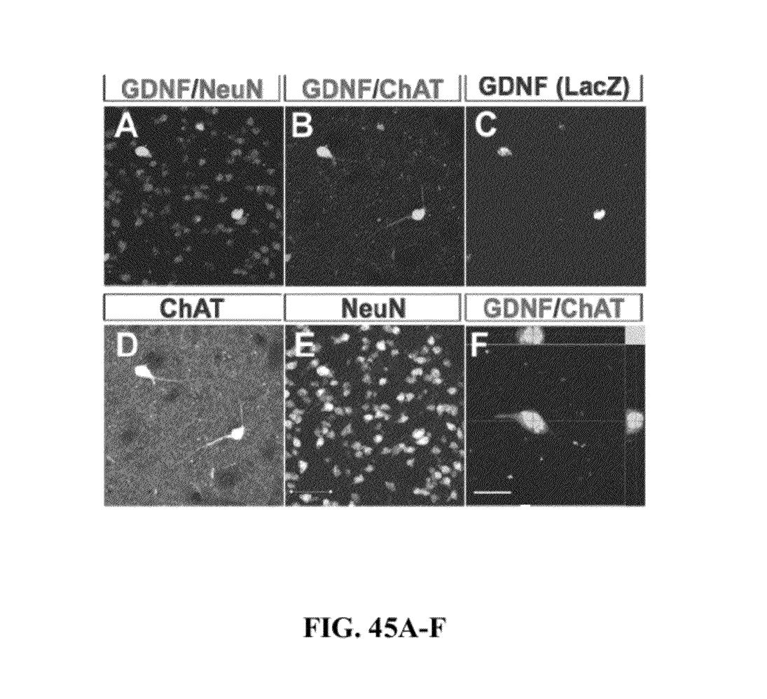

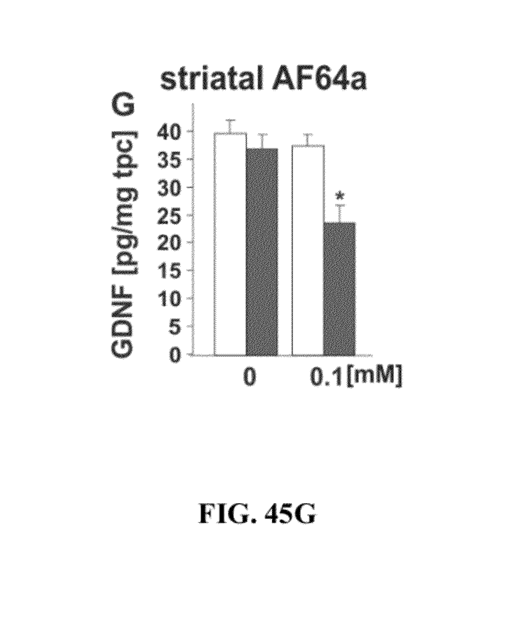

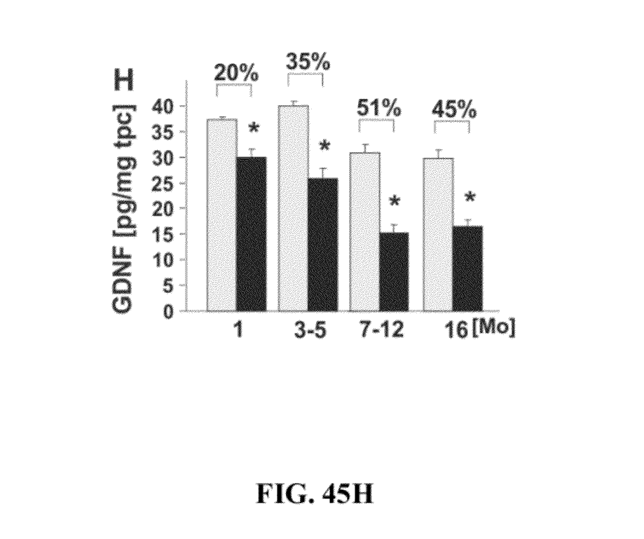

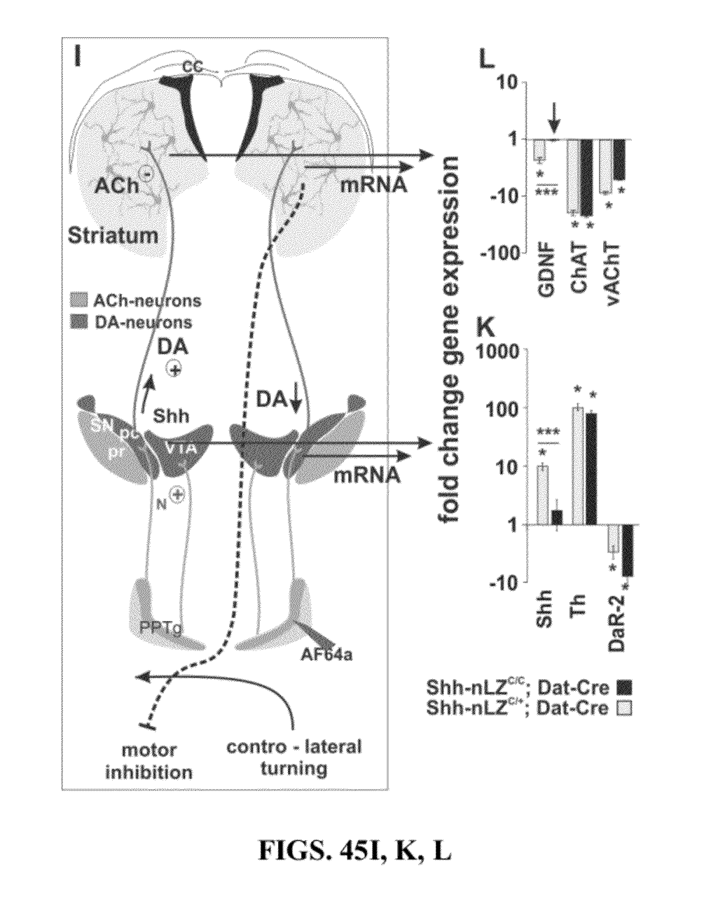

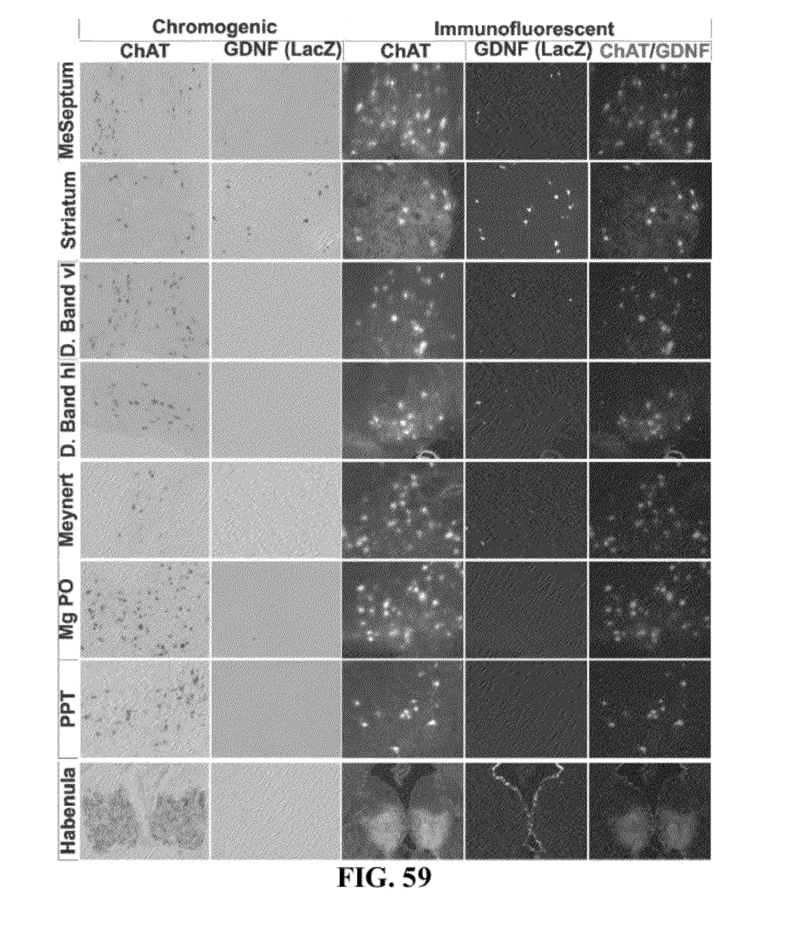

Consistent with previous reports that indicated that cholinergic or large bodied cells in the striatum might express GDNF mRNA (Bizon et al., 1999; Barossa-Chinea et al., 2005), the reduction in GDNF gene expression was found to follow a temporal pattern similar to other cholinergic markers (FIG. 44H(3)). Since previous expression studies of GDNF in the adult striatum were based on hard to quantify immunohistochemical or mRNA in situ procedures, a LacZ based GDNF specific, genetic gene expression tracer mouse line was utilized for quantifying GDNF gene expression in the striatum in the mouse paradigm (GDNF-LZ; Moore et al., 1996). Expression of GDNF was selectively found in 100% of ACh-neurons but not in other NeuN⁺ neurons in the striatum (FIG. 45A-F), nor by other major cholinergic nuclei of the brain (FIG. 59). Unilateral striatal injections of AF64a at 0.1 mM, the lowest dose of the cholinotoxin which causes measurable, transient ipsilateral circling behavior (FIG. 43L), leads to a ˜30% reduction in striatal GDNF protein content over carrier injected controls 36 h post application (FIG. 45G) in 4 month old C57Bl/6 wt animals indicating that striatal GDNF production is sensitive to cholinergic dysfunction. In Shh-nLZ^{C/C}; Dat-Cre mutant animals, a progressive decline in striatal GDNF content that plateaus at ˜50% at 8 months of age relative to control animals is observed, in fair correlation with the observed progressive degeneration of ACh-neurons (FIG. 45H and FIG. 44C). These experiments demonstrate that ACh-neurons are a significant, insult sensitive, source of GDNF in the basal ganglia and that Shh signaling originating from mesencephalic DA neurons is essential for the longterm maintenance of striatal GDNF production.

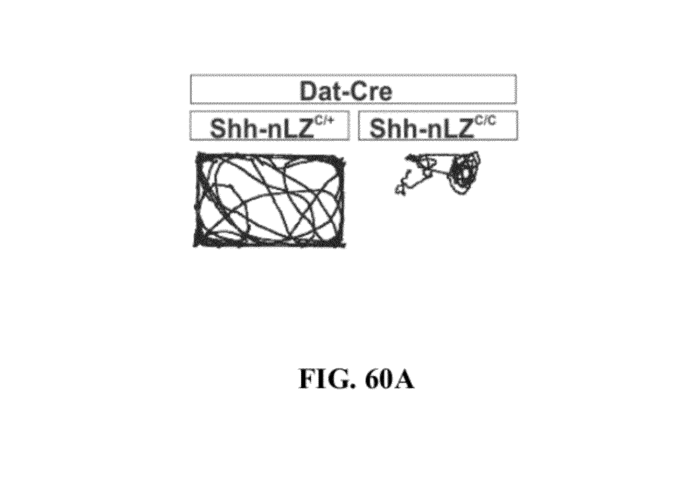

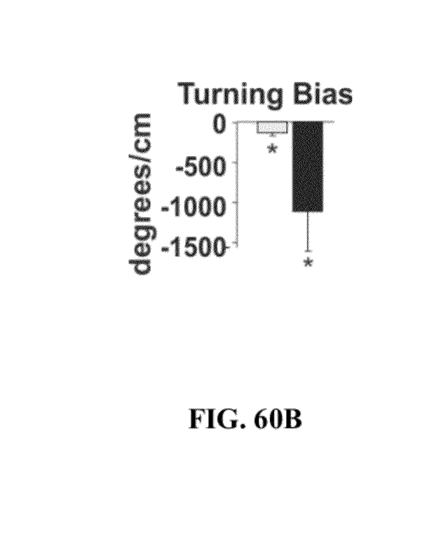

Whether Shh signaling originating from DA-neurons will regulate GDNF expression in the adult striatum utilizing an extension of the unilateral AF64a injection model described herein in conjunction with DA neuron restricted ablation of Shh was explored. The Penduncolo pontine tegmental nucleus (PPTg) provides excitatory, nicotinic receptor mediated cholinergic input to mesencephalic DA neurons (Futami et al., 1995; FIG. 45I). Similar to previous observations upon unilateral exitotoxic ablation of PPTg neurons (Dunbar et al., 1992), AF64a injections into the PPTg of 2 month old Shh-nLZ^{C/C}; Dat-Cre mutant animals or controls are found to elicit a contro-lateral turning bias consistent with reduced cholinergic stimulation of ipsilateral DA neurons (FIG. 45I and FIG. 60). Comparative quantitative rtPCR analysis of dopaminergic markers in the vMB reveals a strong transcriptional up-regulation of Th and a down-regulation of the DA autoreceptor DaR2 in both genotypes, indicating an adaptive up-regulation of mesencephalic DA-signaling ipsilateral to AF64a injection into the PPTg. AF64a injection into the PPTg results in a 10 fold up-regulation of Shh transcription in the ipsilateral compared to the contra lateral control vMB (FIG. 45K) similar to the effect on Shh expression upon AF64a insult to striatal cholinergic neurons (FIG. 43M). Since Shh-nLZ^{C/C}; Dat-Cre mutant animals are unable to express functional Shh, this experimental model allows the investigation of the effect of acute up-regulation of Shh by DA neurons on GDNF expression in the experimentally undisturbed meso-striatal circuit.

Upon AF64a injection into the PPTg, the expression of ChAT and vAChT in the ipsilateral striatum were found to be down-regulated regardless of Shh expression by DA neurons to similar extent compared to the contra lateral striatum (FIG. 45L). However, a ˜4 fold down-regulation of GDNF expression is observed in the ipsilateral striatum upon AF64a injection into the PPTg only in control mice, i.e. mice that express Shh in DA neurons, but not in animals with genetic ablation of Shh from DA neurons (FIG. 45L). These experiments provide in vivo evidence for the dynamic inhibition of GDNF expression in the striatum by Shh signaling originating from mesencephalic DA neurons.

Progressive Loss of Mesencephalic Dopaminergic Neurons.

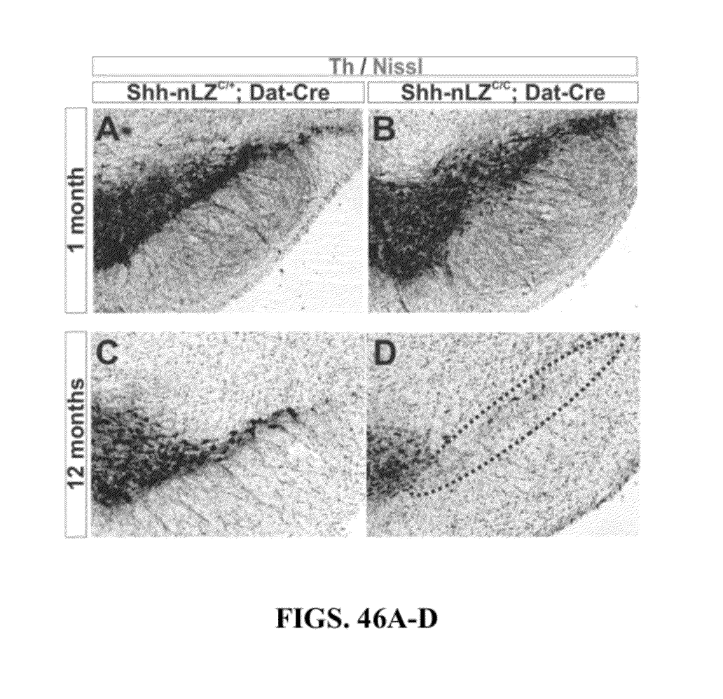

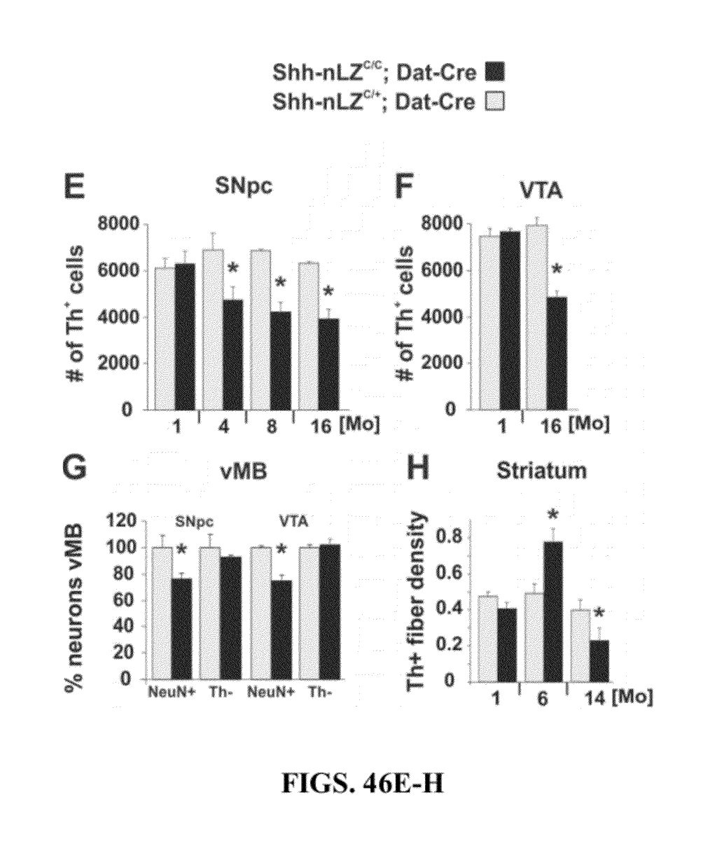

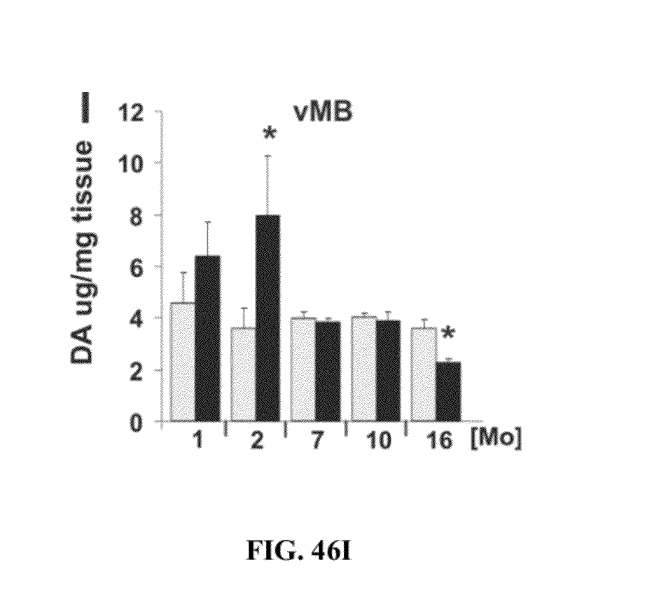

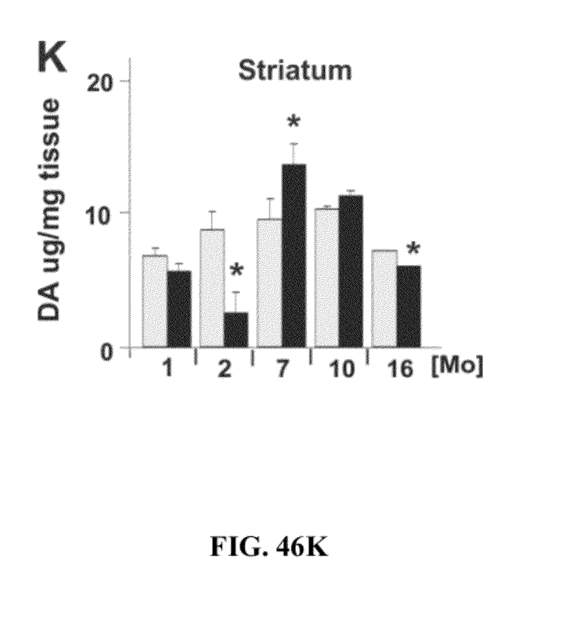

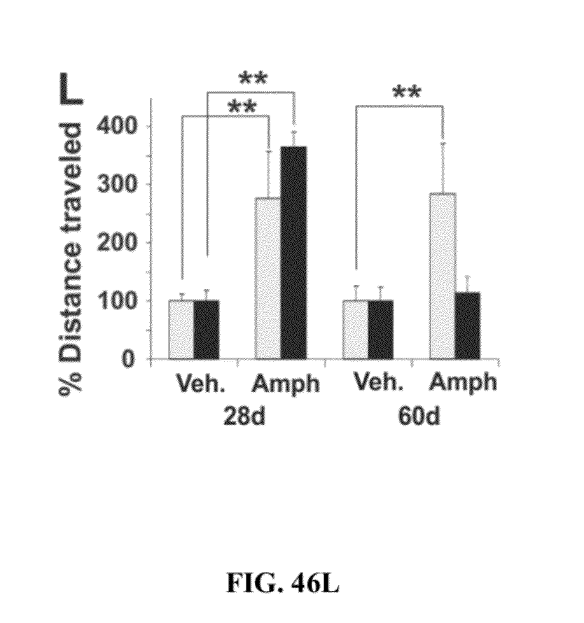

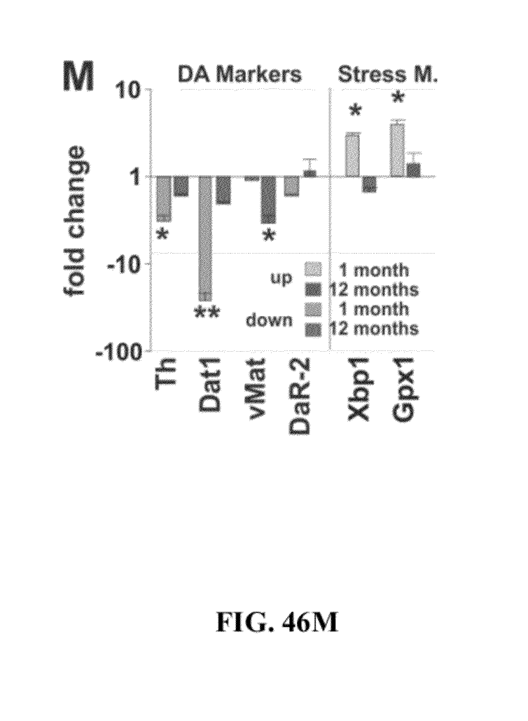

Partial ablation of GDNF expression in the adult brain causes accelerated DA-neuron death (Pascual, 2008). The long term maintenance and physiology of mesencephalic DA neurons was ascertained in Shh-nLZ^{C/C}; Dat-Cre mice. Longitudinal, unbiased sterological cell counting of Th⁺ and Th⁻ neurons in the SNpc and VTA reveals an adult onset, progressive degeneration of DA neurons that plateaus at about 40% in Shh-nLZ^{C/C}; Dat-Cre compared to controls at 8 months of age (FIG. 46A-G). The kinetics and extend of DA neuron decay is correlated to the degeneration of ACh-neuron of the striatum (R2=0.9879; T(12.8), p<0.006; FIG. 44C and FIG. 46E) and qualitatively similar in the rate of progressiveness and absolute extend to the adult onset decay of DA neurons observed in animals with ablation of the GDNF receptor Ret1 from DA neurons (Kramer et al., 2007).

The progressive degeneration of DA neurons is associated with multiphasic distortions of dopaminergic physiology of surviving DA neurons: Striatal Th⁺ fiber density is normal at 1 month of age, increased at 8 months and decreased at 12 months of age in Shh-nLZ^{C/C}; Dat-Cre mice compared to controls (FIG. 46H). Analysis of DA levels in the somato-dendritic and striatal compartments of DA neurons reveals highly dynamic and qualitative opposite disturbances during phenotype progression with a 2 fold increase in the vMB but a 3 fold reduction in the striatum at 2 months of age (FIGS. 46I and K). At 7 months of age, normalized levels of DA are found in the vMB but a 30% increase of DA is found in the striatum (FIGS. 46I and K). These distortions resolve at 10 months and DA levels become eventually diminished in both compartments at 16 months in Shh-nLZ^{C/C}; Dat-Cre mutant animals compared to controls (FIGS. 46I and K). Amphetamine elicited DA mobilization, measured by increased locomotion upon injection of the drug, is normal at 28 days of age but undetectable at 8 weeks of age in Shh-nLZ^{C/C}; Dat-Cre mutant animals compared to litter controls (FIG. 46L), consistent with a reduction in releasable dopamine in the striatum in early adulthood.

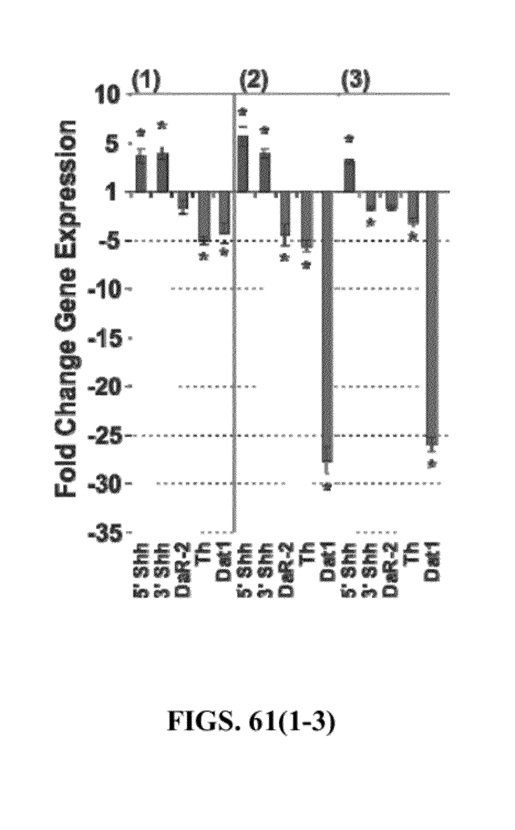

The quantification of relative gene expression of DA markers in the vMB reveals a down regulation of Th, Dat1, and DaR2 at 5 weeks of age, which then becomes normalized by 12 months in Shh-nLZ^{C/C}; Dat-Cre compared to controls (FIG. 46M). Given ongoing DA neuron degeneration, these results indicate an overexpression of these genes and increased DA production by surviving DA neurons at 12 months of age in Shh-nLZ^{C/C}; Dat-Cre animals compared to litter controls. In contrast, the expression of the vesicular monoamine transporter 2 (vMat2) appears normal at 5 weeks but is diminished at 12 months consistent with decreased DA tissue contend at that time. Indicating the activation of physiological cell stress responses, transcriptional up-regulation of Xbp1, an activator of ER based stress response pathways, and Glutathione-peroxidase 1 (Gpx1), a marker for oxidative stress in Shh-nLZ^{C/C}; Dat-Cre animals, is found at 5 weeks but not at 12 months of age (FIG. 46M). Unilateral injection of the cholinotoxin AF64a into 3 month old C57Bl/6 wt animals causes an acute dose dependent, ipsilateral down-regulation of DaR2, Th, and Dat1 qualitatively similar to the observations in Shh-nLZ^{C/C}; Dat-Cre animals (FIG. 61) showing that acute cholinergic dysfunction in the striatum can result in many of the physiological alterations in the vMB observed in Shh-nLZ^{C/C}; Dat-Cre animals.

Collectively, these results reveal a progressive structural and functional corruption of dopaminergic function which is strongly correlated with the degeneration of ACh-neurons and an associated diminishment of GDNF in the striatum in the absence of DA produced Shh.

Shh Expressing DA Neurons Become Enriched in Shh-nLZ^{C/C}; Dat-Cre Mutant Animals During Phenotype Progression

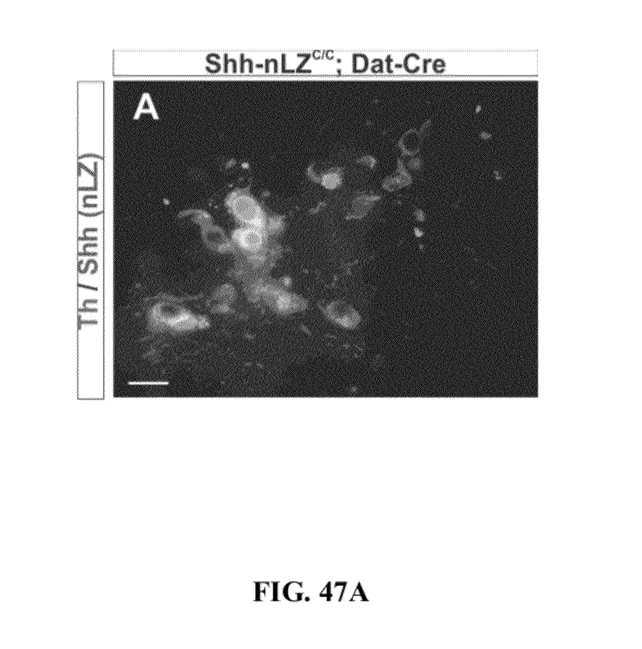

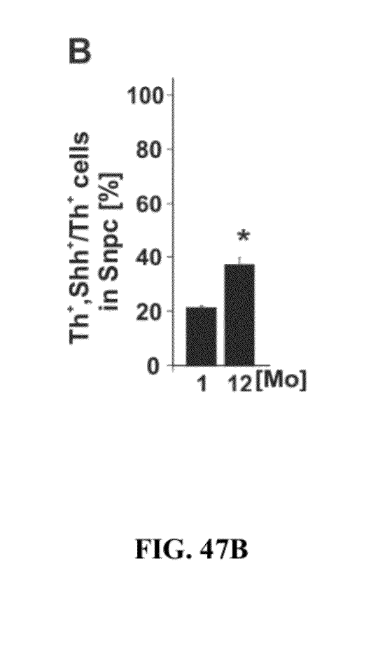

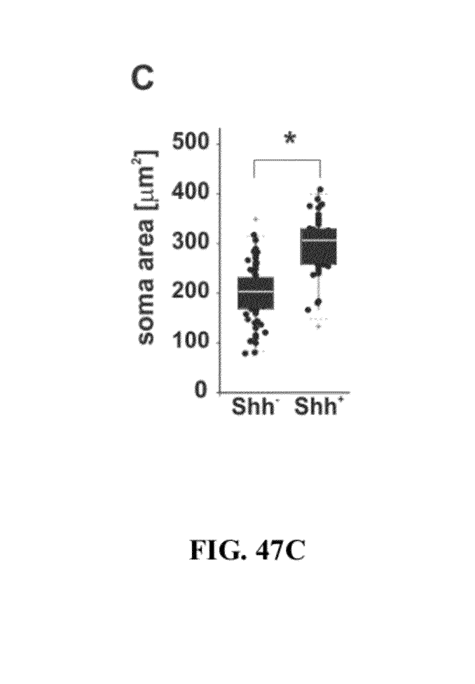

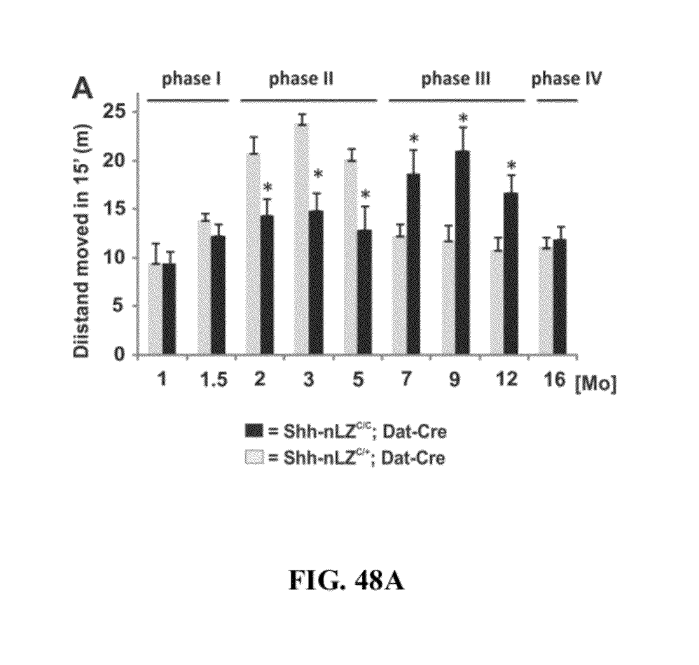

Dat-Cre mediated ablation of the conditional Shh allele is 40% complete in the SNpc at 2 months of age (FIG. 54A). In contrast to further accumulating ablation of the conditional Shh allele during the life time of Shh-nLZ^{C/C}, Dat-Cre mutant animals, an increase in the relative numbers of Shh expressing DA neurons among all DA neurons of the SNpc is observed from ˜20% at 5 weeks of age to ˜37% at 12 months of age in Shh-nLZ^{C/C}; Dat-Cre mutants, indicating that most of the Shh expressing DA neurons that escaped Cre mediated Shh ablation, selectively survive in Shh-nLZ^{C/C}; Dat-Cre mutants (FIG. 47A-B). The soma of Shh⁺ DA neurons is significantly larger than the soma of Shh⁻ DA neurons at 12 months of age (FIG. 47C). These results indicate that Shh expressing cells are less prone to degeneration compared to Shh⁻ DA neurons in Shh-nLZ^{C/C}; Dat-Cre mutants, indicating that Shh exerts a cell autonomous neuro-protective effect on DA neurons.

Progressive Motor Abnormalities in Shh-nLZ^{C/C}; Dat-Cre Mutants.

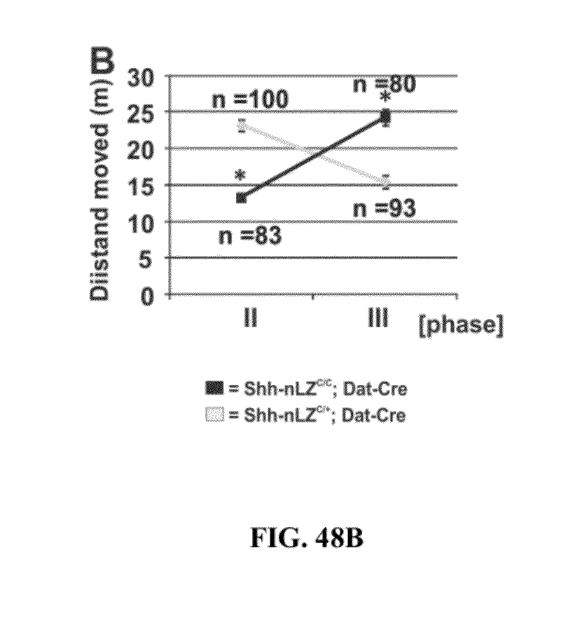

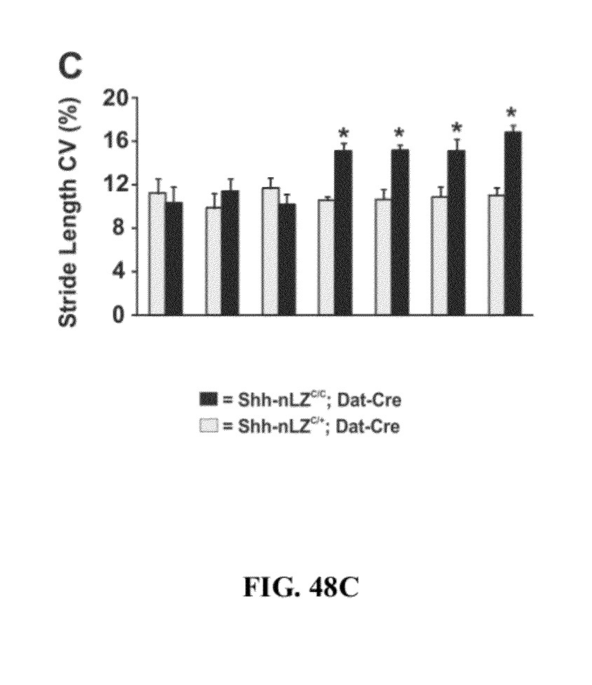

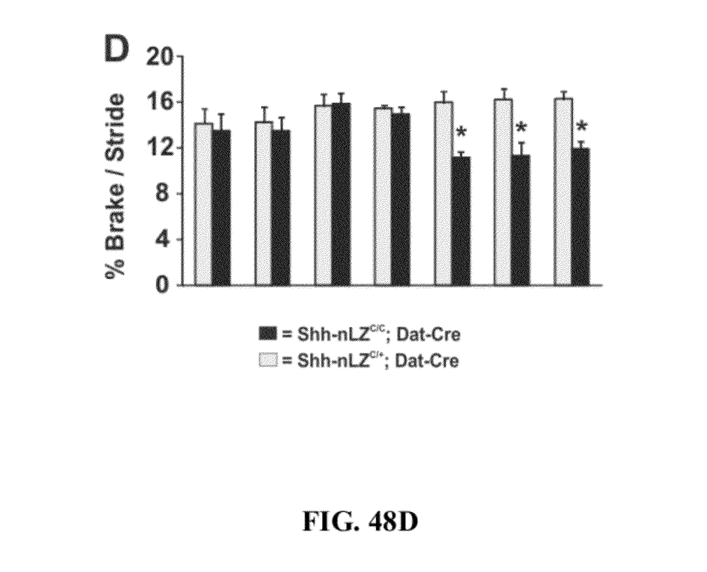

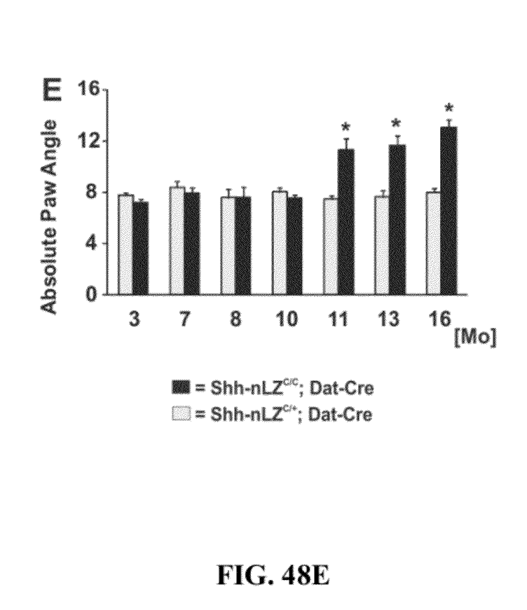

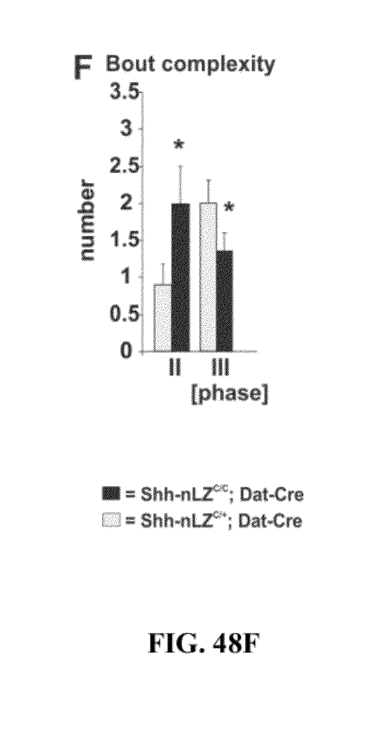

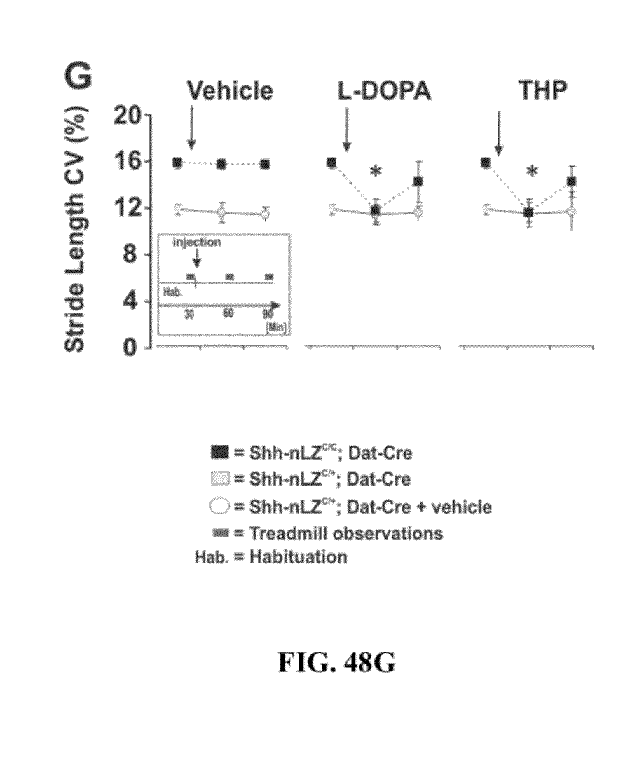

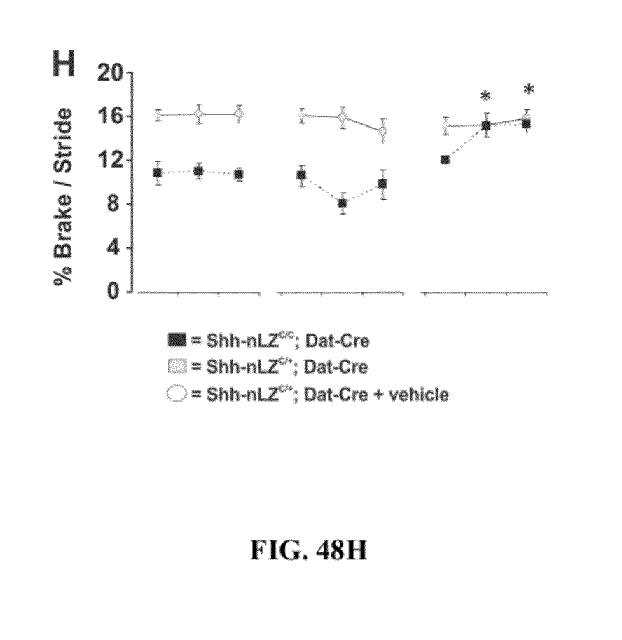

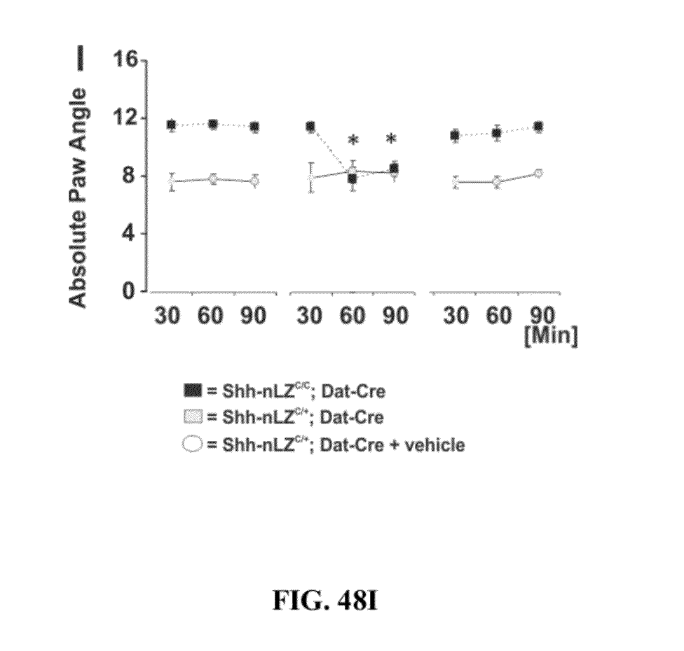

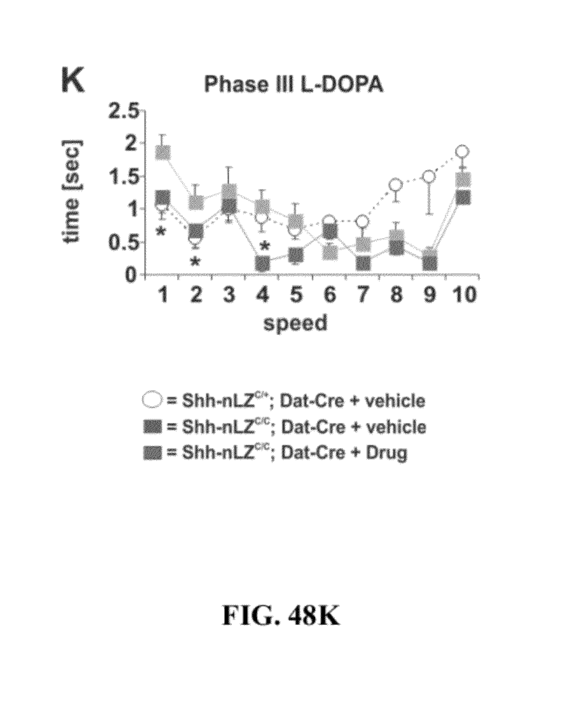

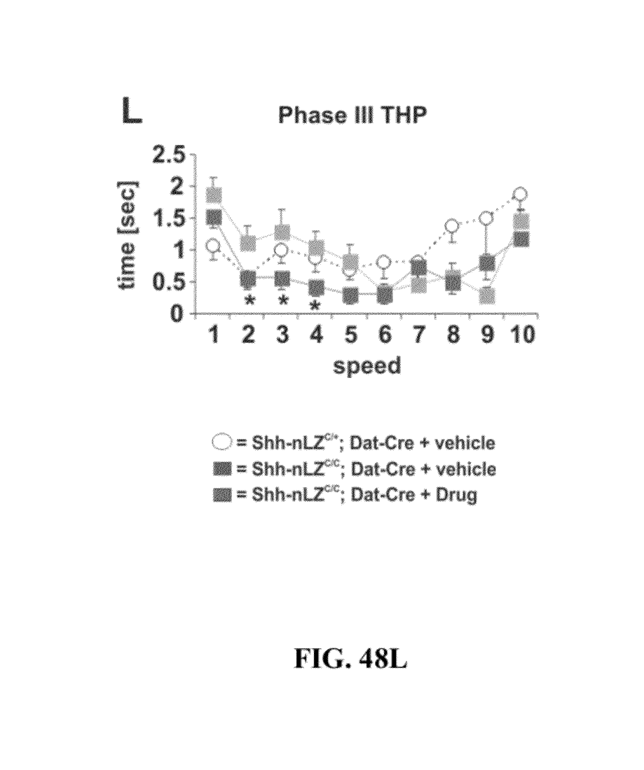

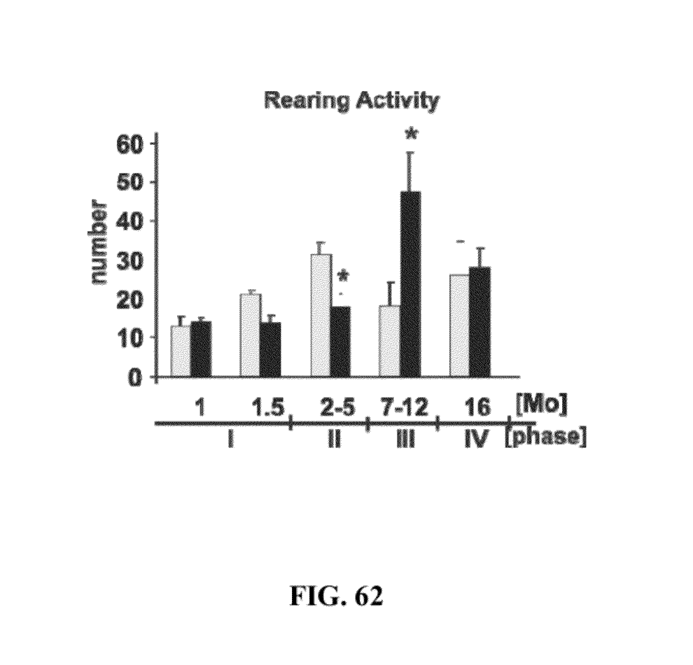

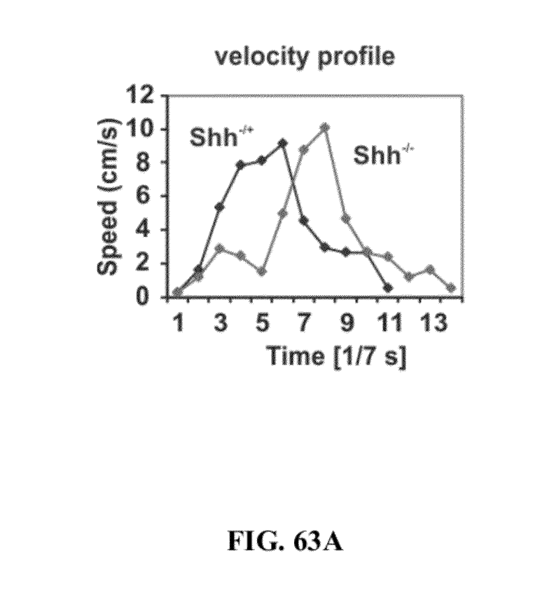

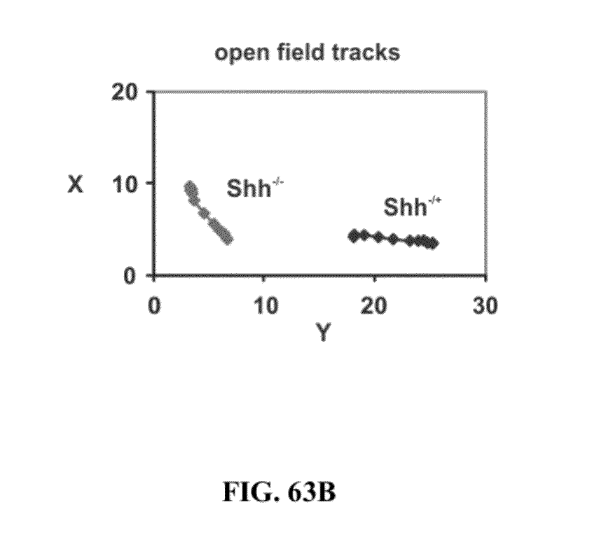

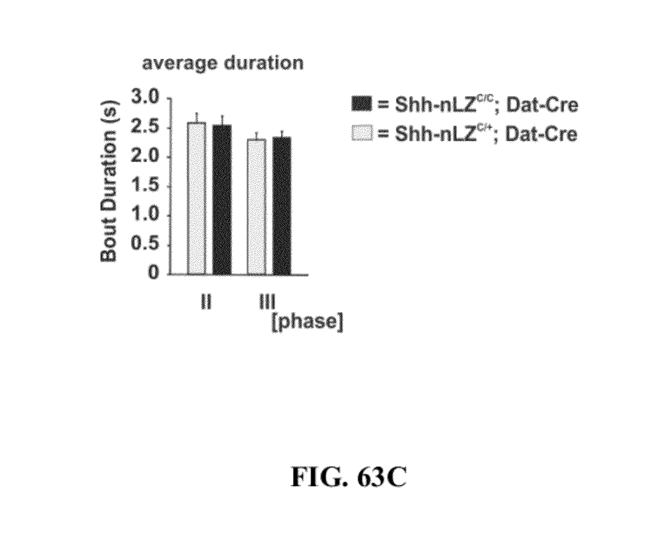

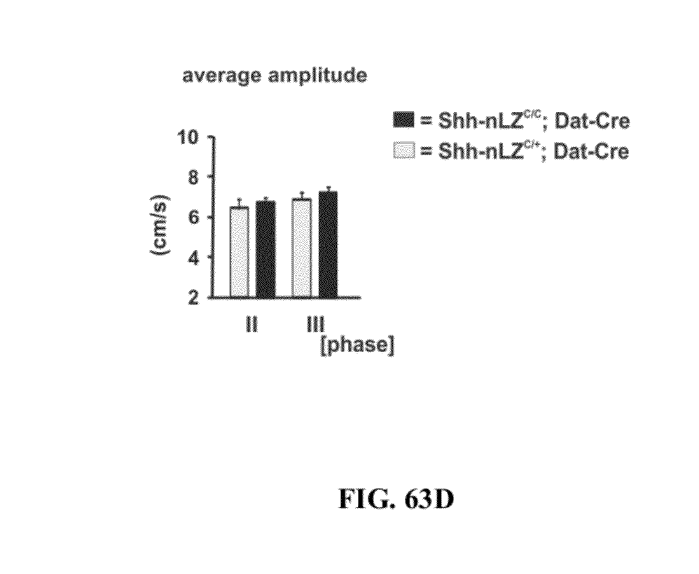

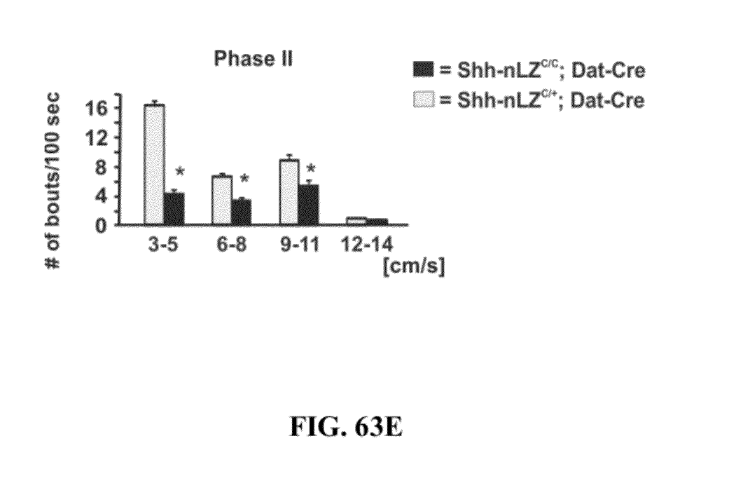

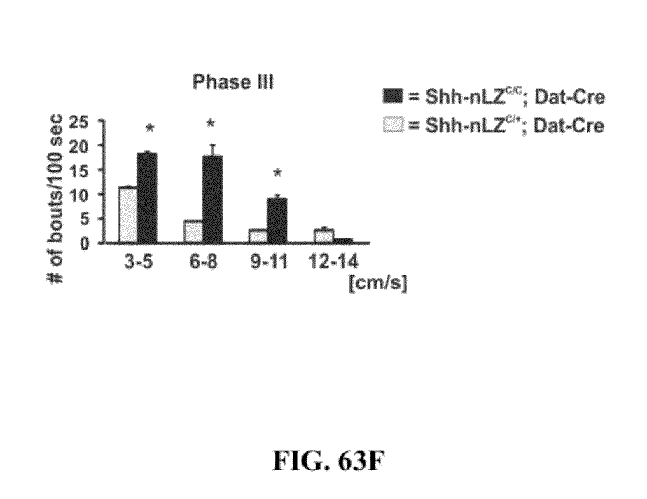

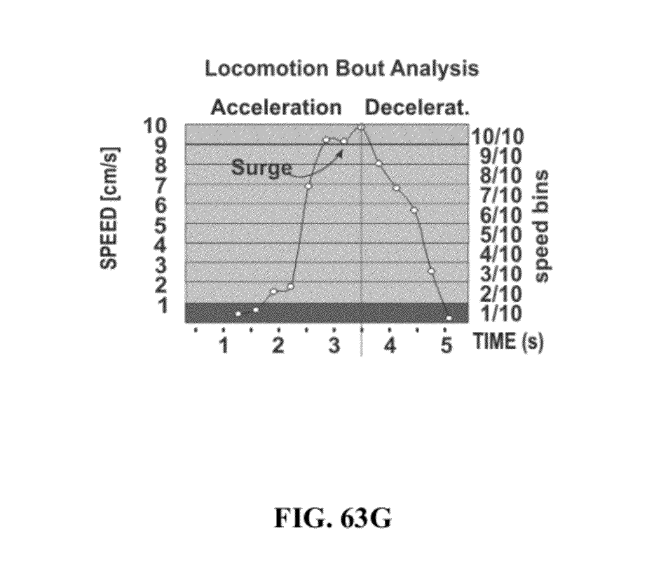

To assess the functional significance of the progressive structural and neurochemical corruption of the meso striatal circuit observed in Shh-nLZ^{C/C}; Dat-Cre mutants, longitudinal analyses of motor performance were used. Comparative analysis of horizontal activity profiles of freely locomoting mice in an “open field” arena reveals that Shh-nLZ^{C/C}; Dat-Cre mutants are maximally active between 7 and 9 months of age whereas control litter mates are maximally active between 2 and 5 months of age. The distinct temporal activity profiles of mutant and control animals let one operationally define successive phases of progressive changes in locomotion (FIG. 48A): up to 6 weeks (phase I) locomotor activity was indistinguishable between Shh-nLZ^{C/C}; Dat-Cre mutant and control animals. In phase II (2-5 months of age) Shh-nLZ^{C/C}; Dat-Cre animals exhibited a 35% reduction in locomotion compared to control animals whereas in phase III (7-12 months of age) Shh-nLZ^{C/C}; Dat-Cre animals increased their activity 60% relative to control littermates. By 16 months of age (phase IV) locomotion activity returns to control levels in Shh-nLZ^{C/C}; Dat-Cre animals, which progress to a phase (V) of rapid neurological decline with first pelvic dragging, then partial hind limb paralysis and premature death at about 18 months of age. In fair agreement with the horizontal movement described above, rearing activity is also altered with similar dynamics (FIG. 62). The switch from relative hypo-activity to relative hyper-activity when compared to control littermates appears in Shh-nLZ; Dat-Cre mutant animals with high temporal specificity around 6 months of age (FIG. 48B). Further kinematic analysis (see Other Results and Discussion) reveals that the average duration and amplitude of individual bouts of locomotion is unaltered between Shh-nLZ^{C/C}; Dat-Cre and control animals in phases II and III (FIGS. 63C and D). However, the frequency of bouts of locomotion is reduced in phase 2 and increased in phase 3 in Shh-nLZ^{C/C}; Dat-Cre mutants relative to controls (FIG. 63C-F). These observations point to alterations in the mechanisms of initiation of locomotion in Shh-nLZ; Dat-Cre mutants.

Gait dynamics by ventral plane videography of mice walking on a translucent treadmill (Digigait system, Mouse specifics, Inc.) were investigated, from which comparative temporal, spatial and force indices of gait were derived (Hampton et al., 2010) of Shh-nLZ^{C/C}; Dat-Cre—and control animals from 3 to 16 months of age (Table 1). Since altered posture can confound comparative gait analysis based on absolute measures, further analysis of locomotion on the tread mill was focused on gait length variability and relative indices of gait dynamics (relative time allotted to Swing, Brake, Propel and Stance phases) and foot angle. In Shh-nLZ^{C/C}; Dat-Cre mutants, gait length coefficient of variability was 30% increased at 10 months of age (FIG. 48C). At 11 months of age Shh-nLZ; Dat-Cre mutants exhibited a 40% reduction in time devoted to braking in each stride (FIG. 48D) and a 50% increase in the absolute paw angle relative to controls (FIG. 48E). These phenotypes did not worsen with increased age and appeared towards the end of phase III defining a distinct progression of phenotype not linked to the relative increase in frequency of locomotion bouts per se.

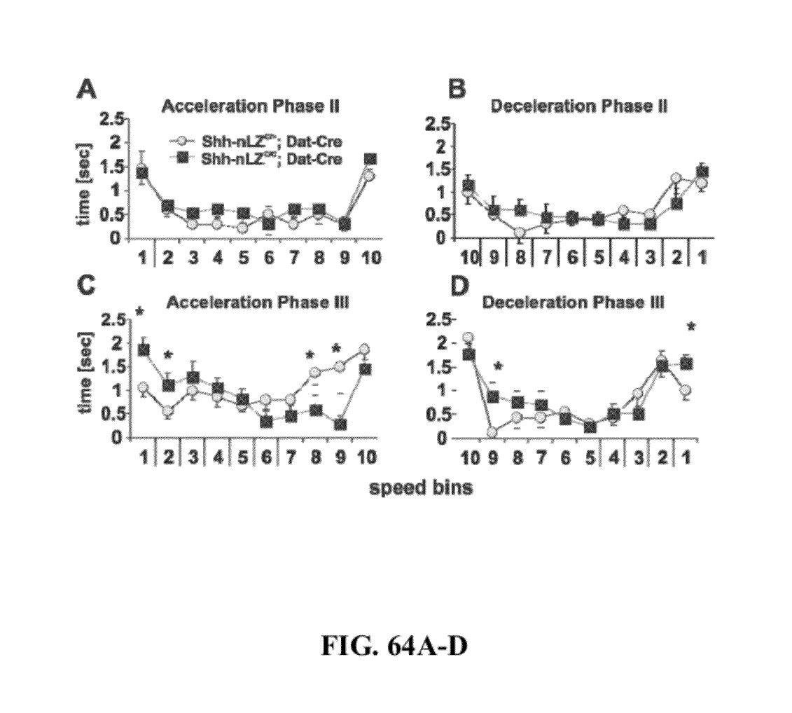

Mesostriatal computations take part in the sequencing of locomotor activity and in the termination of ongoing actions (Jin and Costa, 2010; Ding et al., 2010). These processes can be partially assessed by analysis of the kinematic complexity of individual bouts of locomotion in freely ambulating mice (Benjamini et al. 2011; see also Other Results and Discussion). Quantifying the number of alternations from acceleration to deceleration and back in individual bouts of locomotion of similar duration and overall amplitude (FIG. 63G; see also Other Results and Discussion), a 100% increase in locomotive complexity of Shh-nLZ^{C/C}; Dat-Cre mutants in phase II but a 40% decrease in complexity in phase III compared to control littermates was observed (FIG. 48F). How much time animals spend traveling at different velocities during individual bouts of locomotion of similar length and amplitude was quantified next (see Other Results and Discussion). In phase II mice spend most time locomoting at initial and at top speeds with no discernable differences between Shh-nLZ; Dat-Cre mutants and control littermates (FIG. 64A-B). In phase III, however, Shh-nLZ^{C/C}; Dat-Cre animals spend 50% more time at low velocities and 80% less time at submaximal speed levels before reaching maximal velocity (FIG. 48K and FIG. 64C). Shh-nLZ^{C/C}; Dat-Cre animals also spend more time at sub-maximal speed levels after the animal reached peak velocities in a given bout compared to controls (FIG. 64D). These measures reveal a functional deficit during the initiation of acceleration and deceleration in each bout of locomotion in Shh-nLZ^{C/C}; Dat-Cre mutant animals compared to controls.

Dopamine Substitution and Anticholinergic Pharmacology Normalize Gait and Locomotion Disturbances

Many of the locomotion disturbances observed in Shh-nLZ^{C/C}; Dat-Cre were reminiscent of the functional hall marks of Parkinson's spectrum diseases which are characterized by a general impoverishment of locomotion, reduced fluidity of movement, difficulties in initiating and terminating locomotion, and increased stride length variability (Hausdorff et. al, 1998). Whether dopamine substitution by L-Dopa and the muscarinic antagonist trihexiphenidyl (THP), drugs of proven efficacy in the management of symptoms of PD related diseases, will modify acutely the locomotion deficits of Shh-nLZ^{C/C}; Dat-Cre mice, was tested. L-dopa, THP, or vehicle were systemically injected 30 minutes prior to the analysis of locomotion into 12 month old Shh-nLZ^{C/C}; Dat-Cre and controls. The increased variability in stride length observed in experimental animals was normalized to control levels by L-Dopa and THP (FIG. 48G). THP, but not L-Dopa, normalized Brake-Stride ratios to control levels (FIG. 48H). In contrast L-Dopa, but not THP, normalized the alterations in paw angles (FIG. 48I). L-Dopa and THP administration also ameliorated the deficits in the initiation of acceleration and deceleration observed by kinematic analysis of spontaneous locomotion but neither drug normalized the time Shh-nLZ^{C/C}; Dat-Cre mutant animals spend locomoting at sub-maximal speeds prior to reaching top velocity (FIGS. 48K and L).

### Discussion

Shh signaling originating from dopaminergic neurons of the mesencephalon exerts an indispensible neurotrophic effect on ACh- and FS-interneurons of the striatum and acts as a neuroprotectant for dopaminergic neurons in vivo. The data herein provide evidence that DA-neuron produced Shh acts in the striatum directly and selectively on ACh- and FS-interneurons and that ACh-neuron are a significant source of the dopaminotrophic factor GDNF in the adult basal ganglia linking GDNF mediated trophic support of dopaminergic neurons with Shh signaling from DA neurons to ACh-neuron. The findings herein are consistent with the existence of a reciprocal trophic factor signaling loop between DA neurons and ACh-neuron and show that the regulation of expression of these factors in these neurons in the adult brain has rheostat properties. Evidence that Shh signaling is involved in the determination of the set point of the extra cellular acetylcholine tone in the striatum through the regulation of muscarinic autoreceptor signaling is further provided. Taken together, the results herein reveal a means by which meso-striatal dopaminergic neurons signal selectively to a subset of their striatal neuronal targets and thereby regulate cellular and neurochemical homeostasis in the meso striatal circuit in the adult brain. The data herein also offer insights in the possible causes of the spreading of neuronal demise in idiopathic neurodegenerative conditions that inflict the basal ganglia.

Reciprocal Trophic Factor Signaling Between Dopaminergic and Cholinergic Neurons

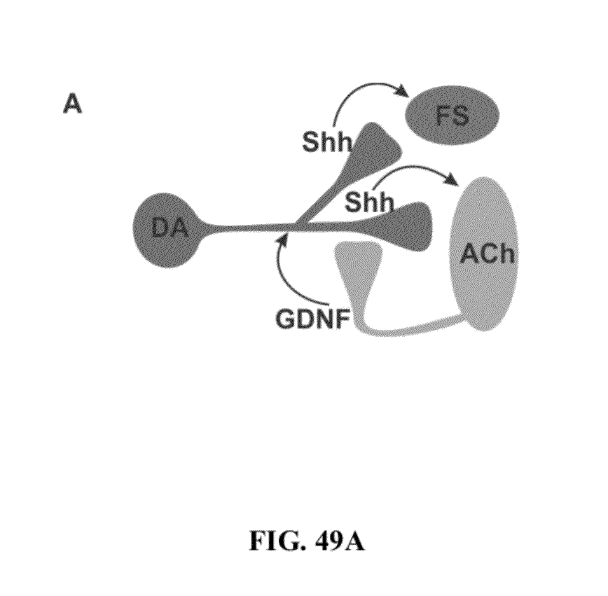

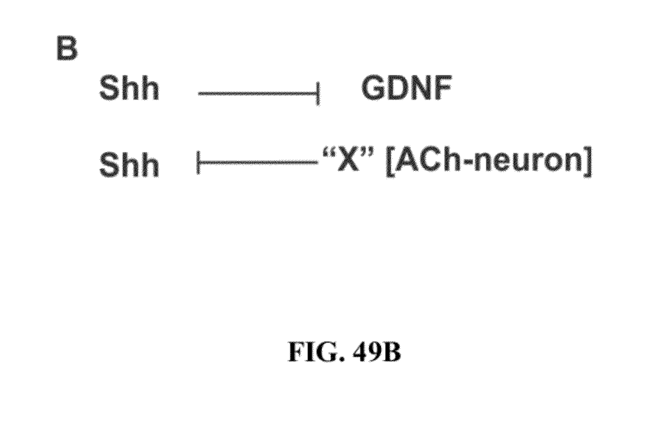

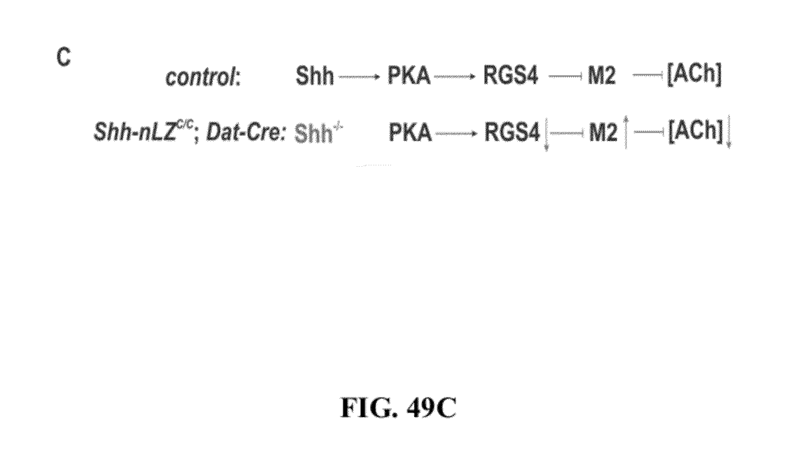

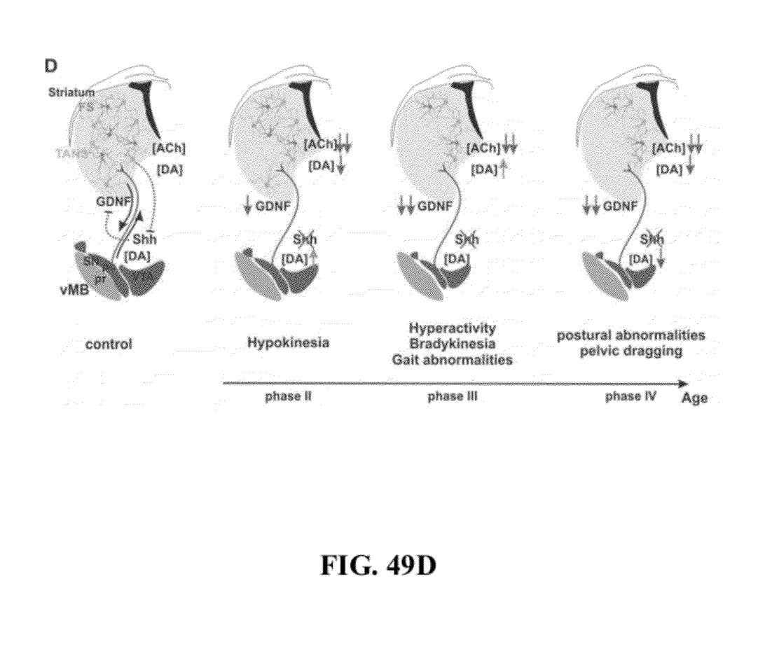

In the mature brain, DA-neurons express Shh while ACh-neurons, one of the main projection targets of mesencephalic DA-neurons (Nastuk and Graybiel, 1985), express GDNF and the receptor for Shh, Ptc1 (FIG. 43). The DA-neuron restricted ablation of Shh described herein results in the progressive degeneration of ACh-neurons. Trophic support of ACh-neurons by DA neuron produced Shh can be provided in a static manner or be induced in response to physiological need. Transcriptional activation of Shh loci is observed in the ventral midbrain upon (1) injection of the dopaminerig toxin 6-OHDA into the mFB, (2) induction of cholinergic dysfunction by injection of the cholinotoxin AF64a into the striatum, and (3) genetic reduction of Shh signaling from DA neurons to the striatum. These results indicate that ACh-neurons are a source of an inhibitory signal for Shh transcription in DA neurons whose delivery to the nucleus of DA neurons requires intact dopaminergic axonal projections into the striatum. Previously, it was observed that Shh expression in facial motor neurons is induced upon nerve axotomy in the adult and that increased Shh signaling promotes the survival of injured motor neurons in this model (Akazawa et al., 2004). These experiments indicated that Shh transcription in the healthy spinal motor neuron system is controlled by inhibitory signals that emanate from peripheral tissues and are relayed back to the soma of motor neurons by their axons. The results herein extend these findings to the mesencephalic DA system of the adult brain in two physiologically relevant ways: (1) Cholinergic neurons that are trophically dependent on Shh from DA neurons are a source of inhibitory signals for the transcription of Shh by DA neurons, and (2) the extent of relief of transcriptional inhibition of Shh expression is correlated to the degree of cholinergic dysfunction when averaged across DA neurons (FIG. 43). This design of control of Shh gene expression gives Shh function within the meso striatal pathway a finely tuned rheostat capability that is linked to the cell physiological status of ACh-neurons: Without being bound by theory, physiological stress in ACh-neurons which attenuate the expression of the inhibitory signal(s) for Shh expression can lead to an increase in trophic signaling to ACh-neurons from DA neurons until cellular homeostasis is regained and gene expression is normalized (FIG. 49B).

It is observed that the induced up-regulation of Shh by DA neurons in control animals causes inhibition of GDNF expression in the striatum without effecting the expression of ChAT and vAChT (FIG. 45). Hence, Shh signaling from mesencephalic DA neurons maintains selectively GDNF expression at tightly controlled rates by acting as an inhibitory signal for GDNF transcription while supporting ACh-neurons trophically at lower and tonic levels. These observations fit well with the established concentration dependence of the functional modes of Shh signaling (discussed in Ullao and Briscoe, 2007): Low levels of Shh signaling is necessary for tissue maintenance and limiting Shh signaling below a critical threshold results in the “sculpting” of the size of neuronal populations in the dorsal half of the developing spinal cord (Mehlen et al., 2005, Cayuso et al., 2006) whereas higher concentrations of Shh regulate in a concentration dependent manner gene expression mediated by either transcriptional-repressor or -activator forms of the Shh signaling components Gli 1, 2, and 3 (Briscoe and Novitch, 2008).

The inefficiency of Cre recombination of the Shh allele created heterogeneity among DA neurons in regard of Shh expression which allowed the investigation of whether Shh expression by DA neurons confers cell autonomic neuro-protection. The results herein reveal a ˜2 fold enrichment of Shh expressing DA neurons during phenotype progression in ShhnLZ^{C/C}; Dat-Cre animals demonstrating that mostly Shh^{-/-} DA neurons degenerate. The studies herein therefore provide evidence for a neuroprotective function of dopaminergic expression of Shh for DA neurons in the adult mesencephalon consistent with a potential autocrine mechanism of Shh signaling. Shh mediated autocrine signaling is observed in neoplasia (Mao et al., 2009) and the differentiation of teeth in Sqamata (Handrigan and Richman, 2009). However, the absence of expression of the Shh receptors Ptc1 and 2 by DA-neurons (FIG. 52), the observed control of dopaminergic expression of Shh by signals from ACh-neurons (FIG. 43) and the strict correlation of the degeneration of ACh-neurons and DA neurons with a kinetics highly similar to the progressive degeneration of DA neurons observed upon the ablation of GDNF in the adult CNS or of the GDNF receptor Ret from DA neurons (Pascual et al, 2008; Kramer et al., 2007) make this scenario unlikely.

An alternative interpretation of the apparent cell autonomic function of Shh in DA neurons is the possibility that individual cartridges of meso-striatal circuits act as autonomic units in regard of reciprocal trophic factor signaling with little spill over between individual cartridges: In this scenario, ACh-neurons continue to supply GDNF to support DA neuron survival in those neuronal cartridges in which DA neurons have escaped Cre mediated recombination of the Shh alleles, but do not signal to constituent DA neurons of other meso-striatal circuits that have lost Shh expression. This interpretation is supported by the quantification of synaptic connectivity in the striatal microcircuit: While ACh-neurons and DA neurons elaborate wide-spread and spatially overlapping arbors, each neuron only contributes to a few hundred of the estimated 2 million meso-striatal circuits in the striatum (Bolam et al., 2006) suggesting that a given DA neuron might be able to signal to only a few ACh-neurons via Shh. Further support of a confinement of Shh action to the vicinity of Shh release sites in the adult striatum comes from Loulier et al., (2005) who found strong expression in the adult striatum of the Hedgehog-interacting protein (Hhip), which inhibits Shh signaling by complexing to secreted Shh, likely further limiting the poor diffusion of Shh once secreted (Ulloa and Briscoe, 2007).

The inability of surviving ACh-neurons to compensate functionally for the reduction in their numbers in regard of extracellular ACh tone in the striatum indicates that Shh signaling can play a role in the regulation of the neurochemical- and/or electro-physiology of ACh-neurons. Extracellular ACh tone in the striatum is variably regulated by DA neuron activity (Threfell et al., 2010). Interestingly however, dopaminergic activity does not exert its effect on autoreceptor function via dopamine receptors expressed on ACh-neurons, but instead through regulation of the coupling of muscarinic auto receptors to K⁺ and Cav2 Ca⁺⁺ channels mediated by altered “regulator of G-protein signaling” (RGS) expression (Ding et al, 2006). This leaves open the possibility that signaling molecules other than dopamine itself emanating from DA neurons are involved.

The data herein link Shh signaling originating from mesencephalic DA neurons to the regulation of cholinergic tone in the striatum. Is the drastic reduction in cholinergic tone far beyond of what will be expected from the observed partial degeneration of ACh-neurons itself a (1) direct result of reduced Shh signaling to ACh-neurons leading to a perversion of meso striatal physiology or an (2) indirect, counterintuitive adaptation, caused by reduced Shh signaling from DA-neurons to the striatum? The longitudinal analysis of autoreceptor and RGS expression supports a direct role of Shh in the regulation of cholinergic tone since an up-regulation of M2 and down-regulation of RGS4 is observed at 5 weeks as well as 12 months of age, indicating that the dysregulation of cholinergic autoreceptor activity occurs prior to the manifestation of the cellular and functional aspects of the progressive corruption of mesostriatal circuitry. Consistent with this interpretation, work on the regulation of muscarinic auto receptor activity in ACh-neurons provides a potential cell autonomous link between Shh signaling and auto receptor activity: Protein-kinase A (PKA) is an activator of RGS4 (Huang et al., 2007), whose inhibition increases autoreceptor function (Ding et al., 2006). Shh signaling reduces the levels of cAMP and PKA activity (Ogden et al., 2008) offering a potential molecular pathway by which reduced or absent Shh signaling can lead to an increase in muscarinic auto receptor activity in surviving ACh-neurons (FIG. 49C).

Lowered cholinergic tone, a reduction of striatal GDNF levels due to progressive degeneration of a significant portion of the population of ACh-neurons, and a lack of functional adaptations in the population of surviving ACh-neurons should influence dopaminergic physiology of surviving DA-neurons since ACh facilitates dopamine signaling (Threlfell et al., 2010) and GDNF signaling increases the quantal size of dopamine release (Pothos et al., 1998). In contrast to a monophasic change of dopamine levels, however, the longitudinal data reveals highly dynamic distortions in dopamine tissue content in the ventral midbrain and striatum with a sharp onset of a striatal dopamine deficiency prior to detectable degeneration of dopamine neurons followed by a normalization of dopamine levels in both compartments for much of adult life despite ongoing neuro degeneration (FIG. 46). The data herein indicate that surviving DA-neurons, in contrast to surviving ACh-neuron, are able to adapt their physiology dynamically in the face of progressive neuro-degeneration and the diminishment of ACh and GDNF signaling early in phenotype progression. However, by 10 months of age, the manifestation of discrete locomotion and gait disturbances is found, many of which can be normalized by pharmacology that impinges on the functional balance of dopamine and acetylcholine (FIG. 48, Lester et al., 2010), indicating that at this time point the progressive corruption of the meso striatal circuit surpasses the compensatory capacity of DA-neurons (FIG. 49D).

Implications for Understanding the Etiology of Diseases of the Basal Ganglia

Similar to the GDNF and Ret ablation studies (Kramer et al., 2007; Pascual et al., 2008), it is observed that the cellular and functional consequences of the ablation of Shh from DA neurons have an adult onset and develop progressively over several months despite an ˜80% efficient and not further progressing recombination of the Shh alleles at 5 weeks of age. These results demonstrate that withdrawal of Shh and/or GDNF signaling in vivo exposes the meso-striatal pathway to increased risk of degeneration, but not to cellular demise a priori. Instead, consistent with the “multiple hit” hypothesis as the mechanistic cause of progressive neurodegeneration observed in PD (Sulzer, 2007), the progressiveness of neuronal degeneration in this model argues for the involvement of additional, cell type specific, aberrant physiological processes that are induced by the absence of growth factor signaling and in turn cause an accumulating burden on the survival of ACh-neuron and DA neurons over time. The data herein demonstrate that Shh signaling plays a pivotal role in cholinergic physiology in addition to acting as a survival factor for ACh neurons and point to the possibility that the distortions in ACh and DA production and secretion, which develop in the absence of Shh signaling from Da neurons, contribute to the demise of cholinergic and dopaminergic neurons.

Archetypes of basal ganglia models predict that an imbalance of cholinergic and dopaminergic signaling in the striatum is responsible for the hyper- and hypo-kinetic manifestations of movement disorders (Albin et al., 1989; Obeso et al., 2000). The results herein demonstrate that the changes in the balance of dopaminergic and cholinergic tone that develop in the absence of Shh signaling from DA neurons impinge on the formation of striatal locomotor output since many of the progressive locomotion anomalies observed can be corrected by dopaminergic substitution by L-Dopa and an antimuscarinic (THP), both of which are used for the amelioration of motor manifestations of PD. Thus, the work herein describes a new mouse model that recapitulates many of the key features of the progressive cellular, neurochemical, and functional pathologies observed in PD (FIGS. 46 and 48). However, the resemblance of the phenotype of Shh-nLZ^{C/C}; Dat-Cre mutants with PD does not extend to the absolute direction of alterations in cholinergic tone: In PD, ACh tone is increased while DA levels fall due to dopamine neuron degeneration (Wooten, 1990), while the experiments herein demonstrate that the loss of Shh signaling, which also must occur in PD due to DA neuron degeneration, decreases ACh secretion consistent with an observed increase in muscarinic autoreceptor expression.

In PD, Shh production can be up-regulated in still functioning dopaminergic neurons. This is supported by several in vivo experiments described herein. It is demonstrated that the transcription of Shh in dopaminergic neurons is strongly up-regulated upon (1) injection of 6-OHDA into the mFB, (2) induction of cholinergic dysfunction in the striatum, (3) induction of cholinergic dysfunction in the PPTg, and (4) the genetic ablation of part of the Shh locus which abrogates the production of functional Shh by dopaminergic neurons. All four experimental manipulations induce pathological states similar to those observed in PD: 6-OHDA injections result in the degeneration of dopamine neurons and serve as an established neurotoxicological model of PD. Corruption of cholinergic function in the striatum has been recognized as a central feature of the pathology in PD and other movement disorders (Bonis et al., 2011). Cholinergic neuron loss in the PPTg has been observed in PD (Rinne et al., 2008) and might occur prior to the involvement of mesencephalic DA neurons according to the hypothesis of a caudal to rostral spread of neuronal dysfunction in PD as judged by Lewy body pathology (Braak et al., 2003). Genetic ablation of Shh from dopaminergic neurons described herein causes progressive dopaminergic and cholinergic neuron degeneration and locomotion deficits which can be normalized in part by standard pharmacological interventions used in the management of PD (FIG. 48). Hence, the data herein are consistent with a scenario in which during PD progression, prior to the whole sale degeneration of DA neurons, dopaminergic neurons express elevated levels of Shh which results in turn in an increase of ACh tone in the striatum mediated by a down regulation of muscarinic auto-receptor efficacy.

Age represents the greatest risk factor for developing PD (de Lau and Breteler, 2006). Although the mechanisms for aging related increases in neuronal vulnerability are not established, reductions in support systems such as growth factors have been postulated to increase the susceptibility of DA neurons to external stressors and toxins (Yurek and Fletscher-Turner, 2000). A subtle, but significant reduction in the frequency of Shh⁺, Th⁺ double positive neurons among DA-neurons during aging in control animals is revealed (FIGS. 46 and 51). The results herein show that those DA-neurons that have lost Shh expression will encounter reduced trophic support because axonally connected ACh-neuron will eventually attenuate gene expression and/or degenerate similar to what is observed in Shh-nLZ^{C/C}; Dat-Cre. If affected ACh-neuron will normally provide GDNF to more DA neurons than those that have lost Shh expression due to age associated, possibly stochiastic, mechanisms, a self-reinforcing cycle of spreading neuronal demise can ensue, providing a potential mechanisms for idiopathic, age dependent, neuro-degeneration. In this scenario, the ˜80% efficient ablation of Shh from DA neurons in this model can be viewed as mimicking “accelerated” aging of the nigro-striatal system.

GDNF expression among cholinergic neuronal populations in the adult brain occurs only in striatal cholinergic interneurons (FIG. 59), revealing an additional and cholinergic neuron subtype specific means by which ACh-neuron, but not other cholinergic neurons, most of which are neuronally connected with DA neurons (Gaykema and Zaborszky, 1996), can communicate with mesencephalic dopaminergic neurons. In turn, however, Shh mediated signaling from mesencephalic DA neurons to various types of cholinergic neurons is likely less selective since basal forebrain cholinergic neurons in general express the Shh receptor Ptc1 and are trophically dependent on Shh signaling in vitro (Reilly et al., 2002). These observations indicate that Shh carried by dopaminergic projections can also influence the physiology and trophic support of extra-striatal cholinergic neurons in vivo including the nucleus of Meynert, which is involved in cognition and the habenula involved in sleep regulation (Bohnen and Albin, 2010). Without being bound by theory, PD associated fronto temporal dementia and sleep disturbances (Obeso et al., 2010) can be caused by extra striatal cholinergic dysfunction that results from altered Shh signaling from mesencephalic DA neurons to cholinergic neurons of the basal forebrain and habenula in disease.

The data herein reinforce the rationale for supporting growth factor signaling as a disease modifying therapeutic strategy in basal ganglia diseases. However, the uncovered negative feedback regulation of endogenous growth factor expression within the meso-striatal circuit indicates that exogenously supplied trophic factors can inhibit endogenous expression of the same factors possibly curtailing the therapeutic benefit of this approach. Instead, the results herein point to the possibility that undercutting the negative feedback regulation of endogenous growth factor expression can result in therapeutically effective increases of trophic factor signaling within the basal ganglia.

### Experimental Procedures

Mouse Strains

Shh-nLZ^{C/+} mice were generated by insertion of a BamH1 and LoxP site containing linker (CTAGGCGCGCCTCTAGAGGATCCATAACTTCGTATAATGTATGCTATACGAAGT ATC) into the XbaI site 5′ to the 2^{nd} exon and by the insertion of a IRES nLacZ cassette followed by the LoxP flanked PGK-Neo cassette into the NcoI site in the 3′ non translated region of the Shh gene followed by gene targeting in ES cells. Additional construction details, mouse strains and genotyping procedures are described below and in FIG. 50. All animal handling and procedures were approved by the Animal Care and Use Committee of Columbia University and performed in accordance with NIH guidelines.

Immunohistochemistry

Immunohistochemistry was performed on 12-100 μm cryosections or free floating sections using primary and secondary antibodies listed below. Images were acquired on BioRad MRC 1024 or Zeiss LSM510 Meta confocal microscopes, or Nikon E600 with epi fluorescence and DIC optics. Quantification of the size of populations of cells was estimated by the optical fractionator method described below.

Quantitation of GDNF Tissue Content

Tissue levels of GDNF were measured with an ELISA kit (GDNF Emax ImmunoAssay System; Promega, Madison, Wis.), according to the protocol provided by the supplier. The levels of GDNF were expressed as pg/mg of total protein determined by Lowry assay for each sample. The assay sensitivity ranged from 16 to 1000 pg/ml.

Quantitation of Gene Expression

Total RNA from striatum and lateral ventral midbrain containing the entire SN and VTA was isolated using RNeasy Mini Kit (Qiagen) and reverse transcribed using oligo(dT) primers and the SuperScript First-Strand Synthesis System (Invitrogen), according to the manufacturer's protocol. Relative mRNA levels were quantified by real-time PCR using TaqMan Gene Expression Assays (Applied Biosystems) with amplicons (listed in Table 2) designed using Primer Express 1.0 (Applied Biosystems). For each gene target triplicate cDNA samples were amplified in 96-well optical plates in an ABI 7700 Real-Time PCR instrument (Applied Biosystems). Gene expression results were normalized against GAPDH which was run as endogenous control for each sample. The ΔΔCt method was used to calculate the expression fold change.

Neurochemical Analysis:

Determination of the concentration of dopamine and acetylcholine in the meso striatal system and neurotoxicological challenges were performed as described in supplemental experimental procedures methods.

Locomotion Analysis

Spontaneous motor activity was measured in an open field arena using automatic tracking at 6 Hz by an EthoVision 3.1 system (Noldus Information Technology, Leesburg, Va.). Derivation of acceleration and deceleration profiles and indices for turning bias and locomotion complexity is described below. Analysis of gait parameters by forced locomotion was performed by ventral plane videography on a translucent tread mill as described (Digigait, Inc.; Hampton et al., 2010).

Statistical Analysis

The mean and SEM of values were calculated and the significance of all pair-wise comparisons was determined by two-tailed distribution homoscedastic Student's t-test and by ANOVA, including a repeated measures factor when necessary. Follow up analysis between groups with multiple comparisons was by Tukey's Post Hoc Test. Nonparametric data were analyzed by Mann-Whitney U test. For all box plots, the box includes data points between the 25^{th} and 75^{th} percentile of all values, with the line representing the median value. The lines and whiskers represent data between the 9^{th} and 91^{st} percentile and individual dots represent outlier points. Data was considered significant for all p values of <0.01.

Other Results and Discussion

Characterization of the Tissue Specificity and Efficiency of Shh Ablation in the Presence of the Dat-Cre Allele

The efficiency and tissue specificity of Dat-Cre mediated recombination of the Shh-nLZ^{C} allele were assessed by quantifying the numbers of cells that lost the expression of nLacZ in mesencephalic DA neurons and in the medial amygdala (MeA, a nucleus with strong expression of Shh which is devoid of dopaminergic neurons) of 6 week old animals (FIG. 54A). An overall 79+/−0.6% reduction (with respect to Dat-Cre negative, Shh-nLZ^{C/+} mice, n=3, each hemisphere counted separately, p<0.01) in the number of nLacZ⁺/Th⁺ double positive neurons in the ventral midbrain is found (FIG. 54A). Shh expression in the medial amygdala (MeA), a brain nucleus of massive Shh expression, was not effected (FIG. 54A). The observed recombination frequency of the conditional Shh alleles is similar with previous reports utilizing the Dat-Cre mouse line (Zhuang et al., 2005, Kramer et al., 2007). To assess the tissue specificity of the recombination of the Shh-nLZ^{C} allele more globally in the adult brain, X-gal was used as an enzymatic substrate for β-Gal activity in combination with “glass brain” whole mount preparations. Comparative analysis of optically flattened images of translucent, X-Gal stained entire brains derived from Dat-Cre; Shh-nLZ^{C/+ and Shh-nLZ}^{C/+} mice reveals overall highly similar patterns of β-gal activity with the exception of a pronounced absence of staining in ventral midbrain regions corresponding to the SN, VTA and retro rubral field (red arrows in FIG. 54B-C).

Cre Mediated Ablation of Shh Causes a Null Allele:

The ablation of exons 2 and 3 of the Shh-nLZ^{C} allele (FIG. 50A) results in a loss of Shh function mutation by Cre-mediated recombination of the conditional allele within the germline using Hsp70-Cre mice (Dietrich et al., 2000). F2 embryos homozygous for the recombined Shh allele (Shh^{N/N}, FIG. 50A) reveal one eye field, a holoprosencephalic forebrain, stunted rotation symmetric limb buds missing distal structures and uncurved tail buds (FIG. 50D). Embryonal lethality and morphological features of mutants are highly similar to the phenotype observed after the unconditional, germline ablation of exon 2 only of the Shh locus (Chiang et al., 1996). Animals heterozygous for the recombined allele(Shh^{N/+}) or homozygous for the conditional allele (Shh-nLZ^{C/C}) are born alive, are fertile and have a normal life span with no signs of any overt morphological and functional phenotype. The data herein demonstrate that Cre mediated recombination of Shh-nLZ^{C} allele leads to a loss of function allele. In Shh-nLZ^{C/C} Dat-Cre animals it was confirmed that the vMB restricted recombination of the conditional Shh allele by PCR using DNA derived from the vMB, olfactory bulb and tail (FIG. 50E).

Characterization of Spontaneous Locomotion in Shh-nLZ^{C/C}; Dat-Cre Mice:

When observed in the open field arena after habituation, rodents locomote in bouts of spontaneous activity alternating resting episodes with periods of spatial progression (Drai et. al., 2000; Drai and Golani, 2001). Kinematic data of locomotion in an open field setting can be derived by collecting data of location (X-Y position) over time (sec) series by automatic, temporal high resolution video tracking systems (reviewed in Benjamini et al., 2011; see method section). In contrast to forced locomotion on the treadmill when the animals have to locomote with the speed of the belt, in each bout of spontaneous locomotion, animals can travel at variable speeds at any time during each bout. The transformation of the collected positional data over time into velocity profiles (speed over time) allows the quantification of average locomotion bout duration, amplitude and complexity (Kafkafi et al., 2003a; 2003b; FIG. 63). Average amplitude and duration of bouts were indistinguishable between Shh-nLZ^{C/C}; Dat-Cre mice in phase II and III and aged matched litter controls (Luis please fill in data for duration and stats, FIG. 63C-D). Further analysis reveals that Shh-nLZ^{C/C}; Dat-Cre mice exhibit in phase II a decrease and in phase III an increase in the frequency of bouts of locomotion compared to aged matched litter controls (FIG. 63E-F). These results indicate that altered frequency of initiation of locomotion bouts is the main determining factor in the relative differences in the accumulative locomotion displacement observed in phase II and III between Shh-nLZ^{C/C}; Dat-Cre mutant animals and their littermate controls (FIG. 48A). In many bouts of locomotion animals accelerate to a maximal speed and then decelerate until rest in single biphasic fashion (FIG. 63A-B). In some locomotion bouts animals show more dynamic behaviors with alternations between acceleration to deceleration or vice versa, so called “surges” or “darts” (Kafkafi et al., 2003a) before and/or after they have reached their maximal speed within a given bout (FIG. 63A, B and G). The quantification of the number of alternations between acceleration and deceleration is used as an expression of the complexity of locomotion (Kafkafi et al., 2003a, Benjamini et. al., 2011, FIG. 48F) Binning velocities into 10 continuous, discrete levels of relative speed allows the determination of the average time across multiple bouts of locomotion that an animal spends locomoting at a particular level of velocity during each bout of activity (FIG. 63G, FIG. 64A-D; FIGS. 48K and L).

### Experimental Procedures

Mouse Strains

Shh-nLZ^{C} mice were genotyped by Southernblot as described in FIG. 50 or by PCR The location of probes and amplicons is depicted in FIG. 50A. Sequences of oligos are SHHL1 (1.1) gta aga gca cat tac cca gag aac tg; SHHL2 (2.1) cct gtt gtt act gca tcc ctt cca tc; SHHR3 (3.1) . . . . . In addition the following mouse strains were used in this study and their genotyping was achieved as described: Dat-Cre mice (Zhuang et al., 2005), Ptc1-LZ (Goodrich et al., 1999), ChAT(BAC)-eGFP mice (Tallini et al., 2006) and GDNF-LZ (Moore et al., 1996).

In Situ Hybridization

In situ hybridization was performed based on the method of Schaeren-Wiemer and Gerfin-Moser (1993) with digoxigenin-labelled riboprobes of 580 to 680 bases in length on 16 μm cryostat sections. Experimental and control tissue was collected on the same slides. Background levels and the specificity of hybridization were determined using sense strand riboprobes in each experiment.

Immunohistochemistry

Mice were perfused intracardially with saline followed by 4% paraformaldehyde in PBS and brains were post-fixed in the same fixative overnight at 4° C. and then equilibrated in 30% sucrose in PBS for 48 hours at 4° C., and stored at −80° C. Cryostat sections (of 16 to 100 μm) through the entire midbrain and striatum were collected as free-floating sections or on glass slides in sets of interleaved series with 300 μm intervals along the anterior-posterior axis.

The following primary antibodies were used in the study: rabbit α β-Gal (Invitrogen), rabbit α TH (Calbiochem), mouse a NeuN (Chemicon), goat α ChAT (Millipore), goat α Parv (Swant), rabbit α GAD 67 (Chemicon). Perinuclear staining patterns were revealed by TOTO-3 (1:2000) or TOPRO-3 (1 μM, Molecular Probes) as previously described (Matamales et al. 2009).

Neurochemical Analysis

Dopamine and its metabolites in micro-dissected and flush frozen striata and lateral ventral midbrains were measured as described in Jackson-Lewis et al. (1995). Tonic levels of extracellular striatal Acetylcholine was determined by HPLC as described in Buchholzer and Klein (2002), with some modifications. A post-column IMER (BAS MF-8903) was used to convert Ach to hydrogen peroxide. The hydrogen peroxide was detected in a wired enzyme electrode (+100 mV vs Ag/AgCl) (Ne) (Huang et al., 1995). The quantification was done using BAS ChromGraph software. Stereotaxic placement of dialysis probe and validation of placement is described herein.

Optical Fractionator Method

The total number of dopaminergic neurons in the SNpc and VTA and ACh-neuron and FS neurons in the striatum were estimated by sterological cell counting method described by Liberatore et al. (1999) using a computer-assisted image analysis system consisting of a Zeiss Axioplan-2 photomicroscope equipped with a MC-XYZ-LS (Applied Scientific Instrumentation, Inc., Eugene, Oreg.) computer-controlled motorized stage, a DAGE-DC330/ATI (Michigan, Ind.) video camera, and NeuroZoom morphometry software (Scripps Research Institute, La Jolla, Calif.). Th⁺- and Niss1-stained neurons were counted in the SNpc throughout the entire extent of the SNpc (12 sections with a 4-section interval). To avoid double counting of neurons with unusual shapes, Th⁺- and Niss1-stained cells were counted only when their nuclei were optimally visualized, which occurred only in one focal plane. In addition, neurons were differentiated from non-neuronal cells, including glia, on the Niss1 stain by the exclusion of cells that did not have a clearly defined nucleus, cytoplasm, and a prominent nucleolus; although some small neurons can be excluded, these criteria reliably exclude all non neuronal cells. The total numbers of Th⁺- and Niss1-stained neurons in the SNpc were calculated by using the formula described by West (West, 1993).

Sections for volume analysis of the striatum and for striatal neuron counting were also counterstained using cresyl violet. For the striatum (12 sections with a 5-section interval) the program calculated the area of the outlined portion and the volume was calculated using the Cavalieri method (Gundersen and Jensen, 1987). ChAT⁺ and Parv⁺ neuron with defined nucleus or NeuN-cells were counted in frames distributed using a sampling grid of 400×400 μm. Counting frame sizes were 50 μm×50 μm×6 μm. Counting frames contained 6-7 cells, and Gundersen coefficients of error were always less than 0.1.

Stereotaxic Injection of the Neurotoxins 6-OHDA and AF64a and Striatal Microdialysis

6-OHDA (Sigma, St. Louis, Mo., USA) was dissolved at a concentration of 3 μg/0.5 μl saline in 0.1% ascorbic acid and injected using a Hamilton syringe into the right median forebrain bundle (mFB; coordinates: 1.2 mm posterior to bregma, 1.1 mm lateral to midline, and 4.5 mm ventral from dura; Paxinos, G. and Franklin, K. B. (2001). AF64a injections (0-5 mM, 0.5 μl) were made into right the striatum (coordinates: 0.5 mm anterior to bregma, 2.4 lateral to midline, 2.5 mm ventral from dura) or right PPTg (coordinates: 4.5 mm posterior to bregma, 1.2 lateral to midline, 2.7 mm ventral from dura) using a Hamilton syringe attached to a syringe pump (World Precision Instruments). The needle was left in place for 5 min after drug injection and then slowly removed. For the microdialysis experiment a guide cannula (26-gauge, 7-mm long) with stylets (PlasticsOne) was implanted and aimed at the striatum (same coordinates as above). Microdialysis took place 3 days after implantation of the guide cannula and was performed as described in Buchholzer and Klein (2002) with slight modifications. Microdialysis probes (Membrane molecular weigh cut-off 13 KD, 2 mm long membrane) were inserted 12 hours before the experiment and artificial cerebrospinal fluid (aCSF) (147 mM NaCl, 4 mM KCl, 1.2 mM MgCl2, 1.2 mM CaCl2 and 0.3 uM neostigmine) was perfused at 0.1 uL/min overnight. 2 hours prior to the collection of dialysate the flow rate was increased to 1.0 uL/min. Dialysate samples were collected every 20 min (20 μl per sample at 1 μl/min flow rate) over three hours. To identify needle/probe lesion locations, sections containing tissue displacement around the injection/implantation site was set-aside during slicing, stained with hematoxylin and mounted on slides.

Injection sites and probe placement was documented on sections by bright field microscopy and marked on a coronal schematics of the striatum or PPTg. Only experimental data from animals with correctly located needles/probes were used for analysis.

Analysis of Locomotion in the “Open Field”

The open field arena was a polycarbonate cage 30×60×25 cm placed in a sound proof chamber. Locomotion of each mouse was recorded and tracked automatically for 10 minutes after habituation. Motor activity was measured as total distance traveled and turning bias was calculated as the net turning angle divided by distance (degrees/cm). Turning bias was calculated by subtracting the sum of ipsi- to contra-lateral movements and is independent of displacement (Spink et al., 2001). Speed profiles of bouts of spontaneous locomotion were derived by plotting speed over time (Mohajeri et al., 2004). As a measure of the complexity of locomotion the alternations between acceleration and deceleration per bout of locomotion were counted (Kafkafi et al, 2003a; 2003b; Benjamini et. al., 2010).

### REFERENCES

1. Airaksinen M S, Saarma M (2002) The GDNF family: signalling, biological functions and therapeutic value. Nat Rev Neurosci. 3: 383-394
2. Akazawa C, Tsuzuki H, Nakamura Y, Sasaki Y, Ohsaki K, Nakamura S, Arakawa Y, Kohsaka S (2004) The upregulated expression of sonic hedgehog in motor neurons after rat facial nerve axotomy. J. Neurosci. 24:7923-7930.
3. Albin R L, Young A B, Penney J B (1989) The functional anatomy of basal ganglia disorders. Trends Neurosci. 12:366-375.
4. Benjamini Y, Fonio E, Galili T, Havkin G Z, Golani I. (2011) Quantification of Behavior Sackler Colloquium: Quantifying the buildup in extent and complexity of free exploration in mice. Proc Natl Acad Sci USA. 2011 May 13. [ Epub ahead of print]
5. Bezard E, Baufreton J, Owens G, Crossman A R, Dudek H, Taupignon A, Brotchie J M (2003) Sonic hedgehog is a neuromodulator in the adult subthalamic nucleus. FASEB J. 17:2337-2338.
6. Boger H A, Middaugh L D, Huang P, Zaman V, Smith A C, Hoffer B J, Tomac A C, Granholm A C (2006) A partial GDNF depletion leads to earlier age-related deterioration of motor function and tyrosine hydroxylase expression in the substantia nigra. Exp Neurol. 202:336-347.
7. Bohnen N I, Albin R L (2010) The cholinergic system and Parkinson disease. Behav Brain Res. 2010 January 7. [ Epub ahead of print]
8. Bolam J P, rapporteur, Bergman H, Graybiel, A. M., Kimura, M., Plenz, D., Seung, H. S., Surmeier, D. J., and Wickens, J. R. (2006). Group report: microcircuits, molecules, and motivated behavior. Microcircuits in the striatum. In: Microcircuits. The interface between neurons and global brain function. S Grillner and A. M. Graybiel eds. (The MIT press). Pp 165-189
9. Bonis P, Cuaomo D., Martell G., Madeo G., Schirinzi T., Puglisi F., Ponterio G., Pisani A., (2011) Centrality of striatal cholinergic transmission in basal ganglia function. Frontiers in Neuroanatomy 5: 1-9
10. Braak H., Del Tredici K., Rub U., de Vos R. A., Jansen Steur E. N., Braak e (2003) Staging of brain pathology related to sporadic Parkinson's disease. Neurobio Agin., 24:197-211
11. Briscoe J, Novitch B G (2008) Regulatory pathways linking progenitor patterning, cell fates and neurogenesis in the ventral neural tube. Philos Trans R Soc Lond B Biol Sci. 363:57-70.
12. Cayuso J, Ulloa F, Cox B, Briscoe J, Marti E. (2006) The Sonic hedgehog pathway independently controls the patterning, proliferation and survival of neuroepithelial cells by regulating Gli activity. Development. 133:517-528.
13. Chu T, Chiu M, Zhang E, Kunes S (2006) A C-terminal motif targets Hedgehog to axons, coordinating assembly of the Drosophila eye and brain. Dev Cell. 10:635-646.
14. Dahlstroem A, Fuxe K (1964) Evidence for the existence of monoamine-containing neurons in the central nervous system. I. demonstration of monoamines in the cell bodies of brain stem neurons. Acta Physiol Scand Suppl. 232:1-55.
15. Dass B, Iravani M M, Huang C, Barsoum J, Engber T M, Galdes A, Jenner P (2005) Sonic hedgehog delivered by an adeno-associated virus protects dopaminergic neurones against 6-OHDA toxicity in the rat. J Neural Transm. 112:763-778.
16. Dass B, Iravani M M, Jackson M J, Engber T M, Galdes A, Jenner P. (2002) Behavioural and immunohistochemical changes following supranigral administration of sonic hedgehog in 1-methyl-4-phenyl-1,2,3,6-tetrahydropyridine-treated common marmosets. Neuroscience 114: 99-109.
17. Ding J, Guzman J N, Tkatch T, Chen S, Goldberg J A, Ebert P J, Levitt P, Wilson C J, Hamm H E, Surmeier D J (2006) RGS4-dependent attenuation of M4 autoreceptor function in striatal cholinergic interneurons following dopamine depletion. Nat. Neurosci. 9:832-842.
18. Ding J B, Guzman J N, Peterson J D, Goldberg J A, Surmeier D J (2010) Thalamic gating of corticostriatal signaling by cholinergic interneurons. Neuron 67: 294-307.
19. Dunbar J S, Hitchcock K, Latimer M, Rugg E L, Ward N, Winn P (1992) Excitotoxic lesions of the pedunculopontine tegmental nucleus of the rat. II. Examination of eating and drinking, rotation, and reaching and grasping following unilateral ibotenate or quinolinate lesions. Brain Res. 589:194-206. Exp Neurol 57:369-384, 1998
20. Futami T, Takakusaki K, Kitai S T (1995) Glutamatergic and cholinergic inputs from the pedunculopontine tegmental nucleus to dopamine neurons in the substantia nigra pars compacta. Neurosci Res. 21:331-342.
21. Gaykema R. P. and Zaborsky L., (1996) Direct catecholaminergic-cholinergic interactions in the basal farebrain. II. Substantia nigra-ventral tegmental area projections to cholinergic neurons. J. Comp. Neurol. 374: 555-577.
22. Gill S S, Patel N K, Hotton G R, O'Sullivan K, McCarter R, Bunnage M, Brooks D J, Svendsen C N, Heywood P (2003) Direct brain infusion of glial cell line-derived neurotrophic factor in Parkinson disease. Nat. Med. 9:589-595.
23. Goodrich L V, Jung D, Higgins K M, Scott M P (1999) Overexpression of ptc1 inhibits induction of Shh target genes and prevents normal patterning in the neural tube. Dev Biol. 211: 323-334.
24. Grillner S, Hellgren J, Ménard A, Saitoh K, Wikström M A (2005) Mechanisms for selection of basic motor programs—roles for the striatum and pallidum. Trends Neurosci. 28:364-370.
25. Hammond R, Blaess S, Abeliovich A. (2009) Sonic hedgehog is a chemoattractant for midbrain dopaminergic axons. PLoS One. 4(9):e7007.
26. Hampton T G, Amende I (2010) Treadmill gait analysis characterizes gait alterations in Parkinson's disease and amyotrophic lateral sclerosis mouse models. J Mot Behav. 42:1-4.
27. Handrigan G R, Richman J M (2009) Autocrine and paracrine Shh signaling are necessary for tooth morphogenesis, but not tooth replacement in snakes and lizards (Squamata). Dev Biol. 337:171-186.
28. Hausdorff J M, Cudkowicz M E, Firtion R, Wei J Y, Goldberger A L (1998) Gait variability and basal ganglia disorders: stride-to-stride variations of gait cycle timing in Parkinson's disease and Huntington's disease. Mov Disord. 13:428-437.
29. Hooper J E, Scott M P (2005) Communicating with Hedgehogs. Nat. Rev Mol Cell Biol 6:306-317.
30. Huang J., Zhou H., Mahavadi S., Sriwal W., Murthy K S. (2007) Inhibition of Galphaq-dependent PLC-beta 1 activity by PKG and PKA is mediated by phosphorylation of RGS4 and GRK2. Am Journal of Physiology and Cell Physiology 292: C200-208.
31. Jin X, Costa R M (2010) Start/stop signals emerge in nigrostriatal circuits during sequence learning. Nature. 466: 457-462.
32. Joksimovic M, Anderegg A, Roy A, Campochiaro L, Yun B, Kittappa R, McKay R, Awatramani R. (2009) Spatiotemporally separable Shh domains in the midbrain define distinct dopaminergic progenitor pools. Proc Natl Acad Sci USA. 106:19185-1990.
33. Kataoka Y, Kalanithi P S, Grantz H, Schwartz M L, Saper C, Leckman J F, Vaccarino F M (2010) Decreased number of parvalbumin and cholinergic interneurons in the striatum of individuals with Tourette syndrome. J Comp Neurol 518:277-291.
34. Kramer E R, Aron L, Ramakers G M, Seitz S, Zhuang X, Beyer K, Smidt M P, Klein R. (2007) Absence of Ret signaling in mice causes progressive and late degeneration of the nigrostriatal system. PLoS Biol. 5:e39.
35. Lang A E, Gill S, Patel N K, Lozano A, Nutt J G, Penn R, Brooks D J, Hotton G, Moro E, Heywood P, Brodsky M A, Burchiel K, Kelly P, Dalvi A, Scott B, Stacy M, Turner D, Wooten V G, Elias W J, Laws E R, Dhawan V, Stoessl A J, Matcham J, Coffey R J, Traub M (2006) Randomized controlled trial of intraputamenal glial cell line-derived neurotrophic factor infusion in Parkinson disease. Ann Neurol. 59:459-466.
36. Lester D B, Rogers T D, Blaha C D (2010) Acetylcholine-dopamine interactions in the pathophysiology and treatment of CNS disorders. CNS Neurosci Ther. 16:137-162.
37. Lin L F, Doherty D H, Lile J D, Bektesh S, Collins F (1993) GDNF: a glial cell line-derived neurotrophic factor for midbrain dopaminergic neurons. Science. 260:1130-1132.
38. Loulier K, Ruat M, Traiffort E (2005) Analysis of hedgehog interacting protein in the brain and its expression in nitric oxide synthase-positive cells. Neuroreport. 16:1959-1962.
39. Maffei A, Fontanini A (2009) Network homeostasis: a matter of coordination. Curr Opin Neurobiol. 19:168-173.
40. Manfredsson F P, Mandel R J (2010) Development of gene therapy for neurological disorders. Discov Med. 9:204-211.
41. Mao L, Xia Y P, Zhou Y N, Dai R L, Yang X, Duan S J, Qiao X, Mei Y W, Hu B, Cui H (2009) A critical role of Sonic Hedgehog signaling in maintaining the tumorigenicity of neuroblastoma cells. Cancer Sci. 100:1848-1855.
42. Matamales M, Bertran-Gonzalez J, Salomon L, Degos B, Deniau J M, Valjent E, Hervé D, Girault J A (2009) Striatal medium-sized spiny neurons: identification by nuclear staining and study of neuronal subpopulations in BAC transgenic mice PLoS One. 2009; 4:e4770.
43. Mehlen P, Mille F, Thibert C (2005) Morphogens and cell survival during development. J. Neurobiol. 64:357-366.
44. Moore M W, Klein R D, Fariñas I, Sauer H, Armanini M, Phillips H, Reichardt L F, Ryan A M, Carver-Moore K, Rosenthal A (1996) Renal and neuronal abnormalities in mice lacking GDNF. Nature 382:76-79.
45. Nastuk M A, Graybiel A M (1985) Patterns of muscarinic cholinergic binding in the striatum and their relation to dopamine islands and striosomes. J Comp Neurol. 237:176-194.
46. Obeso J. A., Rodriguez-Oroz M. C., Goetz C. G. Marin, C., Kordower J. H., Rodriguez M., Hirsch E. C., Farrere M., Schapira H. V., Halliday., G. (2010) Missing pieces in the Parkinson's disease puzzle. Nature Medicine 16: 653-661.
47. Ogden S K, Fel D. L. Shilling N. S., Ahmed Y. F., Hwa J., Robbins, D. J. (2008) G protein Galphai functions immediately downstream of Smoothened in hedgehog signaling. Nature 456: 967-970.
48. Oorschot D E (2010) Cell types in the different nuclei of the basal ganglia. In Handbook of Basal Ganglia Structure and Function, H. Steiner and K. Y. Tseng, eds. (Academic Press & Elsevier, CA), pp 63-68
49. Pascual A, Hidalgo-Figueroa M, Piruat J I, Pintado C O, Gomez-Diaz R, López-Barneo J. (2008) Absolute requirement of GDNF for adult catecholaminergic neuron survival. Nat. Neurosci. 11:755-761.
50. Pascual O, Traiffort E, Baker D P, Galdes A, Ruat M, Champagnat J (2005) Sonic hedgehog signalling in neurons of adult ventrolateral nucleus tractus solitarius. Eur J. Neurosci. 22:389-396.
51. Pothos E N, Davila V, Sulzer D (1998) Presynaptic recording of quanta from midbrain dopamine neurons and modulation of the quantal size. J. Neurosci. 18:4106-4118.
52. Reilly J O, Karavanova I D, Williams K P, Mahanthappa N K, Allendoerfer K L. (2002) Cooperative effects of Sonic Hedgehog and NGF on basal forebrain cholinergic neurons. Mol Cell Neurosci. 19:88-96.
53. Reiner A (2010) The conservative evolution of the basal ganglia. In Handbook of Basal Ganglia Structure and Function, H. Steiner and K. Y. Tseng, eds. (Academic Press & Elsevier, CA), pp 29-62.
54. Rinne, J. O., Ma, S. Y., Lee, M. S., Collan, Y. and Roytta, M. (2008). Loss of cholinergic neurons in the pendunculopontine nucleus in Parkinson's disease is related to disability of the patients. Parkinsonism Rel. disorders 14: 553-557.
55. Ruberg M, Javoy-Agid F, Hirsch E, Scatton B, LHeureux R, Hauw J J, Duyckaerts C, Gray F, Morel-Maroger A, Rascol A, et al. (1985) Dopaminergic and cholinergic lesions in progressive supranuclear palsy. Ann Neurol. 18: 523-529.
56. Sandberg K, Hanin I, Fisher A and JT Coyle (1984) Selective cholinergic neurotoxin: AF64A's effects in rat striatum. Brain Res. 293: 49-55.
57. Silberberg G, Grillner S, LeBeau F E, Maex R, Markram H (2005) Synaptic pathways in neural microcircuits, Trends Neurosci. 28:541-551
58. Slevin J T, Gerhardt G A, Smith C D, Gash D M, Kryscio R, Young B. (2005) Improvement of bilateral motor functions in patients with Parkinson disease through the unilateral intraputaminal infusion of glial cell line-derived neurotrophic factor. J. Neurosurg. 102: 216-222.
59. Tepper J M (2010) Gabaergic interneurons of the striatum. In Handbook of Basal Ganglia Structure and Function, H. Steiner and K. Y. Tseng, eds. (Academic Press & Elsevier, CA), pp 151-167
60. Threlfell S, Clements M A, Khodai T, Pienaar I S, Exley R, Wess J, Cragg S J. (2010) Striatal muscarinic receptors promote activity dependence of dopamine transmission via distinct receptor subtypes on cholinergic interneurons in ventral versus dorsal striatum. J. Neurosci. 30:3398-3408.
61. Traiffort E, Angot E, Ruat M (2010) Sonic Hedgehog signaling in the mammalian brain. J. Neurochem. 113:576-590.
62. Tsuboi K, Shults C W (2002) Intrastriatal injection of sonic hedgehog reduces behavioral impairment in a rat model of Parkinson's disease. Exp Neurol. 173: 95-104.
63. Ulloa F, Briscoe J (2007) Morphogens and the control of cell proliferation and patterning in the spinal cord. Cell Cycle. 6:2640-2649.
64. de Lau and Breteler, 2006 L. M. de Lau and M. M. Breteler, Epidemiology of Parkinson's disease, Lancet Neurology 5 (2006), pp. 525-535.
65. Vonsattel J P, DiFiglia M: (1998) Huntington disease. J Neuropathol; 57(5):369-84.
66. Wallace V A, Raff M C (1999) A role for Sonic hedgehog in axon-to-astrocyte signalling in the rodent optic nerve. Development 126:2901-2909.
67. Wang C Y, Yang F, He X, Chow A, Du J, Russell J T, Lu B (2001) Ca(2+) binding protein frequenin mediates GDNF-induced potentiation of Ca(2+) channels and transmitter release. Neuron. 32:99-112.
68. Yin H H, Mulcare S P, Hilario M R, Clouse E, Holloway T, Davis M I, Hansson A C, Lovinger D M, Costa R M. (2009) Dynamic reorganization of striatal circuits during the acquisition and consolidation of a skill. Nat. Neurosci. 12: 333-341.
69. Yurek D M, Fletcher-Turner A. (2000) Lesion-induced increase of BDNF is greater in the striatum of young versus old rat brain. Exp Neurol. 161:392-396.
70. Zhuang X, Masson J, Gingrich J A, Rayport S, Hen R (2004) Targeted gene expression in dopamine and serotonin neurons of the mouse brain. J Neurosci Methods. 143(1):27-32.
71. Wooten, G. F. Parkinsonism. In: Neurobiology of Disease, edited by A. L. Pearlman and R. C. Collins. New York: Oxford Univ. Press, 1990, p. 454-468.
72. Buchholzer M L, Klein J. (2002) NMDA-induced acetylcholine release in mouse striatum: role of NO synthase isoforms. J. Neurochem. 82(6):1558-60
73. Chiang C, Litingtung Y, Lee E, Young K E, Corden J L, Westphal H, Beachy P A (1996) Cyclopia and defective axial patterning in mice lacking Sonic hedgehog gene function. Nature. 383:407-413.
74. Deumens R., blokland A., Prikaers J., (2002) Modelling Parkinson's disease in rats: an evaluation of 6-OHDA lesions of the nigrostriatal pathway. Experimental neurology. 175: 303-317.
75. Dietrich P, Dragatsis I, Xuan S, Zeitlin S, Efstratiadis A (2000) Conditional mutagenesis in mice with heat shock promoter-driven cre transgenes. Mamm Genome. 11:196-205.
76. Drai D, Benjamini Y, Golani I. (2000) Statistical discrimination of natural modes of motion in rat exploratory behavior. J Neurosci Methods. 96:119-131.
77. Drai D, Golani I. (2001) SEE: a tool for the visualization and analysis of rodent exploratory behavior. Neurosci Biobehav Rev. 25:409-426.
78. Fredriksson A, Plaznik A, Sundström E, Jonsson G, Archer T (1990) MPTP-induced hypoactivity in mice: reversal by L-dopa. Pharmacol Toxicol. 67:295-301.
79. Goldschmidt P L, Savary L, Simon P (1984) Comparison of the stimulatory effects of eight antiparkinsonian drugs. Prog Neuropsychopharmacol Biol Psychiatry. 8:257-261
80. Gundersen H J G, Jensen E B (1987) The efficiency of systematic sampling in stereology and its prediction. J. Microscop. 147:229-263
81. Huang T., Yang L, Gitzen J, Kissinger, PT, Vreeke M, Heller A, (1995) Detection of basal acetylcholine in rat brain microdialysate. Journal of Chromatography B, 670: 323-327
82. Iancu R, Mohapel P, Brundin P, Gesine P (2005) Behavioral characterization of a unilateral 6-OHDA-lesion model of Parkinson's disease in mice. Behav Brain Res. 162:1-10
83. Jackson-Lewis V, Jakowec M, Burke R E, Przedborski S. (1995 Time course and morphology of dopaminergic neuronal death caused by the neurotoxin 1-methyl-4-phenyl-1,2,3,6-tetrahydropyridine. Neurodegeneration. 4(3):257-69
84. Kafkafi N, Lipkind D, Benjamini Y, Mayo C L, Elmer G I, Golani I. (2003a) SEE locomotor behavior test discriminates C57BL/6J and DBA/2J mouse inbred strains across laboratories and protocol conditions. Behav Neurosci. 117:464-477.
85. Kafkafi N, Pagis M, Lipkind D, Mayo C L, Bemjamini Y, Golani I, Elmer G I. (2003b) Darting behavior: a quantitative movement pattern designed for discrimination and replicability in mouse locomotor behavior. Behav Brain Res. 142:193-205.
86. Lang M, Hadzhiev Y, Siegel N, Amemiya C T, Parada C, Strähle U, Becker M B, Müller F, Meyer A. (2010) Conservation of shh cis-regulatory architecture of the coelacanth is consistent with its ancestral phylogenetic position. Evodevo. November 3; 1(1):11.
87. Lindholm D, Wootz H, Korhonen L (2006) ER stress and neurodegenerative diseases. Cell Death Differ. 13:385-392.
88. Mohajeri M H, Madani R. Saini K, Lipp H P, Nitsch R M and Wolfer D P (2004) The impact of genetic background on neurodegeneration and behavior in seizured mice Genes, Brain and Behavior (2004) 3: 228-239.
89. Paxinos, G. and Franklin, K. B. (2001). The Mouse Brain in Stereotaxic Coordinates. (London: Academic Press).
90. Saxena S, Cabuy E, Caroni P (2009) A role for motoneuron subtype-selective ER stress in disease manifestations of FALS mice. Nat. Neurosci. 12:627-636.
91. Schaeren-Wiemers N, Gerfin-Moser A (1993) A single protocol to detect transcripts of various types and expression levels in neural tissue and cultured cells: in-situ hybridization using digoxigenin-labelled cRNA probes. Histochemistry 100, 431-440.
92. Spink A J, Tegelenbosch R A, Buma M O, Noldus L P (2001) The EthoVision video tracking system—a tool for behavioral phenotyping of transgenic mice. Physiol Behav. 73:731-744.
93. Sulzer D., Sonders M. S., Poulsen N. W., Galli A. (2007) Mechanisms of neurotransmitter release by amphetamines: a review. Progress in Neurobiology 75: 406-433.
94. Tallini Y N, Shui B, Greene K S, Deng K Y, Doran R, Fisher P J, Zipfel W, Kotlikoff M I (2006) BAC transgenic mice express enhanced green fluorescent protein in central and peripheral cholinergic neurons. Physiol Genomics 27:391-397.
95. Zhao L, Ackerman S L (2006) Endoplasmic reticulum stress in health and disease. Curr Opin Cell Biol. 18:444-452.

What is claimed is:

1. A method for neuroprotection of neurons in a subject afflicted with or at risk of developing a neurodegenerative disorder, the method comprising administering to a subject an effective amount of a Shh antagonist that increases glial cell-derived neurotrophic factor (GDNF), thereby protecting the neurons.

2. A method of decreasing axonal degeneration in a subject afflicted with or at risk of developing a neurodegenerative disorder, the method comprising administering to a subject an effective amount of a Shh antagonist that increases glial cell-derived neurotrophic factor (GDNF), thereby decreasing axonal degeneration.

3. A method for treating a subject afflicted with or at risk of developing a neurodegenerative disorder, the method comprising administering to a subject an effective amount of a Shh antagonist that increases glial cell-derived neurotrophic factor (GDNF), thereby treating the subject.

4. A method for treating a subject afflicted with or at risk of developing an addiction, the method comprising administering to a subject an effective amount of a Shh antagonist that increases glial cell-derived neurotrophic factor (GDNF), thereby treating the subject.

5. A method of decreasing cholinergic tone in a subject afflicted with or at risk of developing a neurodegenerative disorder, the method comprising:
a) administering to the subject an effective amount of a Shh antagonist; and
b) measuring the concentration of acetylcholine in the extracellular fluid of the brain.

6. A method of decreasing cholinergic tone in a subject afflicted with a hypercholinergic disease, the method comprising:
a) administering to the subject an effective amount of a Shh antagonist; and
b) measuring the concentration of acetylcholine in the extracellular fluid of the brain.

7. A method for treating a subject afflicted with or at risk of developing a dopaminergic-related psychiatric condition, the method comprising administering to a subject an effective amount of a Shh agonist that decreases glial cell-derived neurotrophic factor (GDNF), thereby treating the subject.

8. A method of increasing cholinergic tone in a subject afflicted with or at risk of developing a dopaminergic-related psychiatric condition, the method comprising:
a) administering to the subject an effective amount of a Shh agonist; and
b) measuring the concentration of acetylcholine in the extracellular fluid of the brain.

9. The method of claim 1, 2, 3, 4, or 7, wherein the GDNF is endogenous GDNF.

10. The method of claim 1, 2, 3, or 5, wherein the neurodegenerative disorder comprises Parkinson's Disease (PD), Amyotrophic Lateral Sclerosis (ALS), Alzheimer's Disease (AD), or Supra Nuclear Palsy, spinocereballar ataxias, multiple system atrophy, or corticobasal degeneration.

11. The method of claim 6, wherein the hypercholinergic disease comprises Parkinson's Disease (PD), Amyotrophic Lateral Sclerosis (ALS), Alzheimer's Disease (AD), or Supra Nuclear Palsy, spinocereballar ataxias, multiple system atrophy, or corticobasal degeneration.

12. The method of claim 1, 2, 3, 5, or 6, wherein the antagonist is cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination thereof.

13. The method of claim 7 or 8, wherein the agonist is purmorphamine or SAG.

14. The method of claim 4, wherein the addiction is an addiction to cocaine, alcohol, heroine, methadone, amphetamine, ketamine, or a combination thereof.

15. The method of claim 7 or 8, wherein the condition comprises schizophrenia, bipolar affective disorder, or attention deficit hyperactivity disorder (ADHD).

16. A method for increasing the production of cholinergic neurons by subventricular zone (SVZ) neurogenesis in a subject in need thereof, the method comprising administering to the subject an effective amount of a cholinotoxin to increase Shh expression in adult dopamine neurons, thereby increasing the production of cholinergic neurons.

17. A method for increasing the production of dopamine neurons in the olfactory bulb in a subject in need thereof, the method comprising administering to the subject an effective amount of a Shh antagonist that decreases Shh expression in adult dopamine neurons, thereby increasing the production of dopamine neurons in the olfactory bulb.

18. A method for treating a neurodegenerative disorder in a subject in need thereof, the method comprising administering to the subject an effective amount of a cholinotoxin to increase Shh expression in adult dopamine neurons, wherein increased Shh expression increased the production of cholinergic neurons, thereby treating the neurodegenerative disorder.

19. A method for treating a neurodegenerative disorder in a subject in need thereof, the method comprising administering to the subject an effective amount of a Shh antagonist that decreases Shh expression in adult dopamine neurons, wherein increased Shh expression increased the production of dopamine neurons in the olfactory bulb, thereby treating the neurodegenerative disorder.

20. The method of claim 16 or 18, wherein the cholinotoxin is AF64A.

21. The method of claim 16, 17, 18, or 19, wherein the dopamine neurons are mesencephalic dopamine neurons.

22. The method of claim 18, wherein the neurodegenerative disorder is Alzheimer's Disease or Supra Nuclear Palsy.

23. The method of claim 19, wherein the neurodegenerative disorder is Parkinson's Disease or Amyotrophic Lateral Sclerosis.

24. A method for regenerating neurons in the subventricular zone (SVZ) of a subject afflicted with a neurodegenerative disorder, the method comprising administering to the subject an effective amount of a compound that modulates Shh expression in adult dopamine neurons, thereby regenerating neurons.

25. The method of claim 24, wherein Shh expression is increased.

26. The method of claim 24, wherein the compound is a cholinotoxin.

27. The method of claim 26, wherein the cholinotoxin is AF64A.

28. The method of claim 25, wherein the increase in Shh expression induces the production of cholinergic neurons.

29. The method of claim 24, wherein the neurodegenerative disorder is Alzheimer's Disease or Supra Nuclear Palsy.

30. The method of claim 24, wherein Shh expression is decreased.

31. The method of claim 30, wherein the decrease in Shh expression induces the production of dopamine neurons in the olfactory bulb.

32. The method of claim 24, wherein the compound is a Shh antagonist.

33. The method of claim 24, wherein the neurodegenerative disorder is Parkinson's Disease or Amyotrophic Lateral Sclerosis.

34. The method of claim 17, 19, or 32, wherein the antagonist is cyclopamine, KAAD-cyclopamine, KADAR-cyclopamaine, jervine, SANT 1, SANT 2, SANT 3, SANT 4, Cur-61414, IPI-926, GDC-0449, robotnikinin, or a combination thereof.

35. A method for screening compounds for the treatment of a neurological disease of the basal ganglia, the method comprising:
(a) administering a compound into a non-human animal with genetic ablation of Shh from mesencephalic DA neurons;
(b) observe locomotion of the animal; and
(c) determine if there is a locomotion deficit as compared to a non-human animal without genetic ablation of Shh from mesencephalic DA neurons.

36. A method for testing efficacy of a compound used for the treatment of a neurological disease of the basal ganglia, the method comprising:
(a) administering a compound into a non-human animal with genetic ablation of Shh from mesencephalic DA neurons;
(b) observe locomotion of the animal; and
(c) determine if there is a locomotion deficit as compared to a non-human animal without genetic ablation of Shh from mesencephalic DA neurons.

37. The method of claim 35 or 36, wherein the neurological disease of the basal ganglia is Parkinson's Disease, Huntington's Disease, a movement disorder, or a combination thereof.

38. The method of claim 35 or 36, wherein the non-human animal is a mouse or a rat.

39. The method of claim 35 or 36, wherein the locomotion deficit comprises reduction in gait length, an increases in gait variability, a reduction in break time, movement fluidity, bradykinesia, or a combination thereof.

40. The method of claim 37, wherein the movement disorder comprises dyskinesias, dystonias, myoclonus, chorea, tics, tremor, or a combination thereof.

41. The method of claim 5, 6, or 8, wherein measuring the concentration of acetylcholine in the extracellular fluid of the brain comprises liquid chromatography mass spectrometry of brain microdialysis samples.


SEQUENCE LISTING (3 sequences)

SEQ ID NO: 1 - DNA, 57 bp, Artificial Sequence
  Description of Artificial Sequence Synthetic oligonucleotide
ctaggcgcgcctctagaggatccataacttcgtataatgtatgctatacgaagtatc

SEQ ID NO: 2 - DNA, 26 bp, Artificial Sequence
  Description of Artificial Sequence Synthetic oligonucleotide
gtaagagcacattacccagagaactg

SEQ ID NO: 3 - DNA, 26 bp, Artificial Sequence
  Description of Artificial Sequence Synthetic oligonucleotide
cctgttgttactgcatcccttccatc